(19) United States
(12) Patent Application Publication
(10) Pub. No.: US 2025/0320290 A1
(43) Pub. Date: Oct. 16, 2025

(54) METHODS FOR TRIGGERING SAFETY KILLING MECHANISMS USING A CD47-SIRPalpha BLOCKADE AGENT

(71) Applicant: Sana Biotechnology, Inc., Seattle, WA (US)

(72) Inventor: Sonja Schrepfer, San Mateo, CA (US)

(21) Appl. No.: 18/030,880

(22) PCT Filed: Oct. 9, 2021
(86) PCT No.: PCT/US2021/054326

§ 371 Date: Apr. 7, 2023

Related U.S. Application Data
(60) Provisional application No. 63/090,001, filed on Oct. 9, 2020.
(60) Provisional application No. 63/135,518, filed on Jan. 8, 2021.

(51) Int. Cl. C07K 16/28; A61K 38/00; A61K 39/00; C07K 14/705

(52) U.S. Cl. CPC C07K 16/2803; C07K 14/70503; A61K 38/00; A61K 2039/505; C07K 2317/52; C07K 2319/30

(57) ABSTRACT

The present disclosure provides methods and compositions for administering to a subject in need thereof a CD47-SIRPα blockade agent, wherein the subject was previously administered a population of cells engineered to express an exogenous CD47 polypeptide.

### CROSS REFERENCE TO RELATED APPLICATIONS

This PCT application claims priority to U.S. Provisional Patent Application Ser. No. 63/090,001, filed Oct. 9, 2020, and U.S. Provisional Patent Application Ser. No. 63/135,518, filed Jan. 8, 2021, both of which are incorporated herein by reference in their entirety.

### SEQUENCE LISTING

This application contains a Sequence Listing, which was submitted in ASCII format via EFS-Web and is hereby incorporated by reference in its entirety. The ASCII copy, created on Oct. 9, 2021 is named 2021 Oct. 9 Sana 8007.WO00 sequence listing.txt and is 121 KB in size.

Regenerative medicine (cell therapy) involves the preparation and delivery of cells to a patient. These cells may be pluripotent stem cells (PSCs) which can be differentiated to any cell type, cells differentiated from these PSCs, or primary cells. These cells can be engineered to contain one or more exogenous nucleic acids encoding CD47, a transmembrane protein and known marker of “self” on host cells within an organism, and, optionally, one or more other proteins. When CD47 binds to signal regulatory protein alpha (SIRPα), a transmembrane receptor protein on circulating immune cells, to deliver an inhibitory “don't eat me” signal, the host cell expressing the CD47 evades rejection by the patient's immune system, e.g., through macrophage- and/or natural killer (NK) cell-mediated death. The immunosuppressive characteristics of such engineered cells can render them dangerous to a patient into whom the cells are transplanted, e.g., if unbridled growth occurs, creating a need for the development of safety mechanisms that can modulate, e.g., eliminate via the patient's innate immune system, the transplanted population of cells by acting on the CD47-SIRPα axis or interaction.

### BRIEF SUMMARY

The present disclosure provides methods and compositions for modulating a population of cells previously administered to or transplanted into a subject, comprising administering a CD47-SIRPα blockade agent to the subject, wherein the population of cells contains one or more exogenous nucleic acids encoding CD47 and/or expressing or overexpressing CD47. Such a CD47-SIRPα blockade agent comprises a small molecule, macromolecule, polypeptide, fusion protein, diabody, antibody, or a combination thereof that binds to CD47 or SIRPα, thus acting on, interfering with, blocking, and/or inhibiting a CD47-SIRPα axis or interaction. Modulating a population of cells that overexpress CD47 or otherwise express exogenous CD47 polypeptides comprises triggering innate killing mechanisms in a subject who has been administered such cells. Innate killing mechanisms may be triggered by administration of the CD47-SIRPα blockade agent and can include immune cell-mediated killing of the cells, such as NK-mediated killing, macrophage mediated killing, ADCC and/or CDC.

In one aspect, provided herein is a method comprising administering to a subject in need thereof a CD47-SIRPα blockade agent, wherein the subject was previously administered a population of cells engineered to express an exogenous CD47 polypeptide.

In another aspect, provided herein is a method comprising administering to a subject in need thereof a CD47-SIRPα blockade agent, wherein the subject was previously administered a population of T cells engineered to express an exogenous CD47 polypeptide.

In another aspect, provided herein is a method comprising administering to a subject in need thereof a CD47-SIRPα blockade agent, wherein the subject was previously administered a population of T cells (i) engineered to express an exogenous CD47 polypeptide and at least one chimeric antigen receptor (CAR) and (ii) having reduced expression of MHC class I HLA molecules, MHC class II HLA molecules, T cell receptor (TCR) alpha, and/or TCR beta.

In another aspect, provided herein is a method comprising administering to a subject in need thereof a CD47-SIRPα blockade agent, wherein the subject was previously administered a population of T cells having reduced expression of MHC class I HLA molecules, MHC class II HLA molecules, and TCR alpha and engineered to express an exogenous CD47 polypeptide and a CD19 chimeric antigen receptor (CAR).

In another aspect, provided herein is a method comprising administering to a subject in need thereof a CD47-SIRPα blockade agent, wherein the subject was previously administered a population of pancreatic islet cells engineered to express an exogenous CD47 polypeptide.

In another aspect, provided herein is a method comprising administering to a subject in need thereof a CD47-SIRPα blockade agent, wherein the subject was previously administered a population of pancreatic islet cells (i) engineered to express an exogenous CD47 polypeptide and (ii) having reduced expression of MHC class I HLA and/or MHC class II HLA molecules.

In another aspect, provided herein is a method comprising administering to a subject in need thereof a CD47-SIRPα blockade agent, wherein the subject was previously administered a population of pancreatic islet cells (i) engineered to express exogenous CD47, CD46, and CD59 polypeptides and (ii) having reduced expression of MHC class I HLA and/or MHC class II HLA molecules.

In another aspect, provided herein is a method of reducing a population of cells engineered to express an exogenous CD47 polypeptide in a subject comprising: (a) administering to the subject a first dose of a CD47-SIRPα blockade agent; (b) determining a first outcome of the first dose of the CD47-SIRPα blockade agent administered in (a); (c) optionally administering a second dose of the CD47-SIRPα blockade agent based on the first outcome in (b); and (d) optionally determining a second outcome of the second dose of the CD47-SIRPα blockade agent administered in (c).

In another aspect, provided herein is a method comprising: (a) quantifying a population of cells engineered to express an exogenous CD47 polypeptide in a subject; (b) determining a first dose of a CD47-SIRPα blockade agent that is effective in reducing the population of cells by at least 20%; and (c) administering the first dose of the CD47-SIRPα blockade agent to the subject.

In some embodiments of each or any of the above or below mentioned embodiments, the T cells are primary cells.

In some embodiments of each or any of the above or below mentioned embodiments, the T cells are allogeneic.

In some embodiments of each or any of the above or below mentioned embodiments, the T cells are differentiated from iPSCs.

In some embodiments of each or any of the above or below mentioned embodiments, the T cells are further engineered to express a chimeric antigen receptor (CAR).

In some embodiments of each or any of the above or below mentioned embodiments, the CAR is a CD19 CAR selected from the group consisting of tisagenlecleucel, lisocabtagene maraleucel, axicabtagene ciloleucel, and brexucabtagene autoleucel.

In some embodiments of each or any of the above or below mentioned embodiments, the CAR is a CD19 CAR comprising the amino acid sequence of SEQ ID NO: 117.

In some embodiments of each or any of the above or below mentioned embodiments, the CD19 CAR is encoded by the nucleic acid sequence of SEQ ID NO:116.

In some embodiments of each or any of the above or below mentioned embodiments, the T cells are engineered to express at least one additional factor selected from the group consisting of CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8, and a combination thereof.

In some embodiments of each or any of the above or below mentioned embodiments, the pancreatic islet cells are engineered to express at least one additional factor selected from the group consisting of CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8, and a combination thereof.

The method of any of claim 5, 6, or 7, wherein the pancreatic islet cells are engineered to have reduced expression of CD142.

In some embodiments of each or any of the above or below mentioned embodiments, the pancreatic islet cells are primary cells.

In some embodiments of each or any of the above or below mentioned embodiments, the pancreatic islet cells are differentiated from iPSCs.

In some embodiments of each or any of the above or below mentioned embodiments, the CAR and a gene encoding the exogenous CD47 polypeptide were introduced into the T cells in a bicistronic vector.

In some embodiments of each or any of the above or below mentioned embodiments, the bicistronic vector was introduced into the T cells via a lentivirus.

In some embodiments of each or any of the above or below mentioned embodiments, the CAR and the gene encoding the exogenous CD47 polypeptide are under the control of a single promoter.

In some embodiments of each or any of the above or below mentioned embodiments, the first outcome and second outcome are independently selected from the group consisting of: (i) a reduction in the number of cells by between about 10% and 100%, (ii) a reduction in an adverse event by between about 10% and 100%, and (iii) a combination of (i) and (ii).

In some embodiments of each or any of the above or below mentioned embodiments, the first dose and/or the second dose is administered: (i) at 0.05, 0.1, 0.3, 1, 3, or 10 mg/kg; (ii) once every 12 hours, once every 24 hours, once every 36 hours, or once every 48 hours; and/or (iii) for between 1 day and 3 weeks.

In some embodiments of each or any of the above or below mentioned embodiments, the first dose and the second dose are the same.

In some embodiments of each or any of the above or below mentioned embodiments, the cells are primary cells.

In some embodiments of each or any of the above or below mentioned embodiments, the primary cells are T cells or pancreatic islet cells.

In some embodiments of each or any of the above or below mentioned embodiments, the cells are differentiated from iPSCs.

In some embodiments of each or any of the above or below mentioned embodiments, the differentiated cells are selected from the group consisting of cardiac cells, neural cells, endothelial cells, T cells, pancreatic islet cells, retinal pigmented epithelium cells, hepatocytes, thyroid cells, skin cells, blood cells, primary cells, and epithelial cells.

In some embodiments of each or any of the above or below mentioned embodiments, the cells are engineered to express at least one additional factor selected from the group consisting of CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8, and a combination thereof.

In some embodiments of each or any of the above or below mentioned embodiments, the T cells are engineered to have reduced expression of TCRα and/or TCRβ.

In some embodiments of each or any of the above or below mentioned embodiments, the T cells are engineered to have reduced expression of cytotoxic T-lymphocyte-associated protein 4 (CTLA4) and/or programmed cell death (PD1).

In some embodiments of each or any of the above or below mentioned embodiments, a gene encoding the exogenous CD47 polypeptide was introduced into the cell via homology directed repair (HDR)-mediated insertion into a genomic locus of the cell.

In some embodiments of each or any of the above or below mentioned embodiments, the genomic locus is selected from the group consisting of a B2M locus, a CIITA locus, a TRAC locus, a TRBC locus, and a safe harbor locus.

In some embodiments of each or any of the above or below mentioned embodiments, the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus.

In some embodiments of each or any of the above or below mentioned embodiments, the CAR binds an antigen selected from the group consisting of CD19, CD20, CD22, CD38, CD123, CD138, BCMA, and a combination thereof.

In some embodiments of each or any of the above or below mentioned embodiments, the first outcome and/or second outcome is an adverse event.

In some embodiments of each or any of the above or below mentioned embodiments, the CD47-SIRPα blockade agent is administered at least one day after the subject was administered the cells.

In some embodiments of each or any of the above or below mentioned embodiments, the CD47-SIRPα blockade agent is administered at least one week after the subject was administered the cells.

In some embodiments of each or any of the above or below mentioned embodiments, the CD47-SIRPα blockade agent is administered at least one month after the subject was administered the cells.

In some embodiments of each or any of the above or below mentioned embodiments, the CD47-SIRPα blockade agent is administered after the subject experiences an adverse event related to the administered cells.

In some embodiments of each or any of the above or below mentioned embodiments, the adverse event is selected from the group consisting of hyperproliferation, transformation, tumor formation, cytokine release syndrome, graft-versus-host disease (GVHD), immune effector cell-associated neurotoxicity syndrome (ICANS), inflammation, infection, nausea, vomiting, bleeding, interstitial pneumonitis, respiratory disease, jaundice, weight loss, diarrhea, loss of appetite, cramps, abdominal pain, hepatic veno-occlusive disease (VOD), graft failure, organ damage, infertility, hormonal changes, abnormal growth formation, cataracts, and post-transplant lymphoproliferative disorder (PTLD).

In some embodiments of each or any of the above or below mentioned embodiments, the CD47-SIRPα blockade agent comprises a CD47-binding domain.

In some embodiments of each or any of the above or below mentioned embodiments, the CD47-binding domain comprises signal regulatory protein alpha (SIRPα) or a fragment thereof.

In some embodiments of each or any of the above or below mentioned embodiments, the CD47-SIRPα blockade agent comprises an immunoglobulin G (IgG) Fc domain.

In some embodiments of each or any of the above or below mentioned embodiments, the IgG Fc domain comprises an IgG1 Fc domain.

In some embodiments of each or any of the above or below mentioned embodiments, the IgG1 Fc domain comprises a fragment of a human antibody.

In some embodiments of each or any of the above or below mentioned embodiments, the CD47-SIRPα blockade agent is selected from the group consisting of TTI-621, TTI-622, and ALX148.

In some embodiments of each or any of the above or below mentioned embodiments, the IgG Fc domain comprises an IgG4 Fc domain.

In some embodiments of each or any of the above or below mentioned embodiments, the CD47-SIRPα blockade agent is an antibody.

In some embodiments of each or any of the above or below mentioned embodiments, the antibody is selected from the group consisting of MIAP410, B6H12, and Magrolimab.

In some embodiments of each or any of the above or below mentioned embodiments, the CD47-SIRPα blockade agent is administered at a dose effective to reduce the population of cells.

In some embodiments of each or any of the above or below mentioned embodiments, the population of cells is reduced by between about 10% and 100%.

In some embodiments of each or any of the above or below mentioned embodiments, the population of cells is eliminated.

In some embodiments of each or any of the above or below mentioned embodiments, the reduction of the population of cells occurs via an immune response.

In some embodiments of each or any of the above or below mentioned embodiments, the immune response is NK cell-mediated cell killing, macrophage-mediated cell killing, complement-dependent cytotoxicity (CDC), and/or antibody-dependent cellular cytotoxicity (ADCC) of the cells.

In some embodiments of each or any of the above or below mentioned embodiments, the CD47-SIRPα blockade agent is administered to the subject intravenously, subcutaneously, intraperitonially, intramuscularly, or intracranially.

In some embodiments of each or any of the above or below mentioned embodiments, the CD47-SIRPα blockade agent is administered to the subject at a time interval of between 1-20 days for a period of between 10 days and 6 months.

In some embodiments of each or any of the above or below mentioned embodiments, the CD47-SIRPα blockade agent is administered to the subject: (i) at a dose of 0.05, 0.1, 0.3, 1, 3, or 10 mg/kg; (ii) once every 12 hours, once every 24 hours, once every 36 hours, or once every 48 hours; and/or (iii) for between 1 day and 3 weeks.

In some embodiments of each or any of the above or below mentioned embodiments, the method further comprises administering IL-2 to the subject.

In some embodiments of each or any of the above or below mentioned embodiments, the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that binds SIRPα, an SIRPα containing fusion protein, and a combination thereof.

In some embodiments of each or any of the above or below mentioned embodiments, the antibody or fragment thereof that binds CD47 is selected from the group consisting of magrolimab (Hu5F9-G4), CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

In some embodiments of each or any of the above or below mentioned embodiments, the antibody or fragment thereof that binds CD47 is selected from the group consisting of a single-chain Fv fragment (scFv) against CD47, a Fab against CD47, a VHH nanobody against CD47, a DARPin against CD47, and variants thereof.

In some embodiments of each or any of the above or below mentioned embodiments, the antibody or fragment thereof that binds SIRPα is selected from the group consisting of ADU-1805, CC-95251, OSE-172 (BI 765063), KWAR23, and P362.

In some embodiments of each or any of the above or below mentioned embodiments, the antibody or fragment thereof that binds SIRPα is selected from the group consisting of a single-chain Fv fragment (scFv) against SIRPα, a Fab against SIRPα, a VHH nanobody against SIRPα, a DARPin against SIRPα, and variants thereof.

In some embodiments of each or any of the above or below mentioned embodiments, the SIRPα-containing fusion protein comprises a CD47 binding domain of SIRPα linked to an Fc domain.

In some embodiments of each or any of the above or below mentioned embodiments, the Fc domain comprises an Fc domain or portion thereof selected from the group consisting of IgG1, IgG2, IgG3, and IgG4.

In some embodiments of each or any of the above or below mentioned embodiments, the cells have reduced expression of MHC class I HLA and/or MHC class II HLA molecules.

In some embodiments of each or any of the above or below mentioned embodiments, MHC class I and/or MHC class II expression is knocked out.

In some embodiments of each or any of the above or below mentioned embodiments, the reduced expression of MHC class I HLA is mediated by reduced expression of B2M and reduced expression of MHC class II is mediated by reduced expression of CIITA.

In some embodiments of each or any of the above or below mentioned embodiments, B2M and/or CIITA expression is knocked out.

In some embodiments of each or any of the above or below mentioned embodiments, the exogenous CD47 polypeptide comprises the amino acid sequence of SEQ ID NO:2 or SEQ ID NO:4.

### BRIEF DESCRIPTION OF THE DRAWINGS

FIGS. 1A-1D depict killing by IL-2 stimulated NK cells of human HIP (B2M^{-/-}, CIITA^{-/-}, CD47⁺) cells (FIG. 1A), human dKO (B2M^{-/-}, CIITA^{-/-}) cells (FIG. 1B), human HIP cells treated with an anti-CD47 IgG1 isotype control antibody (FIG. 1C), or human HIP cells treated with an anti-CD47 antibody MIP410 (FIG. 1D).

FIGS. 2A-2D depict killing by macrophages of human HIP (B2M^{-/-}, CIITA^{-/-}, CD47⁺) cells (FIG. 2A), human dKO (B2M^{-/-}, CIITA^{-/-}) cells (FIG. 2B), human HIP cells treated with an anti-CD47 IgG1 isotype control antibody (FIG. 2C), or human HIP cells treated with an anti-CD47 antibody MIP410 (FIG. 2D).

FIGS. 3A and 3B depict bioluminescence measurements of human HIP (B2M^{-/-}, CIITA^{-/-}, CD47⁺) cells injected subcutaneously into NSG mice adoptively transferred human NK cells, following treatment by an IgG1 isotype control antibody (FIG. 3A) or anti-CD47 antibody MIP410 (FIG. 3B).

FIGS. 4A-4J depict real-time cell analysis data of NK cell and macrophage induced killing of human HIP (e.g., B2M^{-/-}, CIITA^{-/-}, TRAC^{-/-}) CAR-T cells expressing exogenous CD47 and CD19-specific CAR constructs (see, for example, “HIP CAR-T, single promoter CD47-CAR”) when exposed to an anti-CD47 antibody (see, for example, FIGS. 4A-4E). The data also shows the extent of NK cell and macrophage induced killing of control CAR-T cells expressing CAR and EGFRt constructs, control CAR-T cells substantially similar to a tisagenlecleucel biosimilar or surrogate, and control mock T cells (FIGS. 4E-4J).

FIGS. 5A and 5B show data of immune evasion in vivo following adoptive transfer of human NK cell and macrophages into immunodeficient NSG mice along with a mixture of human mock T cells and either human HLA-I and HLA-II double knockout CAR-T cells (FIG. 5A) or hypoimmunogenic human HLA-I, HLA-II and TCR triple knockout CAR-T cells (FIG. 5B).

FIG. 6 shows levels of T cell activation and donor-specific antibody binding detected in samples from humanized mice injected with either allogeneic CAR-T cells (such as, CAR-T cells expressing CAR-EGFRt constructs (“CAR (EGFRt)”) and a tisagenlecleucel biosimilar or surrogate (“CAR (tisagenlecleucel)”) or hypoimmunogenic human HLA-I, HLA-II and TCR triple knockout CAR-T cells (“HIP”).

FIGS. 7A and 7B show cell viability in vitro of human HIP iPSCs upon subcutaneous transplantation of HIP PSCs and adoptive transfer of NK cells and macrophages and administration of anti-CD47 magrolimab antibody at 100 μg/ml during the period of Day 0 to Day 10 (D0-D10).

FIGS. 8A and 8B show teratoma formation (HIP iPSC survival) in NSG mice upon subcutaneous transplantation of HIP iPSCs and adoptive transfer of NK cells and administration of IgG4 isotype control.

FIGS. 9A and 9B show cell viability in vivo of human HIP iPSCs upon subcutaneous transplantation of HIP iPSCs and adoptive transfer of NK cells and administration of anti-CD47 magrolimab antibody during the period of Day 0 to Day 10 (D0-D10).

FIGS. 10A and 10B show cell viability in vitro of human HIP iPSCs upon subcutaneous transplantation of HIP iPSCs and adoptive transfer of NK cells and macrophages and administration of anti-CD47 MIAP410 antibody at 100 μg/ml during the period of Day 0 to Day 10 (D0-D10).

FIGS. 11A and 11B show teratoma formation (HIP iPSC survival) in NSG mice upon subcutaneous transplantation of HIP iPSCs and adoptive transfer of NK cells and administration of IgG1 isotype control.

FIGS. 12A and 12B show cell viability in vivo of human HIP iPSCs upon subcutaneous transplantation of HIP iPSCs and adoptive transfer of NK cells and administration of anti-CD47 MIAP410 antibody during the period of Day 0 to Day 10 (D0-D10).

FIGS. 13A and 13B show cell viability in vivo of human HIP iPSCs upon subcutaneous transplantation of 15.5×10⁴ human iPSCs and adoptive transfer of 1×10⁶ human NK cells and administration of anti-CD47 MIAP410 antibody on Day 0, Day 1, and Day 3.

FIGS. 14A and 14B show cell viability in vivo of human HIP iPSCs upon subcutaneous transplantation of 16.5×10⁴ human iPSCs and adoptive transfer of 1×10⁶ human NK cells and intraperitoneal administration of anti-CD47 MIAP410 antibody on Day 0, Day 1, and Day 3.

FIGS. 15A and 15B show teratoma formation (HIP iPSC survival) in the brain of NSG mice upon intracranial transplantation of 5×10⁴ human iPSCs and adoptive transfer of 1×10⁶ NK cells and administration of IgG4 isotype control on Day 0, Day 1, and Day 3.

FIGS. 16A and 16B show cell viability in vivo of human HIP iPSCs in the brain upon intracranial transplantation of 5×10⁴ human iPSCs and adoptive transfer of 1×10⁶ human NK cells and intraperitoneal administration of anti-CD47 MIAP410 antibody on Day 0, Day 1, and Day 3.

FIGS. 17A and 17B show cell viability in vivo of human HIP iPSCs in the brain upon intracranial transplantation of 5×10⁴ human iPSCs and adoptive transfer of 1×10⁶ human NK cells and intraperitoneal administration of anti-CD47 MIAP410 antibody on Day 0, Day 1, and Day 3, with the blood-brain barrier broken by mannitol injections.

FIGS. 18A-18F show killing data in vitro of human HIP iPSCs, with respect to NK cell-ADCC NK cell-, and CDC-mediated killing (A), NK cell-ADCC NK cell-, and CDC-mediated killing upon administration of SIRPα IgG1Fc (B), NK cell-ADCC NK cell-, and CDC-mediated killing upon administration of SIRPα IgG4Fc (C), ADCC macrophage- and macrophage-mediated killing (D), ADCC macrophage- and macrophage-mediated killing upon administration of SIRPα IgG1Fc (E), and ADCC macrophage- and macrophage-mediated killing upon administration of SIRPα IgG4Fc.

FIGS. 19A and 19B show cell viability in vivo of human HIP iPSCs upon subcutaneous transplantation of human iPSCs and adoptive transfer of human NK cells and administration of SIRPα IgG1Fc on Day 0, Day 1, and Day 3, with re-injection of human HIP iPSC performed on D20 and D40, followed by SIRPα IgG1Fc injections (for 3 days).

FIGS. 20A and 20B show cell viability in vivo of human HIP iPSCs upon subcutaneous transplantation of human iPSCs and adoptive transfer of human NK cells and administration of SIRPα IgG4Fc on Day 0, Day 1, and Day 3, with re-injection of human HIP iPSC performed on D20 and D40, followed by SIRPα IgG4Fc injections (for 3 days).

FIGS. 21A and 21B show teratoma formation (HIP iPSC survival) in the brain of NSG mice upon intracranial transplantation of human iPSCs and adoptive transfer of human NK cells and human microglia and administration of IgG1 isotype control on Day 0, Day 1, and Day 3.

FIGS. 22A and 22B show cell viability in vivo of human HIP iPSCs upon intracranial transplantation of human iPSCs and adoptive transfer of human NK cells and human microglia and administration of SIRPα IgG1Fc on Day 0, Day 1, and Day 3.

FIGS. 23A and 23B show teratoma formation (HIP iPSC survival) in the brain of NSG mice upon intracranial transplantation of human iPSCs and adoptive transfer of human NK cells and human microglia and intraperitoneal administration of IgG1 isotype control on Day 0, Day 1, and Day 3.

FIGS. 24A and 24B show cell viability in vivo of human HIP iPSCs in the brain upon intracranial transplantation of human iPSCs and adoptive transfer of human NK cells and human microglia and intraperitoneal administration of SIRPα IgG1Fc on Day 0, Day 1, and Day 3, with the blood-brain barrier broken by mannitol injections.

FIGS. 25A and 25B show teratoma formation (HIP iPSC survival) in the brain of NSG mice upon intracranial transplantation of human iPSCs and adoptive transfer of human NK cells and human microglia and administration of IgG4 isotype control on Day 0, Day 1, and Day 3.

FIGS. 26A and 26B show cell viability in vivo of human HIP iPSCs in the brain upon intracranial transplantation of human iPSCs and adoptive transfer of human NK cells and human microglia and intraperitoneal administration of SIRPα IgG4c on Day 0, Day 1, and Day 3.

FIG. 27 shows cell viability of human HIP iPSCs upon subcutaneous injection of human iPSCs into NSG mice along with adoptive transfer with human NK cells and anti-SIRPα subcutaneously mixed in at 1 mg on D0, D1, and D3. Reinjection with human HIP iPSCs was performed on D20 50,000 cells (50k) subcutaneously (into the left side) along with 1 mg B6H12 on D20 (mixed in), D21, and D23. Reinjection with human HIP iPSCs was performed on D40 50k subcutaneously (into upper middle chest) along with 1 mg B6H12 on D40 (mixed in), D41, and D43.

FIGS. 28A and 28B show CD47 blocking data by SIRPα IgG1Fc or SIRPα IgG4Fc in vitro, with effects studied on NK cells (A) and macrophages (B).

FIGS. 29A and 29B show CD47 blocking data by SIRPα IgG1Fc or SIRPα IgG4Fc in vitro, with effects studied on CD19 HIP CAR and NK cells (A), and CD19 HIP CAR and macrophages (B).

FIG. 30 shows a study of NSG mice using a Nalm6 tumor model. Adoptive transfer of human NK cells and human HIP CAR-T cells was performed intravenously with and without fusion protein intravenously. 100 U/ml IL-2 was thawed overnight before sorting, followed by 100 U/ml IL-2 overnight after sorting and before injection.

FIG. 31 shows a study of NSG mice using a Nalm6 tumor model. Adoptive transfer of human NK cells and human HIP CAR-T cells was performed intravenously with and without fusion protein intravenously. 100 U/ml IL-2 was thawed overnight before sorting, followed by 100 U/ml IL-2 overnight after sorting and before injection. When HIP CARs were eliminated by a safety strategy, Nalm-6 tumor grew.

FIG. 32 shows a study of NSG mice using a Nalm6 tumor model.

FIGS. 33 and 34 shows a study of NSG mice using a Nalm6 tumor model, where HIP CAR T cells are eliminated by IgG1 and IgG4 anti-CD47 fusion proteins, indicating the growth of Nalm-6 tumor.

FIGS. 35A and 35B show in vitro cell viability of mouse HIP primary islets upon administration of anti-CD47 MIAP410 antibody as a result of NK cell-mediated killing (A) and macrophage-mediated killing (B).

FIG. 36 shows a pancreatic islet mouse study model.

FIGS. 37A-37C show cell viability data for allogeneic HIP islets and for diabetes remission in allogeneic mice upon intramuscular administration of IgG1 isotype control.

FIGS. 38A-38C show cell viability data for allogeneic HIP islets and for diabetes remission in allogeneic mice upon intramuscular administration of 5 mg of MIAP410 on D7-D18.

FIGS. 39A-B show cell viability of HIP iPSCs upon injection of human HIP iPSCs into NSG mice with adoptive transfer of human NK cells and human macrophages and administration of MIAP410 with Fc isotype IgG1, with or without in vivo IL-2 stimulation.

FIGS. 40A-B show cell viability of HIP iPSCs upon injection of human HIP iPSCs into NSG mice with adoptive transfer of human NK cells and human macrophages and administration of a high dose of MIAP410 with Fc isotype IgG1 three times.

FIG. 41 shows cell viability of HIP iPSCs in the brain upon local subcutaneous treatment or intraperitoneal treatment with MIAP410.

FIGS. 42A and 42B show teratoma formation (HIP iPSC survival) in NSG mice upon subcutaneous transplantation of human HIP iPSCs and adoptive transfer of NK cells and administration of IgG1 isotype control.

FIGS. 43A and 43B show cell viability in vivo of human HIP iPSCs upon subcutaneous transplantation of HIP iPSCs and adoptive transfer of NK cells and administration of a local low dose (LD; 500 μg) of MIAP410 with Fc isotype IgG1 concurrently with administration of IL-2 to NK cells for activation, during the period of Day 0 to Day 10 (D0-D10).

FIGS. 44A and 44B show cell viability in vivo of human HIP iPSCs upon subcutaneous transplantation of HIP iPSCs and adoptive transfer of NK cells and administration of a local low dose (LD; 500 μg) of MIAP410 with Fc isotype IgG1 concurrently with administration of IL-2 to NK cells for activation, during the period of Day 3 to Day 36.

FIGS. 45A and 45B show cell viability in vivo of human HIP iPSCs upon subcutaneous transplantation of HIP iPSCs and adoptive transfer of NK cells and administration of a local low dose (LD; 500 μg) of MIAP410 with Fc isotype IgG1 concurrently with administration of IL-2 to NK cells for activation, during the period of Day 11 to Day 36.

FIGS. 46A and 46B show cell viability in vivo of human HIP iPSCs upon subcutaneous transplantation of HIP iPSCs and adoptive transfer of NK cells and administration of a local low dose (LD; 500 μg) of MIAP410 with Fc isotype IgG1, during the period of Day 0 to Day 10 (D0-D10).

FIGS. 47A and 47B show cell viability in vivo of human HIP iPSCs upon subcutaneous transplantation of HIP iPSCs and adoptive transfer of NK cells and administration of a local low dose (LD; 500 μg) of MIAP410 with Fc isotype IgG1 during the period of Day 3 to Day 36.

FIGS. 48A and 48B show cell viability in vivo of human HIP iPSCs upon subcutaneous transplantation of HIP iPSCs and adoptive transfer of NK cells and administration of a local low dose (LD; 500 μg) of MIAP410 with Fc isotype IgG1 during the period of Day 11 to Day 36.

FIGS. 49A and 49B show cell viability in vivo of human HIP iPSCs upon subcutaneous transplantation of HIP iPSCs and adoptive transfer of NK cells and administration of a local high dose (HD; 1 mg) of MIAP410 with Fc isotype IgG1 on D0, D1, and D3.

FIGS. 50A and 50B show cell viability in vivo of human HIP iPSCs upon subcutaneous transplantation of HIP iPSCs and adoptive transfer of NK cells and administration of a local high dose (HD; 1 mg) of MIAP410 with Fc isotype IgG1 on D11, D12, and D14.

FIGS. 51A and 51B show cell viability in vivo of human HIP iPSCs upon subcutaneous transplantation of HIP iPSCs and adoptive transfer of NK cells and intraperitoneal administration of a local high dose (HD; 1 mg) of MIAP410 with Fc isotype IgG1 on D0, D1, and D3.

FIGS. 52A and 52B show cell viability in vivo of human HIP iPSCs upon subcutaneous transplantation of HIP iPSCs and adoptive transfer of NK cells and intraperitoneal administration of a local high dose (HD; 1 mg) of MIAP410 with Fc isotype IgG1 on D11, D12, and D14.

FIG. 53 shows cell viability of human iPSCs upon subcutaneous injection of human dKO (B2M−/−/CIITA−/−) cells into NSG mice and adoptive transfer of human NK cells.

FIG. 54 shows cell viability of human iPSCs upon subcutaneous injection of human dKO (B2M−/−/CIITA−/−) cells into NSG mice and adoptive transfer of human NK cells or human microglia.

FIGS. 55A and 55B show cell viability of human iPSCs upon subcutaneous injection of human dKO (B2M−/−/CIITA−/−) cells into NSG mice with or without adoptive transfer of human NK cells.

FIGS. 56A and 56B show cell viability of human iPSCs upon injection of human dKO (B2M−/−/CIITA−/−) cells into the brain of NSG mice with or without adoptive transfer of human NK cells.

FIGS. 57A and 57B show cell viability of human iPSCs upon injection of human dKO (B2M−/−/CIITA−/−) cells into the brain of NSG mice with adoptive transfer of human microglia.

FIG. 58 shows cell viability data for human wt, dKO (B2M−/−CIITA−/−) or HIP 1.0 (B2M−/−CIITA−/−CD47 tg) co-cultured with allogeneic human macrophages or microglia.

FIG. 59 shows cell viability data for human dKO (B2M−/−CIITA−/−) cells co-cultured with allogeneic human macrophages or microglia or mouse dKO (B2M−/−CIITA−/−) cells co-cultured with allogeneic mouse macrophages or microglia.

FIG. 60 shows cell viability data for human dKO (B2M−/−CIITA−/−) cells co-cultured with xenogeneic (cross-species) mouse macrophages or microglia or mouse dKO (B2M−/−CIITA−/−) cells co-cultured with xenogeneic human macrophages or microglia.

FIGS. 61A and 61B show cell viability in vivo of human HIP iPSCs upon intracranial transplantation of HIP iPSCs into NSG mice and adoptive transfer of NK cells and administration of a high dose (HD; 1 mg) of Fc isotype IgG1 control on D0, D1, and D3.

FIGS. 62A and 62B show cell viability in vivo of human HIP iPSCs upon intracranial transplantation of HIP iPSCs into NSG mice and adoptive transfer of NK cells and administration of a high dose (HD; 1 mg) of MIAP410 on D0, D1, and D3.

FIGS. 63A and 63B show cell viability in vivo of human HIP iPSCs upon intracranial transplantation of HIP iPSCs into NSG mice and adoptive transfer of NK cells and administration of a high dose (HD; 1 mg) of MIAP410 on D0, D1, and D3, with the blood-brain barrier broken by mannitol injections.

FIGS. 64A and 64B show cell viability data for human HIP iPSCs in vitro upon administration of 100 g/ml of a B6H12 anti-CD47 antibody with mouse IgG1 Fc domain in the presence of human NK cells (A) or human macrophages (B).

FIG. 65 shows cell viability data for human HIP iPSCs in vivo upon subcutaneous transplantation of human HIP iPSCs with adoptive transfer of human NK cells and administration of B6H12.

FIGS. 66A and 66B show cell viability in vivo of human HIP iPSCs upon subcutaneous transplantation of HIP iPSCs and adoptive transfer of NK cells and administration of a local low dose (LD; 500 μg) of Fc isotype IgG4 control during the period of D0-D40.

FIGS. 67A and 67B show cell viability in vivo of human HIP iPSCs upon subcutaneous transplantation of HIP iPSCs and adoptive transfer of NK cells and administration of a local low dose (LD; 500 μg) of B6H12 anti-CD47 antibody with Fc isotype IgG1 during the period of D0-D96.

FIGS. 68A and 68B show cell viability in vivo of human HIP iPSCs upon subcutaneous transplantation of HIP iPSCs and adoptive transfer of NK cells and administration of a local low dose (LD; 500 μg) of B6H12 anti-CD47 antibody with Fc isotype IgG1 during the period of D3-D40.

FIGS. 69A and 69B show cell viability in vivo of human HIP iPSCs upon subcutaneous transplantation of HIP iPSCs and adoptive transfer of NK cells and administration of a local low dose (LD; 500 μg) of B6H12 anti-CD47 antibody with Fc isotype IgG1 during the period of D11-D44.

FIGS. 70A and 70B show cell viability in vivo of human HIP iPSCs upon subcutaneous transplantation of HIP iPSCs and adoptive transfer of NK cells and administration of a local high dose (HD; 1 mg) of B6H12 anti-CD47 antibody with Fc isotype IgG1 on D0, D1, and D3.

FIGS. 71A and 71B show cell viability in vivo of human HIP iPSCs upon subcutaneous transplantation of HIP iPSCs and adoptive transfer of NK cells and administration of a local high dose (HD; 1 mg) of B6H12 anti-CD47 antibody with Fc isotype IgG1 on D3, D4, and D6.

FIGS. 72A and 72B show cell viability in vivo of human HIP iPSCs upon subcutaneous transplantation of HIP iPSCs and adoptive transfer of NK cells and administration of a local high dose (HD; 1 mg) of B6H12 anti-CD47 antibody with Fc isotype IgG1 on D11, D12, and D14.

FIGS. 73A and 73B show cell viability in vivo of human HIP iPSCs upon subcutaneous transplantation of HIP iPSCs and adoptive transfer of NK cells and intraperitoneal administration of a local high dose (HD; 1 mg) of B6H12 anti-CD47 antibody with Fc isotype IgG1 on D0, D1, and D3.

FIG. 74 shows a small molecule in vitro study of the effect of small molecules Flucytosine and Ganciclovir on cytosine deaminase and HsVtk kill switch, respectively, with respect to protection of cells with sufficient CD47 levels from NK cell and macrophage killing.

FIG. 75 shows pro-drug killing data for a small molecule in vitro study of the effect of small molecules Flucytosine and Ganciclovir on cytosine deaminase and HsVtk kill switch, respectively.

FIGS. 76A and 76B show human HIP-CyD iPSCs forming teratoma in NSG mice.

FIGS. 77A and 77B show cell viability data of human HIP iPSCs (CyD clone 2G11) upon subcutaneous injection into NSG mice and Flucytosine LD (200 mg/kg) treatment administered daily intraperitoneally, with killing of HIP-CyD iPSCs occurring within 16-44 days.

FIGS. 78A and 78B show cell viability data of human HIP iPSCs (CyD clone 2G11) upon subcutaneous injection into NSG mice and Flucytosine HD (500 mg/kg) treatment administered daily intraperitoneally, with killing of HIP-CyD iPSCs occurring within 16-32 days.

FIGS. 79A and 79B show cell viability data of human HIP iPSCs (CyD clone 2G11) upon subcutaneous injection into NSG mice and Flucytosine LD (200 mg/kg) treatment administered daily intraperitoneally beginning on Day 13, with killing of HIP-CyD iPSCs occurring within 3-11 days after starting administration.

FIGS. 80A and 80B show cell viability data of human HIP iPSCs (CyD clone 2G11) upon subcutaneous injection into NSG mice and Flucytosine HD (500 mg/kg) treatment administered daily intraperitoneally beginning on Day 13, with killing of HIP-CyD iPSCs occurring within 3-11 days after starting administration.

FIGS. 81A-81F show cell viability data of human HIP iPSCs (clone 15; no kill switch) upon subcutaneous injection into NSG mice and Flucytosine HD (500 mg/kg) treatment administered daily intraperitoneally, with HIP-CyD iPSC survival impaired despite the absence of a kill switch (A and B) and expansion of the study confirming the results (C-F).

FIGS. 82A and 82B show cell viability data of human HIP iPSCs luc+ (Cytosine deaminase clone 2-G11) upon subcutaneous injection into NSG mice.

FIGS. 83A and 83B show cell viability data of human HIP iPSCs (HSVTk clone 1-B10) upon subcutaneous injection into NSG mice and administration of saline.

FIGS. 84A and 84B show cell viability data of human HIP iPSCs (HSVTk clone 1-B10) upon subcutaneous injection into NSG mice and Ganciclovir LD (50 mg/kg) treatment administered daily intraperitoneally, with killing of HIP-HsVtk iPSCs occurring within 12-24 days.

FIGS. 85A and 85B show cell viability data of human HIP iPSCs (HSVTk clone 1-B10) upon subcutaneous injection into NSG mice and Ganciclovir HD (75 mg/kg) treatment administered daily intraperitoneally, with killing of HIP-HsVtk iPSCs occurring within 12-16 days.

FIGS. 86A and 86B show cell viability data of human HIP iPSCs (HSVTk clone 1-B10) upon subcutaneous injection into NSG mice and Ganciclovir LD (50 mg/kg) treatment administered daily intraperitoneally starting on Day 13, with killing of HIP-HsVtk iPSCs occurring within 7 days after starting administration.

FIGS. 87A and 87B show cell viability data of human HIP iPSCs (HSVTk clone 1-B10) upon subcutaneous injection into NSG mice and Ganciclovir HD (75 mg/kg) treatment administered daily intraperitoneally starting on Day 13, with killing of HIP-HsVtk iPSCs occurring within 7 days after starting administration.

FIGS. 88A and 88B show cell viability data of human HIP iPSCs (clone 15; no kill switch) upon subcutaneous injection into NSG mice and Ganciclovir HD (75 mg/kg) treatment administered daily intraperitoneally starting on Day 0, with no killing of HIP iPSCs occurring.

FIGS. 89A and 89B show cell viability data of human HIP iPSCs luc+ (HSVtk clone 1-B10) upon subcutaneous injection into NSG mice.

### DETAILED DESCRIPTION

Regenerative medicine (cell therapy) involves the preparation and delivery of cells to a patient. Cell therapy, i.e., the transplantation of cells into a subject to replace or repair damaged cells, may provide invaluable in treating diseases that are characterized by the progressive deterioration or absence of cells, tissues, and/or organs. In some embodiments, cell therapy aims to repair, replace, restore, and/or provide cells that are otherwise damaged, dysfunctional, or non-existent. Cells for use in cell therapy may be, e.g., pluripotent stem cells (PSCs) which can be differentiated to any cell type, cells differentiated from these PSCs, or primary cells. Cells for use in cell therapy can be engineered to contain one or more exogenous nucleic acids encoding a tolerogenic factor such as CD47, a transmembrane protein and known marker of “self” on host cells within an organism, and, optionally, one or more other proteins. When CD47 binds to signal regulatory protein alpha (SIRPα), a transmembrane receptor protein on circulating immune cells, to deliver an inhibitory “don't eat me” signal, the host cell expressing the CD47 evades rejection by the patient's immune system, e.g., through macrophage- and/or natural killer (NK) cell-mediated death. The immunosuppressive characteristics of such engineered cells can render them dangerous to a patient into whom the cells are transplanted, e.g., if unbridled growth occurs, creating a need for the development of safety mechanisms that can modulate, e.g., eliminate via the patient's innate immune system, the transplanted population of cells by acting on the CD47-SIRPα axis or interaction. The present disclosure provides methods and compositions for modulating a cell or population of cells previously administered to or transplanted into a subject, wherein the cell or population of cells contains one or more exogenous nucleic acids encoding CD47 and/or expresses or overexpresses an exogenous CD47 polypeptide, by administering a CD47-SIRPα blockade agent to the subject. The CD47-SIRPα blockade agent may comprise a small molecule, macromolecule, polypeptide, fusion protein, diabody, antibody, or a combination thereof that binds to CD47 or SIRPα, thus acting on, interfering with, blocking, and/or inhibiting a CD47-SIRPα axis or interaction. This interaction triggers innate killing mechanisms against the previously administered cells, including immune cell-mediated killing of the cells, such as NK-mediated killing, macrophage mediated killing, ADCC and/or CDC. In this manner, administration of the CD47-SIRPα blockade agent results in a decrease in, and in certain embodiments complete elimination of, the previously administered cells in the subject.

### I. Definitions

The term “antibody” is used to denote, in addition to natural antibodies, genetically engineered or otherwise modified forms of immunoglobulins or portions thereof, including chimeric antibodies, human antibodies, humanized antibodies, or synthetic antibodies. The antibodies may be monoclonal or polyclonal antibodies. In those embodiments wherein an antibody is an immunogenically active portion of an immunoglobulin molecule, the antibody may include, but is not limited to, a single chain variable fragment antibody (scFv), disulfide linked Fv, single domain antibody (sdAb), VHH antibody, antigen-binding fragment (Fab), Fab′, F(ab′)2 fragment, or diabody. An scFv antibody is derived from an antibody by linking the variable regions of the heavy (VH) and light (VL) chains of the immunoglobulin with a short linker peptide. An scFv can comprise Vh-Vl or Vl-Vh. Similarly, a disulfide linked Fv antibody can be generated by linking the VH and VL using an interdomain disulfide bond. On the other hand, sdAbs consist of only the variable region from either the heavy or light chain and usually are the smallest antigen-binding fragments of antibodies. A VHH antibody is the antigen binding fragment of heavy chain only. A diabody is a dimer of scFv fragment that consists of the VH and VL regions noncovalent connected by a small peptide linker or covalently linked to each other. The antibodies disclosed herein, including those that comprise an immunogenically active portion of an immunoglobulin molecule, retain the ability to bind a specific antigen.

The term “safety switch” used herein refers to a system for controlling the expression of a gene or protein of interest that, when downregulated or upregulated, leads to clearance or death of the cell, e.g., through recognition by the host's immune system. A safety switch can be designed to be or include an exogenous molecule administered to prevent or mitigate an adverse clinical event. A safety switch can be engineered by regulating the expression on the DNA, RNA and protein levels. A safety switch may include a protein or molecule that allows for the control of cellular activity in response to an adverse event. In some embodiments, a safety switch refers to an agent (e.g., protein, molecule, etc.) that binds a specific cell and targets it for cell death or elimination. In some instances, the safety switch is a blockade agent that binds a target protein on the surface of a target cell, which in turn, triggers an immune response. In one embodiment, the safety switch is a ‘kill switch’ that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis.

As used herein to characterize a cell, the term “hypoimmunogenic” generally means that such cell is less prone to immune rejection by a subject into which such cells are engrafted or transplanted. For example, relative to an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. In some aspects, genome editing technologies are used to modulate the expression of MHC I and/or MHC II genes, and optionally express a tolerogenic factor such as but not limited to CD47 and thus, generate a hypoimmunogenic cell. In some embodiments, a hypoimmunogenic cell evades immune rejection in an MHC-mismatched allogenic recipient. In some instance, differentiated cells produced from the hypoimmunogenic stem cells outlined herein evade immune rejection when administered (e.g., transplanted or grafted) to an MHC-mismatched allogenic recipient. In some instances, hypoimmunogenic or differentiated cells produced from the hypoimmunogenic stem cells outlined herein evade immune rejection when administered (e.g., transplanted or grafted) to an MHC-mismatched allogenic recipient with a lower level of immune suppression than would be needed with a non-hypoimmunogenic cell. In some embodiments, a hypoimmunogenic cell is protected from adaptive immune rejection and/or innate immune cell rejection.

Hypoimmunogenicity of a cell can be determined by evaluating the immunogenicity of the cell such as the cell's ability to elicit adaptive and innate immune responses. Such immune response can be measured using assays recognized by those skilled in the art. In some embodiments, an immune response assay measures the effect of a hypoimmunogenic cell on T cell proliferation, T cell activation, T cell killing, NK cell proliferation, NK cell activation, and macrophage activity. In some cases, hypoimmunogenic cells and derivatives thereof undergo decreased killing by T cells and/or NK cells upon administration to a subject. In some instances, the cells and derivatives thereof show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

“Immunosuppressive factor” or “immune regulatory factor” as used herein include hypoimmunity factors and complement inhibitors.

“Immune signaling factor” as used herein refers to, in some cases, a molecule, protein, peptide and the like that activates immune signaling pathways.

“Safe harbor locus” as used herein refers to a gene locus that allows safe expression of a transgene or an exogenous gene. Safe harbors or genomic safe harbors are sites in the genome able to accommodate the integration of new genetic material in a manner that permits the newly inserted genetic elements to: (i) function predictably and (ii) do not cause alterations of the host genome posing a risk to the host cell or organism. Exemplary “safe harbor” loci include a CCR5 gene, a CXCR4 gene, a PPP1R12C (also known as AAVS1) gene, an albumin gene, and a Rosa gene.

An exogenous molecule or construct can be the same type of molecule as an endogenous molecule, e.g., an exogenous protein or nucleic acid. In such instances, the exogenous molecule is introduced into the cell at greater concentrations than that of the endogenous molecule in the cell. In some instances, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation of gene expression” refers to a change in the expression level of a gene. Modulation of expression can include, but is not limited to, gene activation and gene repression. Modulation may also be complete, i.e, wherein gene expression is totally inactivated or is activated to wildtype levels or beyond; or it may be partial, wherein gene expression is partially reduced, or partially activated to some fraction of wildtype levels.

As used herein, the term “reduced expression” or “decreased expression” refers to a cell exhibiting an expression level of a gene or protein that is lower (for instance, a level that is at least about 2%, 5%, 10%, 15%, 20%, 25%, 30%, 35%, 40%, 45%, 50%, 55%, 60%, 65%, 70%, 75%, 80%, 85%, 90%, or 95% lower), compared to an unmodified corresponding cell or wild-type cell (e.g., normal, healthy or parental cell).

As used herein, the term “enhanced expression” or “increased expression” refers to a cell exhibiting an expression level of a gene or protein that is higher (for instance, a level that is at least about 2%, 5%, 10%, 15%, 20%, 25%, 30%, 35%, 40%, 45%, 50%, 55%, 60%, 65%, 70%, 75%, 80%, 85%, 90%, or 95% higher), compared to an unmodified corresponding cell or wild-type cell (e.g., normal, healthy or parental cell).

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

“Pluripotent stem cells” or “primary cells” as used herein have the potential to differentiate into any of the three germ layers: endoderm (e.g., the stomach lining, gastrointestinal tract, lungs, etc), mesoderm (e.g., muscle, bone, blood, urogenital tissue, etc) or ectoderm (e.g. epidermal tissues and nervous system tissues). The term “pluripotent stem cells,” as used herein, also encompasses “induced pluripotent stem cells,” or “iPSCs,” a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al, Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

The terms “treat”, “treating”, “treatment”, etc., as applied to an isolated cell, include subjecting the cell to any kind of process or condition or performing any kind of manipulation or procedure on the cell. As applied to a subject, the terms refer to administering a cell or population of cells in which a target polynucleotide sequence (e.g., B2M) has been altered ex vivo according to the methods described herein to an individual. The individual is usually ill or injured, or at increased risk of becoming ill relative to an average member of the population and in need of such attention, care, or management.

As used herein, the term “treating” and “treatment” refers to administering to a subject an effective amount of cells with target polynucleotide sequences altered ex vivo according to the methods described herein so that the subject has a reduction in at least one symptom of the disease or an improvement in the disease, for example, beneficial or desired clinical results. For purposes of the present technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a disorder associated with expression of a polynucleotide sequence, as well as those likely to develop such a disorder due to genetic susceptibility or other factors.

By “treatment” or “prevention” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

As used herein, the terms “administering,” “introducing” and “transplanting” are used interchangeably in the context of the placement of cells, e.g. cells described herein comprising a target polynucleotide sequence altered according to the methods of the present disclosure into a subject, by a method or route which results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e. g. twenty-four hours, to a few days, to as long as several years. In some instances, the cells can also be administered a location other than the desired site, such as in the liver or subcutaneously, for example, in a capsule to maintain the implanted cells at the implant location and avoid migration of the implanted cells.

In additional or alternative aspects, the present technology contemplates altering target polynucleotide sequences in any manner which is available to the skilled artisan, e.g., utilizing a TALEN system. It should be understood that although examples of methods utilizing CRISPR/Cas (e.g., Cas9 and Cpf1) and TALEN are described in detail herein, the technology is not limited to the use of these methods/systems. Other methods of targeting, e.g., B2M, to reduce or ablate expression in target cells known to the skilled artisan can be utilized herein.

The methods of the present disclosure can be used to alter a target polynucleotide sequence in a cell. The present disclosure contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the CRISPR/Cas systems of the present disclosure. In other instances, a “mutant cell” exhibits a wild-type phenotype, for example when a CRISPR/Cas system is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system can be used to induce an indel of any length or a point mutation in a target polynucleotide sequence.

As used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use the CRISPR/Cas systems to knock out a target polynucleotide sequence or a portion thereof based upon the details described herein.

In some embodiments, the alteration results in a knock out of the target polynucleotide sequence or a portion thereof. Knocking out a target polynucleotide sequence or a portion thereof using a CRISPR/Cas system can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

By “knock in” herein is meant a process that adds a genetic function to a host cell. This, in some embodiments, causes increased or decreased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

In some embodiments, the alteration results in reduced expression of the target polynucleotide sequence. The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “exogenous” in intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. An “exogenous” molecule is a molecule, construct, factor and the like that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of neurons is an exogenous molecule with respect to an adult neuron cell. An exogenous molecule can comprise, for example, a functioning version of a malfunctioning endogenous molecule or a malfunctioning version of a normally-functioning endogenous molecule.

An exogenous molecule or factor can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

The term “endogenous” refers to a referenced molecule or polypeptide that is present in the cell. Similarly, the term when used in reference to expression of an encoding nucleic acid refers to expression of an encoding nucleic acid contained within the cell and not exogenously introduced.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Nat'l. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al, infra).

One example of an algorithm that is suitable for determining percent sequence identity and sequence similarity is the BLAST algorithm, which is described in Altschul et al., J. Mol. Biol., 215:403-410 (1990). Software for performing BLAST analyses is publicly available through the National Center for Biotechnology Information.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the present disclosure. Any recited method may be carried out in the order of events recited or in any other order that is logically possible. Although any methods and materials similar or equivalent to those described herein may also be used in the practice or testing of the present technology, representative illustrative methods and materials are now described.

As described herein, the following terms will be employed, and are defined as indicated below.

Before the present technology is further described, it is to be understood that it is not limited to particular embodiments described, as such may, of course, vary. It is also to be understood that the terminology used herein is for the purpose of describing particular embodiments only, and is not intended to be limiting, since the scope of the present technology will be limited only by the appended claims.

Unless defined otherwise, all technical and scientific terms used herein have the same meaning as commonly understood by one of ordinary skill in the art to which this technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the technology. The upper and lower limits of these smaller ranges may independently be included in the smaller ranges and are also encompassed within the technology, subject to any specifically excluded limit in the stated range. Where the stated range includes one or both of the limits, ranges excluding either or both of those included limits are also included in the technology. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the technology described herein is not entitled to antedate such publication by virtue of prior disclosure. Further, the dates of publication provided might be different from the actual publication dates, which may need to be independently confirmed.

### II. CD47, Signal Regulatory Protein Alpha (SIRPα), and the Immune System

Provided herein are methods and compositions for modulating a population of cells expressing CD47 and previously administered to or transplanted into a subject, comprising administering a CD47-SIRPα blockade agent to the subject.

### A. The CD47-SIRPα Axis/Interaction

Cluster of Differentiation 47 (CD47) is a heavily glycosylated, ubiquitously expressed cell surface protein in the immunoglobulin superfamily. CD47 plays roles in important cellular functions like proliferation, adhesion, migration, apoptosis and phagocytosis. The molecular structure of CD47 includes an extracellular immunoglobulin variable (IgV)-like domain, a transmembrane spanning domain, and a short, alternatively spliced cytoplasmic tail. In some embodiments, CD47 interacts in trans with signal regulatory protein alpha (SIRPα) and plays a role in recruitment of granulocytes and T cells to sites of infection. SIRPα encodes an Ig-superfamily receptor expressed on the surface of macrophages and dendritic cells, whose cytoplasmic region contains immunoreceptor tyrosine-based inhibition motifs (ITIMs) that can trigger a cascade to inhibit phagocytosis.

CD47 functions as a marker of “self” on host cells within an organism. In some embodiments, when expressed, CD47 binds to SIRPα on the surface of circulating immune cells to deliver an inhibitory “don't eat me” signal. CD47-SIRPα binding results in phosphorylation of ITIMs on SIRPα, which triggers a series of events, which can ultimately prevent phagocytosis. Phagocytosis of target cells by macrophages is regulated by a balance of activating signals and inhibitory signals (SIRPα-CD47). This balance is tipped by cancer cells, which co-opt the “self” signal and upregulate CD47 expression to evade immune surveillance and subsequent destruction. In some embodiments, a CD47-binding agent and/or SIRPα-binding agent, i.e., a CD47-SIRPα blockade agent, blocks and/or interferes with the inhibitory SIRPα-CD47 signal, thereby triggering phagocytosis and/or other immune system mechanisms.

### B. Immune System-Mediated Killing of Target Cells

Provided herein in certain embodiments are methods of triggering innate killing mechanisms against a cell or population of cells previously administered to or transplanted into a subject, wherein the cells express or overexpress CD47, by administering to the subject one or more CD47-SIRPα blockade agents. In certain of these embodiments, the cells expressing or overexpressing CD47 comprise one or more exogenous nucleic acids encoding CD47. The triggered innate killing mechanisms may be one or more immune cell-mediated killing mechanisms, including NK-mediated killing, macrophage mediated killing, ADC and/or CDCC.

Provided herein in certain embodiments are methods of triggering NK cell-mediated killing of a cell or population of cells previously administered or transplanted into a subject and expressing or overexpressing CD47, including cells engineered to express or overexpress CD47.

Provided herein in certain embodiments are methods of triggering macrophage-mediated killing of a cell or population of cells previously administered or transplanted into a subject and expressing or overexpressing CD47, including cells engineered to express or overexpress CD47. Macrophages are important components of innate immunity, which can inhibit tumor growth through phagocytosis. SIRPα is expressed on the surface of myeloid cells, including macrophages, granulocytes, monocytes, and dendritic cells. When macrophages bind to CD47 on target cells, such as cancer cells or other exogenous cells, via SIRPα, macrophage-mediated killing of target cells is inhibited. In some embodiments, a SIRPα- and/or CD47-binding agent, i.e., a CD47-SIRPα blockade agent, blocks and/or interferes with the inhibition of macrophage-mediated phagocytosis, triggering macrophage-mediated killing of target cells expressing CD47.

Provided herein in certain embodiments are methods of triggering antibody-dependent cellular cytotoxicity (ADCC)-mediated killing of a cell or population of cells previously administered or transplanted into a subject and expressing or overexpressing CD47, including cells engineered to express or overexpress CD47. Some immune cells mediate induction of tumor cell death of antibody-opsonized cancer cells, a process known as ADCC. Some immune cells are endowed with inhibitory receptors, such as SIRPα, which binds to CD47 on target cells, such as cancer cells or other exogenous cells, resulting in the inhibition of immune cell-mediated ADCC. In some embodiments, a SIRPα- and/or CD47-binding agent, i.e., a CD47-SIRPα blockade agent, blocks and/or interferes with the inhibition of immune cell-mediated ADCC, triggering ADCC-mediated killing of target cells expressing CD47. ADCC can be mediated through the activation of different Fc receptors and by different Fc receptor-expressing cells, such as natural killer (NK) cells, macrophages, and neutrophils. In some embodiments, ADCC is effectively triggered by CD47-SIRPα blockade agents comprising IgG1 and/or IgG4.

Provided herein in certain embodiments are methods of triggering complement-dependent cytotoxicity (CDC)-mediated killing of a cell or population of cells previously administered or transplanted into a subject and expressing or overexpressing CD47, including cells engineered to express or overexpress CD47. In some embodiments, the complement system is activated via binding of an Fc domain-containing antibody complexed with an antigen, such as CD47, on a target cell. C1q binds to the antibody's Fc domain in the antibody-antigen complex, triggering the binding of other complement proteins, leading ultimately to the formation of one or more cytolytic membrane attack complexes (MACs), which form pores in the target cell's membrane, leading to cell lysis/death. In some embodiments, CDC is effectively triggered by a CD47-SIRPα blockade agent comprising IgG1.

In some embodiments of the methods provided herein, a cell or population of cells containing one or more nucleic acids encoding CD47 and/or expressing or overexpressing CD47 comprises the nucleotide sequence set forth in SEQ ID NO:1 (coding sequence (CDS) of the nucleotide sequence set forth in NCBI Ref. No. NM_001777.4) or SEQ ID NO:3 (CDS of the nucleotide sequence set forth in NCBI Ref. No. NM_198793.2), or a nucleotide sequence at least 80% identical (e.g., at least 80%, at least 85%, at least 90%, at least 95%, at least 96%, at least 97%, at least 98%, at least 99%, or 100% identical) to SEQ ID NO:1 or SEQ ID NO:3. In certain of these embodiments, the nucleic acid encoding CD47 is exogenous. In certain embodiments, CD47 expressed or overexpressed by the cell comprises, consists, or consists essentially of the amino acid sequence set forth in SEQ ID NO: 2 or SEQ ID NO:4, or an amino acid sequence at least 80% identical (e.g., at least 80%, at least 85%, at least 90%, at least 95%, at least 96%, at least 97%, at least 98%, at least 99%, or 100% identical) to the amino acid sequence set forth in SEQ ID NO:2 or SEQ ID NO: 4. In some embodiments, the nucleotide sequence encoding CD47 is codon-optimized for expression in a mammalian cell, for example, a human cell. In some embodiments, the codon-optimized nucleotide sequence encoding CD47 is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:5.

TABLE 1
Exemplary sequences of CD47

| SEQ | | |
|---|---|---|
| ID | | |
| NO: | Sequence | Description |
| 1 | atgtggcccctggtagcggcgctgttgctgggctcggcgtgctg | Nucleotide sequence |
| | cggatcagctcagctactatttaataaaacaaaatctgtagaat | of NM_001777.4 CDS |
| | tcacgttttgtaatgacactgtcgtcattccatgctttgttactaatat | (nts 124-1095) |
| | ggaggcacaaaacactactgaagtatacgtaaagtggaaatt | |
| | taaaggaagagatatttacacctttgatggagctctaaacaagt | |
| | ccactgtccccactgactttagtagtgcaaaaattgaagtctca | |
| | caattactaaaaggagatgcctctttgaagatggataagagtg | |
| | atgctgtctcacacacaggaaactacacttgtgaagtaacaga | |
| | attaaccagagaaggtgaaacgatcatcgagctaaaatatcg | |
| | tgttgtttcatggttttctccaaatgaaaatattcttattgttattttccc | |
| | aatttttgctatactcctgttctggggacagtttggtattaaaacac | |
| | ttaaatatagatccggtggtatggatgagaaaacaattgctttac | |
| | ttgttgctggactagtgatcactgtcattgtcattgttggagccatt | |
| | cttttcgtcccaggtgaatattcattaaagaatgctactggccttg | |
| | gtttaattgtgacttctacagggatattaatattacttcactactatg | |
| | tgtttagtacagcgattggattaacctccttcgtcattgccatattg | |
| | gttattcaggtgatagcctatatcctcgctgtggttggactgagtc | |
| | tctgtattgcggcgtgtataccaatgcatggccctcttctgatttca | |
| | ggtttgagtatcttagctctagcacaattacttggactagtttatat | |
| | gaaatttgtggcttccaatcagaagactatacaacctcctagga | |
| | aagctgtagaggaaccccttaatgcattcaaagaatcaaaag | |
| | gaatgatgaatgatgaataa | |
| 2 | MWPLVAALLLGSACCGSAQLLFNKTKSVEFT | Amino acid sequence |
| | FCNDTVVIPCFVTNMEAQNTTEVYVKWKFK | encoded by CDS of |
| | GRDIYTFDGALNKSTVPTDFSSAKIEVSQLLK | NM_001777.4 |
| | GDASLKMDKSDAVSHTGNYTCEVTELTREG | |
| | ETIIELKYRVVSWFSPNENILIVIFPIFAILLFWG | |
| | QFGIKTLKYRSGGMDEKTIALLVAGLVITVIVI | |
| | VGAILFVPGEYSLKNATGLGLIVTSTGILILLHY | |
| | YVFSTAIGLTSFVIAILVIQVIAYILAVVGLSLCI | |
| | AACIPMHGPLLISGLSILALAQLLGLVYMKFVA | |
| | SNQKTIQPPRKAVEEPLNAFKESKGMMNDE | |
| 3 | atgtggcccctggtagcggcgctgttgctgggctcggcgtgctg | Nucleotide sequence |
| | cggatcagctcagctactatttaataaaacaaaatctgtagaat | of NM_198793.2 CDS |
| | tcacgttttgtaatgacactgtcgtcattccatgctttgttactaatat | (nts 181-1098) |
| | ggaggcacaaaacactactgaagtatacgtaaagtggaaatt | |
| | taaaggaagagatatttacacctttgatggagctctaaacaagt | |
| | ccactgtccccactgactttagtagtgcaaaaattgaagtctca | |
| | caattactaaaaggagatgcctctttgaagatggataagagtg | |
| | atgctgtctcacacacaggaaactacacttgtgaagtaacaga | |
| | attaaccagagaaggtgaaacgatcatcgagctaaaatatcg | |
| | tgttgtttcatggttttctccaaatgaaaatattcttattgttattttccc | |
| | aatttttgctatactcctgttctggggacagtttggtattaaaacac | |
| | ttaaatatagatccggtggtatggatgagaaaacaattgctttac | |
| | ttgttgctggactagtgatcactgtcattgtcattgttggagccatt | |
| | cttttcgtcccaggtgaatattcattaaagaatgctactggccttg | |
| | gtttaattgtgacttctacagggatattaatattacttcactactatg | |
| | tgtttagtacagcgattggattaacctccttcgtcattgccatattg | |
| | gttattcaggtgatagcctatatcctcgctgtggttggactgagtc | |
| | tctgtattgcggcgtgtataccaatgcatggccctcttctgatttca | |
| | ggtttgagtatcttagctctagcacaattacttggactagtttatat | |
| | gaaatttgtggcttccaatcagaagactatacaacctcctagga | |
| | ataactga | |
| 4 | MWPLVAALLLGSACCGSAQLLFNKTKSVEFT | Amino acid sequence |
| | FCNDTVVIPCFVTNMEAQNTTEVYVKWKFK | encoded by CDS of |
| | GRDIYTFDGALNKSTVPTDFSSAKIEVSQLLK | NM_198793.2 |
| | GDASLKMDKSDAVSHTGNYTCEVTELTREG | |
| | ETIIELKYRVVSWFSPNENILIVIFPIFAILLFWG | |
| | QFGIKTLKYRSGGMDEKTIALLVAGLVITVIVI | |
| | VGAILFVPGEYSLKNATGLGLIVTSTGILILLHY | |
| | YVFSTAIGLTSFVIAILVIQVIAYILAVVGLSLCI | |
| | AACIPMHGPLLISGLSILALAQLLGLVYMKFVA | |
| | SNQKTIQPPRNN | |
| 5 | atgtggcccctggtcgccgccctgttgctgggctcggcatgctg | Codon-optimized |
| | cggatcagctcagctactgtttaataaaacaaaatctgtagaat | nucleotide sequence |
| | tcacgttttgtaacgacactgtcgtgatcccatgctttgttactaat | encoding CD47 |
| | atggaggcacaaaacaccactgaagtgtacgtgaagtggaa | |
| | attcaaaggcagagacatttacacctttgacggcgccctcaac | |
| | aagtccaccgtgcccactgactttagtagcgcaaaaattgagg | |
| | tcagccaattactaaaaggagatgcctctttgaagatggacaa | |
| | gagcgatgctgtcagccacacagggaactacacttgtgaagt | |
| | aacagagttaacccgcgaaggtgaaacgatcatcgagctga | |
| | agtatcgagtggtgtcctggttttctccgaacgagaatatccttat | |
| | cgtaattttcccaattttcgctatcctcctgttctggggccagtttgg | |
| | tatcaagacactcaaatatcggtccggtgggatggatgagaa | |
| | gacaattgccctgcttgttgctggactcgtgatcaccgtcatcgt | |
| | gattgttggggccatccttttcgtcccaggggagtacagcctga | |
| | agaatgctacgggcctgggattaattgtgacctctacagggat | |
| | actcatcctgcttcactactatgtgttcagtaccgcgattggactg | |
| | acctccttcgtcattgccatattggtgattcaggtgatagcctaca | |
| | tcctcgccgtggttggcctgagtctctgtatcgcggcgtgcatac | |
| | ccatgcatggccctcttctgatttcagggttgagtatcctcgcact | |
| | agcacagttgctgggactggtttatatgaaatttgtggcctccaa | |
| | ccagaagactatacagcctcctaggaaggctgtagaggagc | |
| | ccctgaatgcattcaaggaatcaaaaggcatgatgaatgatg | |
| | aa | |

### C. Evading the Immune System Using CD47 Expression on Engineered Cells

Provided herein are engineered cells that exogenously express CD47 and methods of use thereof. In some embodiments, such CD47 expressing cells are administered to a patient, and in some instances, administered prior to the administration of a CD47-SIRPα blockade agent. As will be appreciated, any of the agents described above that can inhibit or block the interaction of CD47 and SIRPα can be used in any combination to serve as safety switches for any of the engineered cells that evade immune recognition described herein.

In some embodiments, cells exogenously expressing CD47 that can evade immune recognition or response (e.g., exhibit reduced immunogenicity or are hypoimmunogenic) are introduced to a recipient subject. Evasion of immune recognition can be achieved through overexpression of one or more immunosuppressive factors or molecules, including tolerogenic factors and complement inhibitors. In some embodiments, the cells also exhibit reduced expression of MHC I or MHC II, or both (e.g., HLA I and/or HLA II). In many embodiments, the cells further exhibit reduced expression or a lack of expression of T-cell receptors (TCRs) (e.g., TCRα and/or TCRβ). Detailed descriptions of such cells and methods of generating such as described herein.

In one embodiment, the expression of an immunosuppressive factor is based on modulating expression of the immune regulatory factor CD47. CD47 is a component of the innate immune system that in some aspects functions as a “do not eat me” signal as part of the innate immune system to block phagocytosis by macrophages. Useful immunosuppressive factors that can be engineered to be expressed by the cells of interest include, but are not limited to, CD47, CD27, CD35, CD46, CD55, CD59, CD200, DUX4, HLA-C, HLA-E, HLA-E heavy chain, HLA-G, PD-L1, IDO1, CTLA4, C1-Inhibitor, IL-10, IL-35, FASL, Serpinb9, CCL21, Mfge8, TGF-β, Cd73, Cd39, LAG3, IL1r2, Ackr2, Tnfrsf22, Tnfrsf23, Tnfrsf10, Dad1, or IFNγRI d39, including those described in WO2018227286 filed Jun. 12, 2018, the contents of which including the sequences provided therein, Table 1, and the sequence listing are herein incorporated by reference in its entirety.

In some embodiments, engineered cells provided herein comprise exogenously expressed CD47 and one or more (e.g., 1, 2, 3, 4, 5, 6, 7, 8, 9, 10 or more) additionally exogenously expressed polypeptides selected from a group that includes DUX4, PD-L1, CD24, CD46, CD55, CD59, CD200, HLA-G (H2-M3), FASL (FASLG), CCL21 (Ccl21 b), Mfge8, Serpin B9 (Spi6), and any combination thereof. In some embodiments, the engineered cells comprise exogenously expressed CD47 and DUX4. In some embodiments, the engineered cells comprise exogenously expressed CD47 and PD-L1. In some embodiments, the engineered cells comprise exogenously expressed CD47 and CD24. In some embodiments, the engineered cells comprise exogenously expressed CD47 and CD46. In some embodiments, the engineered cells comprise exogenously expressed CD47 and CD55. In some embodiments, the engineered cells comprise exogenously expressed CD47 and CD59. In some embodiments, the engineered cells comprise exogenously expressed CD47 and CD200. In some embodiments, the engineered cells comprise exogenously expressed CD47 and HLA-G. In some embodiments, the engineered cells comprise exogenously expressed CD47 and FASL. In some embodiments, the engineered cells comprise exogenously expressed CD47 and CCL21. In some embodiments, the engineered cells comprise exogenously expressed CD47 and Mfge8. In some embodiments, the engineered cells comprise exogenously expressed CD47 and Serpin B9 (Serpinb9). In some embodiments, the engineered cells comprise exogenously expressed CD47, PD-L 1, HLAG, CD200, FASL, CCL21, Mfge8, and Serpin B9.

In some embodiments, the present disclosure provides a method of producing cells or a population thereof that has been modified to express one or more of the immunosuppressive factors selected from a group that includes CD47, PD-L1, CD24, CD27, CD35, CD46, CD55, CD59, CD200, DUX4, HLA-C, HLA-E, HLA-E heavy chain, HLA-G, IDO1, CTLA4, C1-Inhibitor, IDO1, IL-10, IL-35, FASL, CCL21, Mfge8, and Serpin B9. In certain embodiments, the present disclosure provides cells or a population thereof that has been modified to express one or more of the immunosuppressive factors selected from a group that includes CD47, PD-L1, CD24, CD27, CD35, CD46, CD55, CD59, CD200, DUX4, HLA-C, HLA-E, HLA-E heavy chain, HLA-G, IDO1, CTLA4, C1-Inhibitor, IDO1, IL-10, IL-35, FASL, CCL21, Mfge8, and Serpin B9. In other embodiments, the immunosuppressive factor is selected from a group that includes B2M, CIITA, NLRC5, TAP1, HLA-A, HLA-B, HLA-C, RFX-ANK, NFY-A, RFX5, RFX-AP, HLA-G, HLA-E, NFY-B, PD-L1, NFY-C, IRF1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, and HELIOS.

In some embodiments, an immunosuppressive factor is integrated into an endogenous locus to safeguard expression of the factor or the vector cassette harboring the factor. In some embodiments, an immunosuppressive factor is inserted into a site selected from a B2M locus, a CIITA locus, a TRAC locus, a TRBC locus, and a safe harbor locus. Non-limiting examples of safe harbor loci include, but are not limited to, an AAVS1 (also known as PPP1R12C), ABO, CCR5, CLYBL, CXCR4, F3 (also known as CD142), FUT1, HMGB1, KDM5D, LRP1 (also known as CD91), MICA, MICB, RHD, ROSA26, and SHS231 gene locus. The immunosuppressive factor can be inserted in a suitable region of the safe harbor locus, including, for example, an intron, an exon, and/or gene coding region (also known as a CoDing Sequence, or “CDS”). In some embodiments, the safe harbor locus is selected from the group consisting of the AAVS1 locus, the CCR5 locus, and the CLYBL locus. In some embodiments, the insertion occurs in one allele of the specific genomic locus. In some embodiments, the insertion occurs in both alleles of the specific genomic locus. In either of these embodiments, the orientation of the transgene inserted into the target genomic locus can be either the same or the reverse of the direction of the gene in that locus.

Provided herein are engineered cells that represent a viable source for any engrafted cell type. Such cells can be protected from adaptive and innate immune rejection upon administration to a recipient subject by way of expression of one or more immunosuppressive factors. In some embodiments, cells outlined herein are not subject to innate immune cell rejection. In some instances, the cells are not susceptible to NK cell-mediated lysis. In some instances, cells described herein are not susceptible to macrophage engulfment.

In some aspects, the engineered cells are pluripotent stem cells, differentiated cells, or primary T cells. In some embodiments, the differentiated cells are produced from pluripotent stem cells using a selected differentiation protocol for a specific cell type. In some embodiments, the primary T cells are selected from a group that includes cytotoxic T-cells, helper T-cells, memory T-cells, regulatory T-cells, tumor infiltrating lymphocytes, and combinations thereof.

In some embodiments, the primary T cells are from a pool of primary T cells from one or more donor subjects that are different than the recipient subject (e.g., the patient administered the cells). The primary T cells can be obtained from 1, 2, 3, 4, 5, 6, 7, 8, 9, 10, 20, 50, 100 or more donor subjects and, optionally, pooled together. In some embodiments, the primary T cells are harvested from one or a plurality of individuals, and in some instances, the primary T cells or the pool of primary T cells are cultured in vitro. In some embodiments, the primary T cells or the pool of primary T cells are engineered to exogenously express CD47 and cultured in vitro.

In certain embodiments, the primary T cells or the pool of primary T cells are engineered to express a chimeric antigen receptor (CAR). CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The CAR can be any known to those skilled in the art. Useful CARs include those that bind an antigen selected from a group that includes CD19, CD22, CD38, CD123, CD138, and BCMA. In some cases, the CAR is the same or equivalent to those used in FDA-approved CAR-T cell therapies such as, but not limited to, tisagenlecleucel and axicabtagene ciloleucel, or those under investigation in clinical trials. In some embodiments, the CAR is a CD19-specific CAR.

In certain embodiments, the CAR may comprise a signal peptide at the N-terminus. Non-limiting examples of signal peptides include CD8α signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 2 below.

TABLE 2
Exemplary sequences of signal peptides

| SEQ | | |
|---|---|---|
| ID | | |
| NO: | Sequence | Description |
| 6 | MALPVTALLLPLALLLHAARP | CD8α signal peptide |
| 7 | METDTLLLWVLLLWVPGSTG | IgK signal peptide |
| 8 | MLLLVTSLLLCELPHPAFLLIP | GMCSFR-α (CSF2RA) |
| | | signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G₄S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvlll, B7H3, PSMA, PSCA, CAIX, CD171, CEA, CSPG4, EPHA2, FAP, FRα, IL-13Ra, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 3 below.

TABLE 3
Exemplary sequences of hinge domains

| SEQ | | |
|---|---|---|
| ID | | |
| NO: | Sequence | Description |
| 9 | TTTPAPRPPTPAPTIASQPLSLRPEACRPA | CD8α hinge |
| | AGGAVHTRGLDFACD | domain |
| 10 | IEVMYPPPYLDNEKSNGTIIHVKGKHLCPS | CD28 hinge |
| | PLFPGPSKP | domain |
| 113 | AAAIEVMYPPPYLDNEKSNGTIIHVKGKHL | CD28 hinge |
| | CPSPLFPGPSKP | domain |
| 11 | ESKYGPPCPPCP | IgG4 hinge |
| | | domain |
| 12 | ESKYGPPCPSCP | IgG4 hinge |
| | | domain |
| 13 | ESKYGPPCPPCPAPEFLGGPSVFLFPPKP | IgG4 hinge- |
| | KDTLMISRTPEVTCVVVDVSQEDPEVQFN | CH2-CH3 |
| | WYVDGVEVHNAKTKPREEQFNSTYRVVS | domain |
| | VLTVLHQDWLNGKEYKCKVSNKGLPSSIE | |
| | KTISKAKGQPREPQVYTLPPSQEEMTKNQ | |
| | VSLTCLVKGFYPSDIAVEWESNGQPENNY | |
| | KTTPPVLDSDGSFFLYSRLTVDKSRWQEG | |
| | NVFSCSVMHEALHNHYTQKSLSLSLGK | |

In certain embodiments, the transmembrane domain of the CAR may comprise a transmembrane region of the alpha, beta, or zeta chain of a T cell receptor, CD28, CD38, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8a, CD8B, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 4 provides the amino acid sequences of a few exemplary transmembrane domains.

TABLE 4
Exemplary sequences of transmembrane domains

| SEQ | | |
|---|---|---|
| ID | | |
| NO: | Sequence | Description |
| 14 | IYIWAPLAGTCGVLLLSLVITLYC | CD8α transmembrane |
| | | domain |
| 15 | FWVLVVVGGVLACYSLLVTVAFIIFW | CD28 transmembrane |
| | V | domain |
| 114 | MFWVLVVVGGVLACYSLLVTVAFIIF | CD28 transmembrane |
| | WV | domain |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 5 provides the amino acid sequences of a few exemplary intracellular costimulatory and/or signaling domains. In certain embodiments, as in the case of tisagenlecleucel as described below, the CD3ζ signaling domain of SEQ ID NO:18 may have a mutation, e.g., a glutamine (Q) to lysine (K) mutation, at amino acid position 14 (see SEQ ID NO: 115).

TABLE 5
Exemplary sequences of intracellular
costimulatory and/or signaling domains

| SEQ | | |
|---|---|---|
| ID | | |
| NO: | Sequence | Description |
| 16 | KRGRKKLLYIFKQPFMRPVQTTQEEDG | 4-1 BB |
| | CSCRFPEEEEGGCEL | costimulatory |
| | | domain |
| 17 | RSKRSRLLHSDYMNMTPRRPGPTRKH | CD28 |
| | YQPYAPPRDFAAYRS | costimulatory |
| | | domain |
| 18 | RVKFSRSADAPAYQQGQNQLYNELNL | CD3ζ signaling |
| | GRREEYDVLDKRRGRDPEMGGKPRR | domain |
| | KNPQEGLYNELQKDKMAEAYSEIGMK | |
| | GERRRGKGHDGLYQGLSTATKDTYDA | |
| | LHMQALPPR | |
| 115 | RVKFSRSADAPAYKQGQNQLYNELNL | CD3ζ signaling |
| | GRREEYDVLDKRRGRDPEMGGKPRR | domain |
| | KNPQEGLYNELQKDKMAEAYSEIGMK | (with Q to |
| | GERRRGKGHDGLYQGLSTATKDTYDA | K mutation at |
| | LHMQALPPR | position 14) |

In certain embodiments, a CAR is inserted into a T cell or other immune cell using a vector. In certain of these embodiments, the vector contains a single expression cassette for expression of the CAR. In other embodiments, the vector is a polycistronic vector containing two or more expression cassettes, e.g., a bicistronic vector, tricistronic vector, or quadcistronic vector, which allows for simultaneous expression of two or more separate proteins from one mRNA transcript in a host cell. In these embodiments, one expression cassette may express the CAR, while the one or more additional expression cassettes may express an additional factor, including for example CD47, CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8. In certain embodiments, the two or more expression cassettes are under the control of a single promoter and are separated from one another by one or more cleavage sites to achieve co-expression of the proteins of interest from one transcript. In other embodiments, the two or more genes may be under the control of separate promoters. In certain embodiments, the polycistronic vector may further comprise a safety switch. The polycistronic vector can be any type of vector suitable for introduction of nucleotide sequences into a host cell, including, for example, plasmids, adenoviral vectors, retroviral vectors, lentiviral vectors, phages, and homology-directed repair (HDR)-based donor vectors.

In certain embodiments, the two or more expression cassettes of the polycistronic vector may be separated by one or more cleavage sites. In some embodiments, the one or more cleavage sites comprise one or more self-cleaving sites. In some embodiments, the self-cleaving site comprises a 2A site. 2A peptides are a class of 18-22 amino acid-long peptides first discovered in picornaviruses and can induce ribosomal skipping during translation of a protein, thus producing equal amounts of multiple genes from the same mRNA transcript. 2A peptides function to “cleave” an mRNA transcript by making the ribosome skip the synthesis of a peptide bond at the C-terminus, between the glycine (G) and proline (P) residues, leading to separation between the end of the 2A sequence and the next peptide downstream. There are four 2A peptides commonly employed in molecular biology, T2A, P2A, E2A, and F2A, the sequences of which are summarized in Table 6. A glycine-serine-glycine (GSG) linker is optionally added to the N-terminal of a 2A peptide to increase cleavage efficiency. The use of “( )” around a sequence in the present disclosure means that the enclosed sequence is optional.

TABLE 6
Sequences of 2A peptides

| SEQ | | |
|---|---|---|
| ID NO: | Amino Acid Sequence | 2A Peptide |
| 85 | (GSG) EGRGSLLTCGDVEENPGP | T2A |
| 86 | (GSG) ATNFSLLKQAGDVEENPGP | P2A |
| 87 | (GSG) QCTNYALLKLAGDVESNPGP | E2A |
| 88 | (GSG) VKQTLNFDLLKLAGDVESNPGP | F2A |

In some embodiments, the one or more cleavage sites additionally comprise one or more protease sites. The one or more protease sites can either precede or follow the self-cleavage sites (e.g., 2A sites) in the 5′ to 3′ order of the polycistronic vector. The protease site may be cleaved by a protease after translation of the full transcript or after translation of each expression cassette such that the first expression product is released prior to translation of the next expression cassette. In these embodiments, having a protease site in addition to the 2A site, especially preceding the 2A site in the 5′ to 3′ order, may reduce the number of extra amino acid residues attached to the expressed proteins of interest. In some embodiments, the protease site comprises a furin site, also known as a Paired basic Amino acid Cleaving Enzyme (PACE) site. There are at least three furin cleavage sequences, FC1, FC2, and FC3, the amino acid sequences of which are summarized in Table 7. Similar to the 2A sites, one or more optional glycine-serine-glycine (GSG) sequences can be included for cleavage efficiency.

TABLE 7
Sequences of furin sites

| SEQ ID NO: | Amino Acid Sequence | Furin site |
|---|---|---|
| 89 | RRRR (GSG) | FC1 |
| 90 | RKRR (GSG) | FC2 |
| 91 | RKRR (GSG) TPDPW (GSG) | FC3 |

In some embodiments, the one or more cleavage sites comprise one or more self-cleaving sites, one or more protease sites, and/or any combination thereof. For example, the cleavage site can include a 2A site alone. For another example, the cleavage site can include a FC2 or FC3 site, followed by a 2A site. In these embodiments, the one or more self-cleaving sites may be the same or different. Similarly, the one or more protease sites may be the same or different.

In some embodiments, the polycistronic vector comprises a promoter that drives constitutive gene expression in mammalian cells. Those frequently used include, for example, elongation factor 1 alpha (EF1α) promoter, cytomegalovirus (CMV) immediate-early promoter (Greenaway et al., Gene 18:355-360 (1982)), simian vacuolating virus 40 (SV40) early promoter (Fiers et al., Nature 273:113-120 (1978)), spleen focus-forming virus (SFFV) promoter, phosphoglycerate kinase (PGK) promoter (Adra et al., Gene 60 (1): 65-74 (1987)), human beta actin promoter, polyubiquitin C gene (UBC) promoter, and CAG promoter (Nitoshi et al., Gene 108:193-199 (1991)). An example of a promoter that is capable of expressing a CAR transgene in a mammalian cell (e.g., a T cell) is the EF1α promoter. The native EF1α promoter drives expression of the alpha subunit of the elongation factor-1 complex, which is responsible for the enzymatic delivery of aminoacyl tRNAs to the ribosome. The EF1α promoter has been extensively used in mammalian expression plasmids and has been shown to be effective in driving CAR expression from transgenes cloned into a lentiviral vector. See, e.g., Milone et al., Mol. Ther. 17 (8): 1453-1464 (2009).

In other embodiments, the polycistronic vector comprises an inducible promoter. Unlike constitutive promoters, inducible promoters can switch between an on and an off state in response to certain stimuli (e.g., chemical agents, temperature, light) and can be regulated in tissue- or cell-specific manners. Non-limiting examples of frequently used inducible promoters include the tetracycline On (Tet-On) system and the tetracycline Off (Tet-Off) system, which utilize tetracycline response elements (TRE) placed upstream of a minimal promoter (e.g., CMV promoter) (Gossen & Bujard, Proc. Natl. Acad. Sci. USA 89 (12): 5547-5551 (1992)). The TRE is made of 7 repeats of a 19-nucleotide tetracycline operator (tetO) sequence and can be recognized by the tetracycline repressor (tetR). In the Tet-Off system, a tetracycline-controlled transactivator (tTA) was developed by fusing the tetR with the activating domain of virion protein 16 of herpes simplex virus. In the absence of tetracycline or its analogs (e.g., doxycycline), the tTA will bind the tetO sequences of the TRE and drives expression; in the presence of tetracycline, the rTA will bind to tetracycline and not to the TRE, resulting in reduced gene expression. Conversely, in the Tet-On system, a reverse transactivator (rtTA) was generated by mutagenesis of amino acid residues important for tetracycline-dependent repression, and the rtTA binds at the TRE and drives gene expression in the presence of tetracycline or doxycycline (Gossen et al., Science 268 (5218): 1766-1769 (1995)). Other examples of inducible promoters include, for example, AlcA, LexA, and Cre.

In some embodiments, the polycistronic vector comprises a Kozak consensus sequence before the first expression cassette. A Kozak consensus sequence is a nucleic acid motif that functions as the protein translation initiation site in most eukaryotic mRNA transcripts and mediates ribosome assembly and translation initiation. In some embodiments, the Kozak consensus sequence comprises or consists of the sequence set forth in SEQ ID NO:92, wherein r is a purine (i.e., a or g): (gcc)gccrccatgg (SEQ ID NO:92).

In some embodiments, the polycistronic vector comprises a Woodchuck Hepatitis Virus (WHV) Posttranscriptional Regulatory Element (WPRE) after the second expression cassette. A WPRE is a DNA sequence that, when transcribed, creates a tertiary structure enhancing expression. The WPRE sequence is commonly used to increase expression of genes delivered by viral vectors. In some embodiments, the WPRE sequence comprises or consists of an amino acid sequence set forth in SEQ ID NO:93 or an amino acid sequence that is at least 80% identical (e.g., at least 80%, at least 85%, at least 90%, at least 95%, at least 96%, at least 97%, at least 98%, at least 99%, or 100% identical) to the sequence set forth in SEQ ID NO:93:

(SEQ ID NO: 93)
aatcaacctctggattacaaaatttgtgaaagattgactggtattctta
actatgttgctccttttacgctatgtggatacgctgctttaatgccttt
gtatcatgctattgcttcccgtatggctttcattttctcctccttgtat
aaatcctggttgctgtctctttatgaggagttgtggcccgttgtcaggc
aacgtggcgtggtgtgcactgtgtttgctgacgcaacccccactggttg
gggcattgccaccacctgtcagctcctttccgggactttcgctttcccc
ctccctattgccacggcggaactcatcgccgcctgccttgcccgctgct
ggacaggggctcggctgttgggcactgacaattccgtggtgttgtcggg
gaaatcatcgtcctttccttggctgctcgcctgtgttgccacctggatt
ctgcgcgggacgtccttctgctacgtcccttcggccctcaatccagcgg
accttccttcccgcggcctgctgccggctctgcggcctcttccgcgtct
tcgccttcgccctcagacgagtcggatctccctttgggccgcctccccg
c.

In some embodiments, the polycistronic vector comprises homology arms flanking a fragment containing the expression cassettes and/or promoter for use in site-directed insertion (knock-in) into specified loci in a host cell, for example, by homology directed repair (HDR)-based approaches as described. A fragment of the polycistronic vector to be inserted, usually containing at least the expression cassettes and optionally also containing the promoter, would be flanked by homologous sequence immediately upstream and downstream of the target insertion site (i.e., left homology arm (LHA) and right homology arm (RHA)). The homology arms are specifically designed for the target genomic locus for the fragment to serve as a template for HDR. The length of each homology arm is generally dependent on the size of the insert being introduced, with larger insertions requiring longer homology arms.

In certain embodiments, a cell or population of cells expressing an exogenous CAR and an exogenous CD47 polypeptide express the CAR and CD47 from two separate vectors. In other embodiments, the exogenous CAR and the exogenous CD47 polypeptide were introduced into the cell or population of cells via a polycistronic vector, e.g., a bicistronic vector comprising a first expression cassette expressing the exogenous CAR and a second expression cassette expressing the exogenous CD47. In certain of these embodiments, the polycistronic vector may comprise one or more additional expression cassettes expressing one or more additional factors. In certain embodiments where a cell or population of cells comprises a bicistronic vector encoding an exogenous CAR and an exogenous CD47 polypeptide, the bicistronic vector was introduced into the cell or cells via a lentivirus.

### CD19 CAR

In some embodiments, the CAR is a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34 (16-17): 1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 8 below. In some embodiments, the CD19-specific scFv comprises or consists of an amino acid sequence set forth in SEQ ID NO:19, 20, or 25, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19, 20, or 25. In some embodiments, the CD19-specific scFv may comprise one or more CDRs having amino acid sequences set forth in SEQ ID NOs: 21-23 and 26-28. In some embodiments, the CD19-specific scFv may comprise a light chain with one or more CDRs having amino acid sequences set forth in SEQ ID NOs: 21-23. In some embodiments, the CD19-specific scFv may comprise a heavy chain with one or more CDRs having amino acid sequences set forth in SEQ ID NOs: 26-28. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:24. In some embodiments, the Whitlow linker may be replaced by a different linker, for example, a 3×G₄S linker having an amino acid sequence set forth in SEQ ID NO:30, which gives rise to a different FMC63-derived scFv having an amino acid sequence set forth in SEQ ID NO:29. In certain of these embodiments, the CD19-specific scFv comprises or consists of an amino acid sequence set forth in SEQ ID NO:29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29.

TABLE 8
Exemplary sequences of anti-CD19 scFv and components

| SEQ ID NO: | Amino Acid Sequence | Description |
|---|---|---|
| 19 | DIQMTQTTSSLSASLGDRVTISCRA | Anti-CD19 FMC63 scFv |
| | SQDISKYLNWYQQKPDGTVKLLIYH | entire sequence, with |
| | TSRLHSGVPSRFSGSGSGTDYSLTI | Whitlow linker |
| | SNLEQEDIATYFCQQGNTLPYTFGG | |
| | GTKLEITGSTSGSGKPGSGEGSTK | |
| | GEVKLQESGPGLVAPSQSLSVTCT | |
| | VSGVSLPDYGVSWIRQPPRKGLEW | |
| | LGVIWGSETTYYNSALKSRLTIIKDN | |
| | SKSQVFLKMNSLQTDDTAIYYCAKH | |
| | YYYGGSYAMDYWGQGTSVTVSS | |
| 20 | DIQMTQTTSSLSASLGDRVTISCRA | Anti-CD19 FMC63 scFv |
| | SQDISKYLNWYQQKPDGTVKLLIYH | light chain variable |
| | TSRLHSGVPSRFSGSGSGTDYSLTI | region |
| | SNLEQEDIATYFCQQGNTLPYTFGG | |
| | GTKLEIT | |
| 21 | QDISKY | Anti-CD19 FMC63 scFv |
| | | light chain CDR1 |
| 22 | HTS | Anti-CD19 FMC63 scFv |
| | | light chain CDR2 |
| 23 | QQGNTLPYT | Anti-CD19 FMC63 scFv |
| | | light chain CDR3 |
| 24 | GSTSGSGKPGSGEGSTKG | Whitlow linker |
| 25 | EVKLQESGPGLVAPSQSLSVTCTV | Anti-CD19 FMC63 scFv |
| | SGVSLPDYGVSWIRQPPRKGLEWL | heavy chain variable |
| | GVIWGSETTYYNSALKSRLTIIKDNS | region |
| | KSQVFLKMNSLQTDDTAIYYCAKHY | |
| | YYGGSYAMDYWGQGTSVTVSS | |
| 26 | GVSLPDYG | Anti-CD19 FMC63 scFv |
| | | heavy chain CDR1 |
| 27 | IWGSETT | Anti-CD19 FMC63 scFv |
| | | heavy chain CDR2 |
| 28 | AKHYYYGGSYAMDY | Anti-CD19 FMC63 scFv |
| | | heavy chain CDR3 |
| 29 | DIQMTQTTSSLSASLGDRVTISCRA | Anti-CD19 FMC63 scFv |
| | SQDISKYLNWYQQKPDGTVKLLIYH | entire sequence, with |
| | TSRLHSGVPSRFSGSGSGTDYSLTI | 3xG4S linker |
| | SNLEQEDIATYFCQQGNTLPYTFGG | |
| | GTKLEITGGGGSGGGGSGGGGSE | |
| | VKLQESGPGLVAPSQSLSVTCTVS | |
| | GVSLPDYGVSWIRQPPRKGLEWLG | |
| | VIWGSETTYYNSALKSRLTIIKDNSK | |
| | SQVFLKMNSLQTDDTAIYYCAKHYY | |
| | YGGSYAMDYWGQGTSVTVSS | |
| 30 | GGGGSGGGGSGGGGS | 3xG₄S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138 (9): 2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148 (10): 2983-2987 (1992)), and CLB-CD19 (De Rie Cell. Immunol. 118:368-381 (1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO: 12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO: 12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO: 13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 15.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 16. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the CD19 CAR comprises the CD19-specific scFv having sequences set forth in SEQ ID NO:19 or SEQ ID NO:29, the IgG4 hinge domain of SEQ ID NO:11 or SEQ ID NO:12, the CD28 transmembrane domain of SEQ ID NO:15, the 4-1 BB costimulatory domain of SEQ ID NO:16, the CD34 signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the CD19 CAR comprises the CD19-specific scFv having sequences set forth in SEQ ID NO:19 or SEQ ID NO:29, the CD28 hinge domain of SEQ ID NO:10, the CD28 transmembrane domain of SEQ ID NO:15, the CD28 costimulatory domain of SEQ ID NO:17, the CD3 signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the CD19 CAR is encoded by the nucleotide sequence set forth in SEQ ID NO:116 or a nucleotide sequence at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:116 (see Table 9). The encoded CD19 CAR has a corresponding amino acid sequence set forth in SEQ ID NO:117 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:117, with the following components: CD8α signal peptide, FMC63 scFv (V_{L}-Whitlow linker-V_{H}), CD8α hinge domain, CD8α transmembrane domain, 4-1BB costimulatory domain, and CD3ζ signaling domain.

In some embodiments, the CD19 CAR is a commercially available embodiment of CD19 CAR, including but not limited to CD19 CARs expressed and/or encoded by T cells including tisagenlecleucel, lisocabtagene maraleucel, axicabtagene ciloleucel, and brexucabtagene autoleucel. Tisagenlecleucel comprises a CD19 CAR with the following components: CD8α signal peptide, FMC63 scFv (V_{L}-3×G₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 9, with annotations of the sequences provided in Table 10. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 9, with annotations of the sequences provided in Table 11. Axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 9, with annotations of the sequences provided in Table 12. Brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the CD19 CAR is encoded by a nucleotide sequence set forth in SEQ ID NO: 31, 33, or 35, or a nucleotide sequence at least 80% identical (e.g., at least 80%, at least 85%, at least 90%, at least 95%, at least 96%, at least 97%, at least 98%, at least 99%, or 100% identical) to a nucleotide sequence set forth in SEQ ID NO: 31, 33, or 35. The encoded CD19 CAR has a corresponding amino acid sequence set forth in SEQ ID NO: 32, 34, or 36, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32, 34, or 36, respectively.

TABLE 9
Exemplary sequences of CD19 CARs

| SEQ ID NO: | Sequence | Description |
|---|---|---|
| 116 | atggccttaccagtgaccgccttgctcctgccgctggccttg | Exemplary CD19 |
| | ctgctccacgccgccaggccggacatccagatgacacag | CAR nucleotide |
| | actacatcctccctgtctgcctctctgggagacagagtcacc | sequence |
| | atcagttgcagggcaagtcaggacattagtaaatatttaaa | |
| | ttggtatcagcagaaaccagatggaactgttaaactcctga | |
| | tctaccatacatcaagattacactcaggagtcccatcaagg | |
| | ttcagtggcagtgggtctggaacagattattctctcaccatta | |
| | gcaacctggagcaagaagatattgccacttacttttgccaa | |
| | cagggtaatacgcttccgtacacgttcggaggggggacc | |
| | aagctggagatcacaggctccacctctggatccggcaag | |
| | cccggatctggcgagggatccaccaagggcgaggtgaa | |
| | actgcaggagtcaggacctggcctggtggcgccctcaca | |
| | gagcctgtccgtcacatgcactgtctcaggggtctcattacc | |
| | cgactatggtgtaagctggattcgccagcctccacgaaag | |
| | ggtctggagtggctgggagtaatatggggtagtgaaacca | |
| | catactataattcagctctcaaatccagactgaccatcatca | |
| | aggacaactccaagagccaagttttcttaaaaatgaacag | |
| | tctgcaaactgatgacacagccatttactactgtgccaaac | |
| | attattactacggtggtagctatgctatggactactggggcc | |
| | aaggaacctcagtcaccgtctcctcaaccacgacgccag | |
| | cgccgcgaccaccaacaccggcgcccaccatcgcgtcg | |
| | cagcccctgtccctgcgcccagaggcgtgccggccagcg | |
| | gcggggggcgcagtgcacacgagggggctggacttcgc | |
| | ctgtgatatctacatctgggcgcccttggccgggacttgtgg | |
| | ggtccttctcctgtcactggttatcaccctttactgcaaacgg | |
| | ggcagaaagaaactcctgtatatattcaaacaaccatttat | |
| | gagaccagtacaaactactcaagaggaagatggctgtag | |
| | ctgccgatttccagaagaagaagaaggaggatgtgaact | |
| | gagagtgaagttcagcaggagcgcagacgcccccgcgt | |
| | accagcagggccagaaccagctctataacgagctcaatc | |
| | taggacgaagagaggagtacgatgttttggacaagagac | |
| | gtggccgggaccctgagatggggggaaagccgagaag | |
| | gaagaaccctcaggaaggcctgtacaatgaactgcaga | |
| | aagataagatggcggaggcctacagtgagattgggatga | |
| | aaggcgagcgccggaggggcaaggggcacgatggcct | |
| | ttaccagggtctcagtacagccaccaaggacacctacga | |
| | cgcccttcacatgcaggccctgccccctcgc | |
| 117 | MALPVTALLLPLALLLHAARPDIQMTQTTS | Exemplary CD19 |
| | SLSASLGDRVTISCRASQDISKYLNWYQQ | CAR amino acid |
| | KPDGTVKLLIYHTSRLHSGVPSRFSGSGS | sequence |
| | GTDYSLTISNLEQEDIATYFCQQGNTLPYT | |
| | FGGGTKLEITGSTSGSGKPGSGEGSTKGE | |
| | VKLQESGPGLVAPSQSLSVTCTVSGVSLP | |
| | DYGVSWIRQPPRKGLEWLGVIWGSETTY | |
| | YNSALKSRLTIIKDNSKSQVFLKMNSLQTD | |
| | DTAIYYCAKHYYYGGSYAMDYWGQGTSV | |
| | TVSSTTTPAPRPPTPAPTIASQPLSLRPEA | |
| | CRPAAGGAVHTRGLDFACDIYIWAPLAGT | |
| | CGVLLLSLVITLYCKRGRKKLLYIFKQPFM | |
| | RPVQTTQEEDGCSCRFPEEEEGGCELRV | |
| | KFSRSADAPAYQQGQNQLYNELNLGRRE | |
| | EYDVLDKRRGRDPEMGGKPRRKNPQEGL | |
| | YNELQKDKMAEAYSEIGMKGERRRGKGH | |
| | DGLYQGLSTATKDTYDALHMQALPPR | |
| 31 | atggccttaccagtgaccgccttgctcctgccgctggccttg | Tisagenlecleucel |
| | ctgctccacgccgccaggccggacatccagatgacacag | CD19 CAR |
| | actacatcctccctgtctgcctctctgggagacagagtcacc | nucleotide |
| | atcagttgcagggcaagtcaggacattagtaaatatttaaa | sequence |
| | ttggtatcagcagaaaccagatggaactgttaaactcctga | |
| | tctaccatacatcaagattacactcaggagtcccatcaagg | |
| | ttcagtggcagtgggtctggaacagattattctctcaccatta | |
| | gcaacctggagcaagaagatattgccacttacttttgccaa | |
| | cagggtaatacgcttccgtacacgttcggaggggggacc | |
| | aagctggagatcacaggtggcggtggctcgggcggtggt | |
| | gggtcgggtggcggcggatctgaggtgaaactgcaggag | |
| | tcaggacctggcctggtggcgccctcacagagcctgtccg | |
| | tcacatgcactgtctcaggggtctcattacccgactatggtgt | |
| | aagctggattcgccagcctccacgaaagggtctggagtg | |
| | gctgggagtaatatggggtagtgaaaccacatactataatt | |
| | cagctctcaaatccagactgaccatcatcaaggacaactc | |
| | caagagccaagttttcttaaaaatgaacagtctgcaaactg | |
| | atgacacagccatttactactgtgccaaacattattactacg | |
| | gtggtagctatgctatggactactggggccaaggaacctc | |
| | agtcaccgtctcctcaaccacgacgccagcgccgcgacc | |
| | accaacaccggcgcccaccatcgcgtcgcagcccctgtc | |
| | cctgcgcccagaggcgtgccggccagcggcggggggc | |
| | gcagtgcacacgagggggctggacttcgcctgtgatatct | |
| | acatctgggcgcccttggccgggacttgtggggtccttctcc | |
| | tgtcactggttatcaccctttactgcaaacggggcagaaag | |
| | aaactcctgtatatattcaaacaaccatttatgagaccagta | |
| | caaactactcaagaggaagatggctgtagctgccgatttc | |
| | cagaagaagaagaaggaggatgtgaactgagagtgaa | |
| | gttcagcaggagcgcagacgcccccgcgtacaagcagg | |
| | gccagaaccagctctataacgagctcaatctaggacgaa | |
| | gagaggagtacgatgttttggacaagagacgtggccggg | |
| | accctgagatggggggaaagccgagaaggaagaaccc | |
| | tcaggaaggcctgtacaatgaactgcagaaagataagat | |
| | ggcggaggcctacagtgagattgggatgaaaggcgagc | |
| | gccggaggggcaaggggcacgatggcctttaccagggt | |
| | ctcagtacagccaccaaggacacctacgacgcccttcac | |
| | atgcaggccctgccccctcgc | |
| 32 | MALPVTALLLPLALLLHAARPDIQMTQTTS | Tisagenlecleucel |
| | SLSASLGDRVTISCRASQDISKYLNWYQQ | CD19 CAR |
| | KPDGTVKLLIYHTSRLHSGVPSRFSGSGS | amino acid |
| | GTDYSLTISNLEQEDIATYFCQQGNTLPYT | sequence |
| | FGGGTKLEITGGGGSGGGGSGGGGSEVK | |
| | LQESGPGLVAPSQSLSVTCTVSGVSLPDY | |
| | GVSWIRQPPRKGLEWLGVIWGSETTYYN | |
| | SALKSRLTIIKDNSKSQVFLKMNSLQTDDT | |
| | AIYYCAKHYYYGGSYAMDYWGQGTSVTV | |
| | SSTTTPAPRPPTPAPTIASQPLSLRPEACR | |
| | PAAGGAVHTRGLDFACDIYIWAPLAGTCG | |
| | VLLLSLVITLYCKRGRKKLLYIFKQPFMRPV | |
| | QTTQEEDGCSCRFPEEEEGGCELRVKFS | |
| | RSADAPAYKQGQNQLYNELNLGRREEYD | |
| | VLDKRRGRDPEMGGKPRRKNPQEGLYNE | |
| | LQKDKMAEAYSEIGMKGERRRGKGHDGL | |
| | YQGLSTATKDTYDALHMQALPPR | |
| 33 | atgctgctgctggtgaccagcctgctgctgtgcgagctgcc | Lisocabtagene |
| | ccaccccgcctttctgctgatccccgacatccagatgaccc | maraleucel |
| | agaccacctccagcctgagcgccagcctgggcgaccgg | CD19 CAR |
| | gtgaccatcagctgccgggccagccaggacatcagcaa | nucleotide |
| | gtacctgaactggtatcagcagaagcccgacggcaccgt | sequence |
| | caagctgctgatctaccacaccagccggctgcacagcgg | |
| | cgtgcccagccggtttagcggcagcggctccggcaccga | |
| | ctacagcctgaccatctccaacctggaacaggaagatatc | |
| | gccacctacttttgccagcagggcaacacactgccctaca | |
| | cctttggcggcggaacaaagctggaaatcaccggcagca | |
| | cctccggcagcggcaagcctggcagcggcgagggcag | |
| | caccaagggcgaggtgaagctgcaggaaagcggccct | |
| | ggcctggtggcccccagccagagcctgagcgtgacctgc | |
| | accgtgagcggcgtgagcctgcccgactacggcgtgagc | |
| | tggatccggcagccccccaggaagggcctggaatggctg | |
| | ggcgtgatctggggcagcgagaccacctactacaacagc | |
| | gccctgaagagccggctgaccatcatcaaggacaacag | |
| | caagagccaggtgttcctgaagatgaacagcctgcagac | |
| | cgacgacaccgccatctactactgcgccaagcactactac | |
| | tacggcggcagctacgccatggactactggggccagggc | |
| | accagcgtgaccgtgagcagcgaatctaagtacggaccg | |
| | ccctgccccccttgccctatgttctgggtgctggtggtggtcg | |
| | gaggcgtgctggcctgctacagcctgctggtcaccgtggc | |
| | cttcatcatcttttgggtgaaacggggcagaaagaaactcc | |
| | tgtatatattcaaacaaccatttatgagaccagtacaaacta | |
| | ctcaagaggaagatggctgtagctgccgatttccagaaga | |
| | agaagaaggaggatgtgaactgcgggtgaagttcagca | |
| | gaagcgccgacgcccctgcctaccagcagggccagaat | |
| | cagctgtacaacgagctgaacctgggcagaagggaaga | |
| | gtacgacgtcctggataagcggagaggccgggaccctg | |
| | agatgggcggcaagcctcggcggaagaacccccagga | |
| | aggcctgtataacgaactgcagaaagacaagatggccg | |
| | aggcctacagcgagatcggcatgaagggcgagcggag | |
| | gcggggcaagggccacgacggcctgtatcagggcctgtc | |
| | caccgccaccaaggatacctacgacgccctgcacatgca | |
| | ggccctgcccccaagg | |
| 34 | MLLLVTSLLLCELPHPAFLLIPDIQMTQTTS | Lisocabtagene |
| | SLSASLGDRVTISCRASQDISKYLNWYQQ | maraleucel |
| | KPDGTVKLLIYHTSRLHSGVPSRFSGSGS | CD19 CAR |
| | GTDYSLTISNLEQEDIATYFCQQGNTLPYT | amino acid |
| | FGGGTKLEITGSTSGSGKPGSGEGSTKGE | sequence |
| | VKLQESGPGLVAPSQSLSVTCTVSGVSLP | |
| | DYGVSWIRQPPRKGLEWLGVIWGSETTY | |
| | YNSALKSRLTIIKDNSKSQVFLKMNSLQTD | |
| | DTAIYYCAKHYYYGGSYAMDYWGQGTSV | |
| | TVSSESKYGPPCPPCPMFWVLVVVGGVL | |
| | ACYSLLVTVAFIIFWVKRGRKKLLYIFKQPF | |
| | MRPVQTTQEEDGCSCRFPEEEEGGCELR | |
| | VKFSRSADAPAYQQGQNQLYNELNLGRR | |
| | EEYDVLDKRRGRDPEMGGKPRRKNPQE | |
| | GLYNELQKDKMAEAYSEIGMKGERRRGK | |
| | GHDGLYQGLSTATKDTYDALHMQALPPR | |
| 35 | atgcttctcctggtgacaagccttctgctctgtgagttaccac | Axicabtagene |
| | acccagcattcctcctgatcccagacatccagatgacaca | ciloleucel CD19 |
| | gactacatcctccctgtctgcctctctgggagacagagtca | CAR nucleotide |
| | ccatcagttgcagggcaagtcaggacattagtaaatattta | sequence |
| | aattggtatcagcagaaaccagatggaactgttaaactcct | |
| | gatctaccatacatcaagattacactcaggagtcccatcaa | |
| | ggttcagtggcagtgggtctggaacagattattctctcacca | |
| | ttagcaacctggagcaagaagatattgccacttacttttgcc | |
| | aacagggtaatacgcttccgtacacgttcggagggggga | |
| | ctaagttggaaataacaggctccacctctggatccggcaa | |
| | gcccggatctggcgagggatccaccaagggcgaggtga | |
| | aactgcaggagtcaggacctggcctggtggcgccctcac | |
| | agagcctgtccgtcacatgcactgtctcaggggtctcattac | |
| | ccgactatggtgtaagctggattcgccagcctccacgaaa | |
| | gggtctggagtggctgggagtaatatggggtagtgaaacc | |
| | acatactataattcagctctcaaatccagactgaccatcatc | |
| | aaggacaactccaagagccaagttttcttaaaaatgaaca | |
| | gtctgcaaactgatgacacagccatttactactgtgccaaa | |
| | cattattactacggtggtagctatgctatggactactggggtc | |
| | aaggaacctcagtcaccgtctcctcagcggccgcaattga | |
| | agttatgtatcctcctccttacctagacaatgagaagagca | |
| | atggaaccattatccatgtgaaagggaaacacctttgtcca | |
| | agtcccctatttcccggaccttctaagcccttttgggtgctggt | |
| | ggtggttgggggagtcctggcttgctatagcttgctagtaac | |
| | agtggcctttattattttctgggtgaggagtaagaggagcag | |
| | gctcctgcacagtgactacatgaacatgactccccgccgc | |
| | cccgggcccacccgcaagcattaccagccctatgcccca | |
| | ccacgcgacttcgcagcctatcgctccagagtgaagttca | |
| | gcaggagcgcagacgcccccgcgtaccagcagggcca | |
| | gaaccagctctataacgagctcaatctaggacgaagaga | |
| | ggagtacgatgttttggacaagagacgtggccgggaccct | |
| | gagatggggggaaagccgagaaggaagaaccctcagg | |
| | aaggcctgtacaatgaactgcagaaagataagatggcgg | |
| | aggcctacagtgagattgggatgaaaggcgagcgccgg | |
| | aggggcaaggggcacgatggcctttaccagggtctcagt | |
| | acagccaccaaggacacctacgacgcccttcacatgcag | |
| | gccctgccccctcgc | |
| 36 | MLLLVTSLLLCELPHPAFLLIPDIQMTQTTS | Axicabtagene |
| | SLSASLGDRVTISCRASQDISKYLNWYQQ | ciloleucel CD19 |
| | KPDGTVKLLIYHTSRLHSGVPSRFSGSGS | CAR amino acid |
| | GTDYSLTISNLEQEDIATYFCQQGNTLPYT | sequence |
| | FGGGTKLEITGSTSGSGKPGSGEGSTKGE | |
| | VKLQESGPGLVAPSQSLSVTCTVSGVSLP | |
| | DYGVSWIRQPPRKGLEWLGVIWGSETTY | |
| | YNSALKSRLTIIKDNSKSQVFLKMNSLQTD | |
| | DTAIYYCAKHYYYGGSYAMDYWGQGTSV | |
| | TVSSAAAIEVMYPPPYLDNEKSNGTIIHVK | |
| | GKHLCPSPLFPGPSKPFWVLVVVGGVLAC | |
| | YSLLVTVAFIIFWVRSKRSRLLHSDYMNMT | |
| | PRRPGPTRKHYQPYAPPRDFAAYRSRVK | |
| | FSRSADAPAYQQGQNQLYNELNLGRREE | |
| | YDVLDKRRGRDPEMGGKPRRKNPQEGLY | |
| | NELQKDKMAEAYSEIGMKGERRRGKGHD | |
| | GLYQGLSTATKDTYDALHMQALPPR | |

TABLE 10
Annotation of tisagenlecleucel CD19 CAR sequences

| | Nucleotide | Amino Acid |
|---|---|---|
| Feature | Sequence Position | Sequence Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 11
Annotation of lisocabtagene maraleucel CD19 CAR sequences

| | Nucleotide | Amino Acid |
|---|---|---|
| Feature | Sequence Position | Sequence Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 12
Annotation of axicabtagene ciloleucel CD19 CAR sequences

| | Nucleotide | Amino Acid |
|---|---|---|
| Feature | Sequence Position | Sequence Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

### CD20 CAR

In some embodiments, the CAR is a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkin's disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IF5, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14 (12): 1025-1033 (2001). In some embodiments, the linker is a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 13 below. In some embodiments, the CD20-specific scFv comprises or consists of an amino acid sequence set forth in SEQ ID NO:37, 38, or 42, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:37, 38, or 42. In some embodiments, the CD20-specific scFv may comprise one or more CDRs having amino acid sequences set forth in SEQ ID NOs: 39-41, 43 and 44. In some embodiments, the CD20-specific scFv may comprise a light chain with one or more CDRs having amino acid sequences set forth in SEQ ID NOs: 39-41. In some embodiments, the CD20-specific scFv may comprise a heavy chain with one or more CDRs having amino acid sequences set forth in SEQ ID NOs: 43-44. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 13
Exemplary sequences of anti-CD20 scFv and components

| SEQ ID NO: | Amino Acid Sequence | Description |
|---|---|---|
| 37 | DIVLTQSPAILSASPGEKVTMTCRA | Anti-CD20 Leu16 scFv |
| | SSSVNYMDWYQKKPGSSPKPWIYA | entire sequence, with |
| | TSNLASGVPARFSGSGSGTSYSLTI | Whitlow linker |
| | SRVEAEDAATYYCQQWSFNPPTFG | |
| | GGTKLEIKGSTSGSGKPGSGEGST | |
| | KGEVQLQQSGAELVKPGASVKMSC | |
| | KASGYTFTSYNMHWVKQTPGQGL | |
| | EWIGAIYPGNGDTSYNQKFKGKATL | |
| | TADKSSSTAYMQLSSLTSEDSADY | |
| | YCARSNYYGSSYWFFDVWGAGTT | |
| | VTVSS | |
| 38 | DIVLTQSPAILSASPGEKVTMTCRA | Anti-CD20 Leu16 scFv |
| | SSSVNYMDWYQKKPGSSPKPWIYA | light chain variable |
| | TSNLASGVPARFSGSGSGTSYSLTI | region |
| | SRVEAEDAATYYCQQWSFNPPTFG | |
| | GGTKLEIK | |
| 39 | RASSSVNYMD | Anti-CD20 Leu16 scFv |
| | | light chain CDR1 |
| 40 | ATSNLAS | Anti-CD20 Leu16 scFv |
| | | light chain CDR2 |
| 41 | QQWSFNPPT | Anti-CD20 Leu16 scFv |
| | | light chain CDR3 |
| 42 | EVQLQQSGAELVKPGASVKMSCKA | Anti-CD20 Leu16 scFv |
| | SGYTFTSYNMHWVKQTPGQGLEWI | heavy chain |
| | GAIYPGNGDTSYNQKFKGKATLTA | |
| | DKSSSTAYMQLSSLTSEDSADYYC | |
| | ARSNYYGSSYWFFDVWGAGTTVT | |
| | VSS | |
| 43 | SYNMH | Anti-CD20 Leu16 scFv |
| | | heavy chain CDR1 |
| 44 | AIYPGNGDTSYNQKFKG | Anti-CD20 Leu16 scFv |
| | | heavy chain CDR2 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO: 12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO: 12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO: 13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 15.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 18.

In some embodiments, the CD20 CAR comprises the CD20-specific scFv having sequences set forth in SEQ ID NO:37, the CD8α hinge domain of SEQ ID NO:9, the CD8α transmembrane domain of SEQ ID NO:14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CD20 CAR comprises the CD20-specific scFv having sequences set forth in SEQ ID NO:37, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CD20 CAR comprises the CD20-specific scFv having sequences set forth in SEQ ID NO:37, the IgG4 hinge domain of SEQ ID NO:11 or SEQ ID NO:12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CD20 CAR comprises the CD20-specific scFv having sequences set forth in SEQ ID NO:37, the CD8α hinge domain of SEQ ID NO:9, the CD28 transmembrane domain of SEQ ID NO:15, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CD20 CAR comprises the CD20-specific scFv having sequences set forth in SEQ ID NO:37, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CD20 CAR comprises the CD20-specific scFv having sequences set forth in SEQ ID NO:37, the IgG4 hinge domain of SEQ ID NO:11 or SEQ ID NO:1, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### CD22 CAR

In some embodiments, the CAR is a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G₄S linker. In other embodiments, the Whitlow linker may be used instead. In some embodiments, the amino acid sequences of the entire m971-derived scFv (also referred to as m971 scFv) and its different portions are provided in Table 14 below. In some embodiments, the CD22-specific scFv comprises or consists of an amino acid sequence set forth in SEQ ID NO:45, 46, or 50, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:45, 46, or 50. In some embodiments, the CD22-specific scFv may comprise one or more CDRs having amino acid sequences set forth in SEQ ID NOs: 47-49 and 51-53. In some embodiments, the CD22-specific scFv may comprise a heavy chain with one or more CDRs having amino acid sequences set forth in SEQ ID NOs: 47-49. In some embodiments, the CD22-specific scFv may comprise a light chain with one or more CDRs having amino acid sequences set forth in SEQ ID NOs: 51-53. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 14 below. In some embodiments, the CD22-specific scFv comprises or consists of an amino acid sequence set forth in SEQ ID NO: 54, 55, or 59, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:54, 55, or 59. In some embodiments, the CD22-specific scFv may comprise one or more CDRs having amino acid sequences set forth in SEQ ID NOs: 56-58 and 60-62. In some embodiments, the CD22-specific scFv may comprise a heavy chain with one or more CDRs having amino acid sequences set forth in SEQ ID NOs: 56-58. In some embodiments, the CD22-specific scFv may comprise a light chain with one or more CDRs having amino acid sequences set forth in SEQ ID NOs: 60-62. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 14
Exemplary sequences of anti-CD22 scFv and components

| SEQ ID NO: | Amino Acid Sequence | Description |
|---|---|---|
| 45 | QVQLQQSGPGLVKPSQTLSLTCAIS | Anti-CD22 m971 scFv |
| | GDSVSSNSAAWNWIRQSPSRGLE | entire sequence, with |
| | WLGRTYYRSKWYNDYAVSVKSRITI | 3xG₄S linker |
| | NPDTSKNQFSLQLNSVTPEDTAVY | |
| | YCAREVTGDLEDAFDIWGQGTMVT | |
| | VSSGGGGSGGGGSGGGGSDIQMT | |
| | QSPSSLSASVGDRVTITCRASQTIW | |
| | SYLNWYQQRPGKAPNLLIYAASSL | |
| | QSGVPSRFSGRGSGTDFTLTISSLQ | |
| | AEDFATYYCQQSYSIPQTFGQGTKL | |
| | EIK | |
| 46 | QVQLQQSGPGLVKPSQTLSLTCAIS | Anti-CD22 m971 scFv |
| | GDSVSSNSAAWNWIRQSPSRGLE | heavy chain variable |
| | WLGRTYYRSKWYNDYAVSVKSRITI | region |
| | NPDTSKNQFSLQLNSVTPEDTAVY | |
| | YCAREVTGDLEDAFDIWGQGTMVT | |
| | VSS | |
| 47 | GDSVSSNSAA | Anti-CD22 m971 scFv |
| | | heavy chain CDR1 |
| 48 | TYYRSKWYN | Anti-CD22 m971 scFv |
| | | heavy chain CDR2 |
| 49 | AREVTGDLEDAFDI | Anti-CD22 m971 scFv |
| | | heavy chain CDR3 |
| 50 | DIQMTQSPSSLSASVGDRVTITCRA | Anti-CD22 m971 scFv |
| | SQTIWSYLNWYQQRPGKAPNLLIYA | light chain |
| | ASSLQSGVPSRFSGRGSGTDFTLTI | |
| | SSLQAEDFATYYCQQSYSIPQTFG | |
| | QGTKLEIK | |
| 51 | QTIWSY | Anti-CD22 m971 scFv |
| | | light chain CDR1 |
| 52 | AAS | Anti-CD22 m971 scFv |
| | | light chain CDR2 |
| 53 | QQSYSIPQT | Anti-CD22 m971 scFv |
| | | light chain CDR3 |
| 54 | QVQLQQSGPGMVKPSQTLSLTCAI | Anti-CD22 m971-L7 |
| | SGDSVSSNSVAWNWIRQSPSRGLE | scFv entire sequence, |
| | WLGRTYYRSTWYNDYAVSMKSRIT | with 3xG₄S linker |
| | INPDTNKNQFSLQLNSVTPEDTAVY | |
| | YCAREVTGDLEDAFDIWGQGTMVT | |
| | VSSGGGGSGGGGSGGGGSDIQMI | |
| | QSPSSLSASVGDRVTITCRASQTIW | |
| | SYLNWYRQRPGEAPNLLIYAASSLQ | |
| | SGVPSRFSGRGSGTDFTLTISSLQA | |
| | EDFATYYCQQSYSIPQTFGQGTKLE | |
| | IK | |
| 55 | QVQLQQSGPGMVKPSQTLSLTCAI | Anti-CD22 m971-L7 |
| | SGDSVSSNSVAWNWIRQSPSRGLE | scFv heavy chain |
| | WLGRTYYRSTWYNDYAVSMKSRIT | variable region |
| | INPDTNKNQFSLQLNSVTPEDTAVY | |
| | YCAREVTGDLEDAFDIWGQGTMVT | |
| | VSS | |
| 56 | GDSVSSNSVA | Anti-CD22 m971-L7 |
| | | scFv heavy chain |
| | | CDR1 |
| 57 | TYYRSTWYN | Anti-CD22 m971-L7 |
| | | scFv heavy chain |
| | | CDR2 |
| 58 | AREVTGDLEDAFDI | Anti-CD22 m971-L7 |
| | | scFv heavy chain |
| | | CDR3 |
| 59 | DIQMIQSPSSLSASVGDRVTITCRA | Anti-CD22 m971-L7 |
| | SQTIWSYLNWYRQRPGEAPNLLIYA | scFv light chain |
| | ASSLQSGVPSRFSGRGSGTDFTLTI | variable region |
| | SSLQAEDFATYYCQQSYSIPQTFG | |
| | QGTKLEIK | |
| 60 | QTIWSY | Anti-CD22 m971-L7 |
| | | scFv light chain CDR1 |
| 61 | AAS | Anti-CD22 m971-L7 |
| | | scFv light chain CDR2 |
| 62 | QQSYSIPQT | Anti-CD22 m971-L7 |
| | | scFv light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280 (1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO: 12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO: 12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO: 13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 15.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 18.

In some embodiments, the CD22 CAR comprises the CD22-specific scFv having sequences set forth in SEQ ID NO:45 or SEQ ID NO:54, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CD22 CAR comprises the CD22-specific scFv having sequences set forth in SEQ ID NO:45 or SEQ ID NO:54, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CD22 CAR comprises the CD22-specific scFv having sequences set forth in SEQ ID NO:45 or SEQ ID NO:54, the IgG4 hinge domain of SEQ ID NO:11 or SEQ ID NO:12, the CD8α transmembrane domain of SEQ ID NO:14, the 4-1 BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CD22 CAR comprises the CD22-specific scFv having sequences set forth in SEQ ID NO:45 or SEQ ID NO:54, the CD8α hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CD22 CAR comprises the CD22-specific scFv having sequences set forth in SEQ ID NO:45 or SEQ ID NO:54, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CD22 CAR comprises the CD22-specific scFv having sequences set forth in SEQ ID NO:45 or SEQ ID NO:54, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### BCMA CAR

In some embodiments, the CAR is a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19 (8): 2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 15 below. In some embodiments, the BCMA-specific extracellular binding domain comprises or consists of an amino acid sequence set forth in SEQ ID NO:63, 64, or 68, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:63, 64, or 68. In some embodiments, the BCMA-specific extracellular binding domain may comprise one or more CDRs having amino acid sequences set forth in SEQ ID NOs: 65-67 and 69-71. In some embodiments, the BCMA-specific extracellular binding domain may comprise a light chain with one or more CDRs having amino acid sequences set forth in SEQ ID NOs: 65-67. In some embodiments, the BCMA-specific extracellular binding domain may comprise a heavy chain with one or more CDRs having amino acid sequences set forth in SEQ ID NOs: 69-71. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19 (8): 2048-2060 (2013) and PCT Application Publication No. WO2010/104949, the amino acid sequence of which is also provided in Table 15 below. In some embodiments, the BCMA-specific extracellular binding domain comprises or consists of an amino acid sequence set forth in SEQ ID NO:72, 73, or 77, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:72, 73, or 77. In some embodiments, the BCMA-specific extracellular binding domain may comprise one or more CDRs having amino acid sequences set forth in SEQ ID NOs: 74-76 and 78-80. In some embodiments, the BCMA-specific extracellular binding domain may comprise a light chain with one or more CDRs having amino acid sequences set forth in SEQ ID NOs: 74-76. In some embodiments, the BCMA-specific extracellular binding domain may comprise a heavy chain with one or more CDRs having amino acid sequences set forth in SEQ ID NOs: 78-80. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29 (5): 585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11 (1): 141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11 (1): 283 (2020), also referred to as FHVH33. See also, PCT Application Publication No. WO2019/006072. The amino acid sequences of FHVH33 and its CDRs are provided in Table 15 below. In some embodiments, the BCMA-specific extracellular binding domain comprises or consists of an amino acid sequence set forth in SEQ ID NO:81 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:81. In some embodiments, the BCMA-specific extracellular binding domain may comprise one or more CDRs having amino acid sequences set forth in SEQ ID NOs: 82-84. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 15 below. In some embodiments, the BCMA-specific extracellular binding domain comprises or consists of an amino acid sequence set forth in SEQ ID NO:118, 119, or 123, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 118, 119, or 123. In some embodiments, the BCMA-specific extracellular binding domain may comprise one or more CDRs having amino acid sequences set forth in SEQ ID NOs: 120-122 and 124-126. In some embodiments, the BCMA-specific extracellular binding domain may comprise a light chain with one or more CDRs having amino acid sequences set forth in SEQ ID NOs: 120-122. In some embodiments, the BCMA-specific extracellular binding domain may comprise a heavy chain with one or more CDRs having amino acid sequences set forth in SEQ ID NOs: 124-126. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 15
Exemplary sequences of anti-BCMA binder and components

| SEQ ID NO: | Amino Acid Sequence | Description |
|---|---|---|
| 63 | DIVLTQSPASLAMSLGKRATISCRA | Anti-BCMA C11D5.3 |
| | SESVSVIGAHLIHWYQQKPGQPPKL | scFv entire sequence, |
| | LIYLASNLETGVPARFSGSGSGTDF | with Whitlow linker |
| | TLTIDPVEEDDVAIYSCLQSRIFPRT | |
| | FGGGTKLEIKGSTSGSGKPGSGEG | |
| | STKGQIQLVQSGPELKKPGETVKIS | |
| | CKASGYTFTDYSINWVKRAPGKGL | |
| | KWMGWINTETREPAYAYDFRGRFA | |
| | FSLETSASTAYLQINNLKYEDTATYF | |
| | CALDYSYAMDYWGQGTSVTVSS | |
| 64 | DIVLTQSPASLAMSLGKRATISCRA | Anti-BCMA C11D5.3 |
| | SESVSVIGAHLIHWYQQKPGQPPKL | scFv light chain |
| | LIYLASNLETGVPARFSGSGSGTDF | variable region |
| | TLTIDPVEEDDVAIYSCLQSRIFPRT | |
| | FGGGTKLEIK | |
| 65 | RASESVSVIGAHLIH | Anti-BCMA C11D5.3 |
| | | scFv light chain CDR1 |
| 66 | LASNLET | Anti-BCMA C11D5.3 |
| | | scFv light chain CDR2 |
| 67 | LQSRIFPRT | Anti-BCMA C11D5.3 |
| | | scFv light chain CDR3 |
| 68 | QIQLVQSGPELKKPGETVKISCKAS | Anti-BCMA C11D5.3 |
| | GYTFTDYSINWVKRAPGKGLKWMG | scFv heavy chain |
| | WINTETREPAYAYDFRGRFAFSLET | variable region |
| | SASTAYLQINNLKYEDTATYFCALD | |
| | YSYAMDYWGQGTSVTVSS | |
| 69 | DYSIN | Anti-BCMA C11D5.3 |
| | | scFv heavy chain |
| | | CDR1 |
| 70 | WINTETREPAYAYDFRG | Anti-BCMA C11D5.3 |
| | | scFv heavy chain |
| | | CDR2 |
| 71 | DYSYAMDY | Anti-BCMA C11D5.3 |
| | | scFv heavy chain |
| | | CDR3 |
| 72 | DIVLTQSPPSLAMSLGKRATISCRA | Anti-BCMA C12A3.2 |
| | SESVTILGSHLIYWYQQKPGQPPTL | scFv entire sequence, |
| | LIQLASNVQTGVPARFSGSGSRTDF | with Whitlow linker |
| | TLTIDPVEEDDVAVYYCLQSRTIPRT | |
| | FGGGTKLEIKGSTSGSGKPGSGEG | |
| | STKGQIQLVQSGPELKKPGETVKIS | |
| | CKASGYTFRHYSMNWVKQAPGKG | |
| | LKWMGRINTESGVPIYADDFKGRFA | |
| | FSVETSASTAYLVINNLKDEDTASYF | |
| | CSNDYLYSLDFWGQGTALTVSS | |
| 73 | DIVLTQSPPSLAMSLGKRATISCRA | Anti-BCMA C12A3.2 |
| | SESVTILGSHLIYWYQQKPGQPPTL | scFv light chain |
| | LIQLASNVQTGVPARFSGSGSRTDF | variable region |
| | TLTIDPVEEDDVAVYYCLQSRTIPRT | |
| | FGGGTKLEIK | |
| 74 | RASESVTILGSHLIY | Anti-BCMA C12A3.2 |
| | | scFv light chain CDR1 |
| 75 | LASNVQT | Anti-BCMA C12A3.2 |
| | | scFv light chain CDR2 |
| 76 | LQSRTIPRT | Anti-BCMA C12A3.2 |
| | | scFv light chain CDR3 |
| 77 | QIQLVQSGPELKKPGETVKISCKAS | Anti-BCMA C12A3.2 |
| | GYTFRHYSMNWVKQAPGKGLKWM | scFv heavy chain |
| | GRINTESGVPIYADDFKGRFAFSVE | variable region |
| | TSASTAYLVINNLKDEDTASYFCSN | |
| | DYLYSLDFWGQGTALTVSS | |
| 78 | HYSMN | Anti-BCMA C12A3.2 |
| | | scFv heavy chain |
| | | CDR1 |
| 79 | RINTESGVPIYADDFKG | Anti-BCMA C12A3.2 |
| | | scFv heavy chain |
| | | CDR2 |
| 80 | DYLYSLDF | Anti-BCMA C12A3.2 |
| | | scFv heavy chain |
| | | CDR3 |
| 81 | EVQLLESGGGLVQPGGSLRLSCAA | Anti-BCMA FHVH33 |
| | SGFTFSSYAMSWVRQAPGKGLEW | entire sequence |
| | VSSISGSGDYIYYADSVKGRFTISRD | |
| | ISKNTLYLQMNSLRAEDTAVYYCAK | |
| | EGTGANSSLADYRGQGTLVTVSS | |
| 82 | GFTFSSYA | Anti-BCMA FHVH33 |
| | | CDR1 |
| 83 | ISGSGDYI | Anti-BCMA FHVH33 |
| | | CDR2 |
| 84 | AKEGTGANSSLADY | Anti-BCMA FHVH33 |
| | | CDR3 |
| 118 | DIQMTQSPSSLSASVGDRVTITCRA | Anti-BCMA CT103A |
| | SQSISSYLNWYQQKPGKAPKLLIYA | scFv entire sequence, |
| | ASSLQSGVPSRFSGSGSGTDFTLTI | with Whitlow linker |
| | SSLQPEDFATYYCQQKYDLLTFGG | |
| | GTKVEIKGSTSGSGKPGSGEGSTK | |
| | GQLQLQESGPGLVKPSETLSLTCTV | |
| | SGGSISSSSYYWGWIRQPPGKGLE | |
| | WIGSISYSGSTYYNPSLKSRVTISVD | |
| | TSKNQFSLKLSSVTAADTAVYYCAR | |
| | DRGDTILDVWGQGTMVTVSS | |
| 119 | DIQMTQSPSSLSASVGDRVTITCRA | Anti-BCMA CT103A |
| | SQSISSYLNWYQQKPGKAPKLLIYA | scFv light chain |
| | ASSLQSGVPSRFSGSGSGTDFTLTI | variable region |
| | SSLQPEDFATYYCQQKYDLLTFGG | |
| | GTKVEIK | |
| 120 | QSISSY | Anti-BCMA CT103A |
| | | scFv light chain CDR1 |
| 121 | AAS | Anti-BCMA CT103A |
| | | scFv light chain CDR2 |
| 122 | QQKYDLLT | Anti-BCMA CT103A |
| | | scFv light chain CDR3 |
| 123 | QLQLQESGPGLVKPSETLSLTCTVS | Anti-BCMA CT103A |
| | GGSISSSSYYWGWIRQPPGKGLEW | scFv heavy chain |
| | IGSISYSGSTYYNPSLKSRVTISVDT | variable region |
| | SKNQFSLKLSSVTAADTAVYYCARD | |
| | RGDTILDVWGQGTMVTVSS | |
| 124 | GGSISSSSYY | Anti-BCMA CT103A |
| | | scFv heavy chain |
| | | CDR1 |
| 125 | ISYSGST | Anti-BCMA CT103A |
| | | scFv heavy chain |
| | | CDR2 |
| 126 | ARDRGDTILDV | Anti-BCMA CT103A |
| | | scFv heavy chain |
| | | CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO: 12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO: 12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO: 13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 18.

In some embodiments, the BCMA CAR comprises any of the BCMA-specific extracellular binding domains as described, the CD8α hinge domain of SEQ ID NO:9, the CD8α transmembrane domain of SEQ ID NO:14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the BCMA CAR comprises any of the BCMA-specific extracellular binding domains as described, the CD8α hinge domain of SEQ ID NO:9, the CD8α transmembrane domain of SEQ ID NO:14, the CD28 costimulatory domain of SEQ ID NO: 17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the BCMA CAR is encoded by the nucleotide sequence set forth in SEQ ID NO:127 or a nucleotide sequence at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:127 (see Table 16). The encoded BCMA CAR has a corresponding amino acid sequence set forth in SEQ ID NO:128 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:128, with the following components: CD8α signal peptide, CT103A scFv (V_{L}-Whitlow linker-V_{H}), CD8α hinge domain, CD8α transmembrane domain, 4-1BB costimulatory domain, and CD3ζ signaling domain.

In some embodiments, the BCMA CAR is a commercially available embodiment of BCMA CAR, including, for example, idecabtagene vicleucel (ide-cel, also called bb2121). Idecabtagene vicleucel comprises a BCMA CAR with the following components: the BB2121 binder, CD8α hinge domain, CD8α transmembrane domain, 4-1BB costimulatory domain, and CD3ζ signaling domain.

TABLE 16
Exemplary sequences of BCMA CARS

| SEQ ID NO: | Sequence | Description |
|---|---|---|
| 127 | atggccttaccagtgaccgccttgctcctgccgctggcctt | Exemplary BCMA |
| | gctgctccacgccgccaggccggacatccagatgaccc | CAR nucleotide |
| | agtctccatcctccctgtctgcatctgtaggagacagagtc | sequence |
| | accatcacttgccgggcaagtcagagcattagcagctatt | |
| | taaattggtatcagcagaaaccagggaaagcccctaag | |
| | ctcctgatctatgctgcatccagtttgcaaagtggggtccc | |
| | atcaaggttcagtggcagtggatctgggacagatttcactc | |
| | tcaccatcagcagtctgcaacctgaagattttgcaacttac | |
| | tactgtcagcaaaaatacgacctcctcacttttggcggag | |
| | ggaccaaggttgagatcaaaggcagcaccagcggctc | |
| | cggcaagcctggctctggcgagggcagcacaaaggga | |
| | cagctgcagctgcaggagtcgggcccaggactggtgaa | |
| | gccttcggagaccctgtccctcacctgcactgtctctggtg | |
| | gctccatcagcagtagtagttactactggggctggatccg | |
| | ccagcccccagggaaggggctggagtggattgggagta | |
| | tctcctatagtgggagcacctactacaacccgtccctcaa | |
| | gagtcgagtcaccatatccgtagacacgtccaagaacc | |
| | agttctccctgaagctgagttctgtgaccgccgcagacac | |
| | ggcggtgtactactgcgccagagatcgtggagacaccat | |
| | actagacgtatggggtcagggtacaatggtcaccgtcag | |
| | ctcattcgtgcccgtgttcctgcccgccaaacctaccacca | |
| | cccctgcccctagacctcccaccccagccccaacaatcg | |
| | ccagccagcctctgtctctgcggcccgaagcctgtagac | |
| | ctgctgccggcggagccgtgcacaccagaggcctggac | |
| | ttcgcctgcgacatctacatctgggcccctctggccggca | |
| | cctgtggcgtgctgctgctgagcctggtgatcaccctgtact | |
| | gcaaccaccggaacaaacggggcagaaagaaactcc | |
| | tgtatatattcaaacaaccatttatgagaccagtacaaact | |
| | actcaagaggaagatggctgtagctgccgatttccagaa | |
| | gaagaagaaggaggatgtgaactgagagtgaagttca | |
| | gcagatccgccgacgcccctgcctaccagcagggaca | |
| | gaaccagctgtacaacgagctgaacctgggcagacgg | |
| | gaagagtacgacgtgctggacaagcggagaggccgg | |
| | gaccccgagatgggcggaaagcccagacggaagaac | |
| | ccccaggaaggcctgtataacgaactgcagaaagaca | |
| | agatggccgaggcctacagcgagatcggcatgaaggg | |
| | cgagcggaggcgcggcaagggccacgatggcctgtac | |
| | cagggcctgagcaccgccaccaaggacacctacgacg | |
| | ccctgcacatgcaggccctgccccccaga | |
| 128 | MALPVTALLLPLALLLHAARPDIQMTQSP | Exemplary BCMA |
| | SSLSASVGDRVTITCRASQSISSYLNWYQ | CAR amino acid |
| | QKPGKAPKLLIYAASSLQSGVPSRFSGSG | sequence |
| | SGTDFTLTISSLQPEDFATYYCQQKYDLL | |
| | TFGGGTKVEIKGSTSGSGKPGSGEGSTK | |
| | GQLQLQESGPGLVKPSETLSLTCTVSGG | |
| | SISSSSYYWGWIRQPPGKGLEWIGSISYS | |
| | GSTYYNPSLKSRVTISVDTSKNQFSLKLS | |
| | SVTAADTAVYYCARDRGDTILDVWGQGT | |
| | MVTVSSFVPVFLPAKPTTTPAPRPPTPAP | |
| | TIASQPLSLRPEACRPAAGGAVHTRGLDF | |
| | ACDIYIWAPLAGTCGVLLLSLVITLYCNHR | |
| | NKRGRKKLLYIFKQPFMRPVQTTQEEDG | |
| | CSCRFPEEEEGGCELRVKFSRSADAPAY | |
| | QQGQNQLYNELNLGRREEYDVLDKRRG | |
| | RDPEMGGKPRRKNPQEGLYNELQKDKM | |
| | AEAYSEIGMKGERRRGKGHDGLYQGLST | |
| | ATKDTYDALHMQALPPR | |

In some embodiments, the primary T cells or the pool of primary T cells are engineered to exhibit reduced expression of an endogenous T cell receptor compared to unmodified primary T cells. In certain embodiments, the primary T cells or the pool of primary T cells are engineered to exhibit reduced expression of CTLA4, PD1, or both CTLA4 and PD1, as compared to unmodified primary T cells. Methods of genetically modifying a cell including a T cell are described in detail, for example, in WO2016183041, the disclosure is herein incorporated by reference in its entirety including the tables, appendices, sequence listing and figures.

In some embodiments, after the engineered cells described herein are administered to the recipient subject or patient, the cells undergo inappropriate expansion or proliferation in the recipient; present in an inappropriate location in the recipient's body; or undergo a malignant transformation. In certain embodiments, such engineered cells induce cytokine release syndrome, induce neurotoxicity; or induce toxicity such as on-target off tumor toxicity in the recipient. And after which, the recipient subject is administered an agent that blocks, neutralizes, inactivates, interferes with CD47- and SIRPα-binding, signaling, activity and function.

Without wishing to be bound by theory, it is believed that the modifications of the engineered cells “cloak” them from the recipient immune system's effector cells that are responsible for the clearance of infected, malignant or non-self cells. “Cloaking” of a cell from the immune system allows for existence and persistence of specific cells, e.g., allogeneic cells within the body. In some instances, engineered cells described herein may no longer be therapeutically effective or may induce undesired adverse effects in the recipient. Non-limiting examples of an adverse event include hyperproliferation, transformation, tumor formation, cytokine release syndrome, GVHD, immune effector cell-associated neurotoxicity syndrome (ICANS), inflammation, infection, nausea, vomiting, bleeding, interstitial pneumonitis, respiratory disease, jaundice, weight loss, diarrhea, loss of appetite, cramps, abdominal pain, hepatic veno-occlusive disease (VOD), graft failure, organ damage, infertility, hormonal changes, abnormal growth formation, cataracts, and post-transplant lymphoproliferative disorder (PTLD), and the like. As such, the ability to control the presence of the engineered cells in the recipient's body is crucial for safety. As such, “uncloaking” the cells serves as a safety switch and can be achieved by blocking or neutralizing the function of an immunosuppressive factor, such as CD47.

In some embodiments, upon contacting the cells with a CD47-SIRPα blockade agent, the cells are recognized by the recipient's immune system. In some embodiments, the engineered cells express the immunosuppressive factor CD47 such that the cells are hypoimmunogenic or have reduced immunogenicity until one or more CD47-SIRPα blockade agents are administered to the recipient. In the presence of a CD47-SIRPα blockade agent, the cells are uncloaked and are recognized by immune cells to be targeted by cell death or clearance.

1. Modifying Expression of MHC Class I and/or MHC Class II Complexes

Provided herein are cells comprising an exogenous CD47 protein and a modification of one or more targeted polynucleotide sequences that regulate the expression of MHC I human leukocyte antigens and/or MHC II human leukocyte antigens. In some embodiments, the expression of MHC I human leukocyte antigens or MHC II human leukocyte antigens is modulated. In some embodiments, the expression of MHC I human leukocyte antigens and MHC II human leukocyte antigens is modulated. In some embodiments, the cells are genetically modified to reduce or inactivate expression of the MHC class I complex, to reduce or inactivate expression of the MHC class II complex, to prevent direct recognition by CD8 T cells of the recipient subject, and/or to evade NK cell recognition by the recipient subject. In some embodiments, the cells exhibit reduce immunogenicity. Detailed descriptions of genetically modifying cells is found, e.g., in WO2016183041, the disclosure is incorporated herein in its entirety, including the sequence listing, tables, and figures.

In some embodiments, the engineered cell comprises a genomic modification of one or more targeted polynucleotide sequences that regulates the expression of MHC I proteins and MHC II proteins. In certain embodiments, the cell comprises a genomic modification of one or more targeted polynucleotide sequences that regulates the expression of MHC I proteins or MHC II proteins. In some aspects, a genetic editing system is used to modify one or more targeted polynucleotide sequences. In some embodiments, the targeted polynucleotide sequence is one or more selected from a group that includes B2M, CIITA, and NLRC5. In certain embodiments, the genome of the cell has been altered to reduce or delete critical components of HLA expression. In some embodiments, the genetic modification comprises an inactivating mutation (e.g., deletion, addition or substitution).

In some embodiments, the engineered cell comprises genetic modifications in genes selected from one or more from a group that includes B2M, CIITA, NLRC5, B7-1, B7-2, B7-H3, CD27, CD28, CD47, GITR, HLA-A, HLA-B, HLA-C, HLA-E, HLA-F, HLA-G, IRF1, NFY-A, NFY-B, PD-L1, PD-L2, NFY-C, OX40, RFX5, RFX-ANK, RFX-AP, TAP1, HVEM, SLAM, LFA-1, ST2, CD2, CD30, CD58, CD74, CD160, CD226, CD244, 4-1BB, BTLA, ICOS, LAG3, HELIOS, TIGIT, TIM3, TLT, VISTA, and ligands of NKG2D. In some embodiments, ligands of NKG2D are selected from one or more of a group that includes MICA, MICB, Raetl e, Raetl g, Raetl l, UlbpI, Ulbp2, and Ulbp3.

In some aspects, the present disclosure provides a cell or population thereof comprising a genome in which a gene has been edited to delete a contiguous stretch of genomic DNA, thereby reducing or eliminating surface expression of MHC class I molecules in the cell or population thereof. In certain aspects, the present disclosure provides a cell or population thereof comprising a genome in which a gene has been edited to delete a contiguous stretch of genomic DNA, thereby reducing or eliminating surface expression of MHC class II molecules in the cell or population thereof. In particular aspects, the present disclosure provides a cell or population thereof comprising a genome in which one or more genes has been edited to delete a contiguous stretch of genomic DNA, thereby reducing or eliminating surface expression of MHC class I and/or MHC class II molecules in the cell or population thereof.

In certain embodiments, the expression of MHC I or MHC II molecules is modulated by targeting and deleting a contiguous stretch of genomic DNA thereby reducing or eliminating expression of a target gene selected from a group that includes, but is not limited to, B2M, CIITA, and NLRC5.

In some embodiments, the cells and methods described herein include genomically editing human cells to cleave CIITA gene sequences as well as editing the genome of such cells to alter one or more additional target polynucleotide sequences such as, but not limited to, B2M and NLRC5. In some embodiments, the cells and methods described herein include genomically editing human cells to cleave B2M gene sequences as well as editing the genome of such cells to alter one or more additional target polynucleotide sequences such as, but not limited to, CIITA and NLRC5. In some embodiments, the cells and methods described herein include genomically editing human cells to cleave NLRC5 gene sequences as well as editing the genome of such cells to alter one or more additional target polynucleotide sequences such as, but not limited to, B2M and CIITA.

i. CIITA

In some aspects, the present technologies disclosed herein modulate (e.g., reduce, decrease or eliminate) expression of MHC II genes by targeting and modulating (e.g., reducing, decreasing or eliminating) Class II transactivator (CIITA) expression. In some aspects, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence described herein is a variant of CIITA. In some embodiments, the target polynucleotide sequence is a homolog of CIITA. In some embodiments, the target polynucleotide sequence is an ortholog of CIITA.

In some aspects, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the hypoimmunogenic cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from a group that includes SEQ ID NOS: 5184-36352 of Table 12 of WO2016/183041. In some embodiments, the cell has a reduced ability to induce an immune response in a recipient subject.

Assays to test whether the CIITA gene has been inactivated are known and described herein. In one embodiment, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification.

ii. B2M

In certain embodiments, the present method described modulates (e.g., reduce, decrease or eliminate) the expression of MHC-I genes by targeting and modulating (e.g., reducing, decreasing or eliminating) expression of the accessory chain B2M. In some aspects, the modulation occurs using a CRISPR/Cas system. By modulating (e.g., reducing, decreasing or deleting) expression of B2M, surface trafficking of MHC-I molecules is blocked and the cell rendered hypoimmunogenic. In some embodiments, the cell has a reduced ability to induce an immune response in a recipient subject.

In some embodiments, the target polynucleotide sequence disclosed herein is a variant of B2M. In some embodiments, the target polynucleotide sequence is a homolog of B2M. In some embodiments, the target polynucleotide sequence is an ortholog of B2M.

In some aspects, decreased or eliminated expression of B2M reduces or eliminates expression of one or more of the following MHC I molecules-HLA-A, HLA-B, and HLA-C.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from a group that includes SEQ ID NOS: 81240-85644 of Table 15 of WO2016/183041.

Assays to test whether the B2M gene has been inactivated are known and described herein. In one embodiment, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

iii. NLRC5

In certain aspects, the methods disclosed herein modulate (e.g., reduce, decrease or eliminate) the expression of MHC-I genes by targeting and modulating (e.g., reducing, decreasing or eliminating) expression of the NLR family, CARD domain containing 5/NOD27/CLR16.1 (NLRC5). In some aspects, the modulation occurs using a CRISPR/Cas system. NLRC5 is a critical regulator of MHC-I-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-I antigen presentation.

In some embodiments, the target polynucleotide sequence described herein is a variant of NLRC5. In some embodiments, the target polynucleotide sequence is a homolog of NLRC5. In some embodiments, the target polynucleotide sequence is an ortholog of NLRC5.

In some aspects, decreased or eliminated expression of NLRC5 reduces or eliminates expression of one or more of the following MHC I molecules-HLA-A, HLA-B, and HLA-C.

In some embodiments, the hypoimmunogenic cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from a group that includes SEQ ID NOS: 36353-81239 of Table 14 of WO2016/183041. In some embodiments, the cell has a reduced ability to induce an immune response in a recipient subject.

Assays to test whether the NLRC5 gene has been inactivated are known and described herein. In one embodiment, the resulting genetic modification of the NLRC5 gene by PCR and the reduction of HLA-I expression can be assays by FACS analysis. In another embodiment, NLRC5 protein expression is detected using a Western blot of cells lysates probed with antibodies to the NLRC5 protein. In another embodiment, reverse transcriptase polymerase chain reactions (RT-PCR) are used to confirm the presence of the inactivating genetic modification.

In some embodiments, the cells described include a modification to modulate expression of one selected from a group that includes CD24, CD27, CD200, HLA-C, HLA-E, HLA-E heavy chain, HLA-G, PD-L1, IDO1, CTLA4-Ig, C1-Inhibitor, IL-10, IL-35, FASL, Serpinb9, CCL21, and Mfge8. In certain instances, the cell overexpress one or more genes or proteins selected from a group that includes CD24, CD27, CD200, HLA-C, HLA-E, HLA-E heavy chain, HLA-G, PD-L1, IDO1, CTLA4-Ig, C1-Inhibitor, IL-10, IL-35, FASL, Serpinb9, CCL21, and Mfge8. In certain instances, the cell are modified to exhibit reduced expression of one or more genes or proteins selected from a group that includes CD24, CD27, CD200, HLA-C, HLA-E, HLA-E heavy chain, HLA-G, PD-L1, IDO1, CTLA4-Ig, C1-Inhibitor, IL-10, IL-35, FASL, Serpinb9, CCL21, and Mfge8.

In some embodiments, the cells described comprise exogenously expressed CD47 polypeptides and reduced expression of MHC class I molecules. In certain embodiments, the cells described comprise exogenously expressed CD47 polypeptides and reduced expression of MHC class II molecules. In yet other embodiments, the cells described comprise exogenously expressed CD47 polypeptides and reduced expression of MHC class I and MHC class I molecules.

In some embodiments, the cells described comprise exogenously expressed CD47 polypeptides and reduced expression of B2M. In certain embodiments, the cells described comprise exogenously expressed CD47 polypeptides and reduced expression of CIITA In yet other embodiments, the cells described comprise exogenously expressed CD47 polypeptides and reduced expression of B2M and CIITA.

In some embodiments, the cells described comprise exogenously expressed CD47 polypeptides and one or more additional exogenously expressed polypeptides selected from the group that includes CD24, CD46, CD55, CD59, CD200, PD-L1, IDO1, HLA-G, FasL, CCL21, Mfge8, Serpin B9, and any combination thereof. In some embodiments, the cells described comprise exogenously expressed CD47 polypeptides; one or more additional exogenously expressed polypeptides selected from the group that includes CD24, CD46, CD55, CD59, CD200, PD-L1, IDO1, HLA-G, FasL, CCL21, Mfge8, Serpin B9, and any combination thereof; and reduced expression of MHC class I molecules. In some embodiments, the cells described comprise exogenously expressed CD47 polypeptides; one or more additional exogenously expressed polypeptides selected from the group that includes CD24, CD46, CD55, CD59, CD200, PD-L1, IDO1, HLA-G, FasL, CCL21, Mfge8, Serpin B9, and any combination thereof; and reduced expression of MHC class II molecules. In some embodiments, the cells described comprise exogenously expressed CD47 polypeptides; one or more additional exogenously expressed polypeptides selected from the group that includes CD24, CD46, CD55, CD59, CD200, PD-L1, IDO1, HLA-G, FasL, CCL21, Mfge8, Serpin B9, and any combination thereof; and reduced expression of MHC class I and MHC class II molecules.

In some embodiments, the cells described comprise exogenously expressed CD47 polypeptides; one or more additional exogenously expressed polypeptides selected from the group that includes CD24, CD46, CD55, CD59, CD200, PD-L1, IDO1, HLA-G, FasL, CCL21, Mfge8, Serpin B9, and any combination thereof; and reduced expression of B2M. In some embodiments, the cells described comprise exogenously expressed CD47 polypeptides; one or more additional exogenously expressed polypeptides selected from the group that includes CD24, CD46, CD55, CD59, CD200, PD-L1, IDO1, HLA-G, FasL, CCL21, Mfge8, Serpin B9, and any combination thereof; and reduced expression of CIITA. In some embodiments, the cells described comprise exogenously expressed CD47 polypeptides; one or more additional exogenously expressed polypeptides selected from the group that includes CD24, CD46, CD55, CD59, CD200, PD-L1, IDO1, HLA-G, FasL, CCL21, Mfge8, Serpin B9, and any combination thereof; and reduced expression of NLRC5. In some embodiments, the cells described comprise exogenously expressed CD47 polypeptides; one or more additional exogenously expressed polypeptides selected from the group that includes CD24, CD46, CD55, CD59, CD200, PD-L1, IDO1, HLA-G, FasL, CCL21, Mfge8, Serpin B9, and any combination thereof; and reduced expression of any of a group that includes: (a) B2M and CIITA; (b) B2M and NLRC5; (c) CIITA and NLRC5; and (d) B2M, CIITA and NLRC5.

### 2. Modifying Expression of TCR Complexes

Provided herein are cells comprising an exogenous CD47 protein and a modification of one or more targeted polynucleotide sequences that regulate the expression of TCR complexes. In some embodiments, the expression of TCRα proteins or TCRβ proteins is modulated. In some embodiments, the expression of TCRα proteins and TCRβ proteins is modulated. In some embodiments, the cells are genetically modified to reduce or inactivate expression of one or more TCR complexes, to reduce or inactivate expression of TCRα, to reduce or inactivate expression of TCRβ, and/or to reduce immunogenicity. Detailed descriptions of genetically modifying cells is found, e.g., in WO2016183041, the disclosure of which is incorporated herein in its entirety, including the sequence listing, tables, and figures.

In some embodiments, the engineered cell comprises a genomic modification of one or more targeted polynucleotide sequences that regulates the expression of TCRα proteins and TCRβ proteins. In certain embodiments, the cell comprises a genomic modification of one or more targeted polynucleotide sequences that regulates the expression of TCRα proteins or TCRβ proteins. In some aspects, a genetic editing system is used to modify one or more targeted polynucleotide sequences. In some embodiments, the targeted polynucleotide sequence is one or more selected from a group that includes TRAC and TRB. In certain embodiments, the genome of the cell has been altered to reduce or delete critical components of TCR expression such that surface expression of one or more TCR complexes is altered. In some embodiments, the genetic modification comprises an inactivating mutation (e.g., deletion, addition or substitution).

i. TRAC

In certain embodiments, the technologies disclosed herein modulate (e.g., reduce or eliminate) the expression of TCR genes, including the TRAC gene, by targeting and modulating (e.g., reducing or eliminating) expression of the constant region of the T cell receptor alpha chain. In some aspects, the modulation occurs using a CRISPR/Cas system. By modulating (e.g., reducing or deleting) expression of TRAC, surface trafficking of TCR molecules is blocked in a cell modulated in accordance with technologies disclosed herein. In some embodiments, a cell whose genome has been engineered to modulate expression of TCR genes, including a TRAC gene, also has a reduced ability to induce an immune response in a recipient subject. In some embodiments, expression of one or more TCR complexes is altered in a cell with modulated expression of the TRAC gene.

In some embodiments, the target polynucleotide sequence of the present technology is a variant of TRAC. In some embodiments, the target polynucleotide sequence is a homolog of TRAC. In some embodiments, the target polynucleotide sequence is an ortholog of TRAC.

In some aspects, decreased or eliminated expression of TRAC reduces or eliminates TCR surface expression. As such, expression of one or more TCR complexes is decreased compared to expression in an unmodified cell.

In some embodiments, the cells described herein comprise gene modifications at the gene locus encoding the TRAC protein. In other words, the cells comprise a genetic modification at the TRAC locus. In some instances, the nucleotide sequence encoding the TRAC protein is set forth in Genbank No. X02592.1. In some instances, the TRAC gene locus is described in Ref Seq. No. NG_001332.3 and NCBI Gene ID No. 28755. In certain cases, the amino acid sequence of TRAC is depicted as Uniprot No. P01848. Additional descriptions of the TRAC protein and gene locus can be found in Uniprot No. P01848, HGNC Ref. No. 12029, and OMIM Ref. No. 186880.

In some embodiments, the hypoimmunogenic cells outlined herein comprise a genetic modification targeting the TRAC gene. In some embodiments, the genetic modification targeting the TRAC gene by the rare-cutting endonuclease comprises a Cas protein or a polynucleotide encoding a Cas protein, and at least one guide ribonucleic acid sequence for specifically targeting the TRAC gene. In some embodiments, the at least one guide ribonucleic acid sequence for specifically targeting the TRAC gene is selected from the group consisting of SEQ ID NOS: 532-609 and 9102-9797 of US20160348073, which is herein incorporated by reference.

Assays to test whether the TRAC gene has been inactivated are known and described herein. In one embodiment, the resulting genetic modification of the TRAC gene by PCR and the reduction of TCR expression can be assays by FACS analysis. In another embodiment, TCRα protein expression is detected using a Western blot of cells lysates probed with antibodies to the TCRα protein. In another embodiment, reverse transcriptase polymerase chain reactions (RT-PCR) are used to confirm the presence of the inactivating genetic modification.

ii. TRB

In certain embodiments, the technologies disclosed herein modulate (e.g., reduce or eliminate) the expression of TCR genes including the gene encoding T cell antigen receptor, beta chain (e.g., the TRB or TCRβ gene) by targeting and modulating (e.g., reducing or eliminating) expression of the constant region of the T cell receptor beta chain. In some aspects, the modulation occurs using a CRISPR/Cas system. By modulating (e.g., reducing or deleting) expression of TRB, surface trafficking of TCR molecules is blocked in a cell modulated in accordance with technologies disclosed herein. In some embodiments, a cell whose genome has been engineered to modulate expression of TCR genes, including a TRB gene, also has a reduced ability to induce an immune response in a recipient subject. In some embodiments, expression of one or more TCR complexes is altered in a cell with modulated expression of the TRB gene.

In some embodiments, the target polynucleotide sequence of the present technology is a variant of TRB. In some embodiments, the target polynucleotide sequence is a homolog of TRB. In some embodiments, the target polynucleotide sequence is an ortholog of TRB.

In some aspects, decreased or eliminated expression of TRB reduces or eliminates TCR surface expression. As such, expression of one or more TCR complexes is decreased compared to expression in an unmodified cell.

In some embodiments, the cells described herein comprise gene modifications at the gene locus encoding the TRB protein. In other words, the cells comprise a genetic modification at the TRB locus. In some instances, the nucleotide sequence encoding the TRB protein is set forth in UniProt No. PODSE2. In some instances, the TRB gene locus is described in Ref Seq. No. NG_001333.2 and NCBI Gene ID No. 6957. In certain cases, the amino acid sequence of TRB is depicted as Uniprot No. P01848. Additional descriptions of the TRB protein and gene locus can be found in GenBank No. L36092.2, Uniprot No. PODSE2, and HGNC Ref. No. 12155.

In some embodiments, the hypoimmunogenic cells outlined herein comprise a genetic modification targeting the TRB gene. In some embodiments, the genetic modification targeting the TRB gene by the rare-cutting endonuclease comprises a Cas protein or a polynucleotide encoding a Cas protein, and at least one guide ribonucleic acid sequence for specifically targeting the TRB gene. In some embodiments, the at least one guide ribonucleic acid sequence for specifically targeting the TRB gene is selected from the group consisting of SEQ ID NOS: 610-765 and 9798-10532 of US20160348073, which is herein incorporated by reference.

Assays to test whether the TRB gene has been inactivated are known and described herein. In one embodiment, the resulting genetic modification of the TRB gene by PCR and the reduction of TCR expression can be assays by FACS analysis. In another embodiment, TCRβ protein expression is detected using a Western blot of cells lysates probed with antibodies to the TCRβ protein. In another embodiment, reverse transcriptase polymerase chain reactions (RT-PCR) are used to confirm the presence of the inactivating genetic modification.

Provided herein are cells that are engineered for reduced expression or lack of expression of MHC class I and/or MHC class II human leukocyte antigens, reduced expression or lack of expression of TCR complexes, and increased expression of CD47, compared to corresponding wild-type or unmodified cells. In some embodiments, the engineered cells also express a chimeric antigen receptor (CAR). In some instances, the CAR is a CD19-specific CAR. In some embodiments, the CD19-specific CAR exhibits a substantially similar structure and/or function to the CAR expressed in cells of tisagenlecleucel or a biosimilar or surrogate thereof. In some aspects, the cells include a genetically modification into one or more genes selected from the group consisting of B2M, CIITA, TRAC and TRB genes. In some embodiments, the genetic modifications are introduced into the B2M and CIITA genes. In some embodiments, the genetic modifications are introduced into the B2M, CIITA, and TRAC genes. In some embodiments, the genetic modifications are introduced into the B2M, CIITA, and TRB genes. In some embodiments, the genetic modifications are introduced into the B2M, CIITA, TRAC, and TRB genes. In some embodiments, the cells are B2M^{-/-}, CIITA^{-/-}, TRAC/cells. In some embodiments, the cells are B2M^{-/-}, CIITA^{-/-}, TRB^{-/-} cells. In some embodiments, the cells are B2M^{-/-}, CIITA^{-/-}, TRAC^{-/-}, TRB^{-/-} cells.

In many aspects, the cells include a genetically modification into one or more genes selected from the group consisting of B2M, CIITA, TRAC and TRB genes and overexpress CD47. In some embodiments, the cells overexpress CD47 and carry genetic modifications introduced into the B2M and CIITA genes. In some embodiments, the cells overexpress CD47 and carry genetic modifications introduced into the B2M, CIITA, and TRAC genes. In some embodiments, the cells overexpress CD47 and carry genetic modifications introduced into the B2M, CIITA, and TRB genes. In some embodiments, the cells overexpress CD47 and carry genetic modifications introduced into the B2M, CIITA, TRAC, and TRB genes. In some embodiments, the cells are B2M^{-/-}, CIITA^{-/-}, TRAC^{-/-}, CD47tg cells. In some embodiments, the cells are B2M^{-/-}, CIITA^{-/-}, TRB^{-/-}, CD47tg cells. In some embodiments, the cells are B2M^{-/-}, CIITA^{-/-}, TRAC^{-/-}, TRB^{-/-}, CD47tg cells. In some embodiments, expression of exogenous CD47 by the cells described is controlled by a constitutive promoter or an inducible promoter.

In many aspects, the cells include a genetically modification into one or more genes selected from the group consisting of B2M, CIITA, TRAC and TRB genes and overexpress CD47 and a CAR. In some embodiments, the cells overexpress CD47 and a CAR, and carry genetic modifications introduced into the B2M and CIITA genes. In some embodiments, the cells overexpress CD47 and a CAR, and carry genetic modifications introduced into the B2M, CIITA, and TRAC genes. In some embodiments, the cells overexpress CD47 and a CAR, and carry genetic modifications introduced into the B2M, CIITA, and TRB genes. In some embodiments, the cells overexpress CD47 and a CAR, and carry genetic modifications introduced into the B2M, CIITA, TRAC, and TRB genes. In some embodiments, the cells are B2M^{-/-}, CIITA^{-/-}, TRAC^{-/-}, CD47tg cells that also express chimeric antigen receptors. In some embodiments, the cells are B2M^{-/-}, CIITA^{-/-}, TRB^{-/-}, CD47tg cells that also express chimeric antigen receptors. In some embodiments, the cells are B2M^{-/-}, CIITA^{-/-}, TRAC^{-/-}, TRB^{-/-}, CD47tg cells that also express chimeric antigen receptors.

In some embodiments, expression of the CAR is controlled by a constitutive promoter or an inducible promoter. In some embodiments, expression of exogenous CD47 by the cells described is controlled by a constitutive promoter or an inducible promoter. In certain embodiments, expression of the CAR and CD47 are controlled by a single promoter. In many embodiments, expression of the CAR and CD47 are controlled by two promoters. In some embodiments, expression of the CAR is controlled by a first promoter and CD47 is controlled by a second promoter, such that the first promoter and the second promoter are the same type of promoter. In other instances, the first promoter and the second promoter are different types of promoters. In some instances, the expression level of the CAR by the cell is higher (e.g., 5%, 10%, 25%, 50%, 75%, 100%, 200%, 300% or more higher) than the expression of CD47. In some instances, the expression level of CD47 (e.g., exogenous CD47) by the cell is higher (e.g., 5%, 10%, 25%, 50%, 75%, 100%, 200%, 300% or more higher) than the expression of the CAR. In many instances, the expression levels of the CAR and the exogenous CD47 are substantially the same.

### III. Primary Cells: Generation, Engineering, Differentiation, Transplantation

Provided herein are methods and compositions for modulating a population of cells, including primary cells and non-primary cells, containing one or more nucleic acids encoding CD47, and optionally, other proteins, comprising administering to a subject a CD47-SIRPα blockade agent, wherein the population of cells was previously administered to or transplanted into the subject. In some embodiments, primary cells comprise cells that can be differentiated into other non-primary cell types. In some embodiments, primary cells are pluripotent. In some embodiments, primary cells comprise pluripotent stem cells. In some embodiments, primary cells are human primary cells. In some embodiments, human primary cells are human pluripotent stem cells (hPSCs). In some embodiments, non-primary cells are human non-primary cells.

Therapeutic cells including pluripotent stem cells, differentiated cells, primary cells, and primary T cells, can be engineered to express immune regulator proteins and evade rejection by a recipient's immune system. And thus, such cells hold significant promise for allogenic cell therapy. In some aspects, cells of the present technology comprises immunosuppressive (e.g., immunogenicity) factors that function to suppress the recipient's immune response to the engrafted cells. In some embodiments, administration of a CD47-SIRPα blockade agent to the recipient facilitates phagocytosis, cell clearance and/or cell death of these cells and derivatives thereof (e.g., progeny cells). In some aspects, the CD47-SIRPα blockade agent is an agent that neutralizes, blocks, antagonizes, or interferes with the cell surface expression of CD47, SIRPα, or both. In some embodiments, the CD47-SIRPα blockade agent inhibits or blocks the interaction of CD47, SIRPα or both. Such CD47-SIRPα blockade agents are useful as safety switches to modulate the activity of administered or engrafted cells, thereby improving the safety of these cell-based therapies.

Provided herein are methods of using CD47-SIRPα blockade agent to reduce the number of cells expressing CD47 (e.g., CD47 expressing cells that have been administered or introduced to a patient). Also provided are cells and derivatives thereof (e.g., pluripotent stem cells, induced pluripotent stem cell, differentiated cell from a pluripotent stem cell, primary T cells, and progeny thereof) expressing CD47. In some embodiments, the cells comprise exogenously expressed CD47. In some embodiments, the engineered cells described herein are administered to a recipient subject, and afterwards those engineered cells are targeted for cell death and/or cell clearance by the recipient subject's immune system upon administered of a CD47-SIRPα blockade agent to the subject.

In some embodiments, cells outlined herein are subject to an innate immune cell rejection after the recipient subject is administered a CD47-SIRPα blockade agent. In some instances, the cells expressing an immunosuppressive factor (e.g., CD47) are not susceptible to NK cell-mediated lysis prior to administration of the CD47-SIRPα blockade agent. In some instances, cells are not susceptible to macrophage engulfment prior to administration of the CD47-SIRPα blockade agent. In some embodiments, the cells are useful as a source of universally compatible cells or tissues (e.g., universal donor cells or tissues) that can be transplanted into a recipient subject with little to no immunosuppressant agent needed. Such cells retain cell-specific characteristics and features upon transplantation.

In some embodiments, provided herein are cells and/or differentiated derivatives thereof that evade immune rejection in an MHC-mismatched allogenic recipient. In some instances, cells expressing CD47 and progeny thereof including the engrafted cells expressing CD47 and any progeny (e.g., direct or indirect progeny of the cells) can evade immune recognition by a recipient subject. In some embodiments, the cells and/or differentiated cells derived from such cells are hypoimmunogenic. As such, the cells and progeny thereof can evade immune recognition and do not elicit an immune response in the recipient subject. In some embodiments, differentiated cells produced from the stem cells outlined herein evade immune rejection when administered (e.g., transplanted or grafted) to MHC-mismatched allogenic recipient. In some embodiments, the cells and/or differentiated cells derived from such cells are hypoimmunogenic.

A. Generation of Primary and/or Differentiated Cells

The present disclosure provides methods of producing engineered cells comprising exogenously expressed CD47. In some embodiments, the cells comprise pluripotent stem cells, induced pluripotent stem cells, differentiated cells, and cells derived from primary T cells. In some embodiments, the differentiated cells comprise a cell type selected from a group that includes cardiac cells, neural cells, endothelial cells, T cells, pancreatic islet cells, retinal pigmented epithelium cells, hepatocytes, thyroid cells, skin cells, blood cells, and epithelial cells. In some embodiments, the engineered cells or the progeny thereof are cells of any organ or tissue of the body including, but not limited to, the heart, brain, skin, eye, pancreas, bladder, spleen, liver, lung, kidney, thyroid, cardiovascular system, respiratory system, nervous system, and immune system. In some embodiments, the pluripotent stem cells are differentiated into cells of any organ or tissue of the body using a specific differentiation condition.

In some embodiments, the methods described herein comprise primary cells that are produced using methods known by those skilled in the art. In some embodiments, the method described herein comprises pluripotent stem cells that are produced using method known by those skilled in the art. The generation of mouse and human induced pluripotent stem cells (generally referred to as iPSCs; miPSCs for murine cells or hiPSCs for human cells) is generally known in the art. As will be appreciated by those in the art, there are a variety of different methods for the generation of iPSCs. The original induction was done from mouse embryonic or adult fibroblasts using the viral introduction of four transcription factors, Oct3/4, Sox2, c-Myc and Klf4; see Takahashi and Yamanaka, Cell, 126:663-676 (2006), hereby incorporated by reference in its entirety and specifically for the techniques outlined therein. Since then, a number of methods have been developed; see Seki et al, World J. Stem Cells 7 (1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more “reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In general, as is known in the art, iPSCs are made from non-pluripotent cells such as, but not limited to, blood cells, fibroblasts, etc., by transiently expressing the reprogramming factors as described herein.

Once the hypoimmunogenic pluripotent stem cells have been generated, they can be maintained an undifferentiated state as is known for maintaining iPSCs. For example, the cells can be cultured on Matrigel using culture media that prevents differentiation and maintains pluripotency. In addition, they can be in culture medium under conditions to maintain pluripotency.

B. Engineering of Primary and/or Differentiated Cells

Methods provided are useful for inactivation or ablation of one or more genes in cells such as but not limited to pluripotent stem cells, differentiated cells thereof, primary T cells, and the like. In some embodiments, the engineered cells comprise a genetic modification to reduce or eliminate surface expression of any component of the MHC class I complex and/or any component of the MHC class I complex. In some embodiments, the engineered cells comprise a genetic modification of the gene encoding B2M. In some embodiments, the engineered cells comprise a genetic modification of the gene encoding CIITA. In some embodiments, the engineered cells comprise a genetic modification of the gene encoding NLRC5. In some embodiments, the engineered cells comprise a genetic modification of the gene encoding cytotoxic T-lymphocyte-associated protein 4 (CTLA4). In some embodiments, the engineered cells comprise a genetic modification of the gene encoding programmed cell death 1 (PD1). Detailed descriptions of genetically modifying T cells is found, e.g., in WO2016160721, the disclosure is incorporated herein in its entirety, including the sequence listing, tables, and figures.

In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of critical immune genes (e.g., by deleting genomic DNA of critical immune genes) in human stem cells. In certain embodiments, genome editing technologies or other gene modulation technologies are used to insert tolerance-inducing factors in human cells, rendering them and the differentiated cells prepared therefrom hypoimmunogenic (or reduced immunogenic) cells. As such, the hypoimmunogenic cells have reduced or eliminated MHC I and/or MHC II expression. In some embodiments, the cells are nonimmunogenic (e.g., do not induce an immune response) in a recipient subject. In certain embodiments, the cells possess reduce immunogenicity (e.g., decreased likelihood of eliciting an immune response) in a recipient subject.

The genome editing techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

In some embodiments, the rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

The present disclosure contemplates altering target polynucleotide sequences in any manner which is available to the skilled artisan utilizing a CRISPR/Cas system. Any CRISPR/Cas system that is capable of altering a target polynucleotide sequence in a cell can be used. Such CRISPR-Cas systems can employ a variety of Cas proteins (Haft et al. PLOS Comput Biol.; 2005; 1 (6) e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. See, e.g., Jinek et al., Science (2012) 337 (6096): 816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those described to this point. For instance, Cpf1 (CRISPR from Prevotella and Franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA: tracrRNA complexes, including in certain embodiments via a single gRNA. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 17 below.

TABLE 17
Exemplary Cas nuclease variants and their PAM sequences

| CRISPR Nuclease | Source Organism | PAM Sequence (5′→3′) |
|---|---|---|
| SpCas9 | Streptococcus pyogenes | ngg or nag |
| SaCas9 | Staphylococcus aureus | ngrrt or ngrrn |
| NmeCas9 | Neisseria meningitidis | nnnngatt |
| CjCas9 | Campylobacter jejuni | nnnnryac |
| StCas9 | Streptococcus thermophilus | nnagaaw |
| TdCas9 | Treponema denticola | naaaac |
| LbCas12a (Cpf1) | Lachnospiraceae bacterium | tttv |
| AsCas12a (Cpf1) | Acidaminococcus sp. | tttv |
| AacCas12b | Alicyclobacillus acidiphilus | ttn |
| BhCas12b v4 | Bacillus hisashii | attn, tttn, or gttn |

r = a or g;
y = c or t;
w = a or t;
v = a or c or g;
n = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example, the Cas nuclease may have one or more mutations that alter its PAM specificity.

In some embodiments, provided are host cells or compositions of the same having a genomic locus modified by any of the gene editing systems as described. In some embodiments, the genetic modification is by using a site-directed nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, zinc finger nucleases (ZFNs), transcription activator-like effector nucleases (TALENs), meganucleases, and CRISPR-associated transposases. In certain of these embodiments, the genomic locus modified is a B2M locus, a CIITA locus, a TRAC locus, a TRBC locus, or a safe harbor locus. Non-limiting examples of a safe harbor locus include, but are not limited to, an AAVS1, ABO, CCR5, CLYBL, CXCR4, F3, FUT1, HMGB1, KDM5D, LRP1, MICA, MICB, RHD, ROSA26, and SHS231 gene locus.

gRNAs for use in CRISPR editing comprise a crRNA sequence, which in turn comprises a complementary region (also called a spacer) that recognizes and binds a complementary target DNA of interest. The length of the spacer or complementary region is generally between 15 and 30 nucleotides, usually about 20 nucleotides in length, although will vary based on the requirements of the specific CRISPR/Cas system. In certain embodiments, the spacer or complementary region is fully complementary to the target DNA sequence. In other embodiments, the spacer is partially complementary to the target DNA sequence, for example at least 80%, 85%, 90%, 95%, 98%, or 99% complementary.

In certain embodiments, the gRNAs provided herein further comprise a tracrRNA sequence, which comprises a scaffold region for binding to a nuclease. The length and/or sequence of the tracrRNA may vary depending on the specific nuclease being used for editing. In certain embodiments, nuclease binding by the gRNA does not require a tracrRNA sequence. In those embodiments where the gRNA comprises a tracrRNA, the crRNA sequence may further comprise a repeat region for hybridization with complementary sequences of the tracrRNA.

In some embodiments, the gRNAs provided herein comprise two or more gRNA molecules, for example, a crRNA and a tracrRNA, as two separate molecules. In other embodiments, the gRNAs are single guide RNAs (sgRNAs), including sgRNAs comprising a crRNA and a tracrRNA on a single RNA molecule. In certain of these embodiments, the crRNA and tracrRNA are linked by an intervening tetraloop.

In some embodiments, one gRNA can be used in association with a site-directed nuclease for targeted editing of a gene locus of interest. In other embodiments, two or more gRNAs targeting the same gene locus of interest can be used in association with a site-directed nuclease.

In some embodiments, exemplary gRNAs (e.g., sgRNAs) for use with various common Cas nucleases that require both a crRNA and tracrRNA, including Cas9 and Cas12b (C2c1), are provided in Table 18. See, e.g., Jinek et al., Science (2012) 337 (6096): 816-821; Dang et al., Genome Biology (2015) 16:280; Ran et al., Nature (2015) 520:186-191; Strecker et al., Nature Comm. (2019) 10:212. For each exemplary gRNA, sequences for different portions of the gRNA, including the complementary region or spacer, crRNA repeat region, tetraloop, and tracrRNA, are shown. In some embodiments, the gRNA comprises all or a portion of the nucleotide sequences set forth in SEQ ID NOs: 94-97. In some embodiments, the gRNA comprises all or a portion of the nucleotide sequences set forth in SEQ ID NOs: 98-101. In some embodiments, the gRNA comprises all or a portion of the nucleotide sequences set forth in SEQ ID NOs: 102-105. In some embodiments, the gRNA comprises all or a portion of the nucleotide sequences set forth in SEQ ID NOs: 106-109.

In some embodiments, the gRNA comprises a crRNA repeat region comprising, consisting of, or consisting essentially of the nucleotide sequence set forth in SEQ ID NO:95, SEQ ID NO:99, SEQ ID NO:103, or SEQ ID NO:108. In some embodiments, the gRNA comprises a tetraloop comprising, consisting of, or consisting essentially of the nucleotide sequence set forth in SEQ ID NO:96 or SEQ ID NO:107. In some embodiments, the gRNA comprises a tracrRNA comprising, consisting of, or consisting essentially of the nucleotide sequence set forth in SEQ ID NO:97, SEQ ID NO:101, SEQ ID NO:105, or SEQ ID NO:106.

TABLE 18
Exemplary gRNA structure and sequence for CRISPR/Cas

| SEQ ID NO: | Sequence (5′→3′) | Description |
|---|---|---|
| 94 | nnnnnnnnnnnnnnnnnnnn | Exemplary spCas9 1 |
| | | Complementary region |
| | | (spacer) |
| 95 | guuuuagagcua | Exemplary spCas9 1 |
| | | crRNA repeat region |
| 96 | gaaa | Exemplary spCas9 1 |
| | | tetraloop |
| 97 | uagcaaguuaaaauaaggcuaguccguuaucaacuu | Exemplary spCas9 1 |
| | gaaaaaguggcaccgagucggugcuuuuuu | tracrRNA |
| 98 | nnnnnnnnnnnnnnnnnnnn | Exemplary spCas9 2 |
| | | Complementary region |
| | | (spacer) |
| 99 | guuusagagcuaugcug | Exemplary spCas9 2 |
| | | crRNA repeat region |
| 100 | gaaa | Exemplary spCas9 2 |
| | | tetraloop |
| 101 | cagcauagcaaguusaaauaaggcuaguccguuauc | Exemplary spCas9 2 |
| | aacuugaaaaaguggcaccgagucggugcuuuuuu | tracrRNA |
| 102 | nnnnnnnnnnnnnnnnnnnn | Exemplary saCas9 |
| | | Complementary region |
| | | (spacer) |
| 103 | guuuuaguacucug | Exemplary saCas9 |
| | | crRNA repeat region |
| 104 | gaaa | Exemplary saCas9 |
| | | tetraloop |
| 105 | cagaaucuacuaaaacaaggcaaaaugccguguuua | Exemplary saCas9 |
| | ucucgucaacuuguuggcgagauuuuuu | tracrRNA |
| 106 | gucgucuauaggacggcgaggacaacgggaagugcc | Exemplary AkCas12b |
| | aaugugcucuuuccaagagcaaacaccccguuggcuu | tracrRNA |
| | caagaugaccgcucg | |
| 107 | aaaa | Exemplary AkCas12b |
| | | tetraloop |
| 108 | cgagcggucugagaaguggcacu | Exemplary AkCas12b |
| | | crRNA repeat region |
| 109 | nnnnnnnnnnnnnnnnnnnn | Exemplary AkCas12b |
| | | Complementary region |
| | | (spacer) |

S = c or g; n = any base

In some embodiments, the gRNA comprises a complementary region specific to a target gene locus of interest, for example, the B2M locus, the CIITA locus, the TRAC locus, the TRBC locus, or a safe harbor locus selected from the group consisting of an AAVS1, ABO, CCR5, CLYBL, CXCR4, F3, FUT1, HMGB1, KDM5D, LRP1, MICA, MICB, RHD, ROSA26, and SHS231 gene locus. The complementary region may bind a sequence in any region of the target gene locus, including for example, a CDS, an exon, an intron, a sequence spanning a portion of an exon and a portion of an adjacent intron, or a regulatory region (e.g., promoter, enhancer). Where the target sequence is a CDS, exon, intron, or sequence spanning portions of an exon and intron, the CDS, exon, intron, or exon/intron boundary may be defined according to any splice variant of the target gene. In some embodiments, the genomic locus targeted by the gRNA is located within 4000 bp, within 3500 bp, within 3000 bp, within 2500 bp, within 2000 bp, within 1500 bp, within 1000 bp, or within 500 bp of any of the loci or regions thereof as described. Further provided herein are compositions comprising one or more gRNAs provided herein and a Cas protein or a nucleotide sequence encoding a Cas protein. In certain of these embodiments, the one or more gRNAs and a nucleotide sequence encoding a Cas protein are comprised within a vector, for example, a viral vector.

In some embodiments, the gRNAs used herein for site-directed insertion of a transgene comprise a complementary region that recognizes a target sequence in AAVS1. In certain of these embodiments, the target sequence is located in intron 1 of AAVS1. AAVS1 is located at Chromosome 19:55,090,918-55,117,637 reverse strand, and AAVS1 intron 1 (based on transcript ENSG00000125503) is located at Chromosome 19:55,117,222-55,112,796 reverse strand. In certain embodiments, the gRNAs target a genomic locus within 4000 bp, within 3500 bp, within 3000 bp, within 2500 bp, within 2000 bp, within 1500 bp, within 1000 bp, or within 500 bp of a site located anywhere at Chromosome 19:55, 117,222-55,112,796. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19:55,115,674. In certain embodiments, the gRNA is configured to produce a cut site at Chromosome 19:55, 115,674, or at a position within 5, 10, 15, 20, 30, 40, or 50 nucleotides of Chromosome 19:55,115,674. In certain embodiments, the gRNA is GET000046, also known as “sgAAVS1-1,” described in Li et al., Nat. Methods 16:866-869 (2019). This gRNA comprises a complementary region comprising, consisting of, or consisting essentially of a nucleic acid sequence set forth in SEQ ID NO:110 and targets intron 1 of AAVS1 (also known as PPP1R12C).

In some embodiments, the gRNAs used herein for site-directed insertion of a transgene comprise a complementary region that recognizes a target sequence in CLYBL. In certain of these embodiments, the target sequence is located in intron 2 of CLYBL. CLYBL is located at Chromosome 13:99,606,669-99,897,134 forward strand, and CLYBL intron 2 (based on transcript ENST00000376355.7) is located at Chromosome 13:99,773,011-99,858,860 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of a site located anywhere at Chromosome 13:99, 773,011-99,858,860. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13:99,822,980. In certain embodiments, the gRNA is configured to produce a cut site at Chromosome 13:99,822,980, or at a position within 5, 10, 15, 20, 30, 40, or 50 nucleotides of Chromosome 13:99,822,980. In certain embodiments, the gRNA is GET000047, which comprises a complementary region comprising, consisting of, or consisting essentially of a nucleic acid sequence set forth in SEQ ID NO:111 and targets intron 2 of CLYBL. The target site is similar to the target site of the TALENs as described in Cerbini et al., PLOS One, 10 (1): e0116032 (2015).

In some embodiments, the gRNAs used herein for site-directed insertion of a transgene comprise a complementary region that recognizes a target sequence in CCR5. In certain of these embodiments, the target sequence is located in exon 3 of CCR5. CCR5 is located at Chromosome 3:46,370,854-46,376,206 forward strand, and CCR5 exon 3 (based on transcript ENST00000292303.4) is located at Chromosome 3:46,372,892-46,376,206 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of a site located anywhere at Chromosome 3:46,372,892-46,376,206. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3:46,373,180. In certain embodiments, the gRNA is configured to produce a cut site at Chromosome 3:46,373,180, or at a position within 5, 10, 15, 20, 30, 40, or 50 nucleotides of Chromosome 3:46,373,180. In certain embodiments the gRNA is GET000048, also known as “crCCR5_D,” described in Mandal et al., Cell Stem Cell 15:643-652 (2014). This gRNA comprises a complementary region comprising, consisting of, or consisting essentially of a nucleic acid sequence set forth in SEQ ID NO:112 and targets exon 3 of CCR5 (alternatively annotated as exon 2 in the Ensembl genome database). See Gomez-Ospina et al., Nat. Comm. 10 (1): 4045 (2019).

In certain embodiments of the gRNAs used herein, one or more thymines in the complementary region sequences set forth in Table 19 are substituted with uracils.

TABLE 19
Exemplary gRNA complementary region sequences

| SEQ ID NO: | Nucleic Acid Sequence (5′→3′) | Description |
|---|---|---|
| 110 | accccacagtggggccacta | GET000046 guide |
| 111 | tgttggaaggatgaggaaat | GET000047 guide |
| 112 | tcactatgctgccgcccagt | GET000048 guide |

In some embodiments, provided are methods of identifying new loci and/or gRNA sequences for use in the site-directed gene editing approaches as described. For example, for CRISPR/Cas systems, when an existing gRNA for a particular locus (e.g., within a safe harbor locus) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in site-directed insertion of transgenes. Although the CRISPR/Cas system is described as illustrative, any gene editing approaches as described can be used in this method of identifying new loci, including those using ZFNs, TALENs, meganucleases, and transposases.

In some embodiments, the activity, stability, and/or other characteristics of gRNAs can be altered through the incorporation of chemical and/or sequential modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not being bound by a particular theory, it is believed that certain modified gRNAs described herein can exhibit a reduced innate immune response when introduced into a population of cells, particularly the cells of the present technology. As used herein, the term “innate immune response” includes a cellular response to exogenous nucleic acids, including single stranded nucleic acids, generally of viral or bacterial origin, which involves the induction of cytokine expression and release, particularly the interferons, and cell death. Other common chemical modifications of gRNAs to improve stabilities, increase nuclease resistance, and/or reduce immune response include 2′-O-methyl modification, 2′-fluoro modification, 2′-O-methyl phosphorothioate linkage modification, and 2′-O-methyl 3′ thioPACE modification.

One common 3′ end modification is the addition of a poly A tract comprising one or more (and typically 5-200) adenine (A) residues. The poly A tract can be contained in the nucleic acid sequence encoding the gRNA or can be added to the gRNA during chemical synthesis, or following in vitro transcription using a polyadenosine polymerase (e.g., E. coli Poly (A) Polymerase). In vivo, poly-A tracts can be added to sequences transcribed from DNA vectors through the use of polyadenylation signals. Examples of such signals are provided in Maeder. Other suitable gRNA modifications include, without limitations, those described in U.S. Patent Application No. US 2017/0073674 A1 and International Publication No. WO 2017/165862 A1, the entire contents of each of which are incorporated by reference herein.

The CRISPR/Cas systems can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, a CRISPR/Cas systems can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, a CRISPR/Cas system includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to Cas1, Cas2, Cas3, Cas4, Cas5, Cas6, Cas7, Cas8 and Cas9. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6.

In some embodiments, a Cas protein comprises any one of the Cas proteins described herein or a functional portion thereof. As used herein, “functional portion” or “function fragment” refers to a portion of a peptide or protein factor which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from a group that includes a DNA binding domain, at least one RNA binding domain, a helicase domain, and an endonuclease domain. In some embodiments, the functional portion comprises a combination of operably linked Cpf1 protein functional domains selected from a group that includes a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cpf1 protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol.; 2010; 5 (8): 747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP.

In some embodiments, the Cas polypeptide comprises a Cpf1 (Cas12a) protein or a variant thereof. In some embodiments, the Cpf1 (Cas12a) protein comprises a Cpf1 polypeptide fused to a cell-penetrating peptide. In some embodiments, the Cpf1 protein comprises a Cpf1 polypeptide fused to a PTD. In some embodiments, the Cpf1 protein comprises a Cpf1 polypeptide fused to a tat domain. In some embodiments, the Cpf1 protein comprises a Cpf1 polypeptide fused to an oligoarginine domain. In some embodiments, the Cpf1 protein comprises a Cpf1 polypeptide fused to a penetratin domain. In some embodiments, the Cpf1 protein comprises a Cpf1 polypeptide fused to a superpositively charged GFP. Detailed descriptions of Cpf1 proteins can be found, e.g., in Safari et al., Cell & Bioscience, 2019; 9, 36; doi.org/10.1186/s13578-019-0298-7.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods of the present disclosure contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids of the present disclosure can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA sequences useful for CRISPR/Cas-based targeting of genes described herein are provided in Table 20 below. The sequences can be found in WO2016/183041 filed May 9, 2016, the disclosure including the tables, appendices, and sequence listing is incorporated herein by reference in its entirety.

TABLE 20
Exemplary gRNA sequences useful for targeting genes

| Gene | SEQ ID NO in | Table/Appendix No. in |
|---|---|---|
| Name | WO2016183041 | WO2016183041 |
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOS: 3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |
| CD47 | SEQ ID NOS: 200784-231885 | Table 29, Appendix 22 |
| HLA-E | SEQ ID NOS: 189859-193183 | Table 19, Appendix 12 |
| HLA-F | SEQ ID NOS: 688808-699754 | Table 45, Appendix 38 |
| HLA-G | SEQ ID NOS: 188372-189858 | Table 18, Appendix 11 |
| PD-L1 | SEQ ID NOS: 193184-200783 | Table 21, Appendix 14 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |
| CD47 | SEQ ID NOS: 200784-231885 | Table 29, Appendix 22 |
| HLA-C | SEQ ID NOS: 3278-5183 | Table 10, Appendix 3 |
| HLA-E | SEQ ID NOS: 189859-193183 | Table 19, Appendix 12 |
| HLA-F | SEQ ID NOS: 688808-699754 | Table 45, Appendix 38 |
| HLA-G | SEQ ID NOS: 188372-189858 | Table 18, Appendix 11 |
| PD-L1 | SEQ ID NOS: 193184-200783 | Table 21, Appendix 14 |

In some embodiments, the cells described are made using Transcription Activator-Like Effector Nucleases (TALEN) methodologies.

By a “TALE-nuclease” (TALEN) is intended a fusion protein containing a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

In some embodiments, the cells disclosed are made using a homing endonuclease. Such homing endonucleases are well-known to the art (B. L. Stoddard, Q Rev Biophys, 2005;38 (1): 49-95 2005). Homing endonucleases recognize a DNA target sequence and generate a single- or double-strand break. Homing endonucleases contain highly specific, recognizing DNA target sites ranging from 12 to 45 base pairs (bp) in length, usually ranging from 14 to 40 bp in length. The homing endonuclease may, for example, correspond to a LAGLIDADG endonuclease, to a HNH endonuclease, or to a GIY-YIG endonuclease. Preferred homing endonuclease according to the present disclosure can be an I-CreI variant.

In some embodiments, the cells described are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells described herein are made using RNA silencing or RNA interference (RNAi) to knockdown (e.g., decrease, eliminate, or inhibit) the expression of a polypeptide such as an immunosuppressive factor, tolerogenic factor, and the like. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, CIITA can be knocked down in a pluripotent stem cell by introducing a CIITA siRNA or transducing a CIITA shRNA-expressing virus into the cell. In some embodiments, RNA interference is employed to reduce or inhibit the expression of at least one selected from a group that includes CIITA, B2M, and NLRC5.

### 1. Exemplary Expression Constructs

For transferring exogenous genes into cells of interest, well known recombinant techniques are used, to generate recombinant nucleic acids as outlined herein. Many vectors useful for exogenously expressing polypeptides in target cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, and the like. In some embodiments of stem cells, lentiviral vectors are preferred.

In certain embodiments, the recombinant nucleic acids encoding an immunosuppressive factor may be operably linked to one or more regulatory nucleotide sequences in an expression construct. Regulatory nucleotide sequences will generally be appropriate for the host cell and subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al, Nature, 273:113-120 (1978)). The immediate early promoter of the human cytomegalovirus is conveniently obtained as a Hindlll restriction enzyme fragment (Greenaway et al., Gene, 18:355-360 (1982)). The foregoing references are incorporated by reference in their entirety.

The process of introducing the polynucleotides described herein into cells can be achieved by any suitable technique. Suitable techniques include calcium phosphate or lipid-mediated transfection, electroporation, and transduction or infection using a viral vector. In some embodiments, the polynucleotides are introduced into a cell via viral transduction (e.g., lentiviral transduction).

Once altered, the presence of expression of any of the molecule described herein can be assayed using known techniques, such as Western blots, ELISA assays, FACS assays, and the like.

### 2. Hypoimmune Cells

Provided herein are hypoimmune cells, including hypoimmune stem cells, cells differentiated from those stem cells, or primary cells (collectively referred to herein as “HIP cells”) engineered to express immune regulator proteins and evade rejection by a recipient host's immune system upon administration to the recipient subject as part of allogeneic cell therapy. The introduction of safety switches to modulate the activity of such cells upon administration to a recipient subject is an important technology to improve the safety of these cell therapies.

A key feature of HIP cells is their expression of immunosuppressive factors that function to suppress the host cell immune response to the engrafted population of cells. In some embodiments, the hypoimmunity of the cells that are introduced to a recipient subject is achieved through the overexpression of an immunosuppressive molecule including hypoimmunity factors, such as CD47, and complement inhibitors accompanied with the repression or genetic disruption of the HLA-I and HLA-II loci. These modifications cloak the cell from the recipient immune system's effector cells that are responsible for the clearance of infected, malignant or non-self cells, such as T cells, B cells, NK cells and macrophages. Cloaking of a cell from the immune system allows for existence and persistence of allogeneic cells within the body. Controlled removal of the engineered cells from the body is crucial for patient safety and can be achieved by uncloaking the cells from the immune system. Uncloaking serves as a safety switch and can be achieved through blocking and/or interfering with the CD47-SIRPα axis or interaction.

### C. Assays for Hypoimmunogenicity Phenotypes and Retention of Pluripotency

Once the hypoimmunogenic cells or cells that evade immune recognition have been generated, they may be assayed for their immunogenicity and/or retention of pluripotency as is described in WO2016183041, WO2018132783, and WO2018175390.

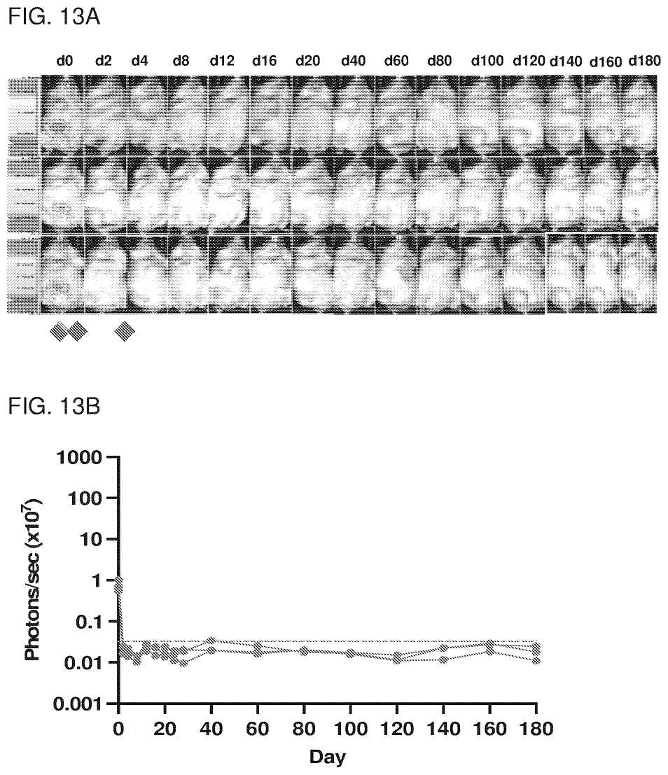

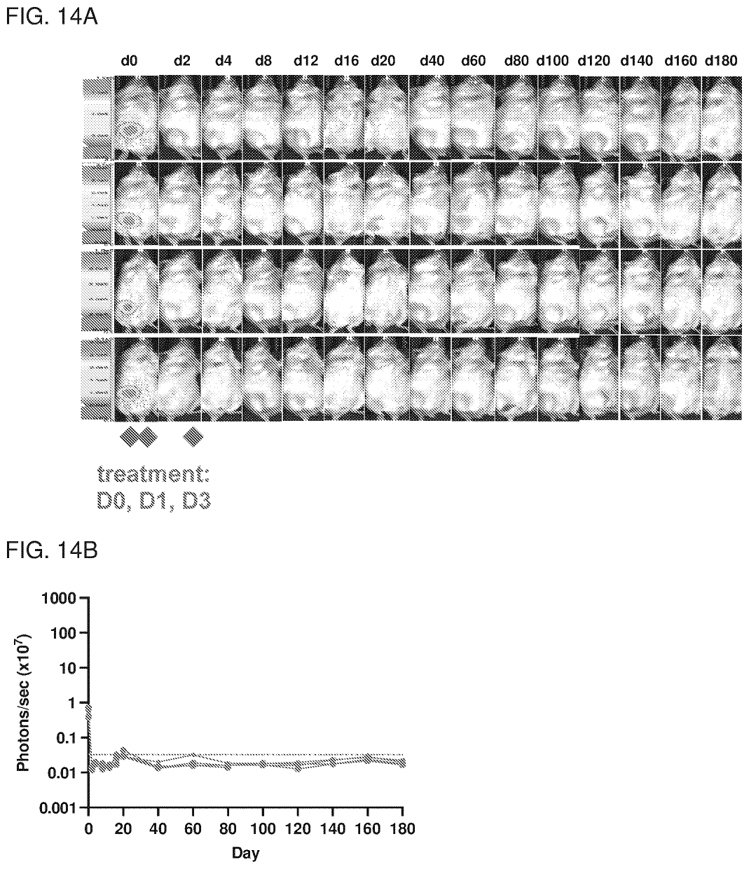

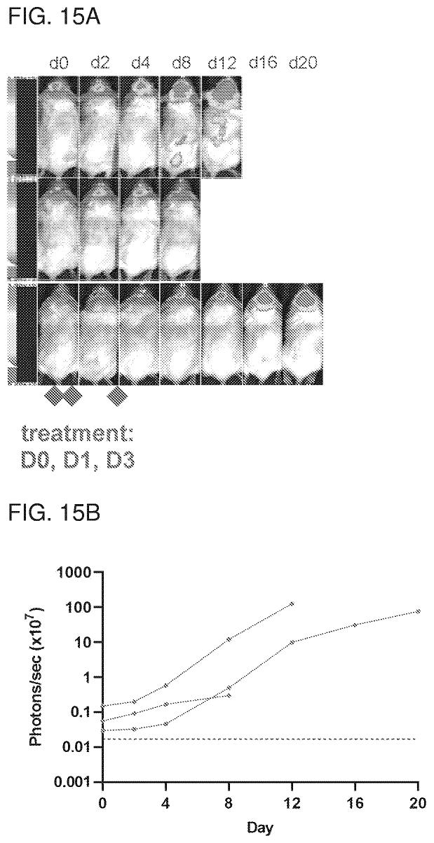

In some embodiments, hypoimmunogenicity is assayed using a number of techniques as exemplified in FIG. 13 and FIG. 15 of WO2018132783. These techniques include transplantation into allogeneic or xenogeneic hosts and monitoring for hypoimmunogenic pluripotent cell growth (e.g. teratomas) that escape the host immune system. In some instances, hypoimmunogenic cell derivatives are transduced to express luciferase and can then followed using bioluminescence imaging. Similarly, the T cell and/or B cell response of the host animal to such cells are tested to confirm that the cells do not cause an immune reaction in the host animal. T cell function is assessed by Elispot, ELISA, FACS, PCR, or mass cytometry (CYTOF). B cell response or antibody response is assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116 (21), 10441-10446, the disclosures including the figures, figure legends, and description of methods are incorporated herein by reference in their entirety.

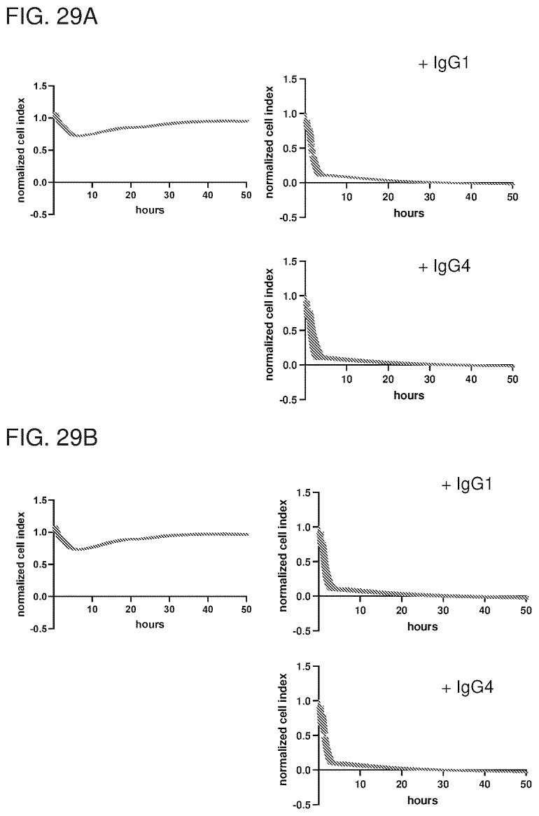

Similarly, the retention of pluripotency is tested in a number of ways. In one embodiment, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, B, C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

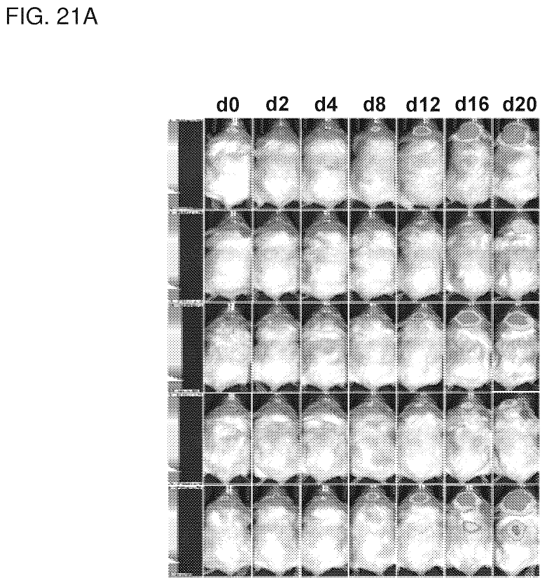

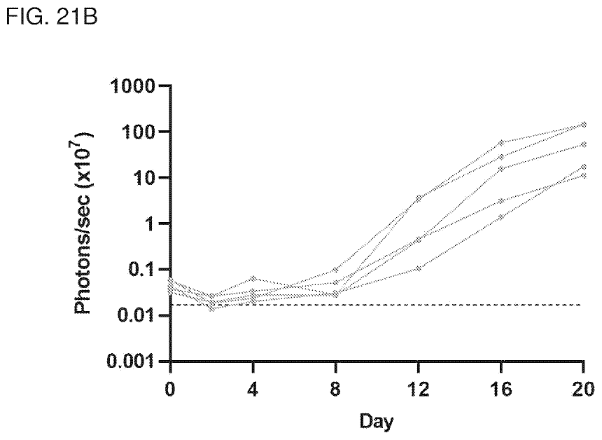

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of HLA I and II (or MHC I and II), the cells disclosed can have a reduced susceptibility to macrophage phagocytosis and NK cell killing. The resulting cells are believed (without wishing to be bound by theory) to evade the immune macrophage and innate pathways due to the expression of one or more CD47 transgenes.

### D. Differentiation of Stem Cells

The present disclosure provides pluripotent cells that can be differentiated into different cell types for subsequent transplantation into subjects. As will be appreciated by those in the art, the methods for differentiation depend on the desired cell type using known techniques. The cells can be differentiated in suspension and then put into a gel matrix form, such as matrigel, gelatin, or fibrin/thrombin forms to facilitate cell survival. In some cases, differentiation is assayed as is known in the art, generally by evaluating the presence of cell-specific markers.

In some embodiments, the pluripotent cells are differentiated into hepatocytes to address loss of the hepatocyte functioning or cirrhosis of the liver. There are a number of techniques that can be used to differentiate hypoimmunogenic pluripotent cells into hepatocytes; see for example Pettinato et al., doi: 10.1038/spre32888, Snykers et al., Methods Mol Biol 698:305-314 (2011), Si-Tayeb et al, Hepatology 51:297-305 (2010) and Asgari et al., Stem Cell Rev (493-504 (2013), all of which are hereby expressly incorporated by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release and glycogen storage.

In some embodiments, the pluripotent cells are differentiated into beta-like cells or islet organoids for transplantation to address type I diabetes mellitus (T1DM). Cell systems are a promising way to address T1DM, see, e.g., Ellis et al., doi/10.1038/nrgastro.2017.93, incorporated herein by reference. Additionally, Pagliuca et al. reports on the successful differentiation of β-cells from human iPSCs (see doi/10.10⁶/j.cell.2014.09.040, hereby incorporated by reference in its entirety and in particular for the methods and reagents outlined there for the large-scale production of functional human B cells from human pluripotent stem cells). Furthermore, Vegas et al. shows the production of human B cells from human pluripotent stem cells followed by encapsulation to avoid immune rejection by the host; (doi: 10.1038/nm.4030, hereby incorporated by reference in its entirety and in particular for the methods and reagents outlined there for the large-scale production of functional human B cells from human pluripotent stem cells).

Differentiation is assayed as is known in the art, generally by evaluating the presence of β cell associated or specific markers, including but not limited to, insulin. Differentiation can also be measured functionally, such as measuring glucose metabolism, see generally Muraro et al, doi: 10.1016/j.cels.2016.09.002, hereby incorporated by reference in its entirety, and specifically for the biomarkers outlined there.

In some embodiments, the pluripotent cells are differentiated into retinal pigment epithelium (RPE) to address sight-threatening diseases of the eye. Human pluripotent stem cells have been differentiated into RPE cells using the techniques outlined in Kamao et al., Stem Cell Reports 2014:2:205-18, hereby incorporated by reference in its entirety and in particular for the methods and reagents outlined there for the differentiation techniques and reagents; see also Mandai et al., doi: 10.1056/NEJMoa1608368, also incorporated in its entirety for techniques for generating sheets of RPE cells and transplantation into patients.

Differentiation can be assayed as is known in the art, generally by evaluating the presence of RPE associated and/or specific markers or by measuring functionally. See for example Kamao et al., doi: 10.1016/j.stemcr.2013.12.007, hereby incorporated by reference in its entirety and specifically for the markers outlined in the first paragraph of the results section.

In some embodiments, the pluripotent cells are differentiated into cardiomyocytes to address cardiovascular diseases. Techniques are known in the art for the differentiation of hiPSCs to cardiomyocytes and discussed in the Examples. Differentiation can be assayed as is known in the art, generally by evaluating the presence of cardiomyocyte associated or specific markers or by measuring functionally; see for example Loh et al., doi: 10.1016/j.cell.2016.06.001, hereby incorporated by reference in its entirety and specifically for the methods of differentiating stem cells including cardiomyocytes.

In some embodiments, the pluripotent cells are differentiated into endothelial colony forming cells (ECFCs) to form new blood vessels to address peripheral arterial disease. Techniques to differentiate endothelial cells are known. See, e.g., Prasain et al., doi: 10.1038/nbt.3048, incorporated by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the pluripotent cells are differentiated into thyroid progenitor cells and thyroid follicular organoids that can secrete thyroid hormones to address autoimmune thyroiditis. Techniques to differentiate thyroid cells are known the art. See, e.g. Kurmann et al., doi: 10.106/j.stem.2015.09.004, hereby expressly incorporated by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

Additional descriptions of methods for differentiating pluripotent cells can be found, for example, in Deuse et al., Nature Biotechnology, 2019, 37, 252-258 and Han et al., Proc Natl Acad Sci USA, 2019, 116 (21), 10441-10446.

E. Administration/Transplantation of Primary Cells and/or Cells Derived from Primary Cells

In some embodiments, the primary cells or non-primary cell derivatives thereof are transplanted or engrafted using techniques known in the art that depends on both the cell type and the ultimate use of these cells. In general, the cells of the present disclosure can be administered either intravenously or by injection at particular locations in the patient. When transplanted at particular locations, the cells may be suspended in a gel matrix to prevent dispersion. In some embodiments, the patient receiving the cells is administered an immune suppressive agent. In other embodiments, the patient receiving the cells are not administered an immune suppressive agent.

In some embodiments, provided herein is a method of treating a patient in need of cell therapy comprising administering a population of cells comprising differentiated cells generated from engineered stem cells comprising an exogenous immunosuppressive factor. In useful embodiments, provided herein is a method of treating a patient in need of cell therapy comprising administering a population of cells comprising differentiated cells generated from stem cells comprising exogenous human CD47. Generally, a safe and effective amount of engineered cells is, for example, an amount that would elicit a desired therapeutic effect in a patient while minimizing undesired adverse effects. In additional embodiments, the patient is administered any of the CD47-SIRPα blockade agents described herein, and thus minimizing undesired adverse effects from the administered engineered cells.

In some embodiments, provided herein is a method of treating a patient in need of cell therapy comprising administering a population of primary T cells comprising primary T cells expressing an exogenous immune signaling factor. In useful embodiments, provided herein is a method of treating a patient in need of cell therapy comprising administering a population of primary T cells comprising primary T cells comprising exogenous human CD47. In some embodiments, the patient is administered any of the CD47-SIRPα blockade agents described herein.

In some embodiments, a CD47-SIRPα blockade agent is administered when cells administered to the patient undergo inappropriate expansion or proliferation in the recipient. In some embodiments, a CD47-SIRPα blockade agent is administered when cells administered to the patient are present in an inappropriate location in the recipient's body. In some embodiments, a CD47-SIRPα blockade agent is administered when cells administered to the patient undergo a malignant transformation. In some embodiments, a CD47-SIRPα blockade agent is administered when cells administered to the patient induce cytokine release syndrome. In some embodiments, a CD47-SIRPα blockade agent is administered when cells administered to the patient induce neurotoxicity. In some embodiments, a CD47-SIRPα blockade agent is administered when cells administered to the patient induce toxicity such as on-target off tumor toxicity.

In one aspect, the method described comprises administering one or more doses of a population of CD47 engineered cells (e.g., a population of cells exogenously expressing CD47) to a recipient subject in need thereof, and afterwards administering of a CD47-SIRPα blockade agent. In some embodiments, the recipient subject receives 1, 2, 3, 4, 5, 6, 7, 8, 9, 10 or more doses of the population of cells. In some embodiments, the patient receives an initial dose of a population of CD47 engineered cells, and then the patient is administered a CD47-SIRPα blockade agent. In particular embodiments, the patient is administered an initial dose of a population of CD47 engineered cells, and then a CD47-SIRPα blockade agent, and then a subsequent population of CD47 engineered cells. In certain embodiments, the patient is administered an initial dose of a population of CD47 engineered cells, and then a first administration of a CD47-SIRPα blockade agent, and then a subsequent population of CD47 engineered cells, and then a second administration of a CD47-SIRPα blockade agent. The initial dose of the population of CD47 engineered cells comprises one or more (e.g., 1, 2, 3, 4, 5, 6, 7, 8, 9, 10 or more) infusions or injections of the cells. The subsequent dose of the population of CD47 engineered cells comprises one or more (e.g., 1, 2, 3, 4, 5, 6, 7, 8, 9, 10 or more) infusions or injections of the cells.

In another aspect, the method comprises performing a therapeutic regimen comprising a treatment cycle comprising administering a population of engineered cells, and then administering a CD47-SIRPα blockade agent. In some embodiments, the therapeutic regimen comprises one or more (e.g., 1, 2, 3, 4, or more) treatment cycles such that each treatment cycle comprises administering a population of engineered cells and then administering a CD47-SIRPα blockade agent. In some embodiments, the step of administering a population of engineered cells to a recipient subject includes administering 1, 2, 3, 4, 5, 6, 7, 8, 9, 10 or more doses of the population of cells. In some embodiments, the recipient subject is administered 1, 2, 3, 4, 5, 6, 7, 8, 9, 10 or more doses of the population of cells prior to receiving a CD47-SIRPα blockade agent.

In some embodiments, method described herein includes administering a population of CD47 engineered cells, and then administering a CD47-SIRPα blockade agent following an interval time period. In some instances, the interval time period is at least 1 week or more. In some instances, the interval time period is at least 1 month or more. In some instances, the interval time period ends if the recipient subject exhibits an adverse effect induced by the administered cells. In some embodiments, the interval time period ends if the administered cells undergo inappropriate expansion or proliferation in the recipient. In certain embodiments, the interval time period ends if the administered cells are present in an inappropriate location in the recipient's body. In particular embodiments, the interval time period ends if the administered cells undergo a malignant transformation. In some embodiments, the interval time period ends if the administered cells induce cytokine release syndrome. In other embodiments, the interval time period ends if the administered cells induce neurotoxicity. In particular embodiments, the interval time period if when the administered cells induce toxicity such as on-target off tumor toxicity.

In some embodiments, the method comprises multiple cycles of CD47-SIRPα blockade agent therapy. In some instances, the therapeutic regimen comprises administering one or a plurality of doses the CD47-SIRPα blockade agent such that the amount of the administered cells and derivatives thereof (e.g., the administered cells and any cells generated from such cells in the recipient subject) is reduced by at least 10% (e.g., 10%, 20%, 30%, 40%, 50%, 60%, 70%, 80%, 90%, 91%, 92%, 93%, 94%, 95%, 96%, 97%, 98%, 99% or more). In some embodiments, the CD47-SIRPα blockade agent is administered such that substantially all of the administered cells undergo cell death and/or cell clearance (e.g., phagocytosis).

Iv. CD47-SIRPα Blockade Agents

The introduction of safety switches improves the safety of cell therapies such as those involving engineered cells comprising CD47. Described herein are methods for reducing an immune evasion effect of CD47 in such cells engrafted in a recipient subject. In some embodiments, a recipient subject is treated with a therapeutic agent that inhibits or blocks the interaction of CD47 and SIRPα. In some embodiments, a CD47-SIRPα blockade agent (e.g., a CD47-SIRPα blocking, inhibiting, reducing, antagonizing, neutralizing, or interfering agent) comprises an agent selected from a group that includes an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof. In one aspect of the present disclosure, provided herein is a method comprising administering a CD47-SIRPα blockade agent to a patient that has been previously administered cells comprising exogenously expressing CD47 proteins. As such, without wishing to be bound by theory, it is believed that the cells can no longer evade immune recognition and thus are recognized by the patient's immune cells and targeted for cell death and/or cell clearance. In some instances, the patient's innate immune cells are activated or mobilized to decrease the number of the previously administered cells and their derivatives (e.g., progeny).

Any of the CD47-SIRPα blockade agents described herein are useful for treating a patient with a condition or disease that is responsive to cell therapy. For instance, such a condition or disease can be characterized by the presence of unhealthy cells or tissue (e.g., diseased cells or tissue) that can be replaced by therapeutic interventions comprising healthy cell, including cells that are not in a diseased state. In some embodiments, the patient having the condition or disease is administered a cell therapy that is expected to ameliorate one or more symptoms of the condition or disease. Any of the CD47-SIRPα blockade agents can be used for the treatment, reduction or amelioration of an adverse effect adverse effect subsequent to administration of a population of cells comprising exogenously expressed CD47 polypeptides. In some embodiments, the agent is used for the control of an effect of a cell therapy in a patient, to modulate an activity of a cell therapy in a patient, or to reduce the number of cells comprising exogenously expressed CD47 polypeptides in the patient.

In some aspects, the CD47-SIRPα blockade agent reduces in the recipient patient the number of cells exogenously expressing CD47 polypeptides, including, but not limited to, cells that also exogenously express one or more chimeric antigen receptors. In some embodiments, the CD47-SIRPα blockade agent decreases the number of CD47-expressing cells in the patient, independent of the level of CAR expression by such cells. In some instances, the level of CAR expression by the cells is less (e.g., 1%, 5%, 10%, 15%, 20%, 25%, 30%, 40%, 50%, 60%, 70%, 80%, 90%, 95%, or 99% less) than the level by a control CAR-T cell, such as, but not limited to, a tisagenlecleucel biosimilar, tisagenlecleucel surrogate and the like. In certain instances, the level of CAR expression by the cells is more (e.g., 1%, 5%, 10%, 15%, 20%, 25%, 30%, 40%, 50%, 60%, 70%, 80%, 90%, 95%, 100%, 150%, 200%, 300%, or a higher percentage more) than the level by a control CAR-T cell, such as, but not limited to, a tisagenlecleucel biosimilar, tisagenlecleucel surrogate and the like.

### A. CD47-Binding Blockade Agents

In some embodiments of the methods provided herein, the CD47-SIRPα blockade agent is an agent that binds CD47. The agent can be a CD47 blocking, neutralizing, antagonizing or interfering agent. In some embodiments, the CD47-SIRPα blockade agent is selected from a group that includes an antibody or fragment thereof that binds CD47, a bispecific antibody that binds CD47, and an immunocytokine fusion protein that binds CD47.

Useful antibodies or fragments thereof that bind CD47 can be selected from a group that includes magrolimab ((Hu5F9-G4)) (Forty Seven, Inc.; Gilead Sciences, Inc.), urabrelimab, CC-90002 (Celgene; Bristol-Myers Squibb), IBI-188 (Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), and IMC-002. (Samsung Biologics; ImmuneOncia Therapeutics). In some embodiments, the antibody or fragment thereof does not compete for CD47 binding with an antibody selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof competes for CD47 binding with an antibody selected from magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof that binds CD47 is selected from a group that includes a single-chain Fv fragment (scFv) against CD47, a Fab against CD47, a VHH nanobody against CD47, a DARPin against CD47, and variants thereof. In some embodiments, the scFv against CD47, a Fab against CD47, and variants thereof are based on the antigen binding domains of any of the antibodies selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

Useful bispecific antibodies that bind CD47 comprise a first antigen binding domain that binds CD47 and a second antigen binding domain that binds an antigen selected from a group that includes CD19, CD20, CD22, CD24, CD25, CD30, CD33, CD38, CD44, CD52, CD56, CD70, CD96, CD97, CD99, CD123, CD279 (PD-1), EGFR, HER2, CD117, c-Met, PTHR2, HAVCR2 (TIM3), and an antigen expressed on a cancer cell.

In some embodiments, a CD47-SIRPα blockade agent is an immunocytokine fusion protein comprising a cytokine and either an antigen binding domain, antibody, or fragment thereof that binds CD47.

Detailed descriptions of exemplary CD47 binding molecules (e.g., antigen binding domains, antibodies, nanobodies, diabodies, antibody mimetic proteins (e.g., DARPins), and fragments thereof that recognize or bind CD47) including sequences of the heavy chain, light chain, VH region, VL region, CDRs, and framework regions can be found, for example, in WO2009091601; WO2011143624; WO2013119714; WO201414947; WO2014149477; WO2015138600; WO2016033201; WO2017049251; Pietsch et al., Blood Cancer J, 2017, 7 (2), e536; van Brommel et al., 2018, 7 (2), e1386361; Yu et al., Biochimie, 2018, 151, 54-66; and Andrechak et al., Phil Trans R Soc, 2019, 374, 20180217; the disclosures such as the sequence listings, specifications, and figures are herein incorporated in their entirety.

### B. SIRPα-Binding Blockade Agents

In some embodiments, the CD47-SIRPα blockade agent administered to the recipient subject is an agent that binds SIRPα. The agent can be an SIRPα blocking, neutralizing, antagonizing or inactivating agent. In some embodiments, the CD47-SIRPα blockade agent is selected from a group that includes, but is not limited to, an antibody or fragment thereof that binds SIRPα, a bispecific antibody that binds SIRPα, and an immunocytokine fusion protein that bind SIRPα.

Useful antibodies or fragments thereof that bind SIRPα can be selected from a group that includes, but is not limited to, ADU-1805 (Aduro Biotech Holdings), OSE-172 (OSE Immunotherapeutics; also known as BI 765063 by Boehringer Ingelheim), CC-95251 (Celgene; Bristol-Myers Squibb), KWAR23 (Leland Stanford Junior University), and P362 (Leland Stanford Junior University). In some embodiments, the antibody or fragment thereof does not compete for SIRPα binding with an antibody selected from a group that includes ADU-1805, CC-95251, OSE-172 (BI 765063), KWAR23, and P362. In some embodiments, the antibody or fragment thereof competes for SIRPα binding with an antibody selected from a group that includes ADU-1805, CC-95251, OSE-172 (BI 765063), KWAR23, and P362.

In some embodiments, the antibody or fragment thereof that binds SIRPα is selected from a group that includes a single-chain Fv fragment (scFv) against SIRPα, a Fab against SIRPα, a VHH nanobody against SIRPα, a DARPin against SIRPα, and variants thereof. In some embodiments, the scFv against SIRPα, a Fab against SIRPα, and variants thereof are based on the antigen binding domains of any of the antibodies selected from a group that includes ADU-1805, CC-95251, OSE-172 (BI 765063), KWAR23, and P362.

In some embodiments, the bispecific antibody that binds SIRPα and an antigen binding domain that binds an antigen selected from a group that includes CD19, CD20, CD22, CD24, CD25, CD30, CD33, CD38, CD44, CD52, CD56, CD70, CD96, CD97, CD99, CD123, CD279 (PD-1), EGFR, HER2, CD117, C-Met, PTHR2, HAVCR2 (TIM3), and an antigen expressed on a cancer cell. In some instances, the bispecific antibody binds SIRPα and a tumor associated antigen. In some instances, the bispecific antibody binds SIRPα and an antigen expressed on the surface of an immune cell.

In some embodiments, a CD47-SIRPα blockade agent is an immunocytokine fusion protein comprises a cytokine and either an antigen binding domain, antibody, or fragment thereof that binds SIRPα.

Detailed descriptions of exemplary SIRPα binding molecules (e.g., antigen binding domains, antibodies, nanobodies, diabodies, antibody mimetic proteins (e.g., DARPins), and fragments thereof that recognize or bind SIRPα) including sequences of the heavy chain, light chain, VH region, VL region, CDRs, and framework regions can be found, for example, in WO2019226973; WO2018190719; WO2018057669; WO2017178653; WO2016205042; WO2016033201; WO2016022971; WO2015138600; and WO2013109752; the disclosures including the sequence listings, specifications, and figures are herein incorporated in their entirety.

C. CD47- and/or SIRPα-Containing Fusion Proteins

As described herein, a CD47-SIRPα blockade agent can comprise a CD47-containing fusion protein that binds SIRPα. In some embodiments, such CD47-containing fusion protein that binds SIRPα is an agent administered to a recipient subject. In some embodiments, the CD47-containing fusion protein comprises a CD47 extracellular domain or variants thereof that bind SIRPα. In some embodiments, the fusion protein comprises an Fc region. Detailed descriptions of exemplary CD47 fusion proteins including sequences can be found, for example, in US20100239579, the disclosure is herein incorporated in its entirety including the sequence listing, specification, and figure.

In some embodiments, a CD47-SIRPα blockade agent can comprise an SIRPα-containing fusion protein that binds CD47. The sequence of SIRPα is set forth in SEQ ID NO: 129 (UniProt P78324). Generally, SIRPα-containing fusion proteins comprise a domain of SIRPα including any one of (a) the immunoglobulin-like domain of human SIRPα (e.g., the membrane distal (D1) loop containing an IgV domain of SIRP, (b) the first membrane proximal loop containing an IgC domain, and (c) the second membrane proximal loop containing an IgC domain). In some instances, the SIRPα domain binds CD47. In some embodiments, the SIRPα-containing fusion protein comprises an SIRPα extracellular domain or variants thereof that bind CD47. In some embodiments, the fusion protein comprises an Fc region, including but not limited to a human IgG1 Fc region (e.g., UniProtKB/Swiss-Prot P01857, SEQ ID NO:130) or IgG4 Fc region (e.g., UniProt P01861, SEQ ID NO:131; GenBank CAC20457.1, SEQ ID NO:132). Optionally, the Fc region may comprise one or more substitutions. In some embodiments, the SIRPα-containing fusion proteins are selected from a group that includes TTI-621 (Trillium Therapeutics), TTI-622 (Trillium Therapeutics), and ALX148 (ALX Oncology). TTI-621 (SEQ ID NO:133) is a fusion protein made up of the N-terminal V domain of human SIRPα fused to a human IgG1 Fc region (Petrova et al. Clin Cancer Res 23 (4): 1068-1079 (2017)), while TTI-622 (SEQ ID NO: 134) is a fusion protein made up of the N-terminal V domain of human SIRPα fused to a human IgG4 Fc region with a single substitution.

TABLE 21
Exemplary sequences of SIRPα, IgG1/IgG4, and CD47 fusion proteins

| SEQ ID NO: | Sequence | Description |
|---|---|---|
| 129 | MEPAGPAPGRLGPLLCLLLAASCAWSGVAG | SIRPα (UniProt |
| | EEELQVIQPDKSVSVAAGESAILHCTVTSLIP | P78324) |
| | VGPIQWFRGAGPARELIYNQKEGHFPRVTTV | |
| | SESTKRENMDFSISISNITPADAGTYYCVKFR | |
| | KGSPDTEFKSGAGTELSVRAKPSAPVVSGP | |
| | AARATPQHTVSFTCESHGFSPRDITLKWFKN | |
| | GNELSDFQTNVDPVGESVSYSIHSTAKVVLT | |
| | REDVHSQVICEVAHVTLQGDPLRGTANLSET | |
| | IRVPPTLEVTQQPVRAENQVNVTCQVRKFYP | |
| | QRLQLTWLENGNVSRTETASTVTENKDGTY | |
| | NWMSWLLVNVSAHRDDVKLTCQVEHDGQP | |
| | AVSKSHDLKVSAHPKEQGSNTAAENTGSNE | |
| | RNIYIVVGVVCTLLVALLMAALYLVRIRQKKA | |
| | QGSTSSTRLHEPEKNAREITQVQSLDTNDIT | |
| | YADLNLPKGKKPAPQAAEPNNHTEYASIQTS | |
| | PQPASEDTLTYADLDMVHLNRTPKQPAPKP | |
| | EPSFSEYASVQVPRK | |
| 130 | ASTKGPSVFPLAPSSKSTSGGTAALGCLVKD | Human IgG1 |
| | YFPEPVTVSWNSGALTSGVHTFPAVLQSSG | (UniProtKB/Swiss- |
| | LYSLSSVVTVPSSSLGTQTYICNVNHKPSNT | Prot P01857) |
| | KVDKKVEPKSCDKTHTCPPCPAPELLGGPS | |
| | VFLFPPKPKDTLMISRTPEVTCVVVDVSHED | |
| | PEVKFNWYVDGVEVHNAKTKPREEQYNSTY | |
| | RVVSVLTVLHQDWLNGKEYKCKVSNKALPA | |
| | PIEKTISKAKGQPREPQVYTLPPSRDELTKNQ | |
| | VSLTCLVKGFYPSDIAVEWESNGQPENNYKT | |
| | TPPVLDSDGSFFLYSKLTVDKSRWQQGNVF | |
| | SCSVMHEALHNHYTQKSLSLSPGK | |
| 131 | ASTKGPSVFPLAPCSRSTSESTAALGCLVKD | Human IgG4 (UniProt |
| | YFPEPVTVSWNSGALTSGVHTFPAVLQSSG | P01861) |
| | LYSLSSVVTVPSSSLGTKTYTCNVDHKPSNT | |
| | KVDKRVESKYGPPCPSCPAPEFLGGPSVFLF | |
| | PPKPKDTLMISRTPEVTCVVVDVSQEDPEVQ | |
| | FNWYVDGVEVHNAKTKPREEQFNSTYRVVS | |
| | VLTVLHQDWLNGKEYKCKVSNKGLPSSIEKT | |
| | ISKAKGQPREPQVYTLPPSQEEMTKNQVSLT | |
| | CLVKGFYPSDIAVEWESNGQPENNYKTTPP | |
| | VLDSDGSFFLYSRLTVDKSRWQEGNVFSCS | |
| | VMHEALHNHYTQKSLSLSLGK | |
| 132 | ASFKGPSVFPLVPCSRSTSESTAALGCLVKD | Human IgG4 |
| | YFPEPVTVSWNSCALTSGVHTFPAVLQSSGL | (GenBank |
| | YSLSSVVTVPSSSLGTKTYTCNVDHKPSNTK | CAC20457.1) |
| | VDKRVESKYGPPCPSCPAPEFLGGPSVFLFP | |
| | PKPKDTLMISRTPEVTCVVVDVSQEDPEVQF | |
| | NWYVDGVEVHNAKTKPREEQFNSTYRVVRV | |
| | LTVLHQDWLNGKEYKCKVSNKGLPSSIEKTI | |
| | SKAKGQPREPQVYTLPPSQEEMTKNQVSLT | |
| | CLVKGFYPSDIAVEWESNGQPEDNYKTTPP | |
| | VLDSDGSFFLYSRLTVDKSRWQEGNVFSCS | |
| | VMHEALHNHYTQKSLSLSPGK | |
| 133 | EEELQVIQPDKSVSVAAGESAILHCTVTSLIP | TTI-621 |
| | VGPIQWFRGAGPARELIYNQKEGHFPRVTTV | |
| | SESTKRENMDFSISISNITPADAGTYYCVKFR | |
| | KGSPDTEFKSGAGTELSVRAKPSDKTHTCP | |
| | PCPAPELLGGPSVFLFPPKPKDTLMISRTPEV | |
| | TCVVVDVSHEDPEVKFNWYVDGVEVHNAKT | |
| | KPREEQYNSTYRVVSVLTVLHQDWLNGKEY | |
| | KCKVSNKALPAPIEKTISKAKGQPREPQVYTL | |
| | PPSRDELTKNQVSLTCLVKGFYPSDIAVEWE | |
| | SNGQPENNYKTTPPVLDSDGSFFLYSKLTVD | |
| | KSRWQQGNVFSCSVMHEALHNHYTQKSLSL | |
| | SPGK | |
| 134 | EEELQVIQPDKSVSVAAGESAILHCTVTSLIP | TTI-622 |
| | VGPIQWFRGAGPARELIYNQKEGHFPRVTTV | |
| | SESTKRENMDFSISISNITPADAGTYYCVKFR | |
| | KGSPDTEFKSGAGTELSVRAKPSESKYGPP | |
| | CPPCPAPEFLGGPSVFLFPPKPKDTLMISRT | |
| | PEVTCVVVDVSQEDPEVQFNWYVDGVEVH | |
| | NAKTKPREEQFNSTYRVVSVLTVLHQDWLN | |
| | GKEYKCKVSNKGLPSSIEKTISKAKGQPREP | |
| | QVYTLPPSQEEMTKNQVSLTCLVKGFYPSDI | |
| | AVEWESNGQPENNYKTTPPVLDSDGSFFLY | |
| | SRLTVDKSRWQEGNVFSCSVMHEALHNHYT | |
| | QKSLSLSLGK | |

TTI-621, TTI-622, and other related fusion proteins are disclosed in PCT Publ. No. WO14/94122, the contents of which are hereby incorporated by reference herein with regard to said proteins. AL148 is a fusion protein made up of the N-terminal D1 domain of SIRPα fused to a modified human IgG1 Fc domain (Kauder et al. PLOS One (13 (8): e0201832 (2018)). Detailed descriptions of exemplary SIRPα fusion proteins including sequences can be found, for example, in PCT Publ. Nos. WO14/94122; WO16/23040; WO17/27422; WO17/177333; and WO18/176132, the disclosures of which are hereby incorporated herein in their entirety, including the sequence listings, specifications, and figures.

SIRPα-containing fusion proteins, including TTI-621, are being developed for the treatment of cancer, such as hematologic malignancies, alone or in combination with other cancer therapy drugs. A phase 1 trial evaluating dosage and safety (NCT02663518) of intravenous TTI-621 administration in patients with relapsed/refractory hematologic malignancies and selected solid tumors found that TTI-621 was well tolerated and demonstrated activity both as a monotherapy and in combination with other cancer treatment agents (Ansell et al. Clin Cancer Res 27 (8): 2190-2199 (2021)). In the initial escalation phase, subjects received TTI-621 at dosages of 0.05, 0.1, 0.3, 1, 3, and 10 mg/kg to evaluate safety and maximum tolerated dose (MTD). In the expansion phase, subjects received the MTD of 0.2 mg/kg as a monotherapy or 0.1 mg/kg in combination with rituximab or nivolumab.

### V. Uses of CD47-SIRPα Blockade Agents and Associated Methods

Disclosed herein are methods and compositions for the use of CD47-SIRPα blockade agents in reducing or eliminating a population of cells engineered to express a tolerogenic factor, such as CD47, wherein the population of cells was previously administered to a subject. In some embodiments, the population of cells is further engineered to express at least one CAR. In some embodiments, the population of cells is further engineered to express an additional factor. In some embodiments, the population of cells is further engineered to express at least one CAR and an additional factor. In some embodiments, the cells are primary cells. In some embodiments, the cells are T cells. In some embodiments, the T cells are differentiated from pluripotent cells, such as induced pluripotent cells (iPSCs). In some embodiments, the T cells are primary T cells. In some embodiments, the T cells are allogeneic T cells. In some embodiments, the cells are pancreatic islet cells. In some embodiments, the pancreatic islet cells are differentiated from pluripotent cells, such as iPSCs. In some embodiments, the pancreatic islet cells are primary pancreatic islet cells. In some embodiments, the pancreatic islet cells are allogeneic pancreatic islet cells.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the cells are differentiated from iPSCs. In some embodiments, the cells are differentiated cells. In some embodiments, differentiated cells are selected from the group consisting of cardiac cells, neural cells, endothelial cells, T cells, pancreatic islet cells, retinal pigmented epithelium cells, hepatocytes, thyroid cells, skin cells, blood cells, primary cells, and epithelial cells. In some embodiments, the cells are primary cells. In some embodiments, the primary cells are T cells or pancreatic islet cells. In some embodiments, the primary cells are T cells. In some embodiments, the primary cells are pancreatic islet cells. In some embodiments, the cells are engineered to express at least one additional factor selected from the group consisting of CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8, and a combination thereof.

In some embodiments, the additional factor is CD16. In some embodiments, the additional factor is CD24. In some embodiments, the additional factor is CD35. In some embodiments, the additional factor is CD39. In some embodiments, the additional factor is CD46. In some embodiments, the additional factor is CD52. In some embodiments, the additional factor is CD55. In some embodiments, the additional factor is CD59. In some embodiments, the additional factor is CD200. In some embodiments, the additional factor is CCL22. In some embodiments, the additional factor is CTLA4-Ig. In some embodiments, the additional factor is C1 inhibitor. In some embodiments, the additional factor is FASL. In some embodiments, the additional factor is IDO1. In some embodiments, the additional factor is HLA-C. In some embodiments, the additional factor is HLA-E. In some embodiments, the additional factor is HLA-E heavy chain. In some embodiments, the additional factor is HLA-G. In some embodiments, the additional factor is IL-10. In some embodiments, the additional factor is IL-35. In some embodiments, the additional factor is PD-1. In some embodiments, the additional factor is PD-L1. In some embodiments, the additional factor is Serpinb9. In some embodiments, the additional factor is CCI21. In some embodiments, the additional factor is Mfge8. In some embodiments, the cells have reduced expression of MHC class I HLA and/or MHC class II HLA molecules.

In some embodiments, a method disclosed herein comprises administering to a subject in need thereof a CD47-SIRPα blockade agent, wherein the subject was previously administered a population of T cells engineered to express an exogenous CD47 polypeptide. In some embodiments, the cells have reduced expression of MHC class I HLA and/or MHC class II HLA molecules. In some embodiments, a method disclosed herein comprises administering to a subject in need thereof a CD47-SIRPα blockade agent, wherein the subject was previously administered a population of T cells (i) engineered to express an exogenous CD47 polypeptide and at least one chimeric antigen receptor (CAR) and (ii) having reduced expression of MHC class I HLA molecules, MHC class II HLA molecules, T cell receptor (TCR) alpha, and/or TCR beta. In some embodiments, a method disclosed herein comprises administering to a subject in need thereof a CD47-SIRPα blockade agent, wherein the subject was previously administered a population of T cells having reduced expression of MHC class I HLA molecules, MHC class II HLA molecules, and TCR alpha and engineered to express an exogenous CD47 polypeptide and a CD19 chimeric antigen receptor (CAR). In some embodiments, a method disclosed herein comprises administering to a subject in need thereof a CD47-SIRPα blockade agent, wherein the subject was previously administered a population of T cells having reduced expression of MHC class I HLA molecules, MHC class II HLA molecules, and TCR beta and engineered to express an exogenous CD47 polypeptide and a CD19 chimeric antigen receptor (CAR). In some embodiments, the CAR binds an antigen selected from the group consisting of CD19, CD20, CD22, CD38, CD123, CD138, BCMA, and a combination thereof. In some embodiments, MHC class I and/or MHC class II expression is knocked out. In some embodiments, the reduced expression of MHC class I HLA is mediated by reduced expression of B2M and reduced expression of MHC class II is mediated by reduced expression of CIITA. In some embodiments, B2M and/or CIITA expression is knocked out. In some embodiments, the CAR binds a CD19 antigen and is a CD19 CAR. In some embodiments, the CAR binds a CD20 antigen and is a CD20 CAR. In some embodiments, the CAR binds a CD22 antigen and is a CD22 CAR. In some embodiments, the CAR binds a CD38 antigen and is a CD38 CAR. In some embodiments, the CAR binds a CD123 antigen and is a CD123 CAR. In some embodiments, the CAR binds a CD138 antigen and is a CD138 CAR. In some embodiments, the CAR binds a BCMA antigen and is a BCMA CAR.

In some embodiments, the T cells are primary cells. In some embodiments, the T cells are allogeneic. In some embodiments, the T cells are differentiated from iPSCs. In some embodiments, the T cells are engineered to have reduced expression of TCRα and/or TCRβ. In some embodiments, the T cells are engineered to have reduced expression of cytotoxic T-lymphocyte-associated protein 4 (CTLA4) and/or programmed cell death (PD1).

In some embodiments, a method disclosed herein comprises administering to a subject in need thereof a CD47-SIRPα blockade agent, wherein the subject was previously administered a population of T cells engineered to express an exogenous CD47 polypeptide. In some embodiments, the T cells are further engineered to express a chimeric antigen receptor (CAR). In some embodiments, the CAR is a CD19 CAR selected from the group consisting of tisagenlecleucel, lisocabtagene maraleucel, axicabtagene ciloleucel, and brexucabtagene autoleucel. In some embodiments, the CD19 CAR is tisagenlecleucel. In some embodiments, the CD19 CAR is lisocabtagene. In some embodiments, the CD19 CAR is maraleucel. In some embodiments, the CD19 CAR is axicabtagene. In some embodiments, the CD19 CAR is ciloleucel. In some embodiments, the CD19 CAR is brexucabtagene autoleucel. In some embodiments, the CAR is a CD19 CAR comprising the amino acid sequence of SEQ ID NO:117. In some embodiments, the CD19 CAR is encoded by the nucleic acid sequence of SEQ ID NO:116. In some embodiments, the T cells are engineered to express at least one additional factor selected from the group consisting of CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8, and a combination thereof. In some embodiments, the additional factor is CD16. In some embodiments, the additional factor is CD24. In some embodiments, the additional factor is CD35. In some embodiments, the additional factor is CD39. In some embodiments, the additional factor is CD46. In some embodiments, the additional factor is CD52. In some embodiments, the additional factor is CD55. In some embodiments, the additional factor is CD59. In some embodiments, the additional factor is CD200. In some embodiments, the additional factor is CCL22. In some embodiments, the additional factor is CTLA4-Ig. In some embodiments, the additional factor is C1 inhibitor. In some embodiments, the additional factor is FASL. In some embodiments, the additional factor is IDO1. In some embodiments, the additional factor is HLA-C. In some embodiments, the additional factor is HLA-E. In some embodiments, the additional factor is HLA-E heavy chain. In some embodiments, the additional factor is HLA-G. In some embodiments, the additional factor is IL-10. In some embodiments, the additional factor is IL-35. In some embodiments, the additional factor is PD-1. In some embodiments, the additional factor is PD-L1. In some embodiments, the additional factor is Serpinb9. In some embodiments, the additional factor is CCI21. In some embodiments, the additional factor is Mfge8. In some embodiments, the CAR and a gene encoding the exogenous CD47 polypeptide were introduced into the T cells in a bicistronic vector. In some embodiments, the bicistronic vector was introduced into the T cells via a lentivirus. In some embodiments, the CAR and the gene encoding the exogenous CD47 polypeptide are under the control of a single promoter.

In some embodiments, a method disclosed herein is a method of reducing a population of cells engineered to express an exogenous CD47 polypeptide in a subject comprising: (a) administering to the subject a first dose of a CD47-SIRPα blockade agent; (b) determining a first outcome of the first dose of the CD47-SIRPα blockade agent administered in (a); (c) optionally administering a second dose of the CD47-SIRPα blockade agent based on the first outcome in (b); and (d) optionally determining a second outcome of the second dose of the CD47-SIRPα blockade agent administered in (c). In some embodiments, the cells are differentiated from iPSCs. In some embodiments, the cells are differentiated cells. In some embodiments, differentiated cells are selected from the group consisting of cardiac cells, neural cells, endothelial cells, T cells, pancreatic islet cells, retinal pigmented epithelium cells, hepatocytes, thyroid cells, skin cells, blood cells, primary cells, and epithelial cells. In some embodiments, the cells are primary cells. In some embodiments, the primary cells are T cells or pancreatic islet cells. In some embodiments, the primary cells are T cells. In some embodiments, the primary cells are pancreatic islet cells. In some embodiments, the cells are engineered to express at least one additional factor selected from the group consisting of CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8, and a combination thereof. In some embodiments, the additional factor is CD16. In some embodiments, the additional factor is CD24. In some embodiments, the additional factor is CD35. In some embodiments, the additional factor is CD39. In some embodiments, the additional factor is CD46. In some embodiments, the additional factor is CD52. In some embodiments, the additional factor is CD55. In some embodiments, the additional factor is CD59. In some embodiments, the additional factor is CD200. In some embodiments, the additional factor is CCL22. In some embodiments, the additional factor is CTLA4-Ig. In some embodiments, the additional factor is C1 inhibitor. In some embodiments, the additional factor is FASL. In some embodiments, the additional factor is IDO1. In some embodiments, the additional factor is HLA-C. In some embodiments, the additional factor is HLA-E. In some embodiments, the additional factor is HLA-E heavy chain. In some embodiments, the additional factor is HLA-G. In some embodiments, the additional factor is IL-10. In some embodiments, the additional factor is IL-35. In some embodiments, the additional factor is PD-1. In some embodiments, the additional factor is PD-L1. In some embodiments, the additional factor is Serpinb9. In some embodiments, the additional factor is CCI21. In some embodiments, the additional factor is Mfge8. In some embodiments, the cells have reduced expression of MHC class I HLA and/or MHC class II HLA molecules. In some embodiments, MHC class I and/or MHC class II expression is knocked out. In some embodiments, the reduced expression of MHC class I HLA is mediated by reduced expression of B2M and reduced expression of MHC class II is mediated by reduced expression of CIITA. In some embodiments, B2M and/or CIITA expression is knocked out.

In some embodiments, a method disclosed herein is a method of reducing a population of cells engineered to express an exogenous CD47 polypeptide in a subject comprising: (a) administering to the subject a first dose of a CD47-SIRPα blockade agent; (b) determining a first outcome of the first dose of the CD47-SIRPα blockade agent administered in (a); (c) optionally administering a second dose of the CD47-SIRPα blockade agent based on the first outcome in (b); and (d) optionally determining a second outcome of the second dose of the CD47-SIRPα blockade agent administered in (c). In some embodiments, the first outcome and second outcome are independently selected from the group consisting of: (i) a reduction in the number of cells by between about 10% and 100%, (ii) a reduction in an adverse event by between about 10% and 100%, and (iii) a combination of (i) and (ii). In some embodiments, the first outcome and/or the second outcome is an adverse event. In some embodiments, the adverse event is selected from the group consisting of hyperproliferation, transformation, tumor formation, cytokine release syndrome, graft-versus-host disease (GVHD), immune effector cell-associated neurotoxicity syndrome (ICANS), inflammation, infection, nausea, vomiting, bleeding, interstitial pneumonitis, respiratory disease, jaundice, weight loss, diarrhea, loss of appetite, cramps, abdominal pain, hepatic veno-occlusive disease (VOD), graft failure, organ damage, infertility, hormonal changes, abnormal growth formation, cataracts, and post-transplant lymphoproliferative disorder (PTLD). In some embodiments, the adverse event is hyperproliferation. In some embodiments, the adverse event is transformation. In some embodiments, the adverse event is tumor formation. In some embodiments, the adverse event is cytokine release syndrome. In some embodiments, the adverse event is graft-versus-host disease (GVHD). In some embodiments, the adverse event is immune effector cell-associated neurotoxicity syndrome (ICANS). In some embodiments, the adverse event is inflammation. In some embodiments, the adverse event is infection. In some embodiments, the adverse event is nausea. In some embodiments, the adverse event is vomiting. In some embodiments, the adverse event is bleeding. In some embodiments, the adverse event is interstitial pneumonitis. In some embodiments, the adverse event is respiratory disease. In some embodiments, the adverse event is jaundice. In some embodiments, the adverse event is weight loss. In some embodiments, the adverse event is diarrhea. In some embodiments, the adverse event is loss of appetite. In some embodiments, the adverse event is cramps. In some embodiments, the adverse event is abdominal pain. In some embodiments, the adverse event is hepatic veno-occlusive disease (VOD). In some embodiments, the adverse event is graft failure. In some embodiments, the adverse event is organ damage. In some embodiments, the adverse event is infertility. In some embodiments, the adverse event is hormonal changes. In some embodiments, the adverse event is abnormal growth formation. In some embodiments, the adverse event is cataracts. In some embodiments, the adverse event is post-transplant lymphoproliferative disorder (PTLD).

In some embodiments, a method disclosed herein is a method of reducing a population of cells engineered to express an exogenous CD47 polypeptide in a subject comprising: (a) administering to the subject a first dose of a CD47-SIRPα blockade agent; (b) determining a first outcome of the first dose of the CD47-SIRPα blockade agent administered in (a); (c) optionally administering a second dose of the CD47-SIRPα blockade agent based on the first outcome in (b); and (d) optionally determining a second outcome of the second dose of the CD47-SIRPα blockade agent administered in (c). In some embodiments, the first outcome comprises a reduction in the number of cells by between about 10% and about 15%. In some embodiments, the first outcome comprises a reduction in the number of cells by between about 15% and about 20%. In some embodiments, the first outcome comprises a reduction in the number of cells by between about 20% and about 25%. In some embodiments, the first outcome comprises a reduction in the number of cells by between about 25% and about 30%. In some embodiments, the first outcome comprises a reduction in the number of cells by between about 30% and about 35%. In some embodiments, the first outcome comprises a reduction in the number of cells by between about 35% and about 40%. In some embodiments, the first outcome comprises a reduction in the number of cells by between about 40% and about 45%. In some embodiments, the first outcome comprises a reduction in the number of cells by between about 45% and about 50%. In some embodiments, the first outcome comprises a reduction in the number of cells by between about 50% and about 55%. In some embodiments, the first outcome comprises a reduction in the number of cells by between about 55% and about 60%. In some embodiments, the first outcome comprises a reduction in the number of cells by between about 60% and about 65%. In some embodiments, the first outcome comprises a reduction in the number of cells by between about 65% and about 70%. In some embodiments, the first outcome comprises a reduction in the number of cells by between about 70% and about 75%. In some embodiments, the first outcome comprises a reduction in the number of cells by between about 75% and about 80%. In some embodiments, the first outcome comprises a reduction in the number of cells by between about 80% and about 85%. In some embodiments, the first outcome comprises a reduction in the number of cells by between about 85% and about 90%. In some embodiments, the first outcome comprises a reduction in the number of cells by between about 90% and about 95%. In some embodiments, the first outcome comprises a reduction in the number of cells by between about 95% and about 100%.

In some embodiments, a method disclosed herein is a method of reducing a population of cells engineered to express an exogenous CD47 polypeptide in a subject comprising: (a) administering to the subject a first dose of a CD47-SIRPα blockade agent; (b) determining a first outcome of the first dose of the CD47-SIRPα blockade agent administered in (a); (c) optionally administering a second dose of the CD47-SIRPα blockade agent based on the first outcome in (b); and (d) optionally determining a second outcome of the second dose of the CD47-SIRPα blockade agent administered in (c). In some embodiments, the first outcome comprises a reduction in an adverse event by between about 10% and about 15%. In some embodiments, the first outcome comprises a reduction in an adverse event by between about 15% and about 20%. In some embodiments, the first outcome comprises a reduction in an adverse event by between about 20% and about 25%. In some embodiments, the first outcome comprises a reduction in an adverse event by between about 25% and about 30%. In some embodiments, the first outcome comprises a reduction in an adverse event by between about 30% and about 35%. In some embodiments, the first outcome comprises a reduction in an adverse event by between about 35% and about 40%. In some embodiments, the first outcome comprises a reduction in an adverse event by between about 40% and about 45%. In some embodiments, the first outcome comprises a reduction in an adverse event by between about 45% and about 50%. In some embodiments, the first outcome comprises a reduction in an adverse event by between about 50% and about 55%. In some embodiments, the first outcome comprises a reduction in an adverse event by between about 55% and about 60%. In some embodiments, the first outcome comprises a reduction in an adverse event by between about 60% and about 65%. In some embodiments, the first outcome comprises a reduction in an adverse event by between about 65% and about 70%. In some embodiments, the first outcome comprises a reduction in an adverse event by between about 70% and about 75%. In some embodiments, the first outcome comprises a reduction in an adverse event by between about 75% and about 80%. In some embodiments, the first outcome comprises a reduction in an adverse event by between about 80% and about 85%. In some embodiments, the first outcome comprises a reduction in an adverse event by between about 85% and about 90%. In some embodiments, the first outcome comprises a reduction in an adverse event by between about 90% and about 95%. In some embodiments, the first outcome comprises a reduction in an adverse event by between about 95% and about 100%.

In some embodiments, a method disclosed herein is a method of reducing a population of cells engineered to express an exogenous CD47 polypeptide in a subject comprising: (a) administering to the subject a first dose of a CD47-SIRPα blockade agent; (b) determining a first outcome of the first dose of the CD47-SIRPα blockade agent administered in (a); (c) optionally administering a second dose of the CD47-SIRPα blockade agent based on the first outcome in (b); and (d) optionally determining a second outcome of the second dose of the CD47-SIRPα blockade agent administered in (c). In some embodiments, the second outcome comprises a reduction in the number of cells by between about 10% and about 15%. In some embodiments, the second outcome comprises a reduction in the number of cells by between about 15% and about 20%. In some embodiments, the second outcome comprises a reduction in the number of cells by between about 20% and about 25%. In some embodiments, the second outcome comprises a reduction in the number of cells by between about 25% and about 30%. In some embodiments, the second outcome comprises a reduction in the number of cells by between about 30% and about 35%. In some embodiments, the second outcome comprises a reduction in the number of cells by between about 35% and about 40%. In some embodiments, the second outcome comprises a reduction in the number of cells by between about 40% and about 45%. In some embodiments, the second outcome comprises a reduction in the number of cells by between about 45% and about 50%. In some embodiments, the second outcome comprises a reduction in the number of cells by between about 50% and about 55%. In some embodiments, the second outcome comprises a reduction in the number of cells by between about 55% and about 60%. In some embodiments, the second outcome comprises a reduction in the number of cells by between about 60% and about 65%. In some embodiments, the second outcome comprises a reduction in the number of cells by between about 65% and about 70%. In some embodiments, the second outcome comprises a reduction in the number of cells by between about 70% and about 75%. In some embodiments, the second outcome comprises a reduction in the number of cells by between about 75% and about 80%. In some embodiments, the second outcome comprises a reduction in the number of cells by between about 80% and about 85%. In some embodiments, the second outcome comprises a reduction in the number of cells by between about 85% and about 90%. In some embodiments, the second outcome comprises a reduction in the number of cells by between about 90% and about 95%. In some embodiments, the second outcome comprises a reduction in the number of cells by between about 95% and about 100%.

In some embodiments, a method disclosed herein is a method of reducing a population of cells engineered to express an exogenous CD47 polypeptide in a subject comprising: (a) administering to the subject a first dose of a CD47-SIRPα blockade agent; (b) determining a first outcome of the first dose of the CD47-SIRPα blockade agent administered in (a); (c) optionally administering a second dose of the CD47-SIRPα blockade agent based on the first outcome in (b); and (d) optionally determining a second outcome of the second dose of the CD47-SIRPα blockade agent administered in (c). In some embodiments, the second outcome comprises a reduction in an adverse event by between about 10% and about 15%. In some embodiments, the second outcome comprises a reduction in an adverse event by between about 15% and about 20%. In some embodiments, the second outcome comprises a reduction in an adverse event by between about 20% and about 25%. In some embodiments, the second outcome comprises a reduction in an adverse event by between about 25% and about 30%. In some embodiments, the second outcome comprises a reduction in an adverse event by between about 30% and about 35%. In some embodiments, the second outcome comprises a reduction in an adverse event by between about 35% and about 40%. In some embodiments, the second outcome comprises a reduction in an adverse event by between about 40% and about 45%. In some embodiments, the second outcome comprises a reduction in an adverse event by between about 45% and about 50%. In some embodiments, the second outcome comprises a reduction in an adverse event by between about 50% and about 55%. In some embodiments, the second outcome comprises a reduction in an adverse event by between about 55% and about 60%. In some embodiments, the second outcome comprises a reduction in an adverse event by between about 60% and about 65%. In some embodiments, the second outcome comprises a reduction in an adverse event by between about 65% and about 70%. In some embodiments, the second outcome comprises a reduction in an adverse event by between about 70% and about 75%. In some embodiments, the second outcome comprises a reduction in an adverse event by between about 75% and about 80%. In some embodiments, the second outcome comprises a reduction in an adverse event by between about 80% and about 85%. In some embodiments, the second outcome comprises a reduction in an adverse event by between about 85% and about 90%. In some embodiments, the second outcome comprises a reduction in an adverse event by between about 90% and about 95%. In some embodiments, the second outcome comprises a reduction in an adverse event by between about 95% and about 100%.

In some embodiments, a method disclosed herein comprises: (a) quantifying a population of cells engineered to express an exogenous CD47 polypeptide in a subject; (b) determining a first dose of a CD47-SIRPα blockade agent that is effective in reducing the population of cells by at least 20%; and (c) administering the first dose of the CD47-SIRPα blockade agent to the subject. In some embodiments, the cells are differentiated from iPSCs. In some embodiments, the cells are differentiated cells. In some embodiments, differentiated cells are selected from the group consisting of cardiac cells, neural cells, endothelial cells, T cells, pancreatic islet cells, retinal pigmented epithelium cells, hepatocytes, thyroid cells, skin cells, blood cells, primary cells, and epithelial cells. In some embodiments, the cells are primary cells. In some embodiments, the primary cells are T cells or pancreatic islet cells. In some embodiments, the primary cells are T cells. In some embodiments, the primary cells are pancreatic islet cells. In some embodiments, the cells are engineered to express at least one additional factor selected from the group consisting of CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8, and a combination thereof. In some embodiments, the additional factor is CD16. In some embodiments, the additional factor is CD24. In some embodiments, the additional factor is CD35. In some embodiments, the additional factor is CD39. In some embodiments, the additional factor is CD46. In some embodiments, the additional factor is CD52. In some embodiments, the additional factor is CD55. In some embodiments, the additional factor is CD59. In some embodiments, the additional factor is CD200. In some embodiments, the additional factor is CCL22. In some embodiments, the additional factor is CTLA4-Ig. In some embodiments, the additional factor is C1 inhibitor. In some embodiments, the additional factor is FASL. In some embodiments, the additional factor is IDO1. In some embodiments, the additional factor is HLA-C. In some embodiments, the additional factor is HLA-E. In some embodiments, the additional factor is HLA-E heavy chain. In some embodiments, the additional factor is HLA-G. In some embodiments, the additional factor is IL-10. In some embodiments, the additional factor is IL-35. In some embodiments, the additional factor is PD-1. In some embodiments, the additional factor is PD-L1. In some embodiments, the additional factor is Serpinb9. In some embodiments, the additional factor is CCI21. In some embodiments, the additional factor is Mfge8. In some embodiments, the cells have reduced expression of MHC class I HLA and/or MHC class II HLA molecules. In some embodiments, MHC class I and/or MHC class II expression is knocked out. In some embodiments, the reduced expression of MHC class I HLA is mediated by reduced expression of B2M and reduced expression of MHC class II is mediated by reduced expression of CIITA. In some embodiments, B2M and/or CIITA expression is knocked out.

In some embodiments, a method disclosed herein comprises: (a) quantifying a population of cells engineered to express an exogenous CD47 polypeptide in a subject; (b) determining a first dose of a CD47-SIRPα blockade agent that is effective in reducing the population of cells by at least 20%; and (c) administering the first dose of the CD47-SIRPα blockade agent to the subject. In some embodiments, the first dose is effective in reducing the population of cells by between about 20% and about 30%. In some embodiments, the first dose is effective in reducing the population of cells by between about 30% and about 40%. In some embodiments, the first dose is effective in reducing the population of cells by between about 40% and about 50%. In some embodiments, the first dose is effective in reducing the population of cells by between about 50% and about 60%. In some embodiments, the first dose is effective in reducing the population of cells by between about 60% and about 70%. In some embodiments, the first dose is effective in reducing the population of cells by between about 70% and about 80%. In some embodiments, the first dose is effective in reducing the population of cells by between about 80% and about 90%. In some embodiments, the first dose is effective in reducing the population of cells by between about 90% and about 100%.

In some embodiments, a method disclosed herein comprises: (a) quantifying a population of cells engineered to express an exogenous CD47 polypeptide in a subject; (b) determining a first dose of a CD47-SIRPα blockade agent that is effective in reducing the population of cells by at least 20%; and (c) administering the first dose of the CD47-SIRPα blockade agent to the subject. In some embodiments, the first dose and/or second dose is administered: (i) at 0.05, 0.1, 0.3, 1, 3, or 10 mg/kg; (ii) once every 12 hours, once every 24 hours, once every 36 hours, or once every 48 hours; and/or (iii) for between 1 day and 3 weeks. In some embodiments, the first and the second dose are the same. In some embodiments, the first dose and/or second dose is administered at 0.05 mg/kg. In some embodiments, the first dose and/or second dose is administered at 0.1 mg/kg. In some embodiments, the first dose and/or second dose is administered at 0.3 mg/kg. In some embodiments, the first dose and/or second dose is administered at 1 mg/kg. In some embodiments, the first dose and/or second dose is administered at 3 mg/kg. In some embodiments, the first dose and/or second dose is administered at 10 mg/kg. In some embodiments, the first dose and/or second dose is administered at between about 0.01 mg/kg and about 20 mg/kg. In some embodiments, the first dose and/or second dose is administered at between about 0.01 mg/kg and about 0.05 mg/kg. In some embodiments, the first dose and/or second dose is administered at between about 0.05 mg/kg and about 0.1 mg/kg. In some embodiments, the first dose and/or second dose is administered at between about 0.1 mg/kg and about 0.5 mg/kg. In some embodiments, the first dose and/or second dose is administered at between about 0.5 mg/kg and about 1 mg/kg. In some embodiments, the first dose and/or second dose is administered at between about 1 mg/kg and about 5 mg/kg. In some embodiments, the first dose and/or second dose is administered at between about 5 mg/kg and about 10 mg/kg. In some embodiments, the first dose and/or second dose is administered at between about 10 mg/kg and about 15 mg/kg. In some embodiments, the first dose and/or second dose is administered at between about 15 mg/kg and about 20 mg/kg.

In some embodiments, a method disclosed herein comprises: (a) quantifying a population of cells engineered to express an exogenous CD47 polypeptide in a subject; (b) determining a first dose of a CD47-SIRPα blockade agent that is effective in reducing the population of cells by at least 20%; and (c) administering the first dose of the CD47-SIRPα blockade agent to the subject. In some embodiments, the first dose and/or second dose is administered once every 6 hours. In some embodiments, the first dose and/or second dose is administered once every 12 hours. In some embodiments, the first dose and/or second dose is administered once every 18 hours. In some embodiments, the first dose and/or second dose is administered once every 24 hours. In some embodiments, the first dose and/or second dose is administered once every 36 hours. In some embodiments, the first dose and/or second dose is administered once every 48 hours. In some embodiments, the first dose and/or second dose is administered once every 3 days. In some embodiments, the first dose and/or second dose is administered once every 4 days. In some embodiments, the first dose and/or second dose is administered once every 5 days. In some embodiments, the first dose and/or second dose is administered once every 6 days. In some embodiments, the first dose and/or second dose is administered once every 7 days. In some embodiments, the first dose and/or second dose is administered once every 2 weeks. In some embodiments, the first dose and/or second dose is administered once every 4 weeks. In some embodiments, the first dose and/or second dose is administered once every 6 weeks. In some embodiments, the first dose and/or second dose is administered once every 8 weeks. In some embodiments, the first dose and/or second dose is administered once every 3 months. In some embodiments, the first dose and/or second dose is administered once every 4 months. In some embodiments, the first dose and/or second dose is administered once every 5 months. In some embodiments, the first dose and/or second dose is administered once every 6 months. In some embodiments, the first dose and/or second dose is administered once every between about 6 months and about 12 months. In some embodiments, the first dose and/or second dose is administered once every 18 months. In some embodiments, the first dose and/or second dose is administered once every 24 months. In some embodiments, the first dose and/or second dose is administered once every 3 years. In some embodiments, the first dose and/or second dose is administered once every 4 years. In some embodiments, the first dose and/or second dose is administered once every 5 years.

In some embodiments, a method disclosed herein comprises: (a) quantifying a population of cells engineered to express an exogenous CD47 polypeptide in a subject; (b) determining a first dose of a CD47-SIRPα blockade agent that is effective in reducing the population of cells by at least 20%; and (c) administering the first dose of the CD47-SIRPα blockade agent to the subject. In some embodiments, the first dose and/or second dose is administered for between about 1 day and about 50 years. In some embodiments, the first dose and/or second dose is administered for between about 1 day and about 1 week. In some embodiments, the first dose and/or second dose is administered for between about 1 week and about 2 weeks. In some embodiments, the first dose and/or second dose is administered for between about 2 weeks and about 3 weeks. In some embodiments, the first dose and/or second dose is administered for between about 3 weeks and about 1 month. In some embodiments, the first dose and/or second dose is administered for between about 1 month and about 2 months. In some embodiments, the first dose and/or second dose is administered for between about 2 months and about 3 months. In some embodiments, the first dose and/or second dose is administered for between about 3 months and about 4 months. In some embodiments, the first dose and/or second dose is administered for between about 4 months and about 5 months. In some embodiments, the first dose and/or second dose is administered for between about 5 months and about 6 months. In some embodiments, the first dose and/or second dose is administered for between about 6 months and about 1 year. In some embodiments, the first dose and/or second dose is administered for between about 1 year and about 2 years. In some embodiments, the first dose and/or second dose is administered for between about 2 years and about 3 years. In some embodiments, the first dose and/or second dose is administered for between about 3 years and about 4 years. In some embodiments, the first dose and/or second dose is administered for between about 4 years and about 5 years. In some embodiments, the first dose and/or second dose is administered for between about 5 years and about 10 years. In some embodiments, the first dose and/or second dose is administered for between about 10 years and about 15 years. In some embodiments, the first dose and/or second dose is administered for between about 15 years and about 20 years. In some embodiments, the first dose and/or second dose is administered for between about 20 years and about 30 years. In some embodiments, the first dose and/or second dose is administered for between about 30 years and about 40 years. In some embodiments, the first dose and/or second dose is administered for between about 40 years and about 50 years. In some embodiments, the first dose and/or second dose is administered for the lifetime of the subject.

In some embodiments, a method disclosed herein comprises administering to a subject in need thereof a CD47-SIRPα blockade agent, wherein the subject was previously administered a population of pancreatic islet cells engineered to express an exogenous CD47 polypeptide. In some embodiments, a method disclosed herein comprises administering to a subject in need thereof a CD47-SIRPα blockade agent, wherein the subject was previously administered a population of pancreatic islet cells (i) engineered to express an exogenous CD47 polypeptide and (ii) having reduced expression of MHC class I HLA and/or MHC class II HLA molecules. In some embodiments, a method disclosed herein comprises administering to a subject in need thereof a CD47-SIRPα blockade agent, wherein the subject was previously administered a population of pancreatic islet cells (i) engineered to express exogenous CD47, CD46, and CD59 polypeptides and (ii) having reduced expression of MHC class I HLA and/or MHC class II HLA molecules. In some embodiments, MHC class I and/or MHC class II expression is knocked out. In some embodiments, the reduced expression of MHC class I HLA is mediated by reduced expression of B2M and reduced expression of MHC class II is mediated by reduced expression of CIITA. In some embodiments, B2M and/or CIITA expression is knocked out. In some embodiments, the pancreatic islet cells are engineered to have reduced expression of CD142. In some embodiments, the pancreatic islet cells are primary cells. In some embodiments, the pancreatic islet cells are differentiated from iPSCs.

In some embodiments, a method disclosed herein comprises administering to a subject in need thereof a CD47-SIRPα blockade agent, wherein the subject was previously administered a population of pancreatic islet cells engineered to express an exogenous CD47 polypeptide. In some embodiments, the pancreatic islet cells are engineered to express at least one additional factor selected from the group consisting of CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8, and a combination thereof. In some embodiments, the additional factor is CD16. In some embodiments, the additional factor is CD24. In some embodiments, the additional factor is CD35. In some embodiments, the additional factor is CD39. In some embodiments, the additional factor is CD46. In some embodiments, the additional factor is CD52. In some embodiments, the additional factor is CD55. In some embodiments, the additional factor is CD59. In some embodiments, the additional factor is CD200. In some embodiments, the additional factor is CCL22. In some embodiments, the additional factor is CTLA4-Ig. In some embodiments, the additional factor is C1 inhibitor. In some embodiments, the additional factor is FASL. In some embodiments, the additional factor is IDO1. In some embodiments, the additional factor is HLA-C. In some embodiments, the additional factor is HLA-E. In some embodiments, the additional factor is HLA-E heavy chain. In some embodiments, the additional factor is HLA-G. In some embodiments, the additional factor is IL-10. In some embodiments, the additional factor is IL-35. In some embodiments, the additional factor is PD-1. In some embodiments, the additional factor is PD-L1. In some embodiments, the additional factor is Serpinb9. In some embodiments, the additional factor is CCI21. In some embodiments, the additional factor is Mfge8.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, a gene encoding the exogenous CD47 polypeptide was introduced into the cell via homology directed repair (HDR)-mediated insertion into a genomic locus of the cell. In some embodiments, the genomic locus is selected from the group consisting of a B2M locus, a CIITA locus, a TRAC locus, a TRBC locus, and a safe harbor locus. In some embodiments, the genomic locus is a B2M locus. In some embodiments, the genomic locus is a CIITA locus. In some embodiments, the genomic locus is a TRAC locus. In some embodiments, the genomic locus is a TRBC locus. In some embodiments, the genomic locus is a safe harbor locus. In some embodiments, the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus. In some embodiments, the safe harbor locus is an AAVS1 locus. In some embodiments, the safe harbor locus is an ABO locus. In some embodiments, the safe harbor locus is a CCR5 locus. In some embodiments, the safe harbor locus is a CLYBL locus. In some embodiments, the safe harbor locus is a CXCR4 locus. In some embodiments, the safe harbor locus is an F3 locus. In some embodiments, the safe harbor locus is a FUT1 locus. In some embodiments, the safe harbor locus is an HMGB1 locus. In some embodiments, the safe harbor locus is a KDM5D locus. In some embodiments, the safe harbor locus is an LRP1 locus. In some embodiments, the safe harbor locus is a MICA locus. In some embodiments, the safe harbor locus is a MICB locus. In some embodiments, the safe harbor locus is an RHD locus. In some embodiments, the safe harbor locus is a ROSA26 locus. In some embodiments, the safe harbor locus is an SHS231 locus. In some embodiments, the cell has reduced expression of MHC class I HLA and/or MHC class II HLA molecules. In some embodiments, MHC class I and/or MHC class II expression is knocked out. In some embodiments, the reduced expression of MHC class I HLA is mediated by reduced expression of B2M and reduced expression of MHC class II is mediated by reduced expression of CIITA. In some embodiments, B2M and/or CIITA expression is knocked out.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is administered at least one day after the subject was administered the cells. In some embodiments, the CD47-SIRPα blockade agent is administered at least two days after the subject was administered the cells. In some embodiments, the CD47-SIRPα blockade agent is administered at least three days after the subject was administered the cells. In some embodiments, the CD47-SIRPα blockade agent is administered at least four days after the subject was administered the cells. In some embodiments, the CD47-SIRPα blockade agent is administered at least five days after the subject was administered the cells. In some embodiments, the CD47-SIRPα blockade agent is administered at least six days after the subject was administered the cells. In some embodiments, the CD47-SIRPα blockade agent is administered at least one week after the subject was administered the cells. In some embodiments, the CD47-SIRPα blockade agent is administered at least two weeks after the subject was administered the cells. In some embodiments, the CD47-SIRPα blockade agent is administered at least three weeks after the subject was administered the cells. In some embodiments, the CD47-SIRPα blockade agent is administered at least one month after the subject was administered the cells. In some embodiments, the CD47-SIRPα blockade agent is administered at least two months after the subject was administered the cells. In some embodiments, the CD47-SIRPα blockade agent is administered at least three months after the subject was administered the cells. In some embodiments, the CD47-SIRPα blockade agent is administered at least four months after the subject was administered the cells. In some embodiments, the CD47-SIRPα blockade agent is administered at least five months after the subject was administered the cells. In some embodiments, the CD47-SIRPα blockade agent is administered at least six months after the subject was administered the cells. In some embodiments, the CD47-SIRPα blockade agent is administered at least 1 year after the subject was administered the cells.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is administered after the subject experiences an adverse event related to the administered cells. In some embodiments, the adverse event is selected from the group consisting of hyperproliferation, transformation, tumor formation, cytokine release syndrome, graft-versus-host disease (GVHD), immune effector cell-associated neurotoxicity syndrome (ICANS), inflammation, infection, nausea, vomiting, bleeding, interstitial pneumonitis, respiratory disease, jaundice, weight loss, diarrhea, loss of appetite, cramps, abdominal pain, hepatic veno-occlusive disease (VOD), graft failure, organ damage, infertility, hormonal changes, abnormal growth formation, cataracts, and post-transplant lymphoproliferative disorder (PTLD). In some embodiments, the adverse event is hyperproliferation. In some embodiments, the adverse event is transformation. In some embodiments, the adverse event is tumor formation. In some embodiments, the adverse event is cytokine release syndrome. In some embodiments, the adverse event is graft-versus-host disease (GVHD). In some embodiments, the adverse event is immune effector cell-associated neurotoxicity syndrome (ICANS). In some embodiments, the adverse event is inflammation. In some embodiments, the adverse event is infection. In some embodiments, the adverse event is nausea. In some embodiments, the adverse event is vomiting. In some embodiments, the adverse event is bleeding. In some embodiments, the adverse event is interstitial pneumonitis. In some embodiments, the adverse event is respiratory disease. In some embodiments, the adverse event is jaundice. In some embodiments, the adverse event is weight loss. In some embodiments, the adverse event is diarrhea. In some embodiments, the adverse event is loss of appetite. In some embodiments, the adverse event is cramps. In some embodiments, the adverse event is abdominal pain. In some embodiments, the adverse event is hepatic veno-occlusive disease (VOD). In some embodiments, the adverse event is graft failure. In some embodiments, the adverse event is organ damage. In some embodiments, the adverse event is infertility. In some embodiments, the adverse event is hormonal changes. In some embodiments, the adverse event is abnormal growth formation. In some embodiments, the adverse event is cataracts. In some embodiments, the adverse event is post-transplant lymphoproliferative disorder (PTLD).

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent comprises a CD47-binding domain. In some embodiments, the CD47-binding domain comprises signal regulatory protein alpha (SIRPα) or a fragment thereof. In some embodiments, the CD47-SIRPα blockade agent comprises an immunoglobulin G (IgG) Fc domain. In some embodiments, the IgG Fc domain comprises an IgG1 Fc domain. In some embodiments, the IgG1 Fc domain comprises a fragment of a human antibody. In some embodiments, the CD47-SIRPα blockade agent is selected from the group consisting of TTI-621, TTI-622, and ALX148. In some embodiments, the CD47-SIRPα blockade agent is TTI-621, TTI-622, and ALX148. In some embodiments, the CD47-SIRPα blockade agent is TTI-622. In some embodiments, the CD47-SIRPα blockade agent is ALX148. In some embodiments, the IgG Fc domain comprises an IgG4 Fc domain. In some embodiments, the CD47-SIRPα blockade agent is an antibody. In some embodiments, the antibody is selected from the group consisting of MIAP410, B6H12, and Magrolimab. In some embodiments, the antibody is MIAP410. In some embodiments, the antibody is B6H12. In some embodiments, the antibody is Magrolimab.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is administered at a dose effective to reduce the population of cells. In some embodiments, the population of cells is reduced by between about 10% and 100%. In some embodiments, the population of cells is reduced by between about 10% and about 20%. In some embodiments, the population of cells is reduced by between about 20% and about 30%. In some embodiments, the population of cells is reduced by between about 30% and about 40%. In some embodiments, the population of cells is reduced by between about 40% and about 50%. In some embodiments, the population of cells is reduced by between about 50% and about 60%. In some embodiments, the population of cells is reduced by between about 60% and about 70%. In some embodiments, the population of cells is reduced by between about 70% and about 80%. In some embodiments, the population of cells is reduced by between about 80% and about 90%. In some embodiments, the population of cells is reduced by between about 90% and about 100%. In some embodiments, the population of cells is eliminated. In some embodiments, the reduction of the population of cells occurs via an immune response. In some embodiments, the immune response is NK cell-mediated cell killing, macrophage-mediated cell killing, complement-dependent cytotoxicity (CDC), and/or antibody-dependent cellular cytotoxicity (ADCC) of the cells. In some embodiments, the immune response is NK cell-mediated cell killing of the cells. In some embodiments, the immune response is macrophage-mediated cell killing of the cells. In some embodiments, the immune response is complement-dependent cytotoxicity (CDC) of the cells. In some embodiments, the immune response is antibody-dependent cellular cytotoxicity (ADCC) of the cells.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is administered to the subject intravenously, subcutaneously, intraperitonially, intramuscularly, or intracranially. In some embodiments, the CD47-SIRPα blockade agent is administered to the subject intravenously. In some embodiments, the CD47-SIRPα blockade agent is administered to the subject subcutaneously. In some embodiments, the CD47-SIRPα blockade agent is administered to the subject intraperitonially. In some embodiments, the CD47-SIRPα blockade agent is administered to the subject intramuscularly. In some embodiments, the CD47-SIRPα blockade agent is administered to the subject or intracranially.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is administered to the subject at a time interval of between 1-20 days for a period of between 10 days and 6 months. In some embodiments, the CD47-SIRPα blockade agent is administered to the subject at a time interval of between 1-2 days for a period of between 10 days and 6 months. the CD47-SIRPα blockade agent is administered to the subject at a time interval of between 2-3 days for a period of between 10 days and 6 months. the CD47-SIRPα blockade agent is administered to the subject at a time interval of between 3-4 days for a period of between 10 days and 6 months. the CD47-SIRPα blockade agent is administered to the subject at a time interval of between 4-5 days for a period of between 10 days and 6 months. the CD47-SIRPα blockade agent is administered to the subject at a time interval of between 5-6 days for a period of between 10 days and 6 months. the CD47-SIRPα blockade agent is administered to the subject at a time interval of between 6-7 days for a period of between 10 days and 6 months. the CD47-SIRPα blockade agent is administered to the subject at a time interval of between 7-10 days for a period of between 10 days and 6 months. the CD47-SIRPα blockade agent is administered to the subject at a time interval of between 10-15 days for a period of between 10 days and 6 months. the CD47-SIRPα blockade agent is administered to the subject at a time interval of between 15-20 days for a period of between 10 days and 6 months.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is administered to the subject at a time interval of between 1-10 days for a period of between 10-15 days. In some embodiments, the CD47-SIRPα blockade agent is administered to the subject at a time interval of between 1-15 days for a period of between 15-20 days. In some embodiments, the CD47-SIRPα blockade agent is administered to the subject at a time interval of between 1-20 days for a period of between 20-25 days. In some embodiments, the CD47-SIRPα blockade agent is administered to the subject at a time interval of between 1-20 days for a period of between 25-30 days. In some embodiments, the CD47-SIRPα blockade agent is administered to the subject at a time interval of between 1-20 days for a period of between 1-2 months. In some embodiments, the CD47-SIRPα blockade agent is administered to the subject at a time interval of between 1-20 days for a period of between 2-3 months. In some embodiments, the CD47-SIRPα blockade agent is administered to the subject at a time interval of between 1-20 days for a period of between 3-4 months. In some embodiments, the CD47-SIRPα blockade agent is administered to the subject at a time interval of between 1-20 days for a period of between 4-5 months. In some embodiments, the CD47-SIRPα blockade agent is administered to the subject at a time interval of between 1-20 days for a period of between 5-6 months.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is administered to the subject: (i) at 0.05, 0.1, 0.3, 1, 3, or 10 mg/kg; (ii) once every 12 hours, once every 24 hours, once every 36 hours, or once every 48 hours; and/or (iii) for between 1 day and 3 weeks. In some embodiments, the CD47-SIRPα blockade agent is administered at 0.05 mg/kg. In some embodiments, the CD47-SIRPα blockade agent is administered to the subject at 0.1 mg/kg. In some embodiments, the CD47-SIRPα blockade agent is administered to the subject at 0.3 mg/kg. In some embodiments, the CD47-SIRPα blockade agent is administered to the subject at 1 mg/kg. In some embodiments, the CD47-SIRPα blockade agent is administered to the subject at 3 mg/kg. In some embodiments, the CD47-SIRPα blockade agent is administered to the subject at 10 mg/kg. In some embodiments, the CD47-SIRPα blockade agent is administered to the subject at between about 0.01 mg/kg and about 20 mg/kg. In some embodiments, the CD47-SIRPα blockade agent is administered to the subject at between about 0.01 mg/kg and about 0.05 mg/kg. In some embodiments, the CD47-SIRPα blockade agent is administered to the subject at between about 0.05 mg/kg and about 0.1 mg/kg. In some embodiments, the CD47-SIRPα blockade agent is administered to the subject at between about 0.1 mg/kg and about 0.5 mg/kg. In some embodiments, the CD47-SIRPα blockade agent is administered to the subject at between about 0.5 mg/kg and about 1 mg/kg. In some embodiments, the CD47-SIRPα blockade agent is administered to the subject at between about 1 mg/kg and about 5 mg/kg. In some embodiments, the CD47-SIRPα blockade agent is administered to the subject at between about 5 mg/kg and about 10 mg/kg. In some embodiments, the CD47-SIRPα blockade agent is administered to the subject at between about 10 mg/kg and about 15 mg/kg. In some embodiments, the CD47-SIRPα blockade agent is administered to the subject at between about 15 mg/kg and about 20 mg/kg.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is administered to the subject once every 6 hours. In some embodiments, the CD47-SIRPα blockade agent is administered to the subject once every 12 hours. In some embodiments, the CD47-SIRPα blockade agent is administered to the subject once every 18 hours. In some embodiments, the CD47-SIRPα blockade agent is administered to the subject once every 24 hours. In some embodiments, the CD47-SIRPα blockade agent is administered to the subject once every 36 hours. In some embodiments, the CD47-SIRPα blockade agent is administered to the subject once every 48 hours. In some embodiments, the CD47-SIRPα blockade agent is administered to the subject once every 3 days. In some embodiments, the CD47-SIRPα blockade agent is administered to the subject once every 4 days. In some embodiments, the CD47-SIRPα blockade agent is administered to the subject once every 5 days. In some embodiments, the CD47-SIRPα blockade agent is administered to the subject once every 6 days. In some embodiments, the CD47-SIRPα blockade agent is administered to the subject once every 7 days. In some embodiments, the CD47-SIRPα blockade agent is administered to the subject once every 2 weeks. In some embodiments, the CD47-SIRPα blockade agent is administered to the subject once every 4 weeks. In some embodiments, the CD47-SIRPα blockade agent is administered to the subject once every 6 weeks. In some embodiments, the CD47-SIRPα blockade agent is administered to the subject once every 8 weeks. In some embodiments, the CD47-SIRPα blockade agent is administered to the subject once every 3 months. In some embodiments, the CD47-SIRPα blockade agent is administered to the subject once every 4 months. In some embodiments, the CD47-SIRPα blockade agent is administered to the subject once every 5 months. In some embodiments, the CD47-SIRPα blockade agent is administered to the subject once every 6 months. In some embodiments, the CD47-SIRPα blockade agent is administered to the subject once every between about 6 months and about 12 months. In some embodiments, the CD47-SIRPα blockade agent is administered to the subject once every 18 months. In some embodiments, the CD47-SIRPα blockade agent is administered to the subject once every 24 months. In some embodiments, the CD47-SIRPα blockade agent is administered to the subject once every 3 years. In some embodiments, the CD47-SIRPα blockade agent is administered to the subject once every 4 years. In some embodiments, the CD47-SIRPα blockade agent is administered to the subject once every 5 years.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is administered to the subject for between about 1 day and about 50 years. In some embodiments, the CD47-SIRPα blockade agent is administered to the subject for between about 1 day and about 1 week. In some embodiments, the CD47-SIRPα blockade agent is administered to the subject for between about 1 week and about 2 weeks. In some embodiments, the CD47-SIRPα blockade agent is administered to the subject for between about 2 weeks and about 3 weeks. In some embodiments, the CD47-SIRPα blockade agent is administered to the subject for between about 3 weeks and about 1 month. In some embodiments, the CD47-SIRPα blockade agent is administered to the subject for between about 1 month and about 2 months. In some embodiments, the CD47-SIRPα blockade agent is administered to the subject for between about 2 months and about 3 months. In some embodiments, the CD47-SIRPα blockade agent is administered to the subject for between about 3 months and about 4 months. In some embodiments, the CD47-SIRPα blockade agent is administered to the subject for between about 4 months and about 5 months. In some embodiments, the CD47-SIRPα blockade agent is administered to the subject for between about 5 months and about 6 months. In some embodiments, the CD47-SIRPα blockade agent is administered to the subject for between about 6 months and about 1 year. In some embodiments, the CD47-SIRPα blockade agent is administered to the subject for between about 1 year and about 2 years. In some embodiments, the CD47-SIRPα blockade agent is administered to the subject for between about 2 years and about 3 years. In some embodiments, the CD47-SIRPα blockade agent is administered to the subject for between about 3 years and about 4 years. In some embodiments, the CD47-SIRPα blockade agent is administered to the subject for between about 4 years and about 5 years. In some embodiments, the CD47-SIRPα blockade agent is administered to the subject for between about 5 years and about 10 years. In some embodiments, the CD47-SIRPα blockade agent is administered to the subject for between about 10 years and about 15 years. In some embodiments, the CD47-SIRPα blockade agent is administered to the subject for between about 15 years and about 20 years. In some embodiments, the CD47-SIRPα blockade agent is administered to the subject for between about 20 years and about 30 years. In some embodiments, the CD47-SIRPα blockade agent is administered to the subject for between about 30 years and about 40 years. In some embodiments, the CD47-SIRPα blockade agent is administered to the subject for between about 40 years and about 50 years. In some embodiments, the CD47-SIRPα blockade agent is administered to the subject for the lifetime of the subject.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the methods disclosed herein further comprise administering IL-2 to the subject.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof. In some embodiments, the CD47-SIRPα blockade agent is an antibody or fragment thereof that binds CD47. In some embodiments, the CD47-SIRPα blockade agent is a bispecific antibody that binds CD47. In some embodiments, the CD47-SIRPα blockade agent is an immunocytokine fusion protein that bind CD47. In some embodiments, the CD47-SIRPα blockade agent is a CD47 containing fusion protein. In some embodiments, the CD47-SIRPα blockade agent is an antibody or fragment thereof that binds SIRPα. In some embodiments, the CD47-SIRPα blockade agent is a bispecific antibody that binds SIRPα. In some embodiments, the CD47-SIRPα blockade agent is an immunocytokine fusion protein that binds SIRPα. In some embodiments, the CD47-SIRPα blockade agent is an SIRPα containing fusion protein.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof. In some embodiments, the antibody or fragment thereof that binds CD47 is selected from the group consisting of magrolimab (Hu5F9-G4), CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof that binds CD47 is magrolimab (Hu5F9-G4). In some embodiments, the antibody or fragment thereof that binds CD47 is CC-90002. In some embodiments, the antibody or fragment thereof that binds CD47 is IBI-188. In some embodiments, the antibody or fragment thereof that binds CD47 is IBI-322. In some embodiments, the antibody or fragment thereof that binds CD47 is TG-1801 (NI-1701). In some embodiments, the antibody or fragment thereof that binds CD47 is ALX148. In some embodiments, the antibody or fragment thereof that binds CD47 is TJ011133. In some embodiments, the antibody or fragment thereof that binds CD47 is FA3M3. In some embodiments, the antibody or fragment thereof that binds CD47 is ZL1201. In some embodiments, the antibody or fragment thereof that binds CD47 is AK117. In some embodiments, the antibody or fragment thereof that binds CD47 is AO-176. In some embodiments, the antibody or fragment thereof that binds CD47 is SRF231. In some embodiments, the antibody or fragment thereof that binds CD47 is GenSci-059. In some embodiments, the antibody or fragment thereof that binds CD47 is C47B157. In some embodiments, the antibody or fragment thereof that binds CD47 is C47B161. In some embodiments, the antibody or fragment thereof that binds CD47 is C47B167. In some embodiments, the antibody or fragment thereof that binds CD47 is C47B222. In some embodiments, the antibody or fragment thereof that binds CD47 is C47B227. In some embodiments, the antibody or fragment thereof that binds CD47 is Vx-1004. In some embodiments, the antibody or fragment thereof that binds CD47 is HMBD004. In some embodiments, the antibody or fragment thereof that binds CD47 is SHR-1603. In some embodiments, the antibody or fragment thereof that binds CD47 is AMMS4-G4. In some embodiments, the antibody or fragment thereof that binds CD47 is RTX-CD47. In some embodiments, the antibody or fragment thereof that binds CD47 is and IMC-002.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof. In some embodiments, the antibody or fragment thereof that binds CD47 is selected from the group consisting of a single-chain Fv fragment (scFv) against CD47, a Fab against CD47, a VHH nanobody against CD47, a DARPin against CD47, and variants thereof. In some embodiments, the antibody or fragment thereof that binds CD47 is a single-chain Fv fragment (scFv) against CD47, and variants thereof. In some embodiments, the antibody or fragment thereof that binds CD47 is a Fab against CD47, and variants thereof. In some embodiments, the antibody or fragment thereof that binds CD47 is a VHH nanobody against CD47, and variants thereof. In some embodiments, the antibody or fragment thereof that binds CD47 is a DARPin against CD47, and variants thereof.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof. In some embodiments, the antibody or fragment thereof that binds SIRPα is selected from the group consisting of ADU-1805, CC-95251, OSE-172 (BI 765063), KWAR23, and P362. In some embodiments, the antibody or fragment thereof that binds SIRPα is ADU-1805. In some embodiments, the antibody or fragment thereof that binds SIRPα is CC-95251. In some embodiments, the antibody or fragment thereof that binds SIRPα is OSE-172 (BI 765063). In some embodiments, the antibody or fragment thereof that binds SIRPα is KWAR23. In some embodiments, the antibody or fragment thereof that binds SIRPα is P362.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof. In some embodiments, the antibody or fragment thereof that binds SIRPα is selected from the group consisting of a single-chain Fv fragment (scFv) against SIRPα, a Fab against SIRPα, a VHH nanobody against SIRPα, a DARPin against SIRPα, and variants thereof. In some embodiments, the antibody or fragment thereof that binds SIRPα is a single-chain Fv fragment (scFv) against SIRPα, and variants thereof. In some embodiments, the antibody or fragment thereof that binds SIRPα is a Fab against SIRPα, and variants thereof. In some embodiments, the antibody or fragment thereof that binds SIRPα is a VHH nanobody against SIRPα, and variants thereof. In some embodiments, the antibody or fragment thereof that binds SIRPα is a DARPin against SIRPα, and variants thereof.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof. In some embodiments, the SIRPα-containing fusion protein comprises a CD47 binding domain of SIRPα linked to an Fc domain. In some embodiments, the Fc domain comprises an Fc domain or portion thereof selected from the group consisting of IgG1, IgG2, IgG3, and IgG4. In some embodiments, the Fc domain comprises an Fc domain or portion thereof that is IgG1. In some embodiments, the Fc domain comprises an Fc domain or portion thereof that is IgG2. In some embodiments, the Fc domain comprises an Fc domain or portion thereof that is IgG3. In some embodiments, the Fc domain comprises an Fc domain or portion thereof that is IgG4.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the exogenous CD47 polypeptide comprises the amino acid sequence of SEQ ID NO:2 or SEQ ID NO:4. In some embodiments, the exogenous CD47 polypeptide comprises the amino acid sequence of SEQ ID NO:2. In some embodiments, the exogenous CD47 polypeptide comprises the amino acid sequence of SEQ ID NO:4. In some embodiments, the exogenous CD47 polypeptide comprises an amino acid sequence that is identical to the amino acid sequence of an endogenous CD47 polypeptide. In some embodiments, the exogenous CD47 polypeptide comprises an amino acid sequence that is similar to the amino acid sequence of an endogenous CD47 polypeptide. In some embodiments, the exogenous CD47 polypeptide comprises an amino acid sequence that is different from the amino acid sequence of an endogenous CD47 polypeptide.

### EMBODIMENTS

Embodiment 1. A method comprising administering a CD47-signal regulatory protein alpha (SIRPα) blockade agent to a patient previously administered a population of cells comprising exogenously expressed CD47 polypeptides.

Embodiment 2. The method of any of the above or below embodiments, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

Embodiment 3. The method of any of the above or below embodiments, wherein the antibody or fragment thereof that binds CD47 is selected from the group consisting of magrolimab (Hu5F9-G4), CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

Embodiment 4. The method of any of the above or below embodiments, wherein the antibody or fragment thereof that binds CD47 is selected from the group consisting of a single-chain Fv fragment (scFv) against CD47, a Fab against CD47, a VHH nanobody against CD47, a DARPin against CD47, and variants thereof.

Embodiment 5. The method of any of the above or below embodiments, wherein the antibody or fragment thereof that binds SIRPα is selected from the group consisting of ADU-1805, CC-95251, OSE-172 (BI 765063), KWAR23, and P362.

Embodiment 6. The method of any of the above or below embodiments, wherein the antibody or fragment thereof that binds SIRPα is selected from the group consisting of a single-chain Fv fragment (scFv) against SIRPα, a Fab against SIRPα, a VHH nanobody against SIRPα, a DARPin against SIRPα, and variants thereof.

Embodiment 7. The method of any of the above or below embodiments, wherein the SIRPα containing fusion protein comprises a CD47 binding domain of SIRPα linked to an Fc domain.

Embodiment 8. The method of any of the above or below embodiments, wherein the Fc domain comprises an Fc domain or portion thereof selected from the group consisting of IgG1, IgG2, IgG3, and IgG4.

Embodiment 9. The method of any of the above or below embodiments, wherein the administration of the CD47-SIRPα blockade agent reduces the amount of the population of cells remaining viable in the patient.

Embodiment 10. The method of any of the above or below embodiments, wherein the administration of the CD47-SIRPα blockade agent reduces the number of cells exogenously expressing CD47 peptides in the patient.

Embodiment 11. The method of any of the above or below embodiments, wherein the administration of the CD47-SIRPα blockade agent occurs after the patient experiences an adverse event subsequent to the administration of the population of cells.

Embodiment 12. The method of any of the above or below embodiments, wherein the adverse event is selected from the group consisting of hyperproliferation, transformation, tumor formation, cytokine release syndrome, graft-versus-host disease (GVHD), and immune effector cell-associated neurotoxicity syndrome (ICANS).

Embodiment 13. The method of any of the above or below embodiments, wherein the administration of the CD47-SIRPα blockade agent is at least 1 week or more after the administration of the population of cells.

Embodiment 14. The method of any of the above or below embodiments, wherein the administration of the CD47-SIRPα blockade agent is at least 1 month or more after the administration of the population of cells.

Embodiment 15. The method of any of the above or below embodiments, wherein the cells further comprise reduced expression of MHC class I and/or MHC II human leukocyte antigens.

Embodiment 16. The method of any of the above or below embodiments, wherein the cells further comprise reduced expression of one or more TCR complexes.

Embodiment 17. The method of any of the above or below embodiments, wherein the cells further comprise one or more transgenes wherein the transgene encodes an exogenously expressed polypeptide selected from the group consisting of CD24, CD46, CD55, CD59, CD200, DUX4, PD-L1, IDO1, HLA-G, CCL21, Mfge8, and Serpin B9.

Embodiment 18. The method of any of the above or below embodiments, wherein the cells comprise exogenously expressed CD47 polypeptides and one or more additional exogenously expressed polypeptides selected from the group consisting of CD24, CD46, CD55, CD59, DUX4, PD-L1, IDO1, HLA-G, CD200, FasL, CCL21, Mfge8, Serpin B9, and any combination thereof.

Embodiment 19. The method of any of the above or below embodiments, wherein the cells comprise exogenously expressed CD47 polypeptides and reduced expression levels of B2M and/or CIITA.

Embodiment 20. The method of any of the above or below embodiments, wherein the cells comprise exogenously expressed CD47 polypeptide and further comprise reduced expression levels of B2M and CIITA.

Embodiment 21. The method of any of the above or below embodiments, wherein the cells comprise exogenously expressed CD47 polypeptides, reduced expression levels of B2M and/or CIITA, and one or more additional exogenously expressed polypeptides selected from the group selected from of CD24, CD46, CD55, CD59, CD200, DUX4, PD-L1, HLA-G, IDO1, FasL, CCL21, Mfge8, Serpin B9, and any combination thereof.

Embodiment 22. The method of any of the above or below embodiments, wherein the cells comprise exogenously expressed CD47 polypeptides, reduced expression levels of B2M and CIITA, and one or more additional exogenously expressed polypeptides selected from the group selected from of CD24, CD46, CD55, CD59, CD200, DUX4, PD-L1, HLA-G, IDO1, FasL, CCL21, Mfge8, Serpin B9, and any combination thereof.

Embodiment 23. The method of any of the above or below embodiments, wherein the cells further comprise a reduced expression level of TCRα, TCRβ, or both.

Embodiment 24. The method of any of the above or below embodiments, wherein the cells are differentiated cells derived from pluripotent stem cells.

Embodiment 25. The method of any of the above or below embodiments, wherein the pluripotent stem cells comprise induced pluripotent stem cells.

Embodiment 26. The method of any of the above or below embodiments, wherein the differentiated cells are selected from the group consisting of cardiac cells, neural cells, endothelial cells, T cells, pancreatic islet cells, retinal pigmented epithelium cells, hepatocytes, thyroid cells, skin cells, blood cells, and epithelial cells.

Embodiment 27. The method of any of the above or below embodiments, wherein the cells comprise cells derived from primary T cells.

Embodiment 28. The method of any of the above or below embodiments, wherein the cells derived from primary T cells are derived from a pool of primary T cells comprising primary T cells from one or more (e.g., two or more, three or more, four or more, five or more, ten or more, twenty or more, fifty or more, or one hundred or more) subjects different from the patient.

Embodiment 29. The method of any of the above or below embodiments, wherein the cells derived from primary T cells comprise a chimeric antigen receptor (CAR).

Embodiment 30. The method of any of the above or below embodiments, wherein the CAR and the exogenously expressed CD47 polypeptides are expressed under the control of a single promoter.

Embodiment 31. The method of any of the above or below embodiments, wherein the CAR binds an antigen selected from the group consisting of CD19, CD22, CD38, CD123, CD138, and BCMA.

Embodiment 32. The method of any of the above or below embodiments, wherein the cells derived from primary T cells comprise reduced expression of an endogenous T cell receptor.

Embodiment 33. The method of any of the above or below embodiments, wherein the cells derived from primary T cells comprise reduced expression of cytotoxic T-lymphocyte-associated protein 4 (CTLA4) and/or programmed cell death (PD1).

Embodiment 34. A method comprising: (a) administering to the patient an amount of a population of cells comprising exogenously expressed CD47; and (b) administering to the patient an amount of a CD47-SIRPα blockade agent effective to reduce the number of the cells and derivatives thereof in the patient.

Embodiment 35. The method of any of the above or below embodiments, wherein the cells further comprise reduced expression of MHC class I and/or MHC class II human leukocyte antigens.

Embodiment 36. The method of any of the above or below embodiments, wherein the cells comprise reduced expression of MHC class I and MHC class II human leukocyte antigens.

Embodiment 37. The method of any of the above or below embodiments, wherein the cells further comprise reduced expression of one or more TCR complexes.

Embodiment 38. The method of any of the above or below embodiments, wherein the cells comprise exogenously expressed CD47 polypeptides and one or more additional exogenously expressed polypeptides selected from the group consisting of CD24, CD46, CD55, CD59, CD200, DUX4, PD-L1, IDO1, HLA-G, FasL, CCL21, Mfge8, Serpin B9, and any combination thereof.

Embodiment 39. The method of any of the above or below embodiments, wherein the cells further comprise one or more transgenes wherein the transgene encodes an exogenously expressed polypeptide selected from the group consisting of CD24, CD46, CD55, CD59, CD200, DUX4, PD-L1, IDO1, HLA-G, FasL, CCL21, Mfge8, and Serpin B9.

Embodiment 40. The method of any of the above or below embodiments, wherein the cells comprise exogenously expressed CD47 polypeptides and reduced expression levels of B2M and/or CIITA.

Embodiment 41. The method of any of the above or below embodiments, wherein the cells comprise exogenously expressed CD47 polypeptides and reduced expression levels of B2M and CIITA.

Embodiment 42. The method of any of the above or below embodiments, wherein the cells comprise exogenously expressed CD47 polypeptides, reduced expression levels of B2M and/or CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 43. The method of any of the above or below embodiments, wherein the cells comprise exogenously expressed CD47 polypeptides, reduced expression levels of B2M and CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 44. The method of any of the above or below embodiments, wherein the cells further comprise a reduced expression level of TCRα, TCRβ, or both.

Embodiment 45. The method of any of the above or below embodiments, wherein the cells are differentiated cells derived from pluripotent stem cells.

Embodiment 46. The method of any of the above or below embodiments, wherein the pluripotent stem cells comprise induced pluripotent stem cells.

Embodiment 47. The method of any of the above or below embodiments, wherein the differentiated cells are selected from the group consisting of cardiac cells, neural cells, endothelial cells, T cells, pancreatic islet cells, retinal pigmented epithelium cells, hepatocytes, thyroid cells, skin cells, blood cells, and epithelial cells.

Embodiment 48. The method of any of the above or below embodiments, wherein the cells comprise cells derived from primary T cells.

Embodiment 49. The method of any of the above or below embodiments, wherein the cells derived from primary T cells are derived from a pool of primary T cells comprising primary T cells from one or more (e.g., two or more, three or more, four or more, five or more, ten or more, twenty or more, fifty or more, or one hundred or more) subjects different from the patient.

Embodiment 50. The method of any of the above or below embodiments, wherein the cells derived from primary T cells comprise a chimeric antigen receptor (CAR).

Embodiment 51. The method of any of the above or below embodiments, wherein the CAR and the exogenously expressed CD47 polypeptides are expressed under the control of a single promoter.

Embodiment 52. The method of any of the above or below embodiments, wherein the CAR binds an antigen selected from the group consisting of CD19, CD22, CD38, CD123, CD138, and BCMA.

Embodiment 53. The method of any of the above or below embodiments, wherein the cells derived from primary T cells comprise reduced expression of an endogenous T cell receptor.

Embodiment 54. The method of any of the above or below embodiments, wherein the cells derived from primary T cells comprise reduced expression of cytotoxic T-lymphocyte-associated protein 4 (CTLA4) and/or programmed cell death (PD1).

Embodiment 55. The method of any of the above or below embodiments, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

Embodiment 56. The method of any of the above or below embodiments, wherein the antibody or fragment thereof that binds CD47 is selected from the group consisting of magrolimab (Hu5F9-G4), CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

Embodiment 57. The method of any of the above or below embodiments, wherein the antibody or fragment thereof that binds CD47 is selected from the group consisting of a single-chain Fv fragment (scFv) against CD47, a Fab against CD47, a VHH nanobody against CD47, a DARPin against CD47 and variants thereof.

Embodiment 58. The method of any of the above or below embodiments, wherein the antibody or fragment thereof that binds SIRPα is selected from the group consisting of ADU-1805, CC-95251, OSE-172 (BI 765063), KWAR23, and P362.

Embodiment 59. The method of any of the above or below embodiments, wherein the antibody or fragment thereof that binds SIRPα is selected from the group consisting of a single-chain Fv fragment (scFv) against SIRPα, a Fab against SIRPα, a VHH nanobody against SIRPα, a DARPin against SIRPα and variants thereof.

Embodiment 60. The method of any of the above or below embodiments, wherein the SIRPα containing fusion protein comprises an CD47 binding domain of SIRPα linked to an Fc domain.

Embodiment 61. The method of any of the above or below embodiments, wherein the Fc domain comprises an Fc domain or portion thereof selected from the group consisting of IgG1, IgG2, IgG3, and IgG4.

Embodiment 62. The method of any of the above or below embodiments, wherein the administration of the CD47-SIRPα blockade agent occurs when the patient experiences an adverse event after the administration of the population of cells.

Embodiment 63. The method of any of the above or below embodiments, wherein the adverse event is selected from the group consisting of hyperproliferation, transformation, tumor formation, cytokine release syndrome, graft-versus-host disease (GVHD), and immune effector cell-associated neurotoxicity syndrome (ICANS).

Embodiment 64. The method of any of the above or below embodiments, wherein the administration of the CD47-SIRPα blockade agent is at least 1 week or more after the administration of the population of cells.

Embodiment 65. The method of any of the above or below embodiments, wherein the administration of the CD47-SIRPα blockade agent is at least 1 month or more after the administration of the population of cells.

Embodiment 66. A method comprising: (a) administering a population of cells to the patient, wherein the cells comprise exogenously expressed CD47 polypeptides; and (b) administering a CD47-SIRPα blockade agent to the patient following an interval period after step (a), wherein the interval period comprises at least 1 week or more.

Embodiment 67. The method of any of the above or below embodiments, wherein the cells comprise reduced expression of MHC class I and/or MHC class II human leukocyte antigens.

Embodiment 68. The method of any of the above or below embodiments, wherein the cells comprise reduced expression of MHC class I and MHC class II human leukocyte antigens.

Embodiment 69. The method of any of the above or below embodiments, wherein the cells further comprise reduced expression of one or more TCR complexes.

Embodiment 70. The method of any of the above or below embodiments, wherein the cells comprise exogenously expressed CD47 polypeptides and one or more additional exogenously expressed polypeptides selected from the group consisting of CD24, CD46, CD55, CD59, CD200, DUX4, PD-L1, IDO1, HLA-G, FasL, CCL21, Mfge8, Serpin B9, and any combination thereof.

Embodiment 71. The method of any of the above or below embodiments, wherein the cells further comprise one or more transgenes wherein the transgene encodes an exogenously expressed polypeptide selected from the group consisting of CD24, CD46, CD55, CD59, CD200, DUX4, PD-L1, IDO1, HLA-G, FasL, CCL21, Mfge8, and Serpin B9.

Embodiment 72. The method of any of the above or below embodiments, wherein the cells express the exogenous CD47 polypeptides and further comprise reduced expression levels of B2M and/or CIITA.

Embodiment 73. The method of any of the above or below embodiments, wherein the cells express the exogenous CD47 polypeptides and further comprise reduced expression levels of B2M and CIITA.

Embodiment 74. The method of any of the above or below embodiments, wherein the cells comprise exogenously expressed CD47 polypeptides, reduced expression levels of B2M and/or CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 75. The method of any of the above or below embodiments, wherein the cells comprise exogenously expressed CD47 polypeptides, reduced expression levels of B2M and CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 76. The method of any of the above or below embodiments, wherein the cells further comprise a reduced expression level of TCRα, TCRβ, or both.

Embodiment 77. The method of any of the above or below embodiments, wherein the cells comprise differentiated cells derived from pluripotent stem cells.

Embodiment 78. The method of any of the above or below embodiments, wherein the pluripotent stem cells comprise induced pluripotent stem cells.

Embodiment 79. The method of any of the above or below embodiments, wherein the differentiated cells are selected from the group consisting of cardiac cells, neural cells, endothelial cells, T cells, pancreatic islet cells, retinal pigmented epithelium cells, hepatocytes, thyroid cells, skin cells, blood cells, and epithelial cells.

Embodiment 80. The method of any of the above or below embodiments, wherein the cells comprise cells derived from primary T cells.

Embodiment 81. The method of any of the above or below embodiments, wherein the cells derived from primary T cells are derived from a pool of primary T cells comprising primary T cells from one or more (e.g., two or more, three or more, four or more, five or more, ten or more, twenty or more, fifty or more, or one hundred or more) subjects different from the patient.

Embodiment 82. The method of any of the above or below embodiments, wherein the cells derived from primary T cells comprise a chimeric antigen receptor (CAR).

Embodiment 83. The method of any of the above or below embodiments, wherein the CAR and the exogenously expressed CD47 polypeptides are expressed under the control of a single promoter.

Embodiment 84. The method of any of the above or below embodiments, wherein the CAR binds an antigen selected from the group consisting of CD19, CD22, CD38, CD123, CD138, and BCMA.

Embodiment 85. The method of any of the above or below embodiments, wherein the cells derived from primary T cells comprise reduced expression of an endogenous T cell receptor.

Embodiment 86. The method of any of the above or below embodiments, wherein the cells derived from primary T cells comprise reduced expression of cytotoxic T-lymphocyte-associated protein 4 (CTLA4) and/or programmed cell death (PD1).

Embodiment 87. The method of any of the above or below embodiments, wherein the interval period comprises at least 1 month or more.

Embodiment 88. The method of any of the above or below embodiments, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

Embodiment 89. The method of any of the above or below embodiments, wherein the antibody or fragment thereof that binds CD47 is selected from the group consisting of magrolimab (Hu5F9-G4), CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

Embodiment 90. The method of any of the above or below embodiments, wherein the antibody or fragment thereof that binds CD47 is selected from the group consisting of a single-chain Fv fragment (scFv) against CD47, a Fab against CD47, a VHH nanobody against CD47, a DARPin against CD47 and variants thereof.

Embodiment 91. The method of any of the above or below embodiments, wherein the antibody or fragment thereof that binds SIRPα is selected from the group consisting of ADU-1805, CC-95251, OSE-172 (BI 765063), KWAR23, and P362.

Embodiment 92. The method of any of the above or below embodiments, wherein the antibody or fragment thereof that binds SIRPα is selected from the group consisting of a single-chain Fv fragment (scFv) against SIRPα, a Fab against SIRPα, a VHH nanobody against SIRPα, a DARPin against SIRPα and variants thereof.

Embodiment 93. The method of any of the above or below embodiments, wherein the SIRPα containing fusion protein comprises a CD47 binding domain of SIRPα linked to an Fc domain.

Embodiment 94. The method of any of the above or below embodiments, wherein the Fc domain comprises an Fc domain or portion thereof selected from the group consisting of IgG1, IgG2, IgG3, and IgG4.

Embodiment 95. The method of any of the above or below embodiments, wherein the step (b) reduces the amount of the population of cells remaining viable in the patient.

Embodiment 96. The method of any of the above or below embodiments, wherein the step (b) reduces the number of cells exogenously expressing CD47 peptides in the patient.

Embodiment 97. The method of any of the above or below embodiments, further comprising administering a second population of the cells after the step (b).

Embodiment 98. A method of modulating activity of a cell therapy in a patient, wherein the patient has received at least one dose of a therapeutically effective population of cells comprising exogenously expressed CD47 polypeptides, the method comprising administering to the patient a CD47-SIRPα blockade agent in an amount effective to modulate an activity of the population of cells.

Embodiment 99. The method of any of the above or below embodiments, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

Embodiment 100. The method of any of the above or below embodiments, wherein the antibody or fragment thereof that binds CD47 is selected from the group consisting of magrolimab (Hu5F9-G4), CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

Embodiment 101. The method of any of the above or below embodiments, wherein the antibody or fragment thereof that binds CD47 is selected from the group consisting of a single-chain Fv fragment (scFv) against CD47, a Fab against CD47, a VHH nanobody against CD47, a DARPin against CD47 and variants thereof.

Embodiment 102. The method of any of the above or below embodiments, wherein the antibody or fragment thereof that binds SIRPα is selected from the group consisting of ADU-1805, CC-95251, OSE-172 (BI 765063), KWAR23, and P362.

Embodiment 103. The method of any of the above or below embodiments, wherein the antibody or fragment thereof that binds SIRPα is selected from the group consisting of a single-chain Fv fragment (scFv) against SIRPα, a Fab against SIRPα, a VHH nanobody against SIRPα, a DARPin against SIRPα and variants thereof.

Embodiment 104. The method of any of the above or below embodiments, wherein the SIRPα containing fusion protein comprises a CD47 binding domain of SIRPα linked to an Fc domain.

Embodiment 105. The method of any of the above or below embodiments, wherein the Fc domain comprises an Fc domain or portion thereof selected from the group consisting of IgG1, IgG2, IgG3, and IgG4.

Embodiment 106. The method of any of the above or below embodiments, wherein the cells comprise reduced expression of MHC class I and/or MHC class II human leukocyte antigens.

Embodiment 107. The method of any of the above or below embodiments, wherein the cells comprise reduced expression of MHC class I and MHC class II human leukocyte antigens.

Embodiment 108. The method of any of the above or below embodiments, wherein the cells further comprise reduced expression of one or more TCR complexes.

Embodiment 109. The method of any of the above or below embodiments, wherein the cells comprise exogenously expressed CD47 polypeptides and one or more additional exogenously expressed polypeptides selected from the group consisting of CD24, CD46, CD55, CD59, CD200, DUX4, PD-L1, IDO1, HLA-G, FasL, CCL21, Mfge8, Serpin B9, and any combination thereof.

Embodiment 110. The method of any of the above or below embodiments, wherein the cells further comprise one or more transgenes, wherein the transgene encodes an exogenously expressed polypeptide selected from the group consisting of CD24, CD46, CD55, CD59, CD200, DUX4, PD-L1, IDO1, HLA-G, FasL, CCL21, Mfge8, and Serpin B9.

Embodiment 111. The method of any of the above or below embodiments, wherein the cells comprise exogenously expressed CD47 polypeptides and reduced expression levels of B2M and/or CIITA.

Embodiment 112. The method of any of the above or below embodiments, wherein the cells comprise exogenously expressed CD47 polypeptides and reduced expression levels of B2M and CIITA.

Embodiment 113. The method of any of the above or below embodiments, wherein the cells comprise exogenously expressed CD47 polypeptides, reduced expression levels of B2M and/or CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 114. The method of any of the above or below embodiments, wherein the cells comprise exogenously expressed CD47 polypeptides, reduced expression levels of B2M and CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 115. The method of any of the above or below embodiments, wherein the cells further comprise a reduced expression level of TCRα, TCRβ, or both.

Embodiment 116. The method of any of the above or below embodiments, wherein the at least one dose of the therapeutically effective population of cells comprises 1, 2, 3, 4, 5, 6, 7, 8, 9, 10, or more doses of the population.

Embodiment 117. The method of any of the above or below embodiments, wherein the modulating comprises decreasing the number of the therapeutically effective population of cells in the patient.

Embodiment 118. The method of any of the above or below embodiments, wherein the cells comprise differentiated cells derived from pluripotent stem cells.

Embodiment 119. The method of any of the above or below embodiments, wherein the pluripotent stem cells comprise induced pluripotent stem cells.

Embodiment 120. The method of any of the above or below embodiments, wherein the differentiated cells are selected from the group consisting of cardiac cells, neural cells, endothelial cells, T cells, pancreatic islet cells, retinal pigmented epithelium cells, hepatocytes, thyroid cells, skin cells, blood cells, and epithelial cells.

Embodiment 121. The method of any of the above or below embodiments, wherein the cells comprise cells derived from primary T cells.

Embodiment 122. The method of any of the above or below embodiments, wherein the cells derived from primary T cells are derived from a pool of primary T cells comprising primary T cells from one or more (e.g., two or more, three or more, four or more, five or more, ten or more, twenty or more, fifty or more, or one hundred or more) subjects different from the patient.

Embodiment 123. The method of any of the above or below embodiments, wherein the cells derived from primary T cells comprise a chimeric antigen receptor (CAR).

Embodiment 124. The method of any of the above or below embodiments, wherein the CAR and the exogenously expressed CD47 polypeptides are expressed under the control of a single promoter.

Embodiment 125. The method of any of the above or below embodiments, wherein the CAR binds an antigen selected from the group consisting of CD19, CD22, CD38, CD123, CD138, and BCMA.

Embodiment 126. The method of any of the above or below embodiments, wherein the cells derived from primary T cells comprise reduced expression of an endogenous T cell receptor.

Embodiment 127. The method of any of the above or below embodiments, wherein the cells derived from primary T cells comprise reduced expression of cytotoxic T-lymphocyte-associated protein 4 (CTLA4) and/or programmed cell death (PD1).

Embodiment 128. The method of any of the above or below embodiments, wherein the activity of the population of cells in the patient comprises an unwanted activity of the cells.

Embodiment 129. The method of any of the above or below embodiments, wherein the unwanted activity is selected from the group consisting of hyperproliferation, transformation, tumor formation, cytokine release syndrome, graft-versus-host disease (GVHD), and immune effector cell-associated neurotoxicity syndrome (ICANS).

Embodiment 130. A method of controlling an effect of cell therapy in a patient, the method comprising: (a) administering a composition comprising a population of cells to the patient, wherein the cells comprise exogenously expressed CD47 polypeptides; (b) after an interval of time subsequent to step (a), further administering to the patient a CD47-SIRPα blockade agent in an amount effective to induce an immune response against the population of cells administered in step (a), thereby controlling the effects of the population of cells in the patient.

Embodiment 131. The method of any of the above or below embodiments, wherein the cells comprise reduced expression of MHC class I and/or MHC class II human leukocyte antigens.

Embodiment 132. The method of any of the above or below embodiments, wherein the cells comprise reduced expression of MHC class I and MHC class II human leukocyte antigens.

Embodiment 133. The method of any of the above or below embodiments, wherein the cells further comprise reduced expression of one or more TCR complexes.

Embodiment 134. The method of any of the above or below embodiments, wherein the cells comprise exogenously expressed CD47 polypeptides and one or more additional exogenously expressed polypeptides selected from the group consisting of CD24, CD46, CD55, CD59, CD200, DUX4, PD-L1, IDO1, HLA-G, FasL, CCL21, Mfge8, Serpin B9, and any combination thereof.

Embodiment 135. The method of any of the above or below embodiments, wherein the cells further comprise one or more transgenes wherein the transgene encodes an exogenously expressed polypeptide selected from the group consisting of CD24, CD46, CD55, CD59, CD200, DUX4, PD-L1, IDO1, HLA-G, FasL, CCL21, Mfge8, and Serpin B9.

Embodiment 136. The method of any of the above or below embodiments, wherein the cells comprise the exogenously expressed CD47 polypeptides and reduced expression levels of B2M and/or CIITA.

Embodiment 137. The method of any of the above or below embodiments, wherein the cells comprise the exogenously expressed CD47 polypeptides and reduced expression levels of B2M and CIITA.

Embodiment 138. The method of any of the above or below embodiments, wherein the cells comprise exogenously expressed CD47 polypeptides, reduced expression levels of B2M and/or CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 139. The method of any of the above or below embodiments, wherein the cells comprise exogenously expressed CD47 polypeptides, reduced expression levels of B2M and CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 140. The method of any of the above or below embodiments, wherein the cells further comprises a reduced expression level of TCRα, TCRβ, or both.

Embodiment 141. The method of any of the above or below embodiments, wherein the cells comprise differentiated cells derived from pluripotent stem cells.

Embodiment 142. The method of any of the above or below embodiments, wherein the pluripotent stem cells comprise induced pluripotent stem cells.

Embodiment 143. The method of any of the above or below embodiments, wherein the differentiated cells are selected from the group consisting of cardiac cells, neural cells, endothelial cells, T cells, pancreatic islet cells, retinal pigmented epithelium cells, hepatocytes, thyroid cells, skin cells, blood cells, and epithelial cells.

Embodiment 144. The method of any of the above or below embodiments, wherein the cells comprise cells derived from primary T cells.

Embodiment 145. The method of any of the above or below embodiments, wherein the cells derived from primary T cells are derived from a pool of primary T cells comprising primary T cells from one or more (e.g., two or more, three or more, four or more, five or more, ten or more, twenty or more, fifty or more, or one hundred or more) subjects different from the patient.

Embodiment 146. The method of any of the above or below embodiments, wherein the cells derived from primary T cells comprise a chimeric antigen receptor (CAR).

Embodiment 147. The method of any of the above or below embodiments, wherein the CAR and the exogenously expressed CD47 polypeptides are expressed under the control of a single promoter.

Embodiment 148. The method of any of the above or below embodiments, wherein the CAR binds an antigen selected from the group consisting of CD19, CD22, CD38, CD123, CD138, and BCMA.

Embodiment 149. The method of any of the above or below embodiments, wherein the cells derived from primary T cells comprise reduced expression of an endogenous T cell receptor.

Embodiment 150. The method of any of the above or below embodiments, wherein the cells derived from primary T cells comprise reduced expression of cytotoxic T-lymphocyte-associated protein 4 (CTLA4) and/or programmed cell death (PD1).

Embodiment 151. The method of any of the above or below embodiments, wherein the interval of time comprises at least 1 week or more.

Embodiment 152. The method of any of the above or below embodiments, wherein the interval of time comprises at least 1 month or more.

Embodiment 153. The method of any of the above or below embodiments, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

Embodiment 154. The method of any of the above or below embodiments, wherein the antibody or fragment thereof that binds CD47 is selected from the group consisting of magrolimab (Hu5F9-G4), CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

Embodiment 155. The method of any of the above or below embodiments, wherein the antibody or fragment thereof that binds CD47 is selected from the group consisting of a single-chain Fv fragment (scFv) against CD47, a Fab against CD47, a VHH nanobody against CD47, a DARPin against CD47 and variants thereof.

Embodiment 156. The method of any of the above or below embodiments, wherein the antibody or fragment thereof that binds SIRPα is selected from the group consisting of ADU-1805, CC-95251, OSE-172 (BI 765063), KWAR23, and P362.

Embodiment 157. The method of any of the above or below embodiments, wherein the antibody or fragment thereof that binds SIRPα is selected from the group consisting of a single-chain Fv fragment (scFv) against SIRPα, a Fab against SIRPα, a VHH nanobody against SIRPα, a DARPin against SIRPα and variants thereof.

Embodiment 158. The method of any of the above or below embodiments, wherein the SIRPα containing fusion protein comprises a CD47 binding domain of SIRPα linked to an Fc domain.

Embodiment 159. The method of any of the above or below embodiments, wherein the Fc domain comprises an Fc domain or portion thereof selected from the group consisting of IgG1, IgG2, IgG3, and IgG4.

Embodiment 160. The method of any of the above or below embodiments, wherein prior to administering step (b), step (a) is repeated at least 1-10 times.

Embodiment 161. The method of any of the above or below embodiments, wherein the effect of the population of cells in the patient comprises an adverse effect or an unwanted effect of the cells.

Embodiment 162. The method of any of the above or below embodiments, wherein the adverse effect is selected from the group consisting of hyperproliferation, transformation, tumor formation, cytokine release syndrome, graft-versus-host disease (GVHD), and immune effector cell-associated neurotoxicity syndrome (ICANS).

Embodiment 163. A method of controlling an effect of cell therapy in a patient, the method comprising administering a CD47-SIRPα blockade agent to the patient previously administered cells comprising exogenously expressed CD47 polypeptides.

Embodiment 164. The method of any of the above or below embodiments, wherein the cells comprise reduced expression of MHC class I and/or MHC class II human leukocyte antigens.

Embodiment 165. The method of any of the above or below embodiments, wherein the cells comprise reduced expression of MHC class I and MHC class II human leukocyte antigens.

Embodiment 166. The method of any of the above or below embodiments, wherein the cells further comprise reduced expression of one or more TCR complexes.

Embodiment 167. The method of any of the above or below embodiments, wherein the cells comprise exogenously expressed CD47 polypeptides and one or more additional exogenously expressed polypeptides selected from the group consisting of CD24, CD46, CD55, CD59, CD200, DUX4, PD-L1, IDO1, HLA-G, FasL, CCL21, Mfge8, Serpin B9, and any combination thereof.

Embodiment 168. The method of any of the above or below embodiments, wherein the cells further comprise one or more transgenes, wherein the transgene encodes an exogenously expressed polypeptide selected from the group consisting of CD24, CD46, CD55, CD59, CD200, DUX4, PD-L1, IDO1, HLA-G, FasL, CCL21, Mfge8, and Serpin B9.

Embodiment 169. The method of any of the above or below embodiments, wherein the cells comprise the exogenously expressed CD47 polypeptides and reduced expression levels of B2M and/or CIITA.

Embodiment 170. The method of any of the above or below embodiments, wherein the cells comprise the exogenously expressed CD47 polypeptides and reduced expression levels of B2M and CIITA.

Embodiment 171. The method of any of the above or below embodiments, wherein the cells comprise exogenously expressed CD47 polypeptides, reduced expression levels of B2M and/or CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 172. The method of any of the above or below embodiments, wherein the cells comprise exogenously expressed CD47 polypeptides, reduced expression levels of B2M and CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 173. The method of any of the above or below embodiments, wherein the cells further comprise a reduced expression level of TCRα, TCRβ, or both.

Embodiment 174. The method of any of the above or below embodiments, wherein the cells comprise differentiated cells derived from pluripotent stem cells.

Embodiment 175. The method of any of the above or below embodiments, wherein the pluripotent stem cells comprise induced pluripotent stem cells.

Embodiment 176. The method of any of the above or below embodiments, wherein the differentiated cells are selected from the group consisting of cardiac cells, neural cells, endothelial cells, T cells, pancreatic islet cells, retinal pigmented epithelium cells, hepatocytes, thyroid cells, skin cells, blood cells, and epithelial cells.

Embodiment 177. The method of any of the above or below embodiments, wherein the cells comprise cells derived from primary T cells.

Embodiment 178. The method of any of the above or below embodiments, wherein the cells derived from primary T cells are derived from a pool of primary T cells comprising primary T cells from one or more (e.g., two or more, three or more, four or more, five or more, ten or more, twenty or more, fifty or more, or one hundred or more) subjects different from the patient.

Embodiment 179. The method of any of the above or below embodiments, wherein the cells derived from primary T cells comprise a chimeric antigen receptor (CAR).

Embodiment 180. The method of any of the above or below embodiments, wherein the CAR and the exogenously expressed CD47 polypeptides are expressed under the control of a single promoter.

Embodiment 181. The method of any of the above or below embodiments, wherein the CAR binds an antigen selected from the group consisting of CD19, CD22, CD38, CD123, CD138, and BCMA.

Embodiment 182. The method of any of the above or below embodiments, wherein the cells derived from primary T cells comprise reduced expression of an endogenous T cell receptor.

Embodiment 183. The method of any of the above or below embodiments, wherein the cells derived from primary T cells comprise reduced expression of cytotoxic T-lymphocyte-associated protein 4 (CTLA4) and/or programmed cell death (PD1).

Embodiment 184. The method of any of the above or below embodiments, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

Embodiment 185. The method of any of the above or below embodiments, wherein the antibody or fragment thereof that binds CD47 is selected from the group consisting of magrolimab (Hu5F9-G4), CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

Embodiment 186. The method of any of the above or below embodiments, wherein the antibody or fragment thereof that binds CD47 is selected from the group consisting of a single-chain Fv fragment (scFv) against CD47, a Fab against CD47, a VHH nanobody against CD47, a DARPin against CD47 and variants thereof.

Embodiment 187. The method of any of the above or below embodiments, wherein the antibody or fragment thereof that binds SIRPα is selected from the group consisting of ADU-1805, CC-95251, OSE-172 (BI 765063), KWAR23, and P362.

Embodiment 188. The method of any of the above or below embodiments, wherein the antibody or fragment thereof that binds SIRPα is selected from the group consisting of a single-chain Fv fragment (scFv) against SIRPα, a Fab against SIRPα, a VHH nanobody against SIRPα, a DARPin against SIRPα and variants thereof.

Embodiment 189. The method of any of the above or below embodiments, wherein the SIRPα containing fusion protein comprises a CD47 binding domain of SIRPα linked to an Fc domain.

Embodiment 190. The method of any of the above or below embodiments, wherein the Fc domain comprises an Fc domain or portion thereof selected from the group consisting of IgG1, IgG2, IgG3, and IgG4.

Embodiment 191. The method of any of the above or below embodiments, wherein the effect of the previously administered cells comprise an adverse effect or an unwanted effect in the patient.

Embodiment 192. The method of any of the above or below embodiments, wherein the adverse effect is selected from the group consisting of hyperproliferation, transformation, tumor formation, cytokine release syndrome, graft-versus-host disease (GVHD), and immune effector cell-associated neurotoxicity syndrome (ICANS).

Embodiment 193. A CD47-SIRPα blockade agent for the treatment of an adverse effect subsequent to administration of a population of cells comprising exogenously expressed CD47 polypeptides.

Embodiment 194. A CD47-SIRPα blockade agent for the treatment of an adverse effect subsequent to administration of a population of cells comprising exogenously expressed CD47 polypeptides and reduced expression of MHC class I and MHC II human leukocyte antigens.

Embodiment 195. A CD47-SIRPα blockade agent for the treatment of an adverse effect subsequent to administration of a population of cells comprising exogenously expressed CD47 polypeptides and reduced expression of MHC class I and MHC II human leukocyte antigens and one or more TCR complexes.

Embodiment 196. Use of a CD47-SIRPα blockade agent in the manufacture of a medicament for cell therapy in a patient in need thereof, wherein the patient has been administered cells comprising exogenously expressed CD47 polypeptides.

Embodiment 197. Use of a CD47-SIRPα blockade agent in the manufacture of a medicament for cell therapy in a patient in need thereof, wherein the patient has been administered cells comprising exogenously expressed CD47 polypeptides and reduced expression of MHC class I and MHC II human leukocyte antigens.

Embodiment 198. Use of a CD47-SIRPα blockade agent in the manufacture of a medicament for cell therapy in a patient in need thereof, wherein the patient has been administered cells comprising exogenously expressed CD47 polypeptides and reduced expression of MHC class I and MHC II human leukocyte antigens and one or more TCR complexes.

Embodiment 199. Use of a CD47-SIRPα blockade agent in the manufacture of a medicament for modulating activity of a cell therapy in a patient, wherein the patient has received at least one dose of a therapeutically effective population of cells comprising exogenously expressed CD47 polypeptides.

Embodiment 200. Use of a CD47-SIRPα blockade agent in the manufacture of a medicament for modulating activity of a cell therapy in a patient, wherein the patient has received at least one dose of a therapeutically effective population of cells comprising exogenously expressed CD47 polypeptides and reduced expression of MHC class I and MHC II human leukocyte antigens.

Embodiment 201. Use of a CD47-SIRPα blockade agent in the manufacture of a medicament for modulating activity of a cell therapy in a patient, wherein the patient has received at least one dose of a therapeutically effective population of cells comprising exogenously expressed CD47 polypeptides and reduced expression of MHC class I and MHC II human leukocyte antigens and one or more TCR complexes.

Embodiment 202. Use of a CD47-SIRPα blockade agent in the manufacture of a medicament for controlling an effect of cell therapy in a patient, wherein the patient has been administered cells comprising exogenously expressed CD47 polypeptides.

Embodiment 203. The CD47-SIRPα blockade agent or use of any of the above or below embodiments, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

Embodiment 204. The CD47-SIRPα blockade agent or use of any of the above or below embodiments, wherein the antibody or fragment thereof that binds CD47 is selected from the group consisting of magrolimab (Hu5F9-G4), CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

Embodiment 205. The CD47-SIRPα blockade agent or use of any of the above or below embodiments, wherein the antibody or fragment thereof that binds CD47 is selected from the group consisting of a single-chain Fv fragment (scFv) against CD47, a Fab against CD47, a VHH nanobody against CD47, a DARPin against CD47, and variants thereof.

Embodiment 206. The CD47-SIRPα blockade agent or use of any of the above or below embodiments, wherein the antibody or fragment thereof that binds SIRPα is selected from the group consisting of ADU-1805, CC-95251, OSE-172 (BI 765063), KWAR23, and P362.

Embodiment 207. The CD47-SIRPα blockade agent or use of any of the above or below embodiments, wherein the antibody or fragment thereof that binds SIRPα is selected from the group consisting of a single-chain Fv fragment (scFv) against SIRPα, a Fab against SIRPα, a VHH nanobody against SIRPα, a DARPin against SIRPα, and variants thereof.

Embodiment 208. The CD47-SIRPα blockade agent or use of any of the above or below embodiments, wherein the SIRPα containing fusion protein comprises a CD47 binding domain of SIRPα linked to an Fc domain.

Embodiment 209. The CD47-SIRPα blockade agent or use of any of the above or below embodiments, wherein the Fc domain comprises an Fc domain or portion thereof selected from the group consisting of IgG1, IgG2, IgG3, and IgG4.

Embodiment 210. The CD47-SIRPα blockade agent or use of any of the above or below embodiments, wherein the cells further comprise reduced expression of MHC class I and/or MHC class II human leukocyte antigens.

Embodiment 211. The CD47-SIRPα blockade agent or use of any of the above or below embodiments, wherein the cells comprise reduced expression of MHC class I and MHC class II human leukocyte antigens.

Embodiment 212. The CD47-SIRPα blockade agent or use of any of the above or below embodiments, wherein the cells comprise exogenously expressed CD47 polypeptides and one or more additional exogenously expressed polypeptides selected from the group consisting of CD24, CD46, CD55, CD59, CD200, DUX4, PD-L1, IDO1, HLA-G, FasL, CCL21, Mfge8, Serpin B9, and any combination thereof.

Embodiment 213. The CD47-SIRPα blockade agent or use of any of the above or below embodiments, wherein the cells further comprise one or more transgenes wherein the transgene encodes an exogenously expressed polypeptide selected from the group consisting of CD24, CD46, CD55, CD59, CD200, DUX4, PD-L1, IDO1, HLA-G, FasL, CCL21, Mfge8, and Serpin B9.

Embodiment 214. The CD47-SIRPα blockade agent or use of any of the above or below embodiments, wherein the cells comprise exogenously expressed CD47 polypeptides and reduced expression levels of B2M and/or CIITA.

Embodiment 215. The CD47-SIRPα blockade agent or use of any of the above or below embodiments, wherein the cells comprise exogenously expressed CD47 polypeptides and reduced expression levels of B2M and CIITA.

Embodiment 216. The CD47-SIRPα blockade agent or use of any of the above or below embodiments, wherein the cells comprise exogenously expressed CD47 polypeptides and reduced expression levels of B2M and CIITA.

Embodiment 217. The CD47-SIRPα blockade agent or use of any of the above or below embodiments, wherein the cells comprise exogenously expressed CD47 polypeptides, reduced expression levels of B2M and/or CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 218. The CD47-SIRPα blockade agent or use of any of the above or below embodiments, wherein the cells comprise exogenously expressed CD47 polypeptides, reduced expression levels of B2M and CIITA and one or more additional exogenously expressed polypeptides selected from the group selected from of CD24, CD46, CD55, CD59, CD200, DUX4, PD-L1, IDO1, HLA-G, FasL, CCL21, Mfge8, Serpin B9, and any combination thereof.

Embodiment 219. The CD47-SIRPα blockade agent or use of any of the above or below embodiments, wherein the cells further comprise reduced expression level of TCRα, TCRβ or both.

Embodiment 220. The CD47-SIRPα blockade agent or use of any of the above or below embodiments, wherein the cells are differentiated cells derived from pluripotent stem cells.

Embodiment 221. The CD47-SIRPα blockade agent or use of any of the above or below embodiments, wherein the pluripotent stem cells comprise induced pluripotent stem cells.

Embodiment 222. The CD47-SIRPα blockade agent or use of any of the above or below embodiments, wherein the differentiated cells are selected from the group consisting of cardiac cells, neural cells, endothelial cells, T cells, pancreatic islet cells, retinal pigmented epithelium cells, hepatocytes, thyroid cells, skin cells, blood cells, and epithelial cells.

Embodiment 223. The CD47-SIRPα blockade agent or use of any of the above or below embodiments, wherein the cells comprise cells derived from primary T cells.

Embodiment 224. The CD47-SIRPα blockade agent or use of any of the above or below embodiments, wherein the cells derived from primary T cells are derived from a pool of primary T cells comprising primary T cells from one or more (e.g., two or more, three or more, four or more, five or more, ten or more, twenty or more, fifty or more, or one hundred or more) subjects different from the patient.

Embodiment 225. The CD47-SIRPα blockade agent or use of any of the above or below embodiments, wherein the cells derived from primary T cells comprise a chimeric antigen receptor (CAR).

Embodiment 226. The CD47-SIRPα blockade agent or use of any of the above or below embodiments, wherein the CAR and the exogenously expressed CD47 polypeptides are expressed under the control of a single promoter.

Embodiment 227. The CD47-SIRPα blockade agent or use of any of the above or below embodiments, wherein the CAR binds an antigen selected from the group consisting of CD19, CD22, CD38, CD123, CD138, and BCMA.

Embodiment 228. The CD47-SIRPα blockade agent or use of any of the above or below embodiments, wherein the cells derived from primary T cells comprise reduced expression of an endogenous T cell receptor.

Embodiment 229. The CD47-SIRPα blockade agent or use of any of the above or below embodiments, wherein the cells derived from primary T cells comprise reduced expression of cytotoxic T-lymphocyte-associated protein 4 (CTLA4) and/or programmed cell death (PD1).

Embodiment 230. A CD47-SIRPα blockade agent for the treatment of an adverse effect subsequent to administration of a population of cells comprising exogenously expressed CD47 polypeptides and reduced expression of B2M, CIITA and TCRα.

Embodiment 231. A CD47-SIRPα blockade agent for the treatment of an adverse effect subsequent to administration of a population of cells comprising exogenously expressed CD47 polypeptides and reduced expression of B2M, CIITA and TCRβ.

Embodiment 232. Use of a CD47-SIRPα blockade agent in the manufacture of a medicament for cell therapy in a patient in need thereof, wherein the patient has been administered cells comprising exogenously expressed CD47 polypeptides and reduced expression of B2M, CIITA and TCRα.

Embodiment 233. Use of a CD47-SIRPα blockade agent in the manufacture of a medicament for cell therapy in a patient in need thereof, wherein the patient has been administered cells comprising exogenously expressed CD47 polypeptides and reduced expression of B2M, CIITA and TCRβ.

Embodiment 234. Use of a CD47-SIRPα blockade agent in the manufacture of a medicament for modulating activity of a cell therapy in a patient, wherein the patient has received at least one dose of a therapeutically effective population of cells comprising exogenously expressed CD47 polypeptides and reduced expression of B2M, CIITA and TCRα.

Embodiment 235. Use of a CD47-SIRPα blockade agent in the manufacture of a medicament for modulating activity of a cell therapy in a patient, wherein the patient has received at least one dose of a therapeutically effective population of cells comprising exogenously expressed CD47 polypeptides and reduced expression of B2M, CIITA and TCRβ.

### EXAMPLES

### Example 1. CD47 Blockade In Vitro

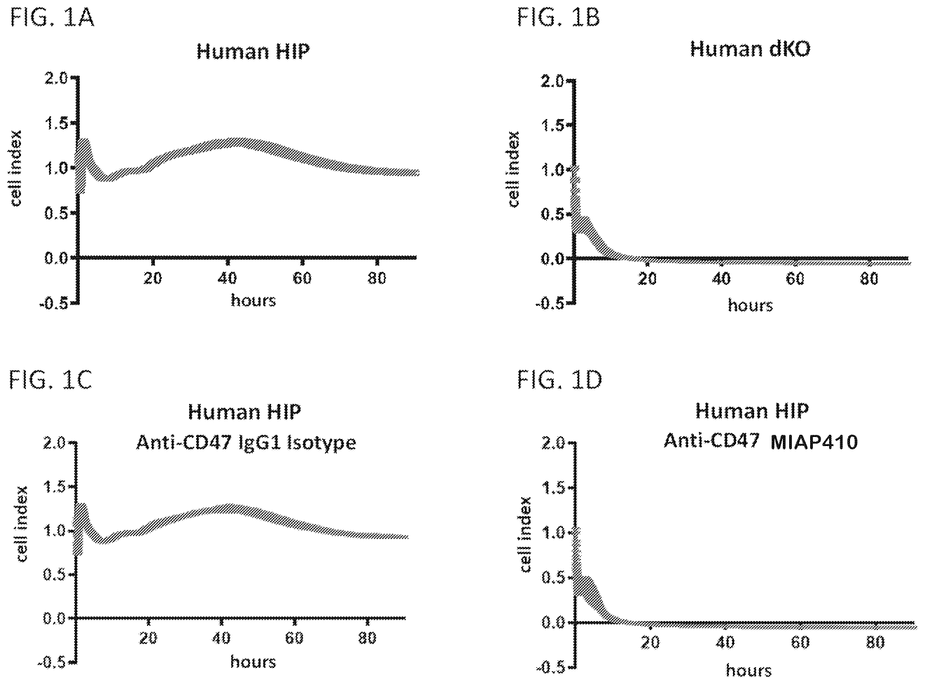

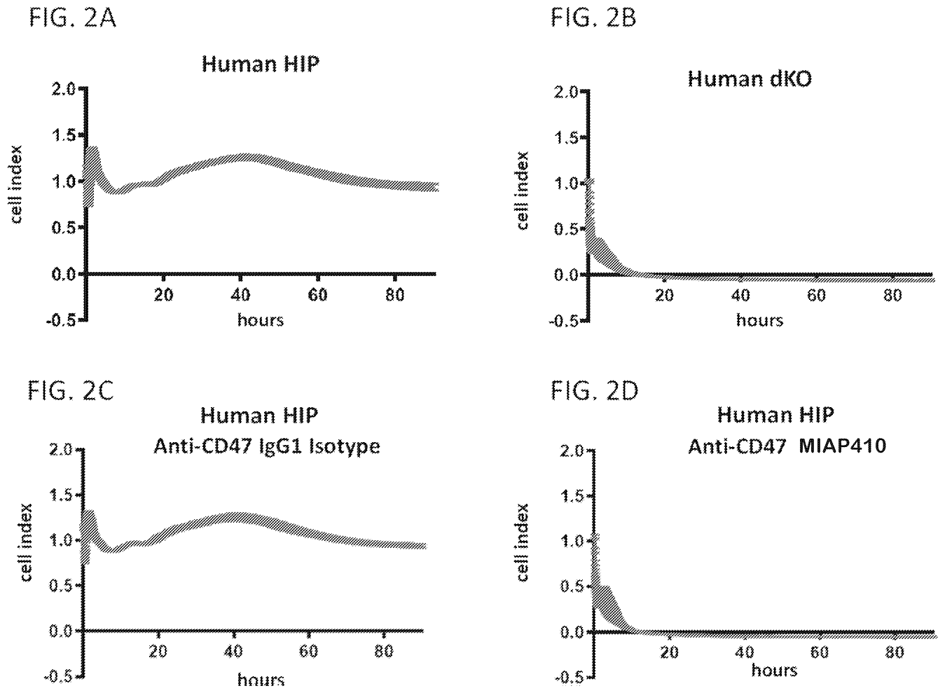

The ability of CD47 blockade agents (also referred to herein as CD47-SIRPα blockade agents) to eliminate CD47's inhibitory effect on killing by NK cells and macrophages was assayed on the XCELLIGENCE MP platform (ACEA BioSciences). 96-well E-plates (ACEA BioSciences) were coated with collagen (Sigma-Aldrich) and 4×105 human HIP (B2M−/−, CIITA−/−, CD47+) or double knockout (B2M−/−, CIITA−/−) cells were plated in 100 μL cell specific media. The human cells were plated and treated one day later with 100 μg/ml anti-CD47 antibody MIP410 (BioXCell, Lebanon, NH) or an IgG1 isotype control (BioXCell). After the Cell Index value reached 0.7, human NK cells or human macrophages (Lonza) were added with an E:T ratio of 1:1, either with or without 1 ng/ml human IL-2 (PeproTech). As a killing control, cells were treated with 2% TRITON X100. No killing was observed by stimulated or unstimulated NK cells (FIG. 1A) or macrophages (FIG. 2A) on HIP cells expressing CD47, whereas the double knockout cells were rapidly killed by both NK cells (FIG. 1B) and macrophages (FIG. 2B). By contrast, the HIP cells treated with anti-CD47 antibody were rapidly killed by NK cells (FIG. 1D) and macrophages (FIG. 2D), whereas no killing was seen following IgG1 isotype control treatment (FIGS. 1C and 2C). This example demonstrates that CD47 blockade removed the protective effect of CD47 against NK cell and macrophage killing.

### Example 2. CD47 Blockade In Vivo

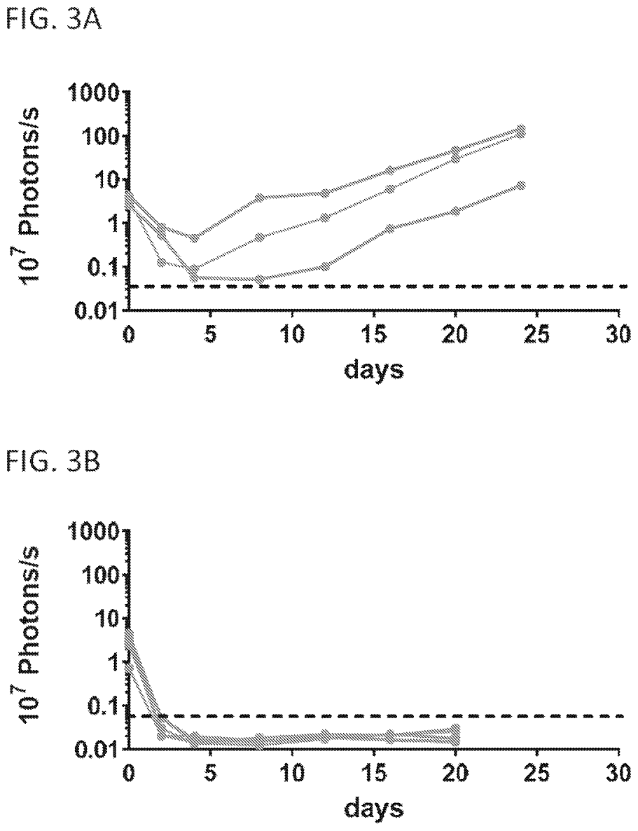

NSG mice have functional macrophages, but lack other immune cells. 1 million human NK cells were therefore transferred i.v. into NSG mice and at the same time 50 thousand human HIP (B2M−/−, CIITA−/−, CD47+) iPSCs were injected subcutaneously into the same mice. 500 μg/kg anti-CD47 antibody (MIP410; BioXCell) was injected s.c. daily (n=5). Control mice received an IgG1 isotype control (BioXCell) (n=3). For bioluminescence imaging (BLI), D-luciferin firefly potassium salt (375 mg/kg; Biosynth) was dissolved in PBS (pH 7.4) (Gibco, Invitrogen) and was injected intraperitoneally (250 μL per mouse) into anesthetized mice. Animals were imaged using an AMI imager (Spectral Instruments). Region of interest (ROI) bioluminescence was quantified in units of maximum photons per second per centimeter square per steradian (p s-1 cm-2 sr-1). The maximum signal from an ROI was measured using Living Image software (MediaCybernetics). Mice were monitored on day 0, day 2 and every 4th day until signal reached background or teratoma reached >2000 mm3 volume. Bioluminescence from the HIP iPSCs in mice administered the IgG1 isotype control antibody increased over the course of 25 days, indicating survival of the iPSCs (FIG. 3A). On the contrary, bioluminescence of the HIP iPSCs decreased rapidly following administration of the anti-CD47 antibody, falling below background by day 4 (FIG. 3B). This example demonstrates that CD47 blockade can induce killing of hypoimmune cells that express CD47.

### Example 3. Creating Hypoimmunogenic CAR-T Cells to Evade Immune Recognition for Allogeneic Therapies

### A. Abstract

Off-the-shelf CAR-T cells could offer advantages over autologous strategies, including ease of manufacturing, quality control and avoidance of malignant contamination and T cell dysfunction. However, the vigorous host-versus-graft immune response against histo-incompatible T cells prevents expansion and persistence of allogeneic CAR-T cells and mitigates the efficacy of this approach. Described herein are methods for engineering and generating human immune evasive CAR-T cells using a novel hypoimmune editing platform. The system is based, in part, on the finding that T cells lose their immunogenicity when human leukocyte antigen (HLA) class I and II genes are inactivated and CD47 is overexpressed. Additionally, it has been found that TCR knockout is useful to control the risk of graft-versus-host disease.

This example describes hypoimmunogenic CD19-specific CAR-T cells (also referred to herein as HIP CD19CAR-T cells) and the effect of exogenous CD47 expression on the activity of such cells compared to control CD19-specific CAR-T cells in in vitro tumor efficacy experiments. In the experiments, CD19⁺ tumor cells were used as the target cell and HIP CD19CAR-T cells (such as the test and control CAR-T cells) were used as the effector cell. In the experiments described, the HIP CD19CAR-T cells (also referred to as “HIP CAR-T cells”) were T cells containing (a) genome edits of the B2M, CIITA and TRAC genes and (b) transgenes carrying a polynucleotide encoding a CD19-specific CAR and a polynucleotide encoding CD47. Such HIP CD19CAR-T cells are also referred to as B2M^{-/-}, CIITA^{-/-}, TRAC^{-/-}, CD19-specific CAR-CD47 T cells. Also, in the experiments, the control CAR-T cells included (a) immunogenic T cells expressing a polynucleotide encoding a CD19-specific CAR, including T cells expressing the same CAR construct as tisagenlecleucel or a biosimilar/surrogate thereof, (b) immunogenic T cells expressing polynucleotides encoding a CD19-specific CAR and EGFRt, and (c) mock transfected T cells, namely T cells that were mock transfected.

When transplanted into allogeneic humanized mice, the HIP CD19CAR-T cells (e.g., B2M^{-/-}, CIITA, TRACY, CD19-specific CAR-CD47 T cells) evaded immune recognition by T and B cells compared to immunogenic CD19-positive CAR T cells generated from the same human donor. Innate immune cell (e.g., NK cell or macrophage) assays showed that CD47 overexpression protects HIP CD19CAR-T cells from innate immune cell killing in vitro and in vivo. CD47 expression levels were analyzed to assess threshold levels for protection by using a method for flow cytometric estimation of antibodies bound per cell. A blocking antibody against CD47 made the HIP CD19CAR-T cells susceptible to macrophage and NK cell killing, confirming the importance of CD47 overexpression to evade innate immune clearance. Accordingly, the use of a blocking antibody against CD47 is envisioned as a strategy for providing a safety switch to the HIP CD19CAR-T cells described herein.

Neither isolated CD47 overexpression nor genetic modifications to inactivate B2M, CIITA and TRAC genes showed any effect on the cytotoxic potential of the CAR-T cells. HIP CD19CAR-T cells (e.g., B2M^{-/-}, CIITA^{-/-}, TRAC^{-/-}, CD19-specific CAR-CD47 T cells) retained their antitumor activity in the CD19⁺ tumor model in vitro as well as in immunodeficient NSG mice across a range of tumor cell: HIP CD19CAR-T cell ratios. It did not appear that the introduction of the hypoimmune gene edits (e.g., B2M/CIITA/TRAC gene inactivation) changed their cytokine independent growth as compared to immunogenic CAR-T cells. These findings show that HIP CD19CAR-T cells are functionally immune evasive in allogeneic recipients with prolonged cytotoxic anti-tumor capacity. Accordingly, the results suggest that the HIP CD19CAR-T cells can provide universal immunotherapeutic options for cancer patients.

### B. Methods

Production of CAR-CD47 engineered pan T cells: Pan T cells were thawed and cultured overnight in standard T cell media supplemented with IL-2. Viable T cells were enumerated and activated with CD3/CD28 beads at a bead: cell ratio of 1:1. T cell transduction was performed using a CD19-specific CAR and CD47 expressing lentivirus (e.g., a lentivirus expressing a CD19-specific CAR and CD47 transgenes under the control of a single promoter) at an MOI of 100 in the presence of protamine sulfate to generate CD47 engineered pan T cells. Also, mock transfected pan T cells were generated, wherein T cells were mock transfected. Cells were expanded in standard T cell media supplemented with IL-2 and frozen in standard freezing medium for later use.

Production of B2M/CIITA double knockout pan T cells and B2M/CIITA double knockout/CAR-CD47 engineered pan T cells: Frozen mock transfected T cells, namely T cells that were mock transfected, and CD47 engineered T cells were thawed and cultured overnight, following which, the cells were re-activated with CD3/CD28 beads. The beads were removed from the cells after about Day 4. Ribonucleoprotein (RNP) complexes containing Cas9 and the guide RNAs targeting B2M and CIITA were formed according to standard protocol. T cells were resuspended in a nucleofection buffer and added to the RNP complexes prior to electroporation using a standard cell electroporating device. After electroporation, cells were recovered in standard T cell media supplemented with IL-2. Editing efficiency was assessed by FACS analysis for CD47, HLA-ABC, and HLA-DR.

Cytokine independent growth assay: engineered T cells were washed twice in 1×PBS prior to use and resuspended in standard T cell media overnight prior to use in the assay. Cells were resuspended in standard culture medium supplemented with or without IL-2 on Day 1. Thereafter, viable cells were counted twice a week by trypan blue exclusion. Throughout the assay, cell concentration was adjusted using the IL-2 supplemented medium.

NK cell culture: Human primary NK cells were stimulated with human IL-2 in standard NK cell culture media (e.g., RPMI 1640 plus 10% heat-inactivated FCS and 1% Pen/Strep) before the cells were used in assays.

Macrophage culture: PBMCs were isolated by Ficoll separation from fresh blood and were resuspended in standard macrophage culture media (e.g., RPMI 1640 with 10% heat-inactivated FCS and 1% Pen/Strep). Cells were plated and cultured in human M-CSF. Media was changed every second day. From day 6 onward, human IL-2 was added into the media for about 24 hours before the cells were used in experimental assays.

CD19⁺ tumor culture: CD19⁺ tumor cells were cultured in standard cell culture media (e.g., RPMI 1640 with 10% heat-inactivated FCS and 1% Pen/Strep) before being used in experimental assays.

Innate killing using a real-time cell analysis system: NK cell killing assays and macrophage phagocytosis assays were performed on a real-time cell analysis instrument (e.g., XCelligence® MP platform, Agilent). Briefly, T-cells were plated in CD19⁺ tumor cell specific media. After cell index value reached 0.7, human NK cells or human macrophages were added with an E:T ratio of 1:1 with human IL-2 for NK cells and without IL-2 for macrophages. Some wells were pretreated with a human CD47 blocking antibody or a human EGFR antibody (e.g., cetuximab) and again during the course of the real-time analysis assay. Data were standardized and analyzed with the real-time cell analysis instrument software.

After about 48 hours, T-cells were collected and stained with components of a live/dead cell viability staining assay. Briefly, cells were stained with calcein AM and ethidium homodimer-1 according to the manufacturer's protocol. The analysis was performed by flow cytometry and the results were expressed as percentage of dead cells.

In vitro CD19⁺ tumor cell killing using a real-time cell analysis system: in vitro CD19⁺ tumor cell killing assays were performed on a real-time cell analysis instrument (e.g., XCelligence® MP platform, Agilent). Briefly, T-cells were plated in CD19⁺ tumor cell specific media. After cell index value reached 0.7, human NK cells or human macrophages were added with an E:T ratio of 0.125:1, 0.25:1, 0.5:1, 1:1, 3:1 or 7:1 in standard T cell media. Data were standardized and analyzed with the real-time cell analysis instrument software.

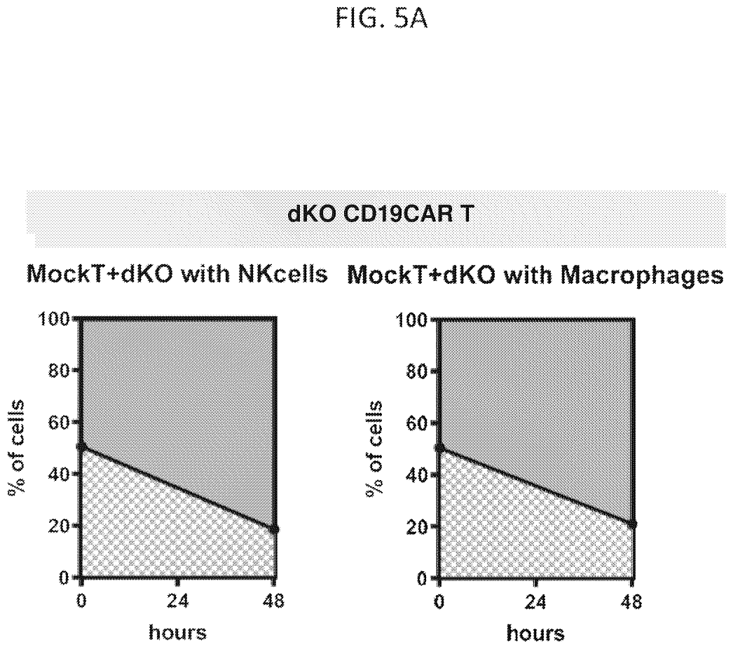

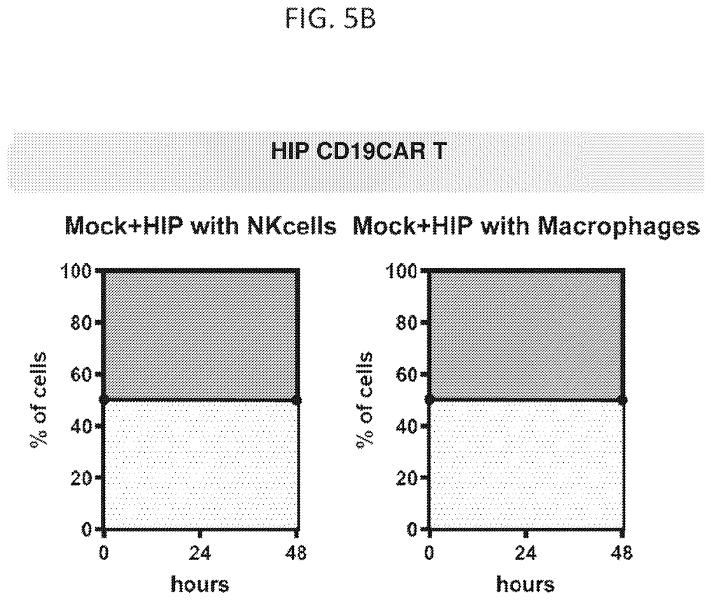

In vivo cytotoxicity assay with adoptive transfer: (1) Unmodified T cells and modified B2M−/−CIITA−/− T cells or (2) unmodified T cells and modified B2M−/−CIITA−/− CD47 tg T cells were mixed and stained with different fluorophore dyes using a multicolor cell-labeling kit. The cells were diluted in saline and human IL-2. Then, the cells were combined with either human primary NK cells or human macrophages and injected i.p. into immunodeficient NSG mice. The human primary NK cells were pretreated with human IL-2 in vitro before injection. Approximately 48 hours after injection, peritoneal cells were collected from the experimental mice and analyzed by flow cytometry. The ratio was compared between the unmodified T cells and the modified T cells. The results are shown in FIGS. 5A-5B. The adoptive transfer data showed that CAR-T cells carrying inactivated HLA-I/II genes were killed in vivo by NK cells and macrophages. Yet, CAR-T cells carrying inactivated HLA-I/II and TCR genes and overexpressing CD47 were not killed by innate immune cells.

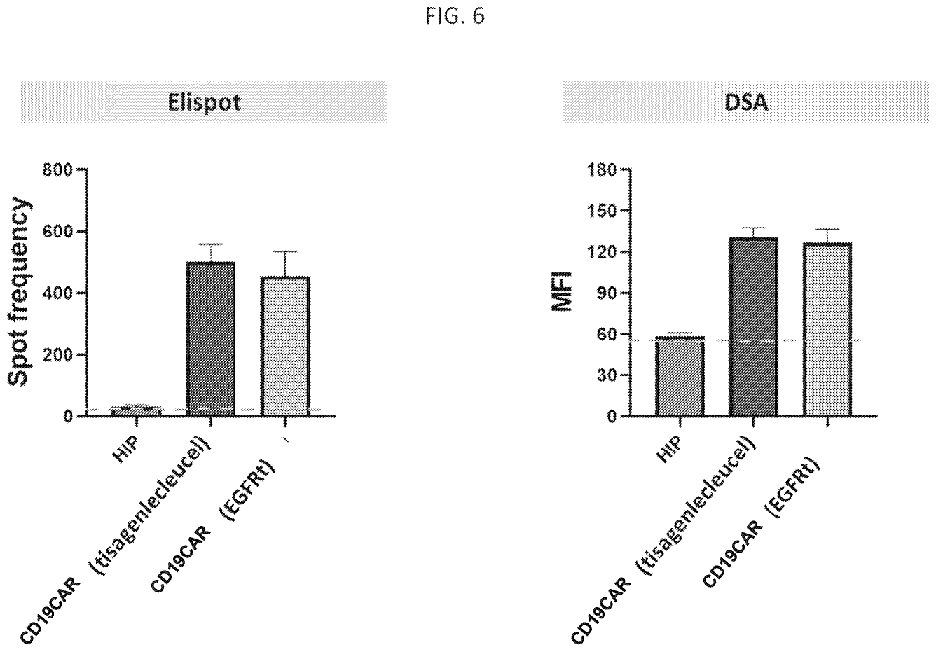

Enzyme-linked immune absorbent spot ELISpot assay: hypoimmunogenic CAR-T cells or allogeneic CAR-T cells T cells were injected into NSG-SGM3 humanized mice with n=3 per group. Recipient splenocytes were isolated from the spleen approximately 6 days after cell injection and were used as responder cells. Donor T cells were mitomycin-treated and used as stimulator cells. Stimulator cells were incubated with recipient responder T cells for 48 hours and IFN-γ spot frequencies were enumerated using an ELISpot plate reader. The results are shown in FIG. 6. The hypoimmunogenic CAR-T cells did not induce T cell activation, however, activated T cells the allogeneic CAR-T cells were detected in the humanized mice.

Donor specific antibodies assay: sera from the recipient mice (e.g., NSG-SGM3 humanized mice injected with hypoimmunogenic CAR-T cells or allogeneic CAR-T cells) were de-complemented by heating to 56° C. for approximately 30 min. Equal amounts of sera and cell suspensions were incubated for approximately 45 min at 4° C. Cells were labeled with FITC-conjugated goat anti-human IgM and analyzed by flow cytometry. The results are shown in FIG. 6. The hypoimmunogenic CAR-T cells did not induce donor-specific antibody binding, yet the IgM binding against allogeneic CAR-T cells was detected.

### C. Results

The hypoimmunogenic T cells described herein appeared to lack immunogenicity as a result of human leukocyte antigen (HLA) class I and II and TCR gene inactivation (e.g., knockout of B2M, CIITA and TRAC genes) and CD47 overexpression. Such hypoimmune T cells were engineered to express CD19-specific CAR molecules. The introduction of the hypoimmune gene edits did not change the cytokine independent growth of the cells, as compared to corresponding immunogenic CAR-T cells.

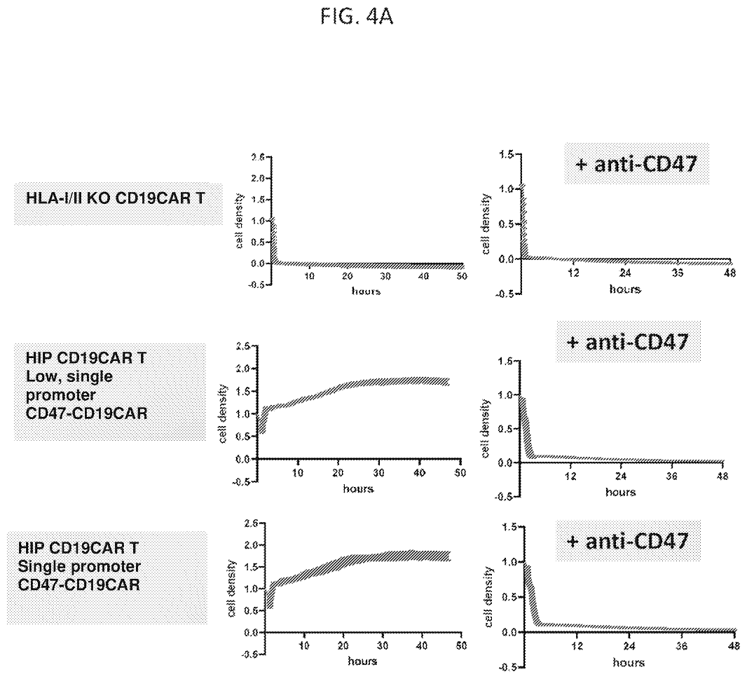

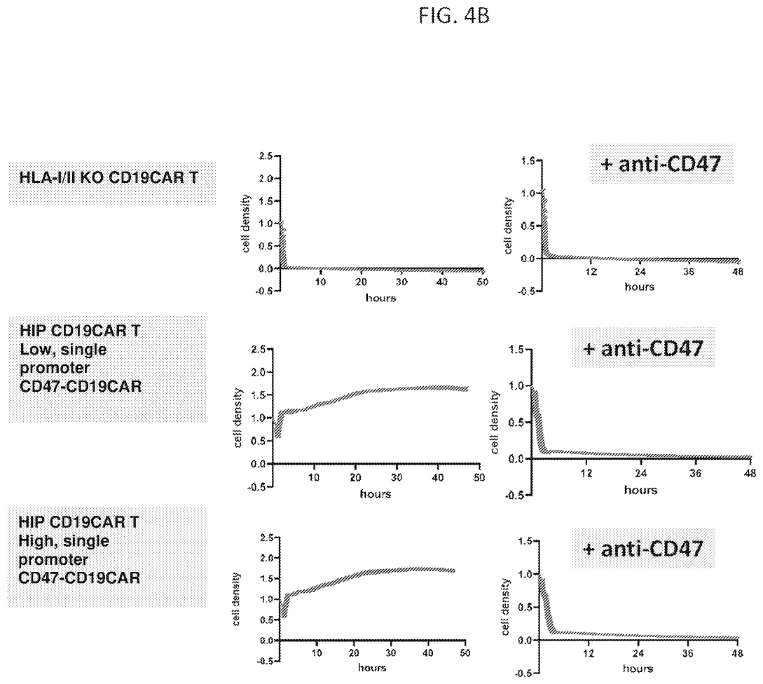

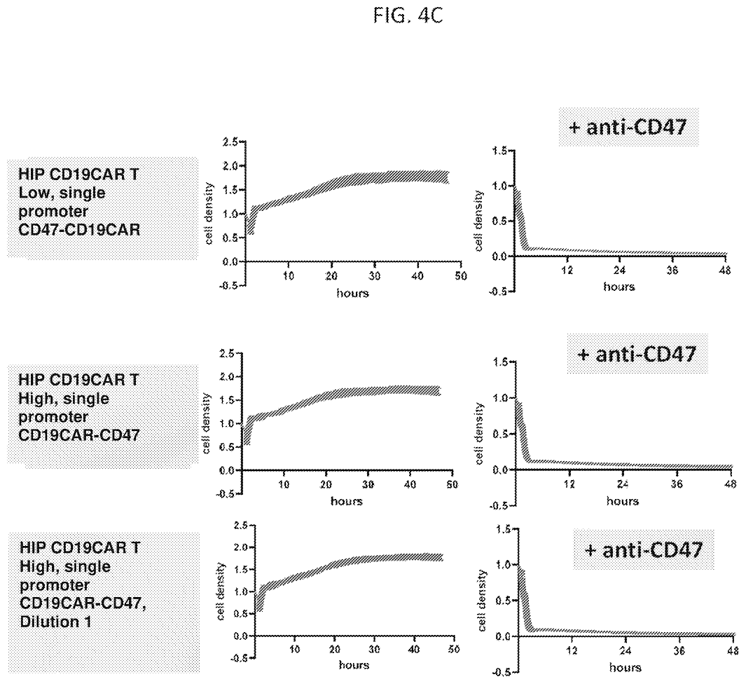

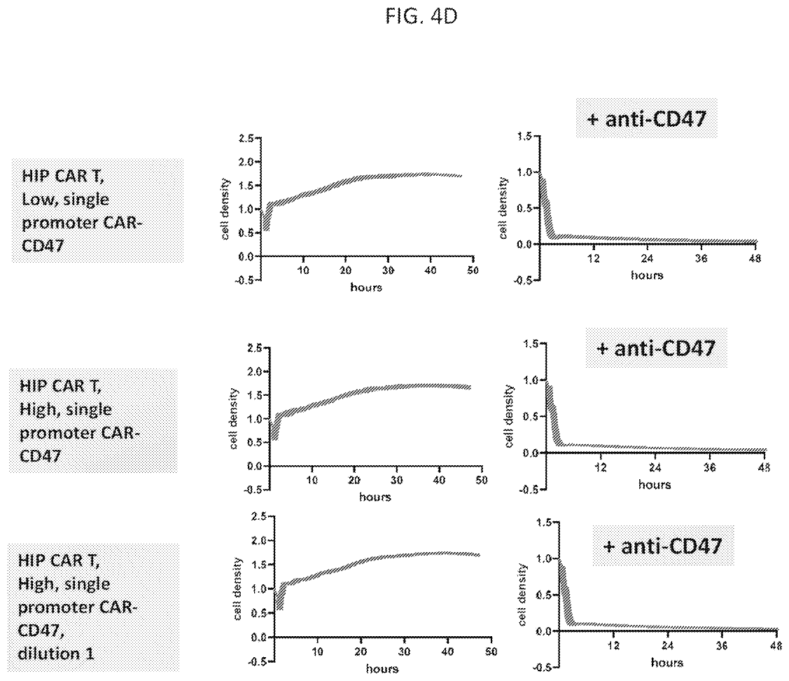

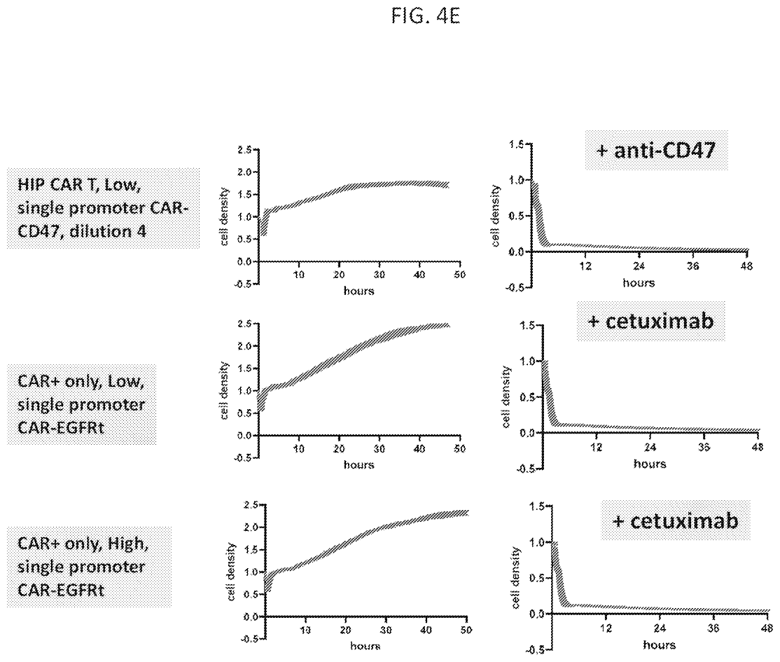

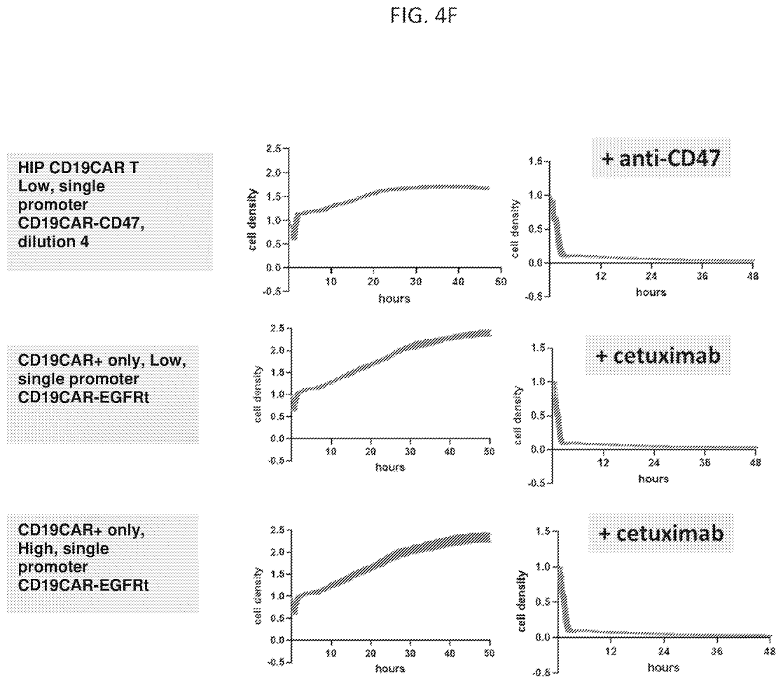

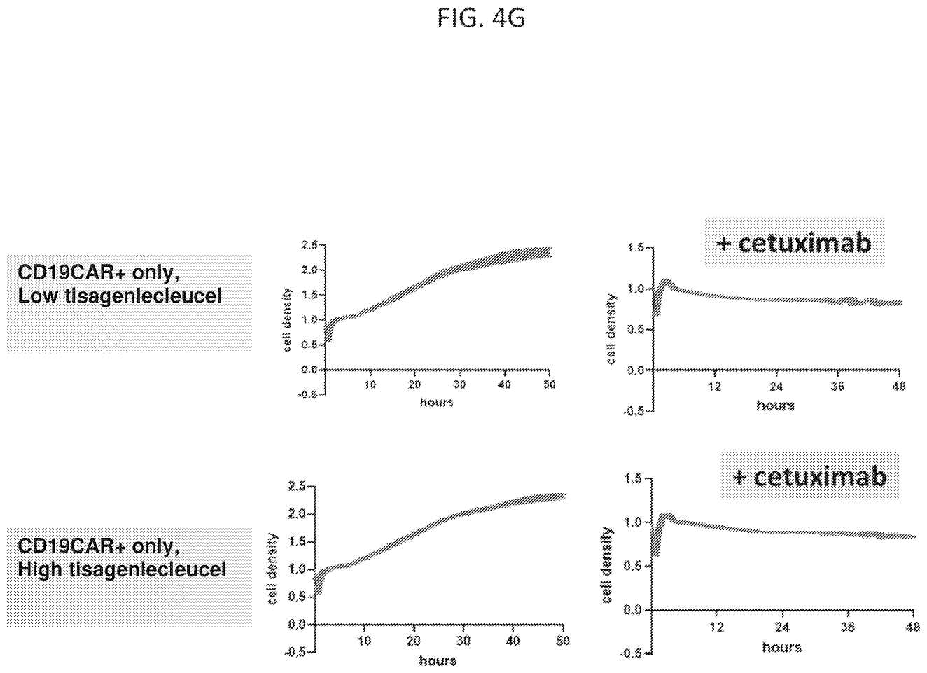

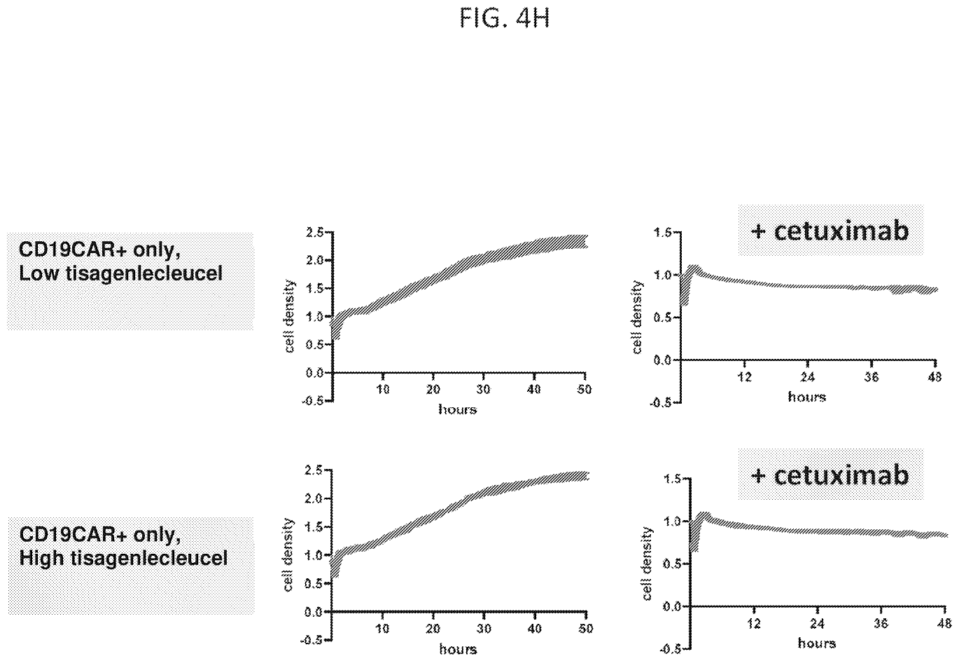

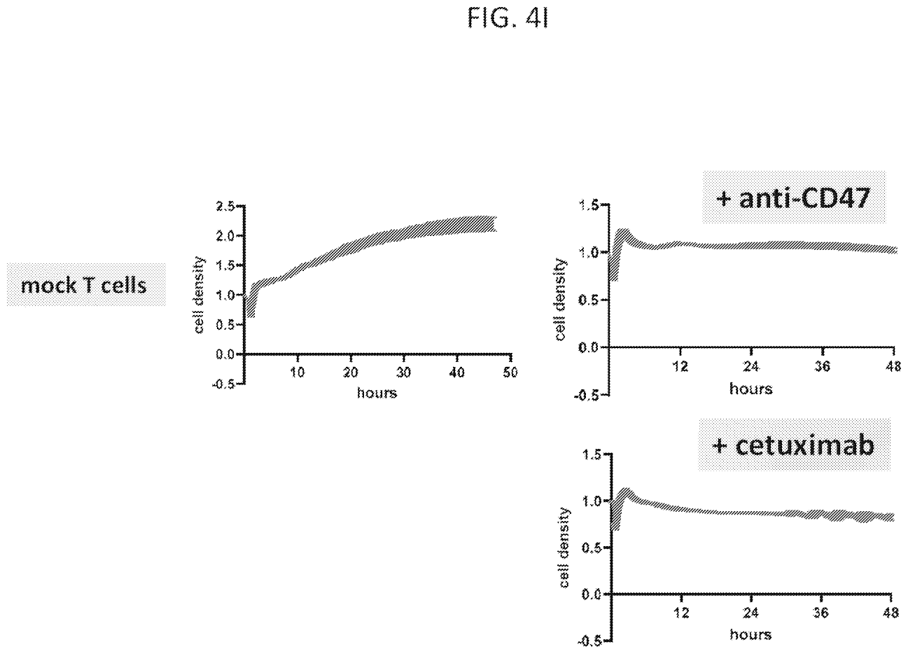

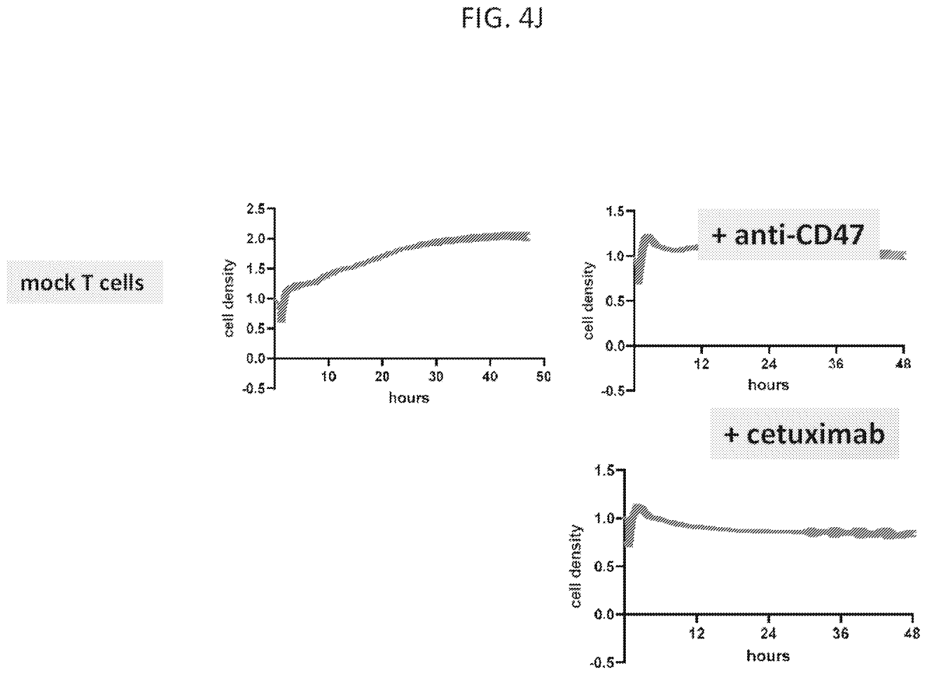

Further, exposure to a blocking antibody against CD47 (e.g., magrolimab) made hypoimmunogenic CAR-T cells (e.g., B2M^{-/-}, CIITA^{-/-}, TRAC^{-/-}, CD19-specific CAR-CD47 T cells) susceptible to macrophage and NK cell killing, confirming the importance of CD47 overexpression to evade innate immune clearance. See, for example, FIGS. 4A-4J and 5A-5B. The data showed that the CD19-specific CAR and CD47 constructs were expressed at low or high levels in the experimental cells. Also, such constructs were expressed under the control of a single promoter (e.g., a single constitutive active promoter). The results showed that the hypoimmunogenic CAR-T cells were not killed by innate immune cells in an in vitro assay. In contrast, blocking of CD47 by macrolimab induced killing of the hypoimmunogenic CD19-specific CAR-T cells by innate immune cells in vitro. As expected, killing of the control CAR-T cells (such as T cells expressing CAR-EGFRt constructs or tisagenlecleucel biosimilars or surrogates) by innate immune cells was not detected. Innate cell killing of control CAR-T cells expressing CAR-EGFRt constructs was induced using a blocking antibody against EGFR (e.g., cetuximab). The data also showed that mock transfected T cells, namely T cells that were mock transfected, were not killed by NK cell and macrophages in the absence or presence of magrolimab or cetuximab.

In summary, treatment with a CD47 antibody could be used as a safety strategy to abrogate the hypoimmunogenic nature of the hypoimmunogenic CAR-T cells described herein, thus allowing the recipient subject's body to remove them.

### Example 4. Administering CD47-SIRPα Blockade Agents Results in Killing of Hypoimmune Cells In Vitro and In Vivo

Safety mechanism studies using hypoimmune (B2M−/−, CIITA−/−, and CD47tg+) cells in vitro and in vivo were conducted using the protocols described herein.

### A. Safety Mechanism Study Protocols

### 1. Cell Implantation

Subcutaneous. Healthy male NSG mice were anesthetized via isoflurane inhalation anesthesia in a knockdown chamber. The anesthetized mice were removed from the chamber and placed in a supine position. The injection site (right inguinal area) was sprayed with 70% EtOH. Cells were injected subcutaneously proximal to the inguinal region.

Brain. 24 to 48 hours prior to scheduled surgery, animals were provided with MediGel (Clear H2O, Portland, ME) with carprofen (5 mg/Kg/day). The amount calculated was based on standard intake. To each cup of MediGel (sucralose), 1.5 mg of carprofen was added. Alternatively, buprenorphine (0.05-0.1 mg/Kg IM or IP) BID every 12 hours for three days or Buprenorphine SR (0.5-1.0 mg/Kg SC) was administered once prior to the start of surgery. Buprenorphine SR or buprenorphine was not used post-operatively in conjunction with MediGel.

On surgery day, animals were anesthetized and maintained via isoflurane inhalation (3% and 1.5%, respectively). The top of the head was shaved and scrubbed with chlorhexidine and wiped with 70% EtOH. The animal was placed on a stereotaxic device. An incision was made midline through the scalp long enough to easily expose midline suture from bregma to lambda.

At pre-determined coordinates (as identified by the Allen Brain Atlas or George Paxinos' Mouse Brain Atlas), the cranium was drilled through without penetrating the dura. A micro-drill was exchanged for a micro-infusion pump with a Hamilton syringe and needle. The needly was “dropped” to the depth required to reach the target area of the brain and injectate (differentiated stem cells in 1 μl-5 μl saline) was infused. The incision was closed with absorbable suture and mouse recovered on a warming pad. Animals were maintained on MediGel (sucralose gel with carprofen) for 3 days.

### 2. Bioluminescent Imaging (BLI)

Mice were anesthetized via isoflurane inhalation anesthesia and injected intraperitoneally (IP) with 250 μl luciferin (45.45 mg/ml). The mice were then positioned in a nose cone in the bioluminescence chamber (anesthesia was maintained at 1.5% with 500 ml oxygen/min). The mice were imaged for 180 seconds. A circle was drawn around the region of interest (ROI) giving a reading of photons/sec of emitted light.

### 3. Antibody (Ab) Dosing

IP dosing. The antibody (100 μl) was injected into the peritoneal cavity from the ventral surface by piercing the skin and muscle layers.

SC dosing. Mice were anesthetized via isoflurane inhalation anesthesia and placed in a supine position. The injection site (right inguinal area) was sprayed with 70% EtOH and the antibody (100 μl) was injected into the area of the implanted cells.

IV dosing. Mice were warmed under a heat lamp and antibody was injected into a dilated lateral vein.

B. Human iPSCs: Magrolimab In Vitro and In Vivo

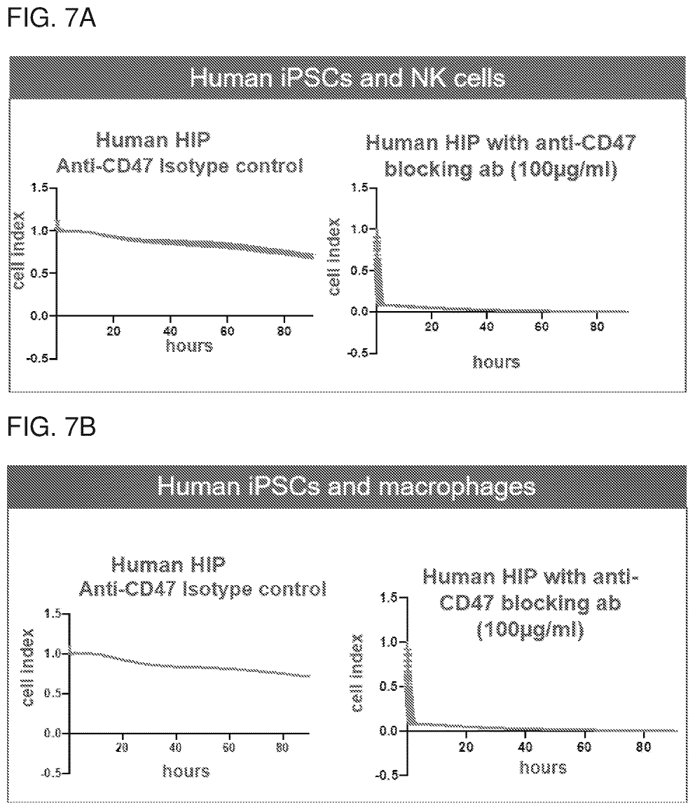

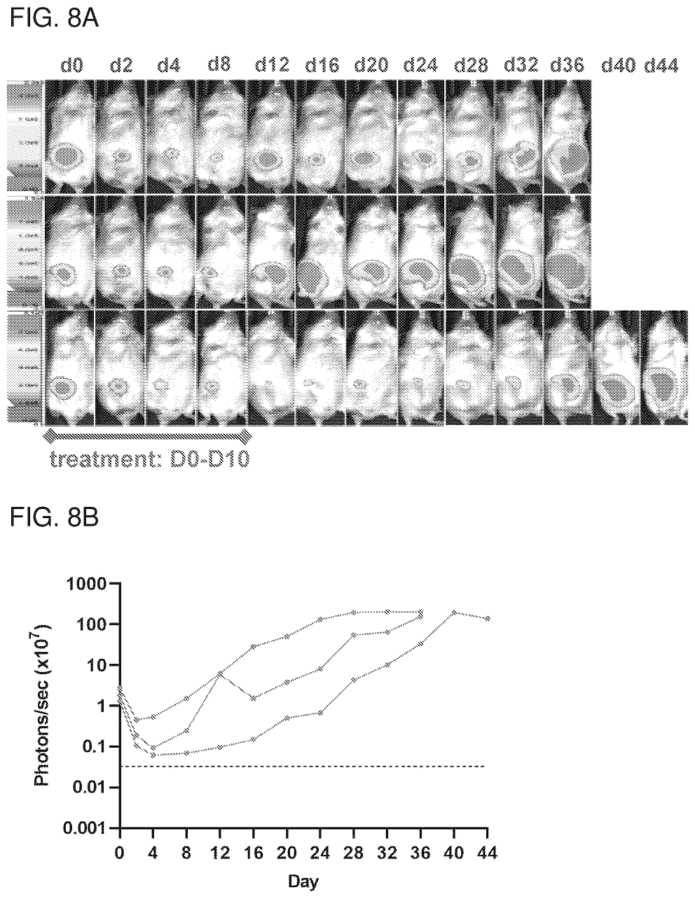

As shown in FIG. 7A-7B, killing of human HIP iPSCs by NK cells and macrophages occurred when anti-CD47 magrolimab antibody was added. Human HIP iPSCs were injected subcutaneously into NSG mice with adoptive transfer of human NK cells. A local isotype control (mouse IgG4) was used and treatment was performed during the period of Day 0 to Day 10 (D0-D10). As shown in FIG. 8A-B, human HIP iPSCs formed teratoma in NSG mice with adoptive transferred NK cells. Treatment with IgG4 isotype control did not affect HIP survival.

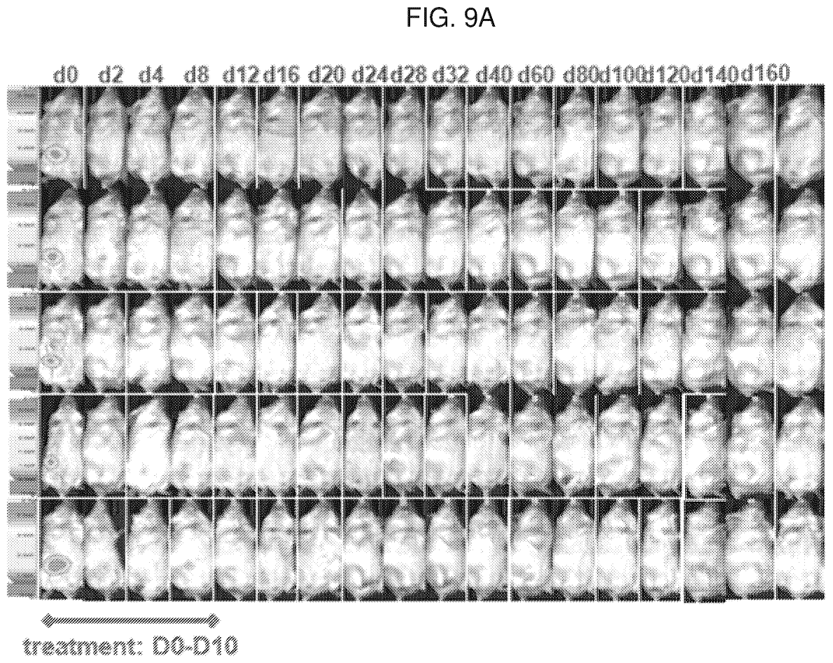

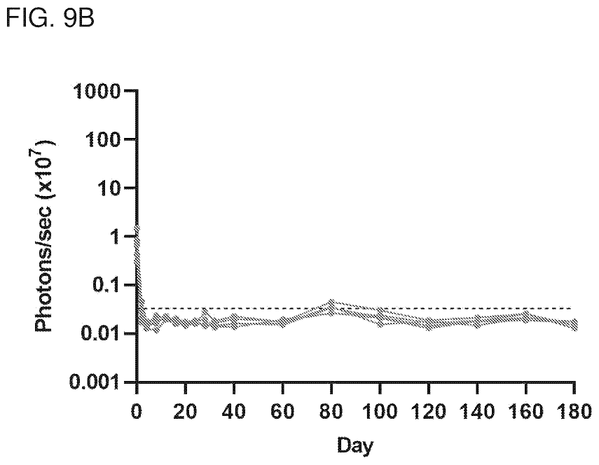

5×10⁴ Human HIP iPSCs were injected subcutaneously into NSG mice with adoptive transfer of 1×10⁶ human NK cells. A local isotype control (mouse IgG4) was used and treatment was performed during the period of Day 0 to Day 10 (D0-D10). As shown in FIG. 9A-B, blocking of CD47 in vivo resulted in killing of HIP iPSCs. Magrolimab treatment was stopped after D10. iPSCs didn't re-appear when checked during a 6-month follow up.

C. Human iPSCs: MIAP410 In Vitro and In Vivo

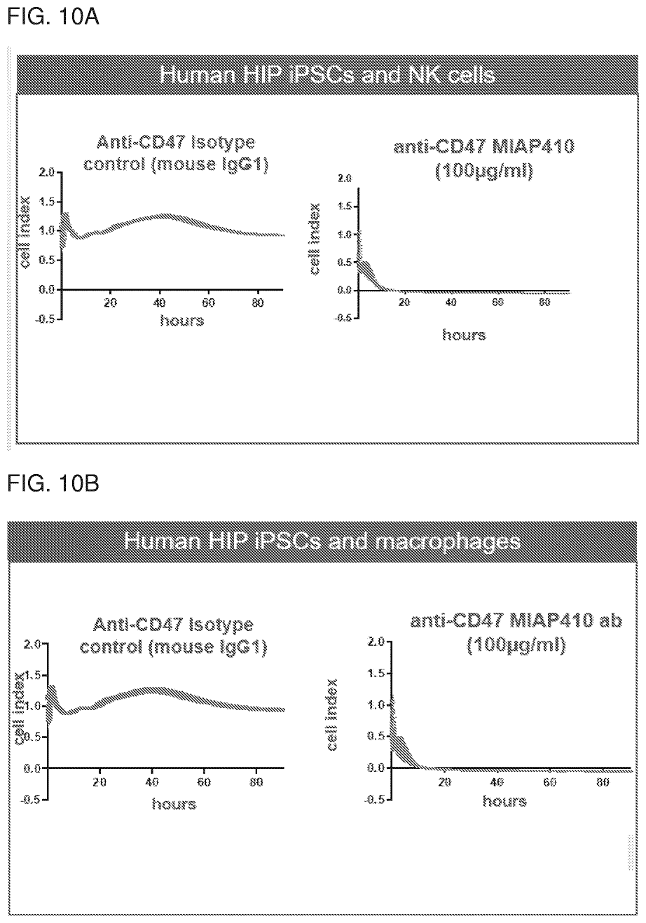

CD47 blocking by anti-CD47 antibody MIAP410 was observed in vitro in human immune cells. As shown in FIG. 10A-B, killing of human HIP iPSCs by human NK cells and human macrophages was observed when anti-CD47 MIAP410 antibody was added.

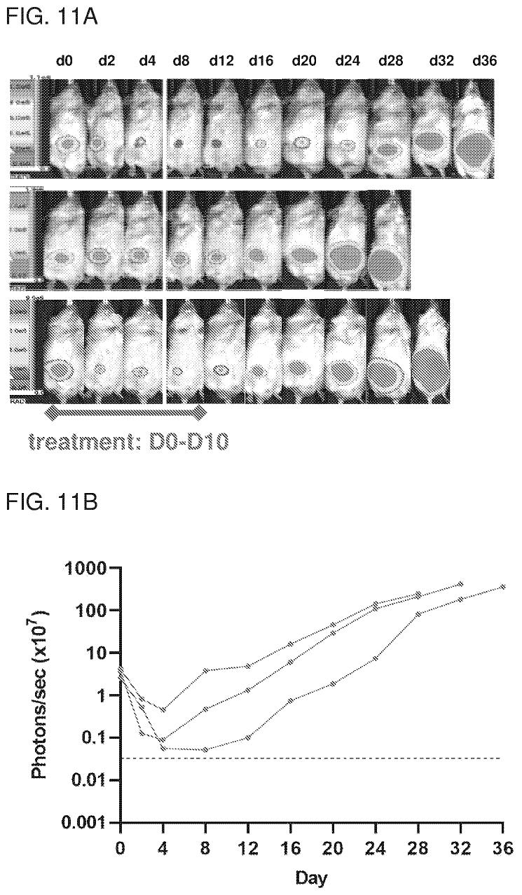

CD47 blocking by anti-CD47 antibody MIAP410 was observed in vivo. 2.5×10⁴ human HIP iPSCs were injected subcutaneously into NSG mice with adoptive transfer of 1×10⁶ human NK cells. A local isotype control (mouse IgG1) was used and treatment was performed during the period of Day 0 to Day 10 (D0-D10). As shown in FIG. 11A-B, human iPSCs formed teratoma in NSG mice with adoptive transferred NK cells. Treatment with IgG1 isotype control did not affect HIP survival.

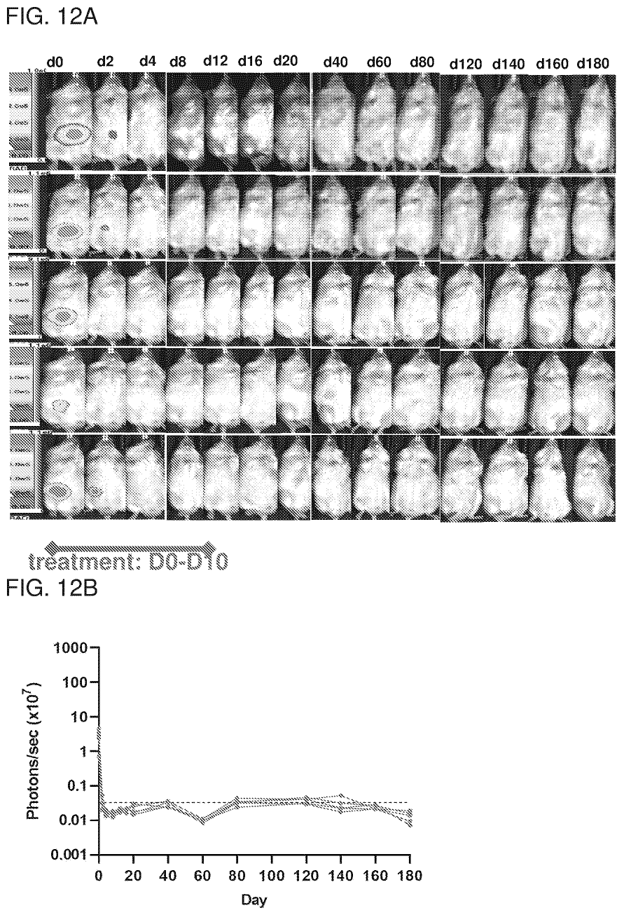

1.5×10⁴ human HIP iPSCs were injected subcutaneously into NSG mice with adoptive transfer of 1×10⁶ human NK cells. IL-2 was administered intraperitoneally (i.p.) to NK cells for activation. Local anti-CD47 low dose (LD) (500 μg) treatment was performed during the period of D0-D10. As shown in FIG. 12A-B, blocking of CD47 in vivo resulted in killing of HIP iPSCs. Anti-CD47 treatment was stopped after D10. iPSCs didn't re-appear when checked during a 6-month follow up.

15.5×10⁴ human HIP iPSCs were injected subcutaneously into NSG mice with adoptive transfer of 1×10⁶ human NK cells (no activation by IL-2). Local anti-CD47 MIAP410 at a high dose (HD; 1 mg) treatment was performed on D0, D1, and D3. As shown in FIG. 13A-B, blocking of CD47 in vivo resulted in killing of HIP iPSCs. Anti-CD47 treatment was given 3 times (on D0, D1, D3). iPSC didn't reappear in any mouse when checked during a 6-month follow up.

16.5×10⁴ human HIP iPSCs were injected subcutaneously into NSG mice with adoptive transfer of 1×10⁶ human NK cells (no activation by IL-2). Anti-CD47 HD (1 mg) treatment was performed on D0, D1, D3 intraperitoneally. As shown in FIG. 14A-B, blocking of CD47 in vivo resulted in killing of HIP iPSCs. Anti-CD47 treatment was given 3 times (on DO, D1, D3) intraperitoneally. iPSC didn't reappear in any mouse when checked during a 6-month follow up.

CD47 blocking by anti-CD47 antibody MIAP410 was observed in vivo in the brain. 5×10⁴ human HIP iPSCs were injected intracranially into NSG mice with adoptive transfer of 1×10⁶ human NK cells. As shown in FIG. 15A-B, human iPSCs formed teratoma in NSG mice with adoptive transferred NK cells. Intracranial treatment with IgG4 isotype control did not affect HIP survival. A local IgG4 isotype control HD (1 mg) treatment was performed on D0, D1, and D3.

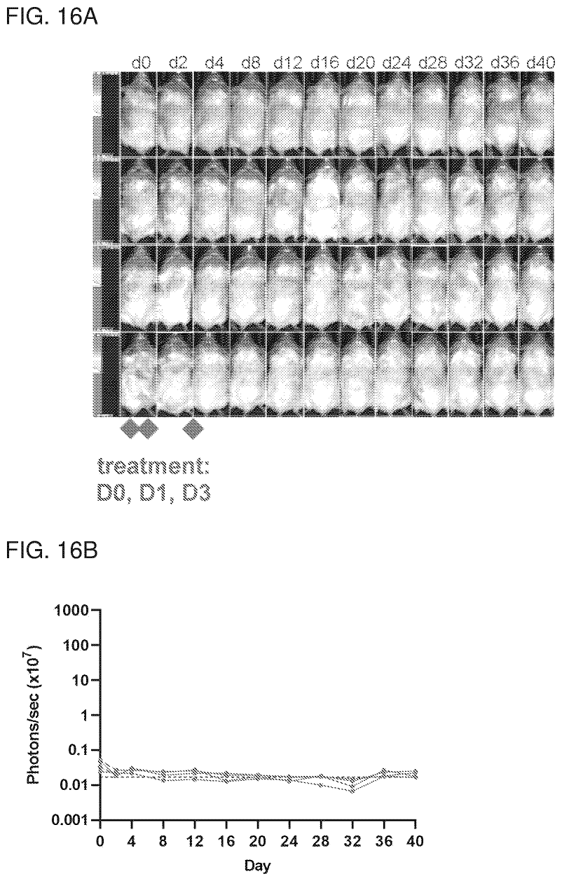

5×10⁴ human HIP iPSCs were injected intracranially into NSG mice with adoptive transfer of 1×10⁶ human NK cells. Local anti-CD47 HD (1 mg) treatment was performed on D0, D1, D3. As shown in FIG. 16A-B, intracranial blocking of CD47 resulted in killing of HIP iPSCs in the brain. Anti-CD47 treatment was stopped after three times. iPSCs didn't re-appear when mice were checked during a 40-day follow up.

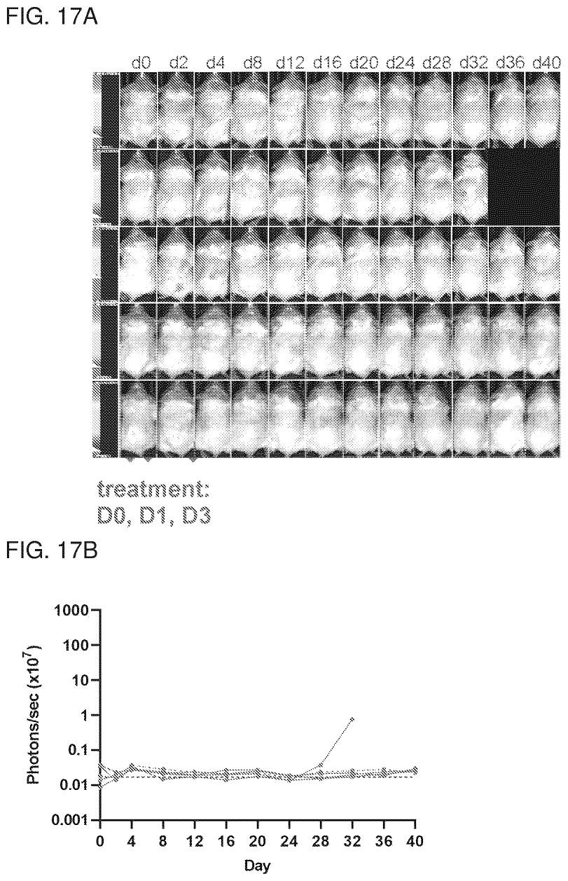

5×10⁴ human HIP iPSCs were injected intracranially into NSG mice with adoptive transfer of 1×10⁶ human NK cells. Anti-CD47 HD (1 mg) treatment was performed intraperitoneally on D0, D1, and D3, with the blood-brain barrier broken by mannitol injections. As shown in FIG. 17A-B, intracranial blocking of CD47 resulted in killing of HIP iPSCs in the brain. Anti-CD47 treatment (i.p.) was stopped after three times. iPSCs didn't re-appear when checked during a 40-day follow up in 4 out of 5 mice.

D. Human iPSCs: SIRPα IgG1Fc or IgG4Fc (Fusion Proteins) In Vitro and In Vivo

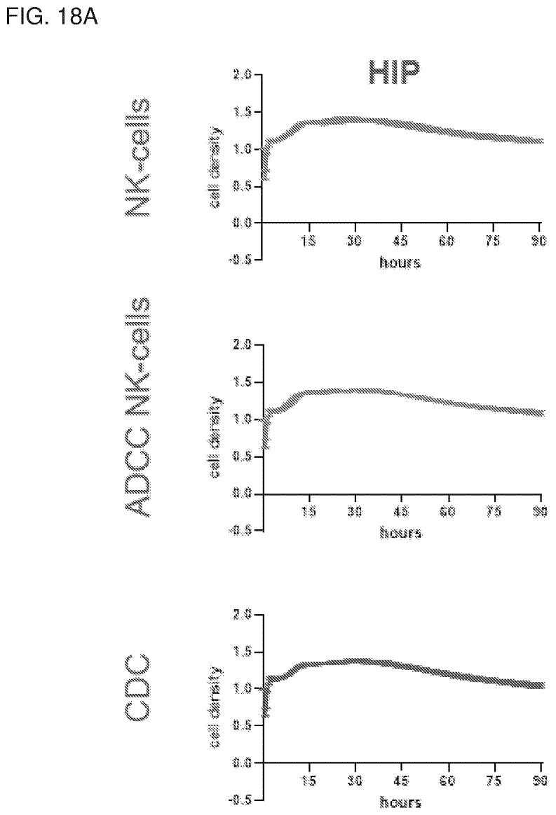

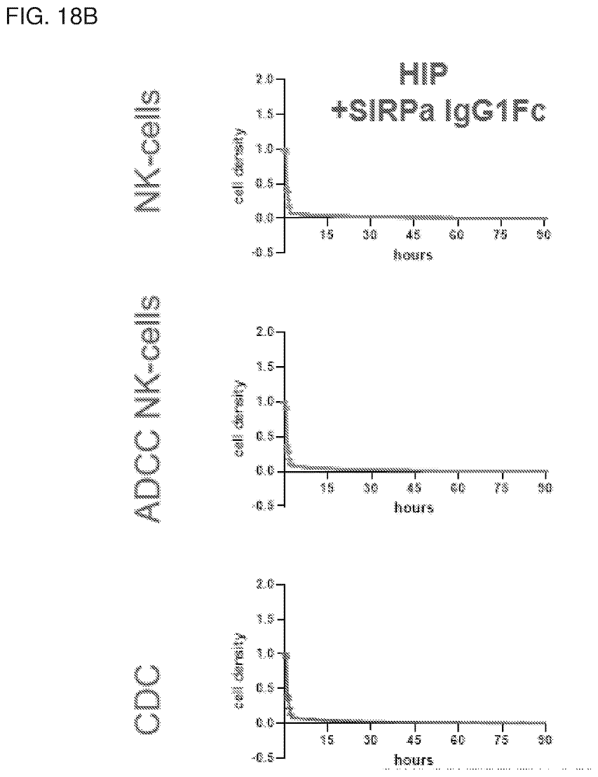

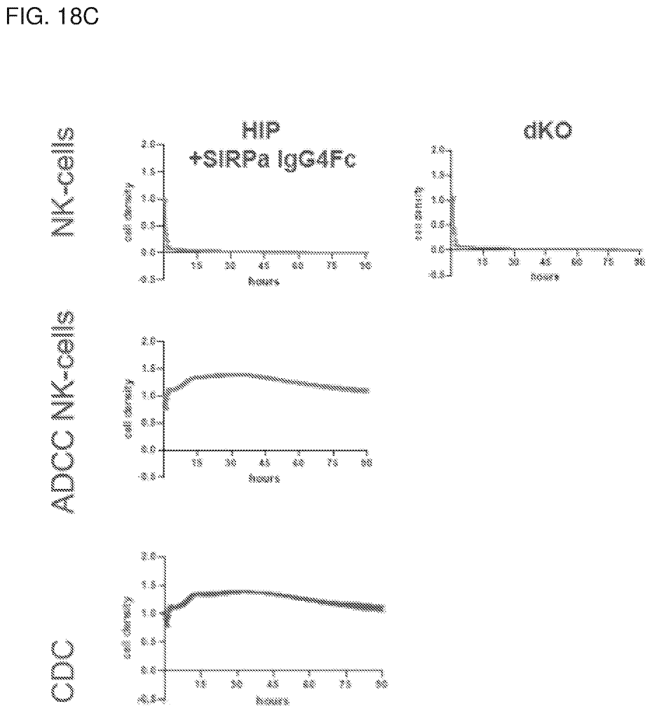

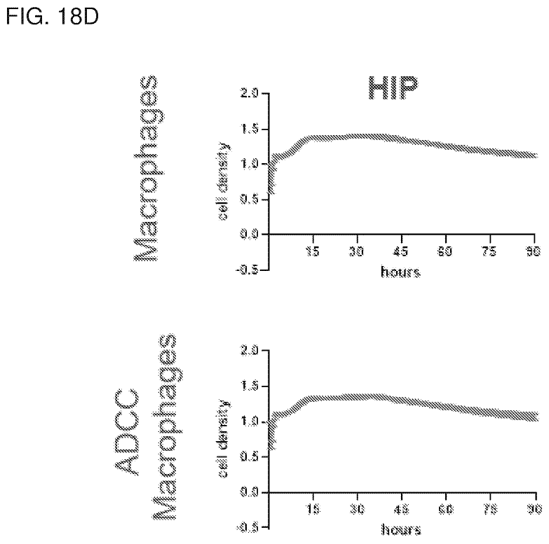

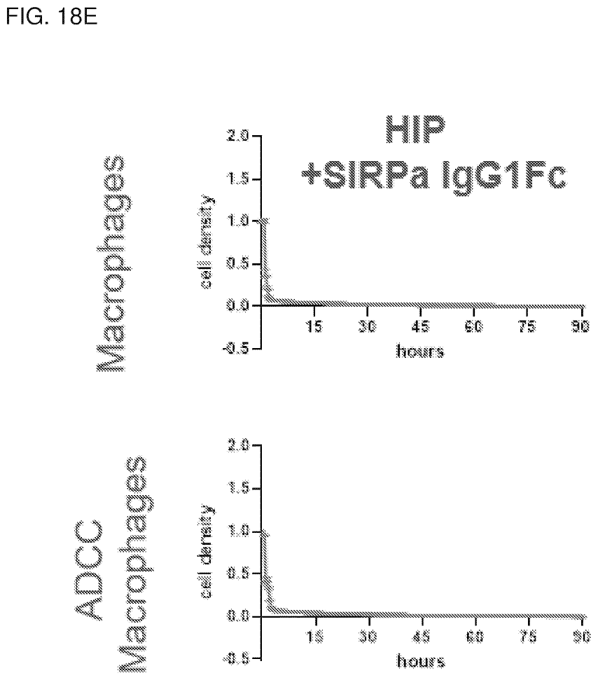

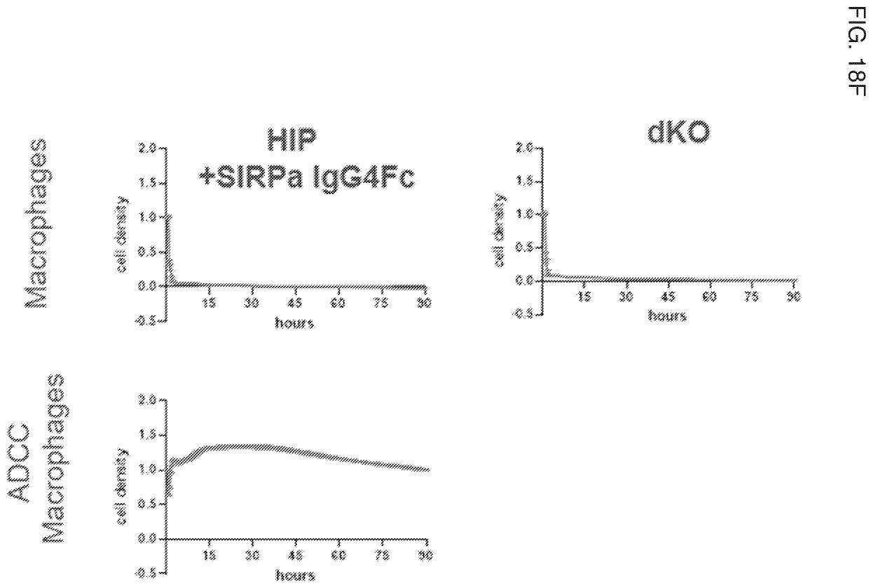

SIRPαFc fusion proteins were studied in vitro for their effects on human iPSCs. Effects on HIP CD19CAR-T cells were studied with respect to killing mediated by NK cells, ADCC NK cells, and CDC (FIG. 18A). Effects on HIP cells with SIRPα IgG1Fc were studied with respect to killing mediated by NK cells, ADCC NK cells, and CDC (FIG. 18B). Effects on HIP cells with SIRPα IgG4Fc (including a dKO killing control) were studied with respect to killing mediated by NK cells, ADCC NK cells, and CDC (FIG. 18C). Effects on HIP cells were studied with respect to killing mediated by macrophages and ADCC macrophages (FIG. 18D). Effects on HIP cells with SIRPα IgG1Fc were studied with respect to killing mediated by macrophages and ADCC macrophages (FIG. 18E). Effects on HIP cells with SIRPα IgG4Fc (including a dKO killing control) were studied with respect to killing mediated by macrophages and ADCC macrophages (FIG. 18F). As expected, IgG4 was observed to mediate killing by blocking of CD47. Furthermore, IgG1 was observed to additionally induce CDC and ADCC.

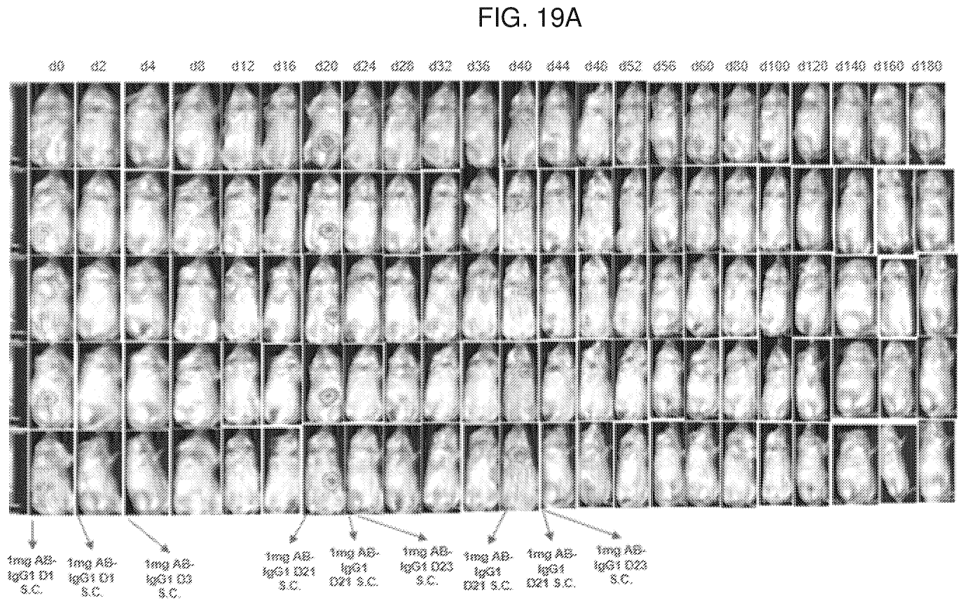

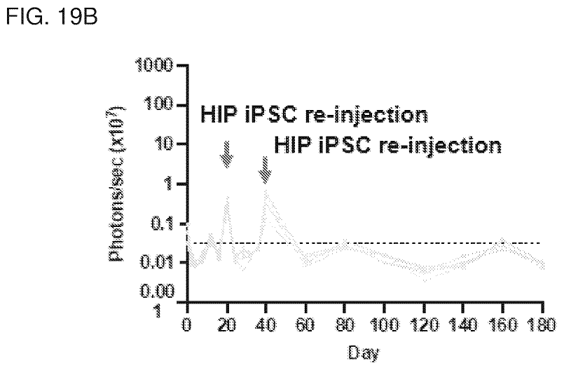

CD47 blocking and ADCC by SIRPα IgG1Fc was observed in vivo. Human HIP iPSCs were injected subcutaneously into NSG mice with adoptive transfer of human NK cells. Local SIRPα IgG1Fc (1 mg) treatment was performed on D0, D1, D3. Re-injection with human HIP iPSC was performed on D20 and D40, followed with SIRPα IgG1Fc injections (for 3 days). As shown in FIG. 19A-B, treatment with SIRPα IgG1Fc resulted in killing of HIP iPSCs repeatedly in all mice. Follow-up was performed for 6 months.

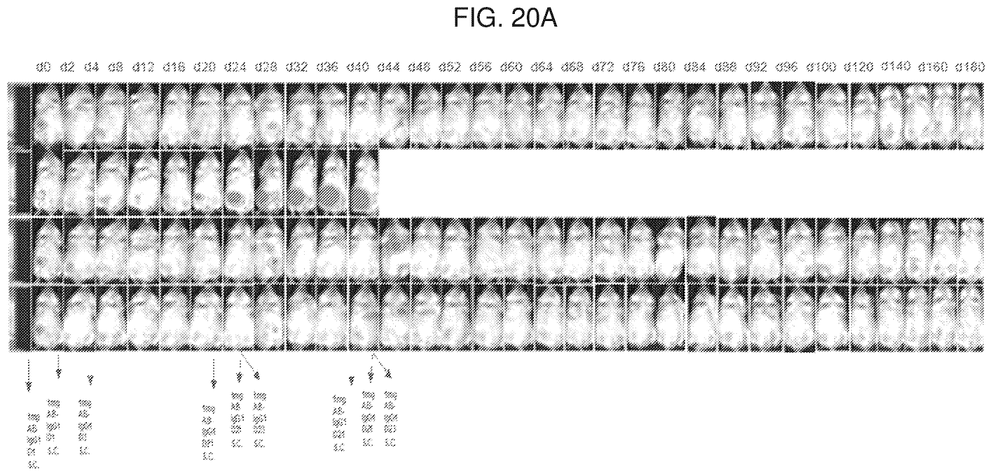

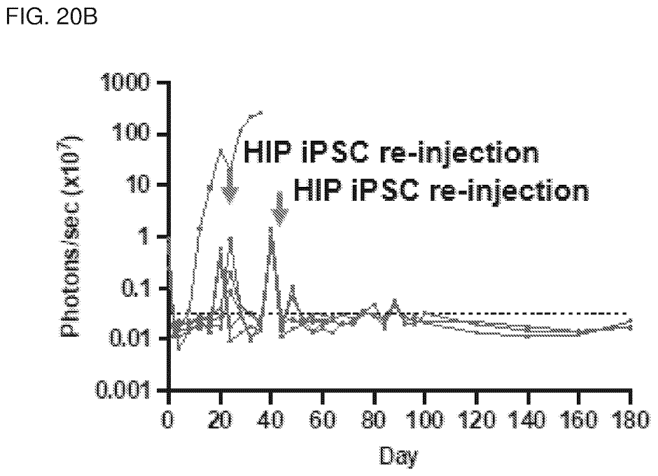

CD47 blocking by SIRPα IgG4Fc was observed in vivo. Human HIP iPSCs were injected subcutaneously into NSG mice with adoptive transfer of human NK cells. Local SIRPα IgG4Fc (1 mg) treatment was performed on D0, D1, D3. Re-injection with human HIP iPSC was performed on D20 and D40, followed with SIRPα IgG4Fc injections (for 3 days) (FIG. 20A-B).

CD47 blocking and ADCC by SIRPα IgG1Fc was observed in vivo. Human HIP iPSCs were injected intracranially into NSG mice with adoptive transfer of human NK cells and human microglia. Local IgG1 isotype control (1 mg) treatment was performed on D0, D1, D3. As shown in FIG. 21A-B, human HIP iPSCs formed teratoma in NSG mice with adoptive transferred NK cells and microglia. Treatment with IgG1 isotype control did not affect HIP survival.

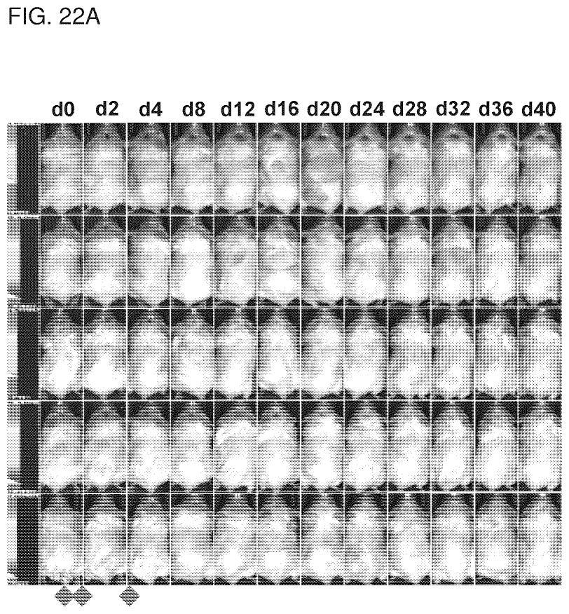

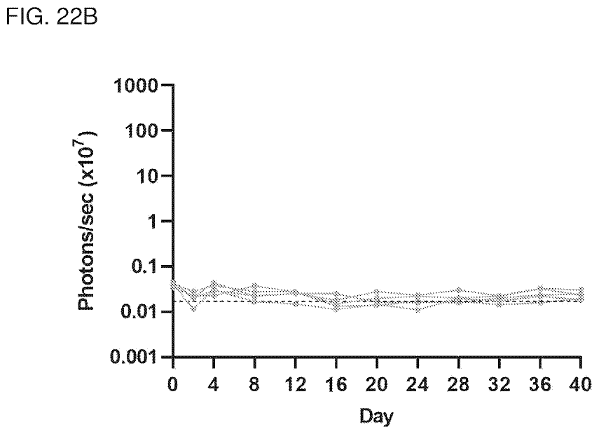

CD47 blocking and ADCC by SIRPα IgG1Fc was observed in vivo. Human HIP iPSCs were injected intracranially into NSG mice with adoptive transfer of human NK cells and microglia. Local SIRPα IgG1Fc (1 mg) treatment was performed on D0, D1, D3. As shown in FIG. 22A-B, intracranial SIRPα IgG1 Fc resulted in killing of HIP iPSCs in the brain.

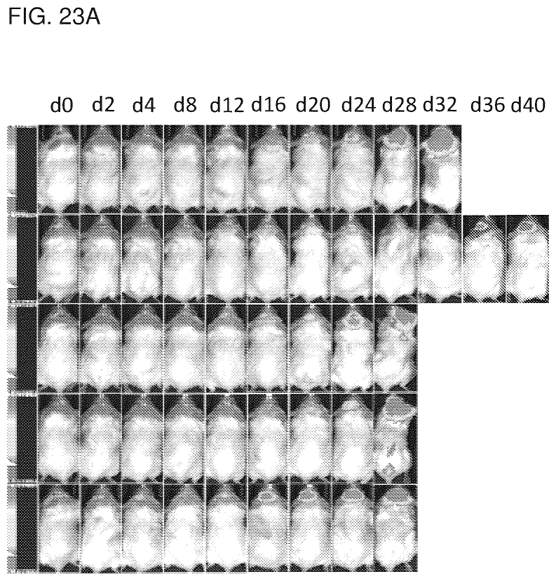

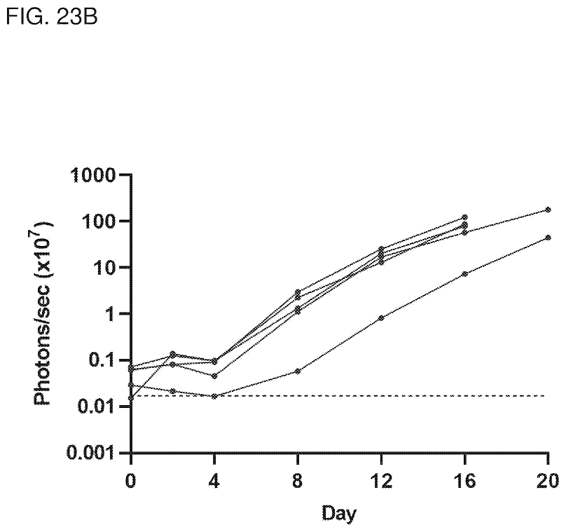

CD47 blocking and ADCC by SIRPα IgG1Fc was observed in vivo. Human HIP iPSCs were injected intracranially into NSG mice with adoptive transfer of human NK cells and human microglia. IgG1 Isotype control (1 mg) treatment was performed on D0, D1, D3 intraperitoneally. As shown in FIG. 23A-B, human HIP iPSCs formed teratoma in NSG mice with adoptive transferred NK cells and microglia. Treatment with IgG1 isotype control intraperitoneally did not affect HIP survival.

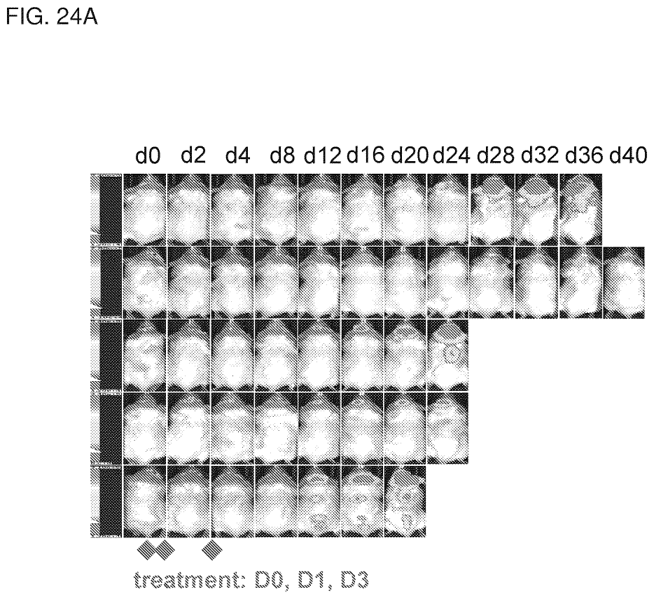

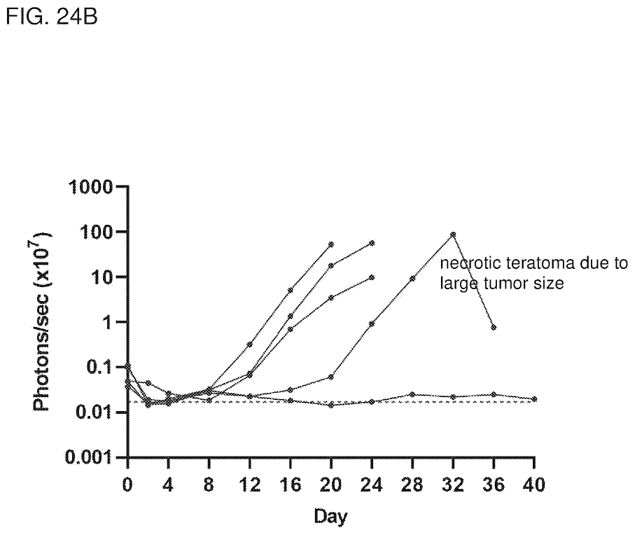

CD47 blocking and ADCC by SIRPα IgG1Fc was observed in vivo. Human HIP iPSCs were injected intracranially into NSG mice with adoptive transfer of human NK cells and microglia. SIRPα IgG1Fc (1 mg) treatment was performed on D0, D1, D3 and administered intraperitoneally with the blood brain barrier broken by mannitol. As shown in FIG. 24A-B, systemic SIRPα IgG1Fc induced killing of HIP iPSCs in the brain in 1 out of 5 mice. Intraperitoneal application appeared to be less efficient compared to local administration.

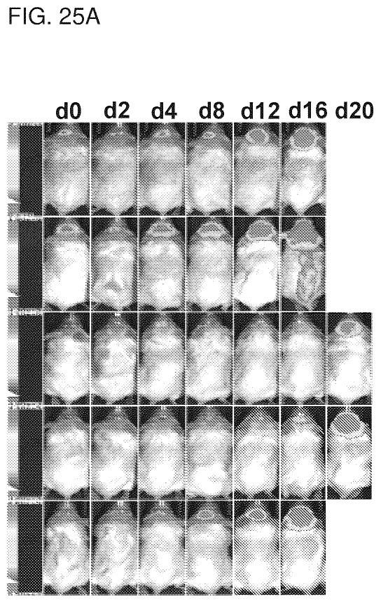

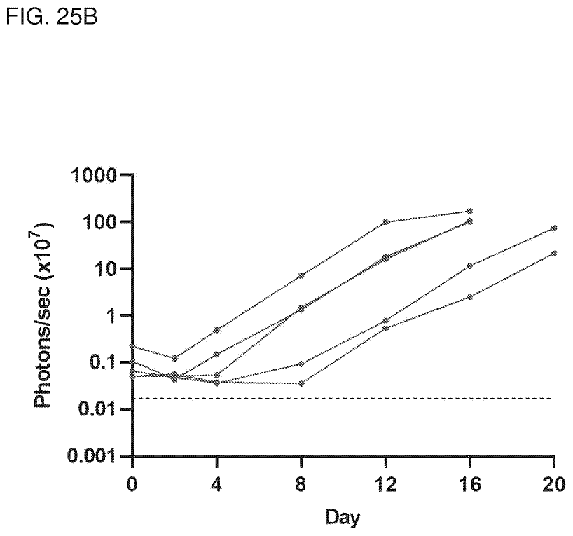

CD47 blocking by SIRPα IgG4Fc was observed in vivo. Human HIP iPSCs were injected intracranially into NSG mice with adoptive transfer of human NK cells and human microglia. Local IgG4 isotype control (1 mg) treatment was performed on D0, D1, D3. As shown in FIG. 25A-B, human HIP iPSCs formed teratoma in NSG mice with adoptive transferred NK cells and microglia. Treatment with IgG4 isotype control did not affect HIP survival. Mice were euthanized due to teratoma size/symptoms at D16 and D20.

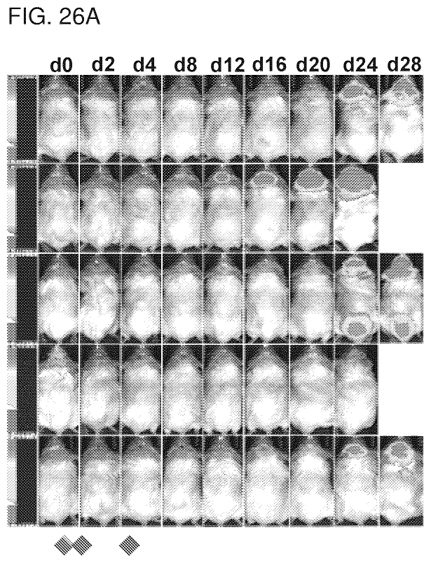

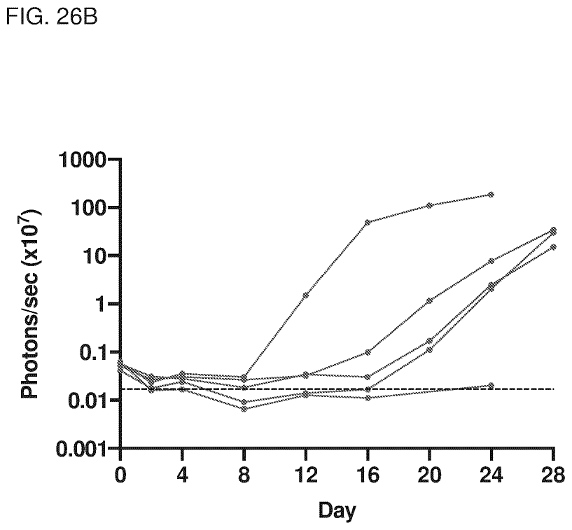

CD47 blocking by SIRPα IgG4Fc was observed in vivo. Human HIP iPSCs were injected intracranially into NSG mice with adoptive transfer of human NK cells and microglia. Local SIRPα IgG4Fc (1 mg) treatment was performed on D0, D1, D3. As shown in FIG. 26A-B, intracranial SIRPα IgG4Fc treatment resulted in killing of HIP iPSCs in the brain in 1 out of 5 mice. IgG1Fc appeared to be more potent.

Human HIP iPSCs were subcutaneously injected into NSG mice along with adoptive transfer with human NK cells and anti-SIRPα subcutaneously mixed in at 1 mg on DO, D1, and D3. Reinjection with human HIP iPSCs was performed on D20 50,000 cells (50k) subcutaneously (into the left side) along with 1 mg B6H12 on D20 (mixed in), D21, and D23. Reinjection with human HIP iPSCs was performed on D40 50k subcutaneously (into upper middle chest) along with 1 mg B6H12 on D40 (mixed in), D41, and D43 (FIG. 27).

### E. Human CD19 Car T: SIRPα IgG1Fc or IgG4Fc (Fusion Proteins) In Vitro and In Vivo

The functional endpoint was: (a) T cell killing by NK cells or macrophages; and (b) Induction of HIP T cell killing by anti-CD47 antibodies (magrolimab/IgG1 fusion protein/IgG4 fusion protein), which confirmed relevance of CD47 to protect from NK cell/macrophage killing. In vitro read-out was performed with Xcelligence.

TABLE 22
Cell types and groups

| | |
|---|---|
| 1 | HIP-CAR |
| 2 | EGFRt CAR |
| 3 | EGFRt CAR + tKO* |
| 4 | Mock transfected T cells (i.e., T cells that were mock |
| | transfected) |

* confirmed relevance of CD47 to protect from NK cell/macrophage killing

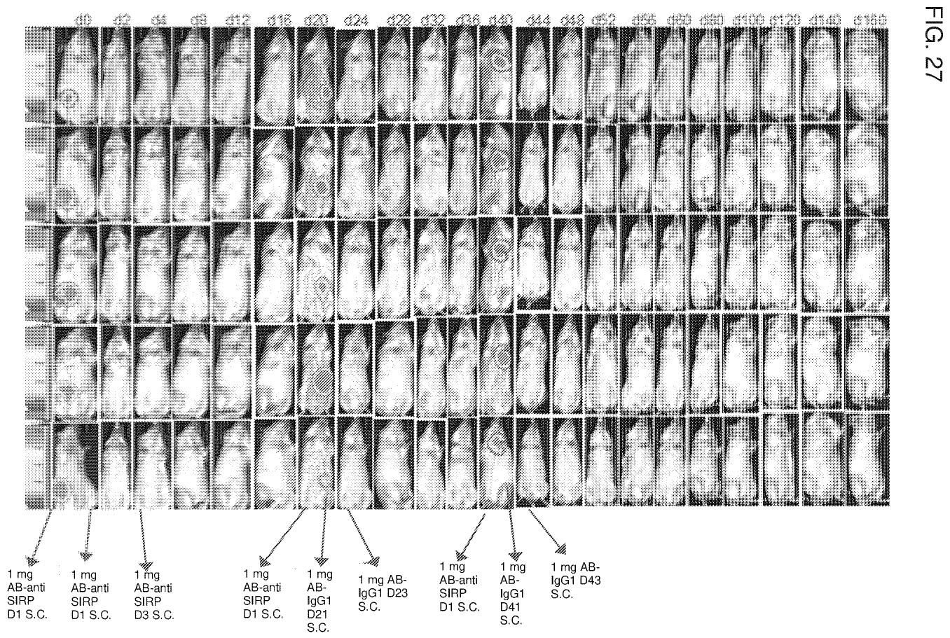

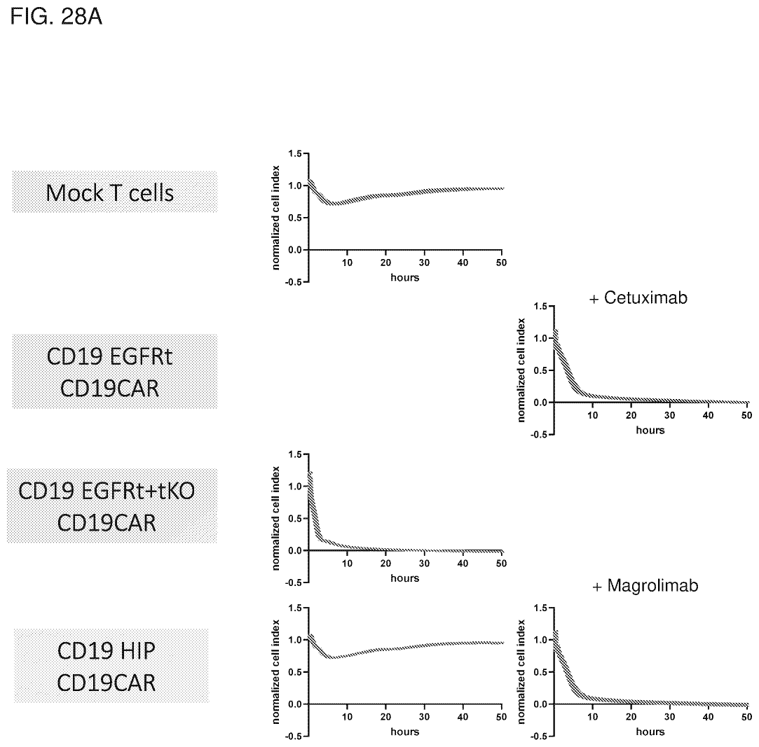

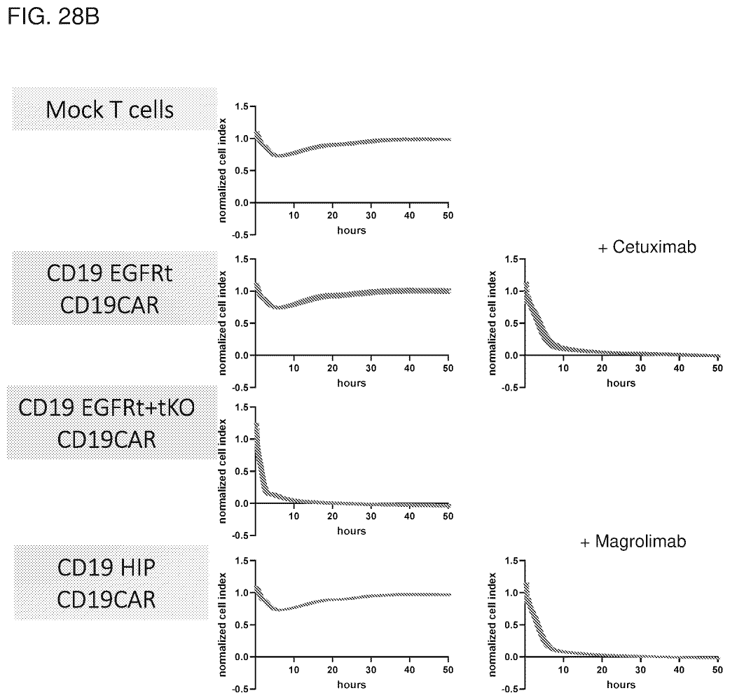

CD47 blocking by SIRPα IgG1Fc or SIRPα IgG4Fc was observed in vitro. Effects were studied on NK cells (FIG. 28A), macrophages (FIG. 28B), CD19 HIP CAR and NK cells (FIG. 29A), and CD19 HIP CAR and macrophages (FIG. 29B).

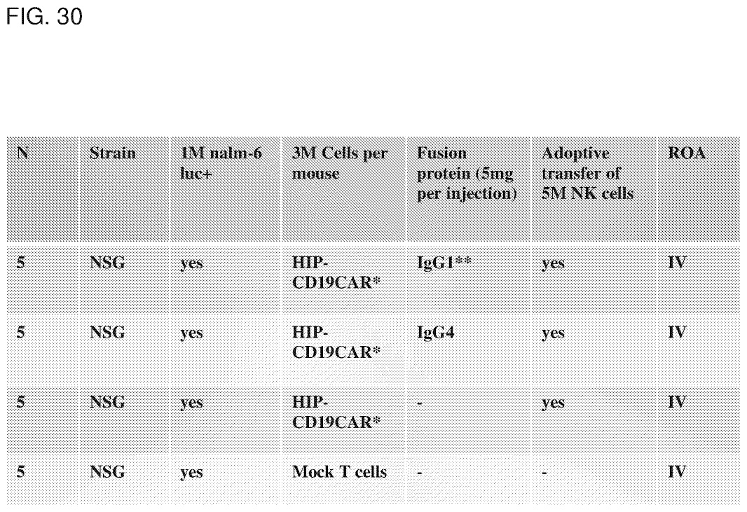

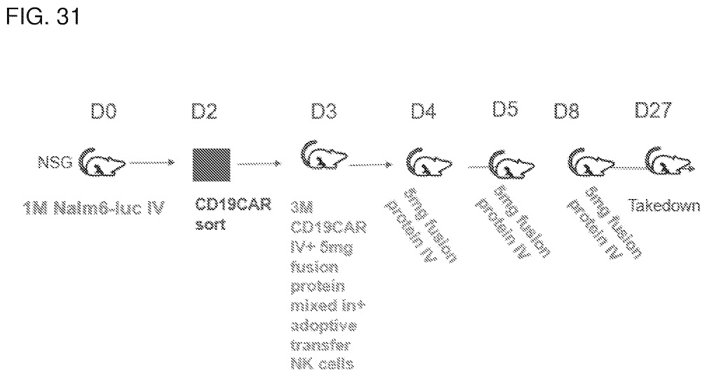

NSG mice were studied using a Nalm6 tumor model. Adoptive transfer of human NK cells and human HIP CAR-T cells was performed intravenously with and without fusion protein intravenously. 100U/ml IL-2 was thawed overnight before sorting, followed by 100U/ml IL-2 overnight after sorting and before injection (FIG. 30). When HIP CARs were eliminated by a safety strategy, Nalm-6 tumor grew (FIG. 31).

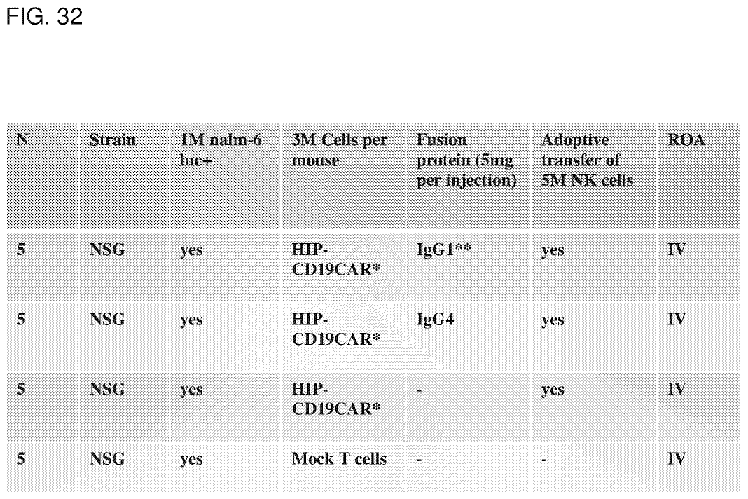

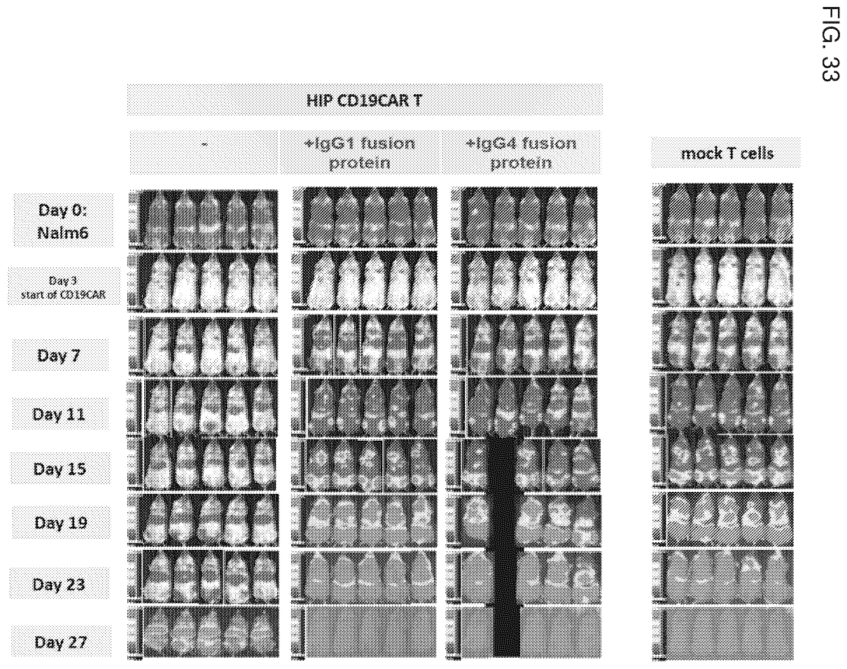

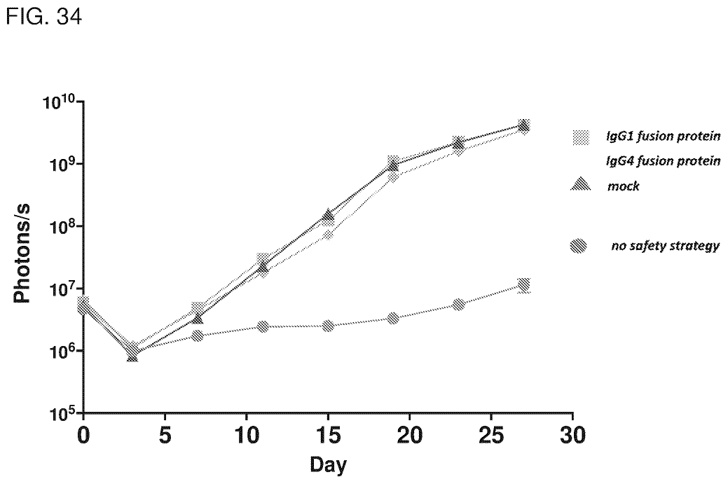

As shown in FIG. 32, (a) mice treated with mock transfected T cells, namely T cells that were mock transfected, developed tumors; (b) mice treated with HIP CAR T cells showed tumor clearance; and (c) IgG1 and IgG4 fusion protein treatment resulted in killing of HIP CAR T cells due to CD47 blocking and therefore tumor growth appeared to be comparable to mock transfected T cell group. HIP CAR T cells were eliminated by IgG1 and IgG4 anti-CD47 fusion proteins, indicating that Nalm-6 tumor was growing (FIGS. 33 and 34).

### F. Mouse Primary HIP Islets: MIAP410 In Vitro and In Vivo

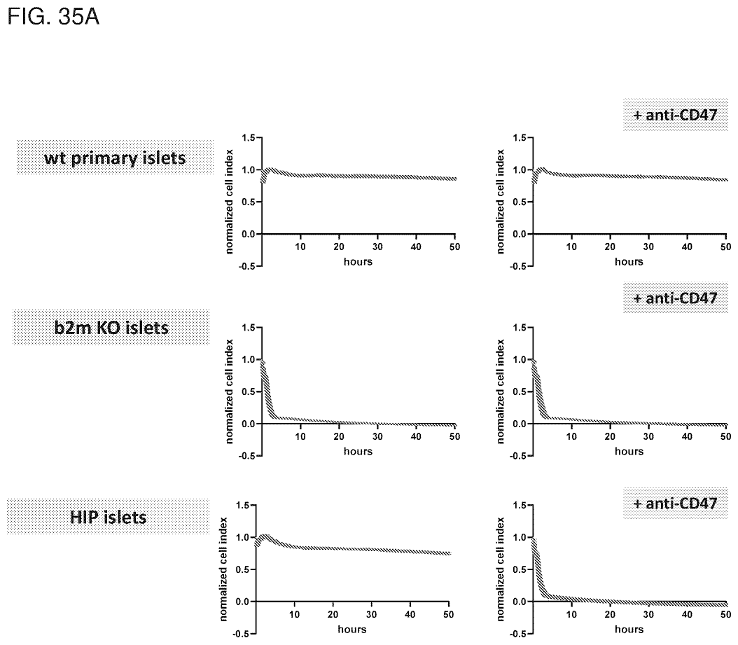

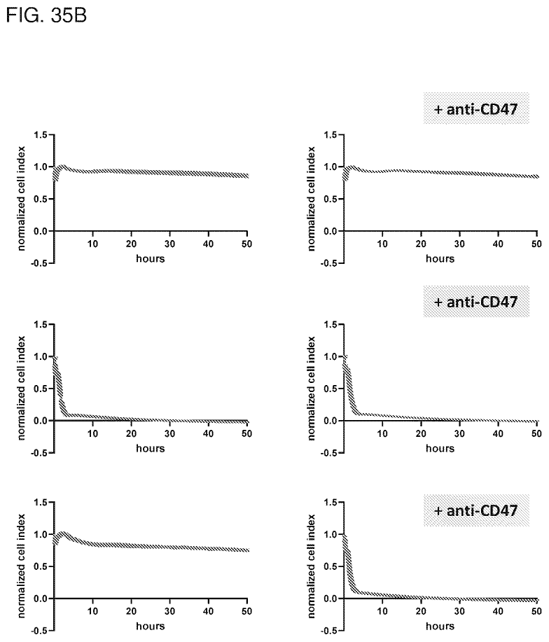

Blocking by anti-CD47 antibody MIAP410 was observed in vitro in mouse HIP primary islets. As shown in FIG. 35A, NK cell killing resulted from MIAP410. As shown in FIG. 35B, macrophage killing was observed.

Blocking by MIAP410 was observed in vivo in mouse HIP primary islets. 1 mouse corresponded to ˜150 islet clusters. 1 islet corresponded to ˜1500 cells. During transplantation 300 clusters were used per mouse (18 g-20 g), i.e., about 450,000 cells (450k) intramuscularly (i.m.).

TABLE 23
HIP islet cell study

| Group | Cell line | N | Strain | Cell dose | ROA | Safety |
|---|---|---|---|---|---|---|
| 1 | HIP* | 5 | BALB/c | 300 | i.m. | MIAP410 5 mg; D 7, 8, 9, 10, |
| | islets luc+ | | | clusters | | until cells are dead |
| 2 | HIP* | 5 | BALB/c | 300 | i.m. | IgG1 Isotype control |
| | islets luc+ | | | clusters | | |

*HIP islet from B2M−/− mice; CD47 delivered by lentivirus

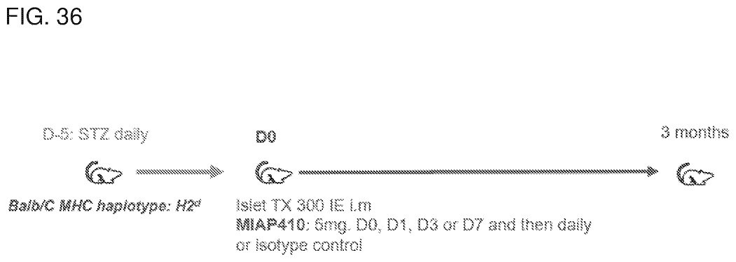

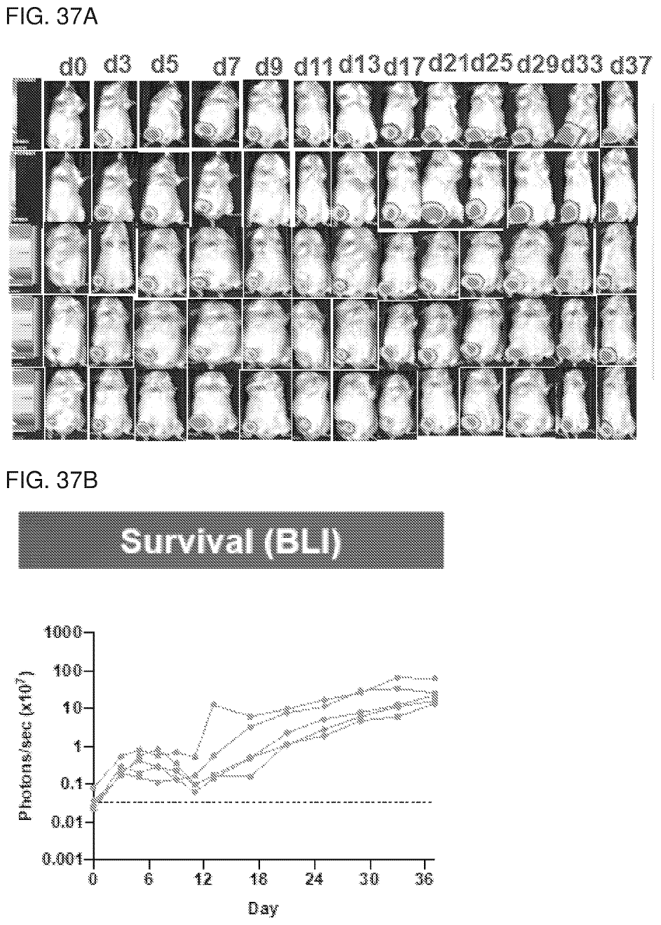

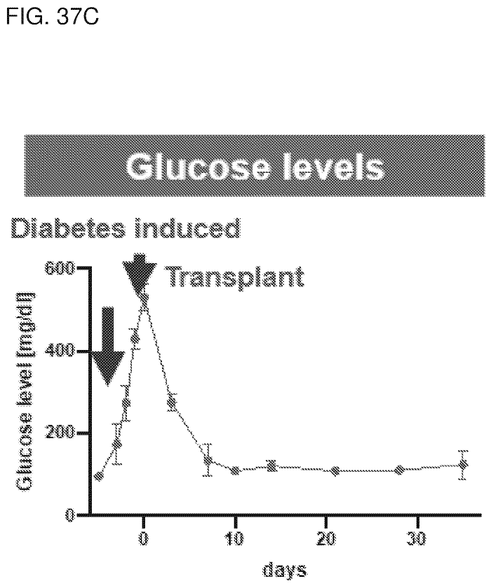

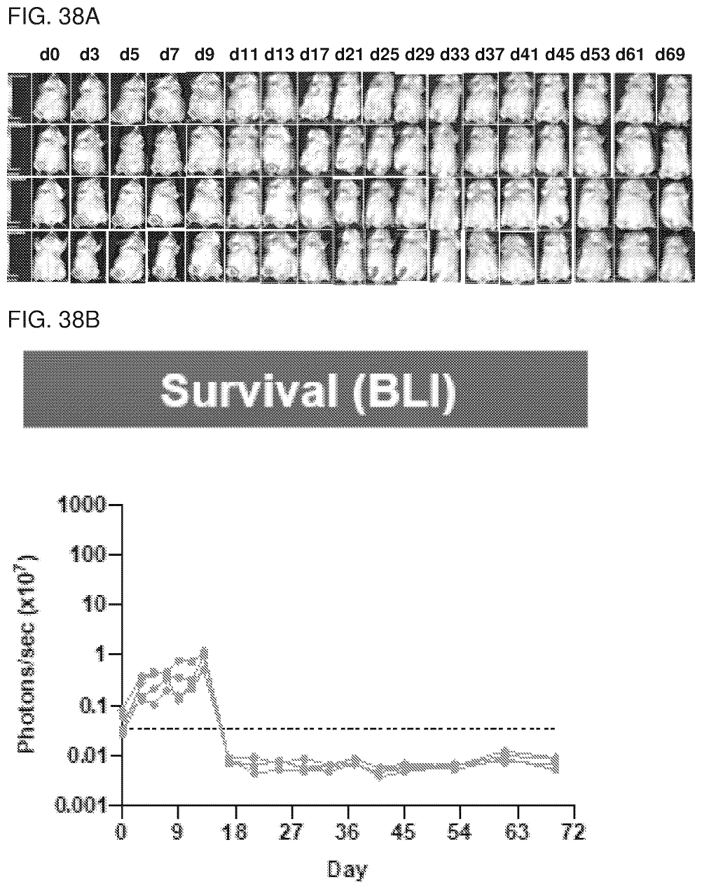

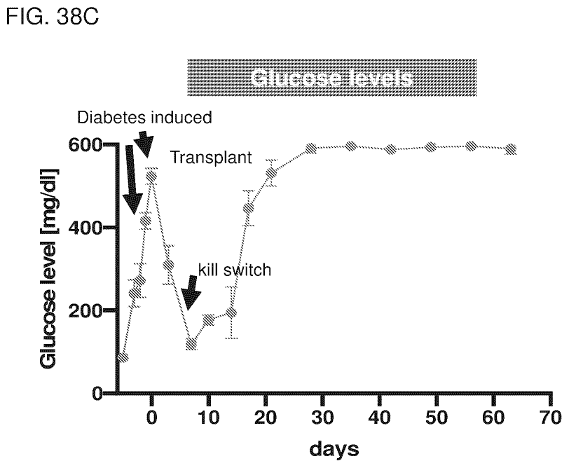

Diabetes was induced in mouse cells using STZ. After 6 days, allogeneic HIP islet cells (MHC haplotype: H2^{b}; MOI 20 for luc and CD47 (CAG promoter)) were injected/transplanted into the mice. (FIG. 36). A safety strategy study was performed using primary islet transplantation in allogeneic Balb/C mice. As shown in FIG. 37A-C, allogeneic HIP islets survived and cured diabetes in allogeneic mice. No killing of HIP islets was observed when 5 mg intramuscular IgG1 isotype control was used. As shown in FIG. 38A-C, HIP islets survived in allogeneic mice and cured diabetes when 5 mg of MIAP410 was injected intramuscularly on D7-D18. When anti-CD47 was used (“safety strategy”), HIP islets were killed and mice became diabetic again.

### Example 5. Administration of MIAP410 Anti-CD47 Antibody with Mouse IgG1 Fc Domain Blocks CD47 in Human Cells In Vivo with Adoptive Transfer of Human NK Cells

Protocols were followed as in Example 4.

### A. No IL-2 Dependence

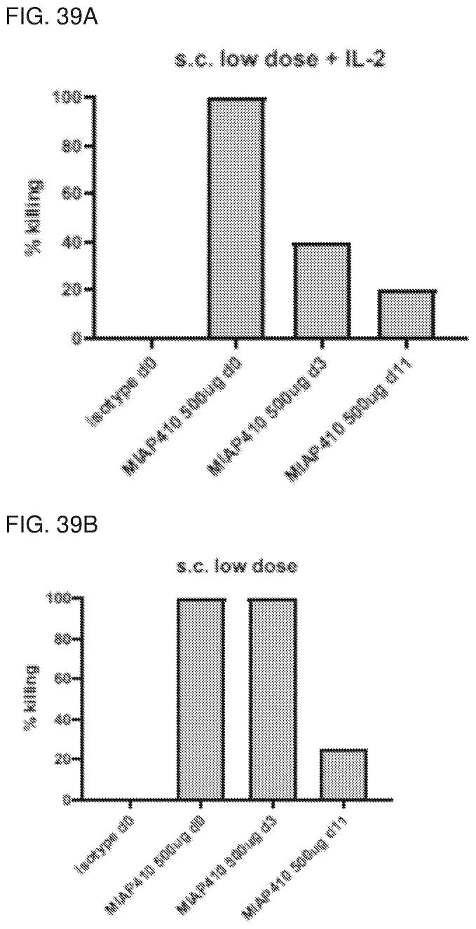

As shown in FIG. 39A-B, when human HIP iPSCs were injected into NSG mice with adoptive transfer of human NK cells and human macrophages, administration of MIAP410 with Fc isotype IgG1 resulted in time-dependent killing by innate cells, with or without in vivo IL-2 stimulation, possibly through activation of NSG macrophages.

B. Variation of Results Depending on when Treatment Begins

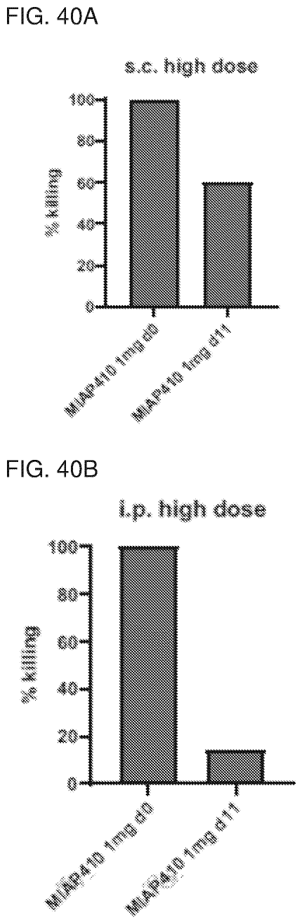

As shown in FIG. 40A-B, a local subcutaneous higher dose (HD; three times) of MIAP410 beginning on Day 0 (DO) appeared to be more effective than beginning treatment on Day 11. An intraperitoneal dose was as effective as a local dose on DO, but not on D11.

### C. Local Treatment in the Brain

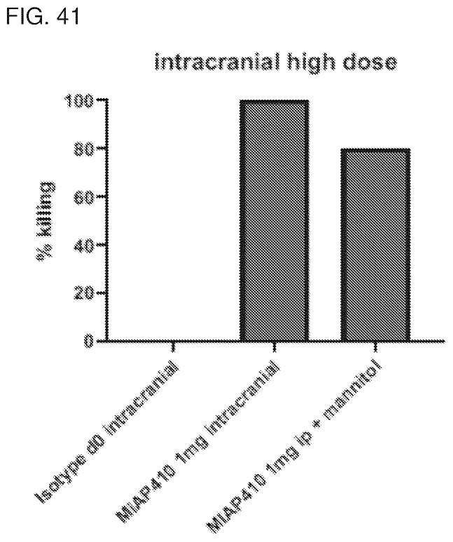

As shown in FIG. 41, local treatment in the brain was as effective as local subcutaneous treatment. Intraperitoneal treatment appeared to be effective when the blood-brain barrier was broken, allowing the MIAP410 easier access.

D. Blocking of CD47 In Vivo with Concomitant Administration of IL-2 Results in Killing of HIP iPSCs

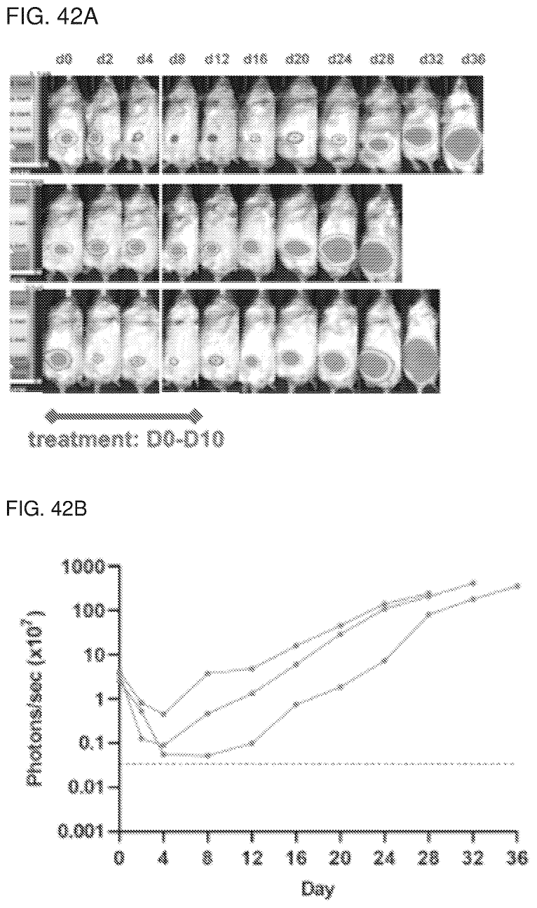

As shown in FIG. 42A-B, human HIP iPSCs formed teratoma in NSG mice with adoptively transferred NK cells. When human HIP iPSCs were injected subcutaneously into NSG mice with adoptive transfer of human NK cells, administration of Fc isotype IgG1 control did not affect HIP survival.

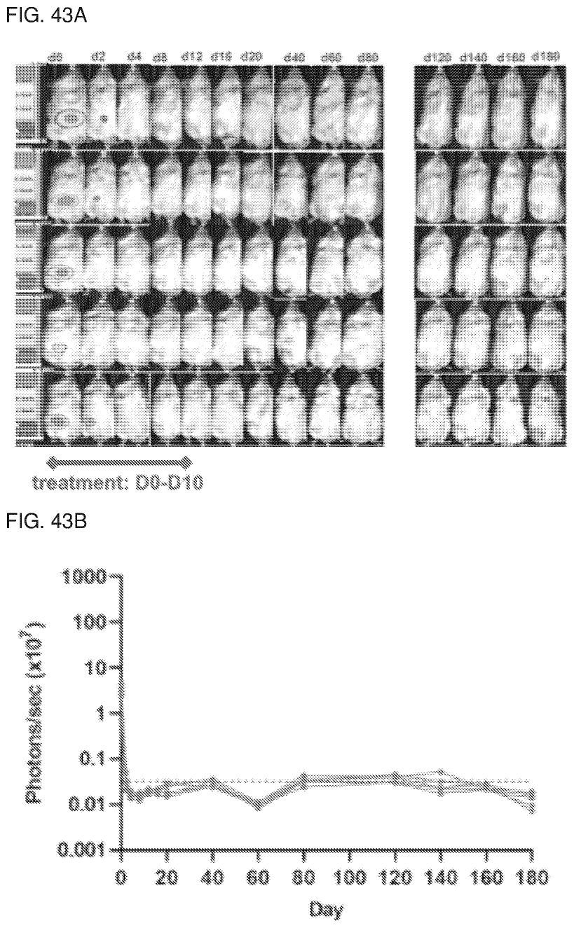

As shown in FIG. 43A-B, when human HIP iPSCs were injected subcutaneously into NSG mice with adoptive transfer of human NK cells, local LD 500 μg administration of MIAP410 with Fc isotype IgG1 concurrently with administration of IL-2 to NK cells for activation resulted in killing of HIP iPSCs. MIAP410 antibody treatment occurred during the period of D0-D10 and was stopped after Day 10 (D10). It was observed that iPSCs did not re-appear when follow-up was performed 6 months after treatment.

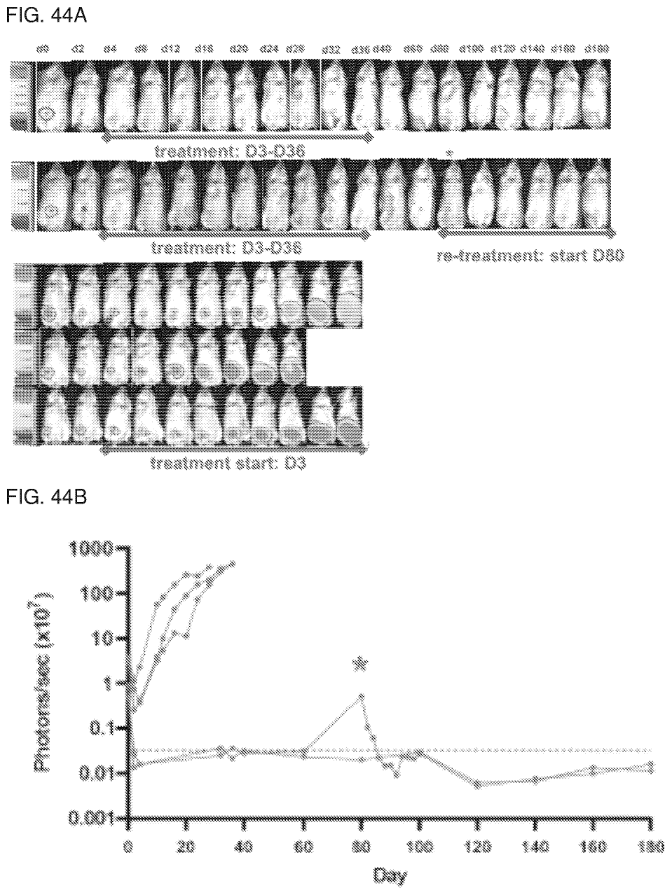

As shown in FIG. 44A-B, human HIP iPSCs were injected subcutaneously into NSG mice with adoptive transfer of human NK cells, and MIAP410 with Fc isotype IgG1 was administered locally (LD 500 μg) with concurrent administration of IL-2 to NK cells for activation. MIAP410 antibody treatment began on Day 3 (D3) and occurred during the period of D3-D36. For a subset of mice, re-treatment began on D80. When follow-up was performed 6 months after beginning of treatment, it was observed that iPSCs were eliminated in 1 mouse, while teratoma developed in 4 mouse, with re-treatment eliminating iPSCs in 1 mouse

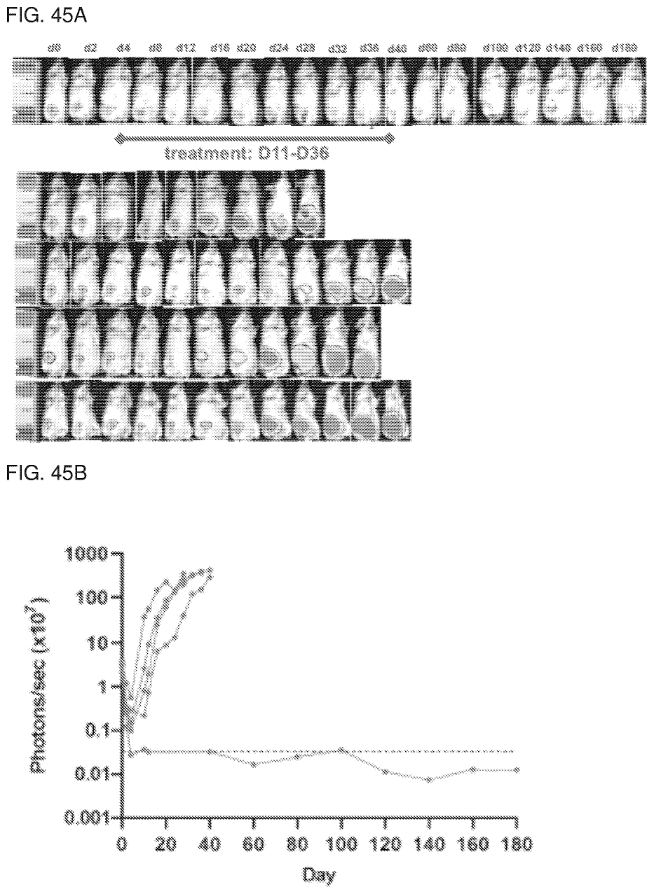

As shown in FIG. 45A-B, human HIP iPSCs were injected subcutaneously into NSG mice with adoptive transfer of human NK cells, and MIAP410 with Fc isotype IgG1 was administered locally (LD 500 μg) with concurrent administration of IL-2 to NK cells for activation. MIAP410 antibody treatment began on Day 11 (D11) and occurred during the period of D11-D36. When follow-up was performed 6 months after beginning of treatment, it was observed that iPSCs were eliminated in 1 mouse, while teratoma developed in 4 mice.

E. Blocking of CD47 In Vivo without Concomitant Administration of IL-2 Results in Killing of HIP iPSCs

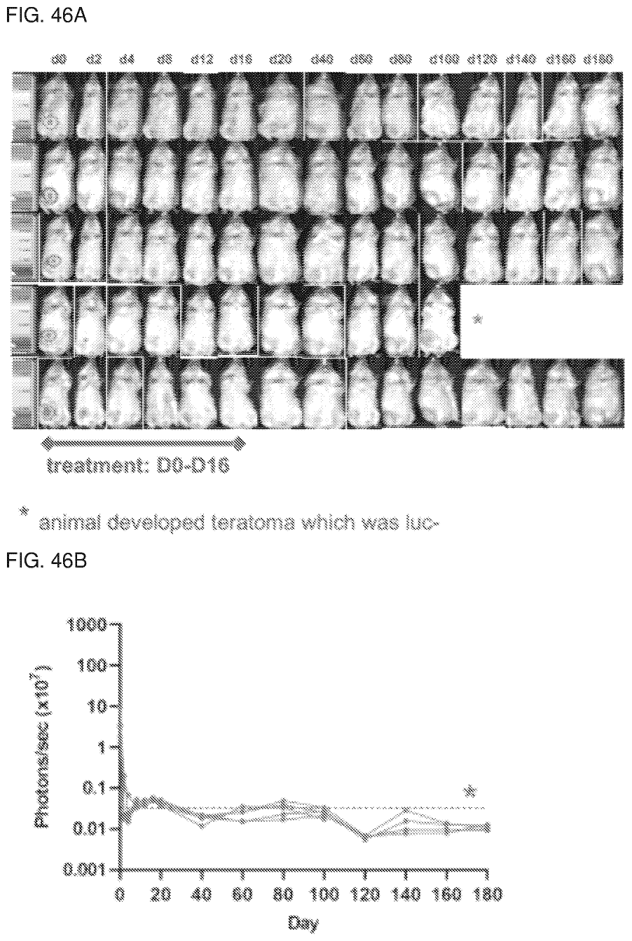

As shown in FIG. 46A-B, when human HIP iPSCs were injected subcutaneously into NSG mice with adoptive transfer of human NK cells, local LD 500 μg administration of MIAP410 with Fc isotype IgG1 resulted in killing of HIP iPSCs. MIAP410 antibody treatment began on Day 0 (DO) and occurred during the period of D0-D10, with treatment stopped after D16. When follow-up was performed 6 months after beginning of treatment, it was observed that iPSCs did not reappear in 4 mice, while teratoma developed in 1 mouse, which was luciferase-.

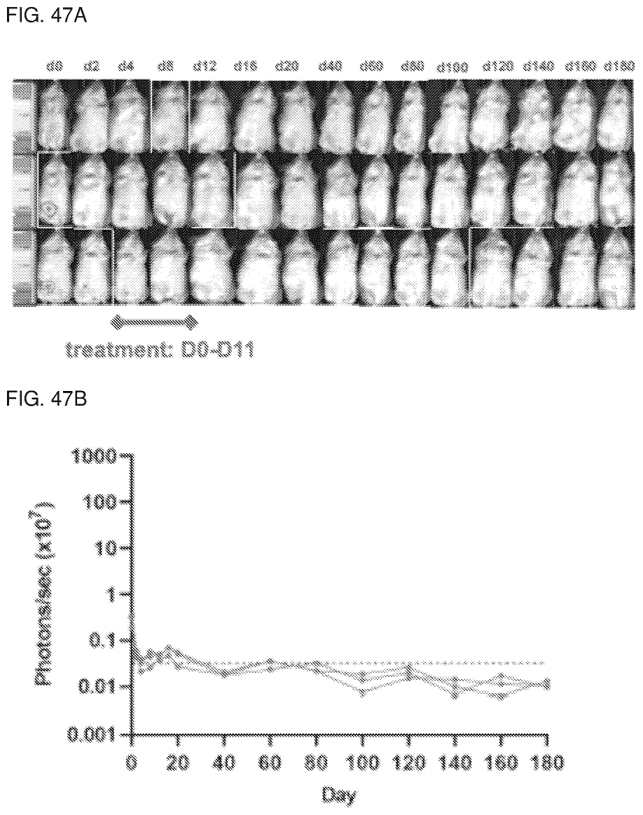

As shown in FIG. 47A-B, when human HIP iPSCs were injected subcutaneously into NSG mice with adoptive transfer of human NK cells, local LD 500 μg administration of MIAP410 with Fc isotype IgG1 resulted in killing of HIP iPSCs. MIAP410 antibody treatment began on Day 3 (D3) and occurred during the period of D3-D11, with treatment stopped after D11. When follow-up was performed 6 months after beginning of treatment, it was observed that iPSCs did not reappear in any of the mice.

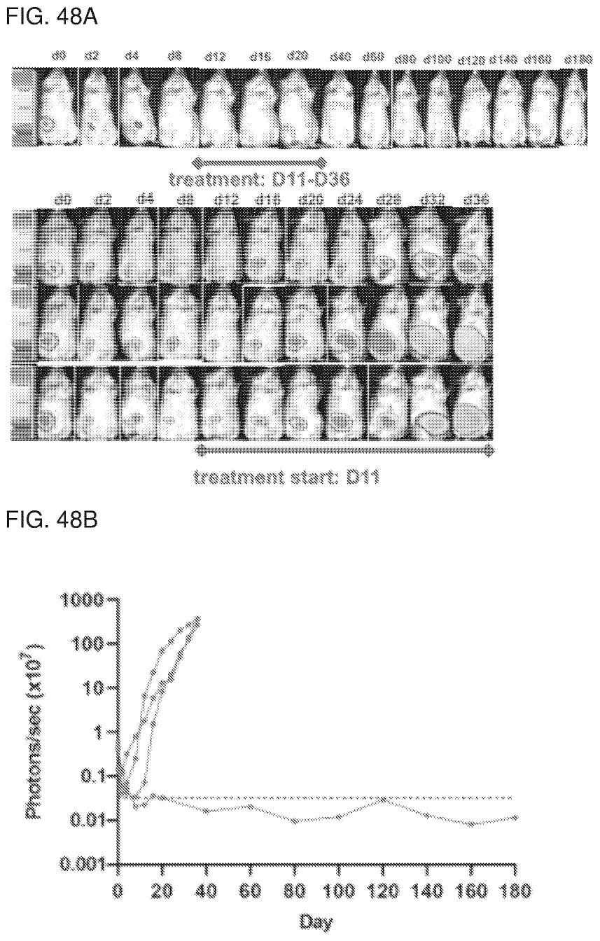

As shown in FIG. 48A-B, human HIP iPSCs were injected subcutaneously into NSG mice with adoptive transfer of human NK cells, and MIAP410 with Fc isotype IgG1 was administered locally (LD 500 μg). MIAP410 antibody treatment began on Day 11 (D11) and occurred during the period of D11-D36, with treatment stopped after D36. When follow-up was performed 6 months after beginning of treatment, it was observed that iPSCs were eliminated in 1 mouse, while teratoma developed in 3 mice.

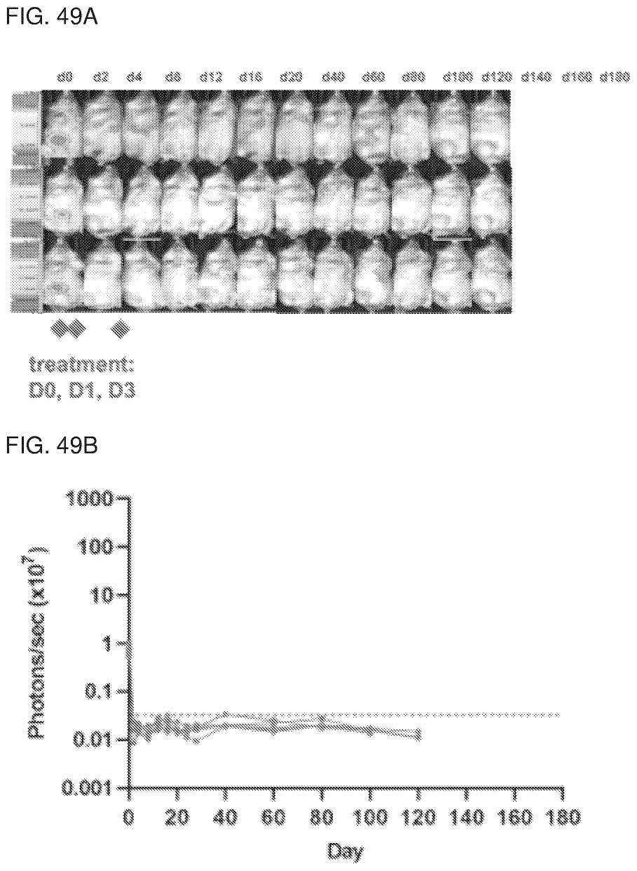

As shown in FIG. 49A-B, when human HIP iPSCs were injected subcutaneously into NSG mice with adoptive transfer of human NK cells, local HD 1 mg administration of MIAP410 with Fc isotype IgG1 resulted in killing of HIP iPSCs. MIAP410 antibody treatment occurred on D0, D1, and D3. When follow-up was performed 120 days after beginning of treatment, it was observed that iPSCs did not reappear in any mouse.

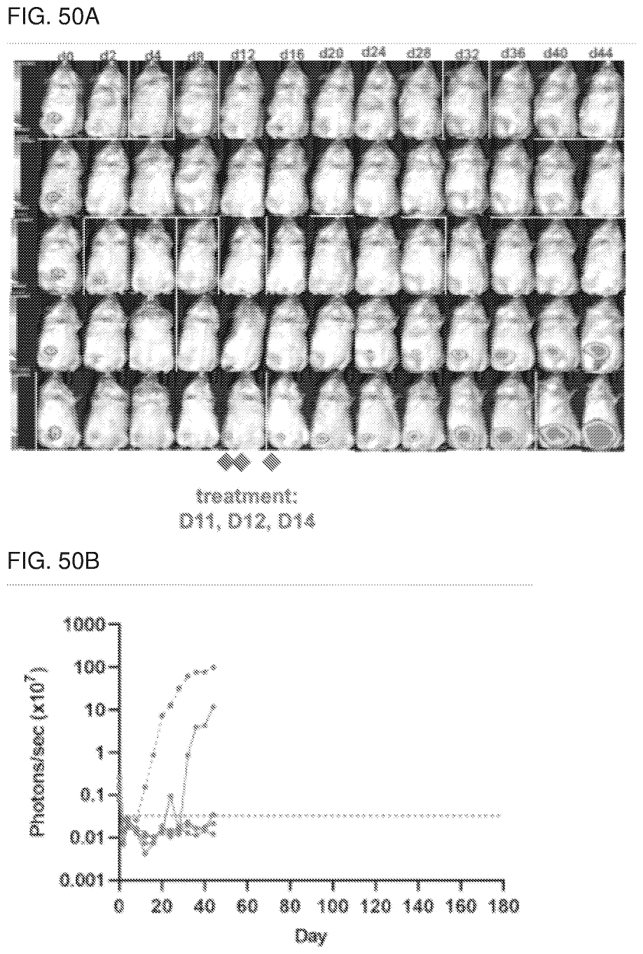

As shown in FIG. 50A-B, human HIP iPSCs were injected subcutaneously into NSG mice with adoptive transfer of human NK cells, and MIAP410 with Fc isotype IgG1 was administered locally (HD 1 mg). MIAP410 antibody treatment occurred on D11, D12, and D14. When follow-up was performed 44 days after beginning of treatment, it was observed that iPSCs did not reappear in 3 mice and teratoma developed in 2 mice.

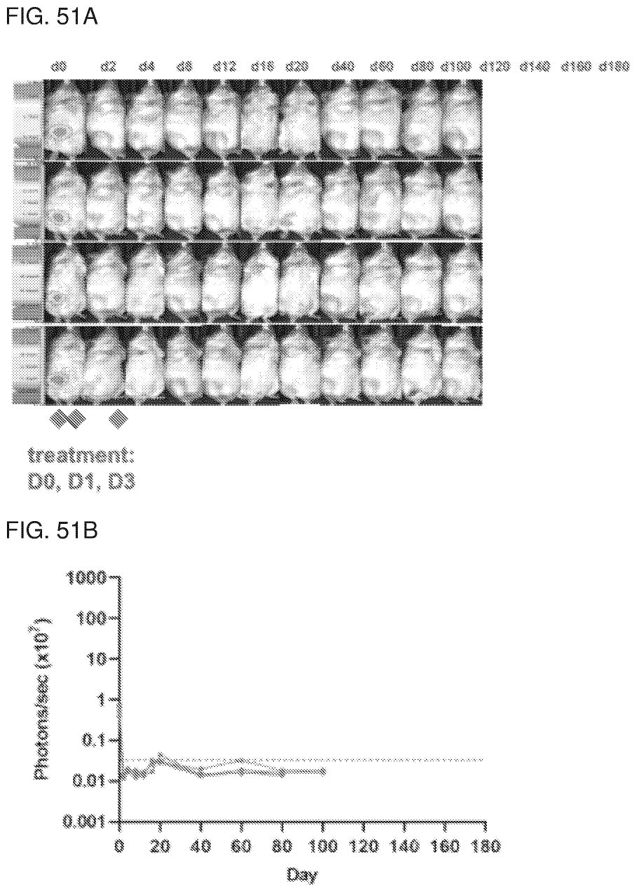

As shown in FIG. 51A-B, when human HIP iPSCs were injected subcutaneously into NSG mice with adoptive transfer of human NK cells, intraperitoneal HD 1 mg administration of MIAP410 with Fc isotype IgG1 resulted in killing of HIP iPSCs. MIAP410 antibody treatment occurred on D0, D1, and D3. When follow-up was performed 100 days after beginning of treatment, it was observed that iPSCs did not reappear in any mouse.

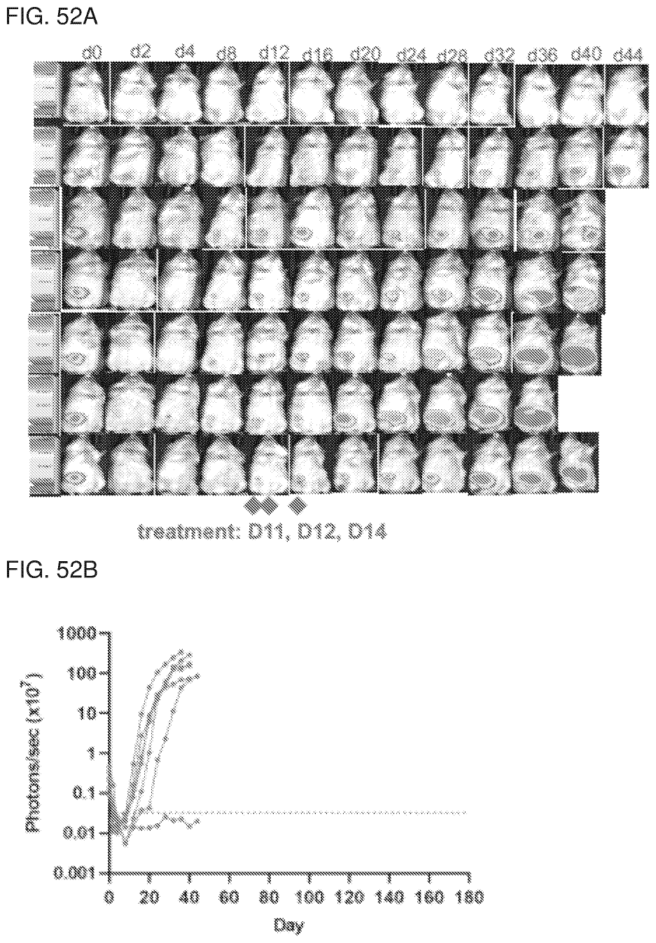

As shown in FIG. 52A-B, human HIP iPSCs were injected subcutaneously into NSG mice with adoptive transfer of human NK cells, and MIAP410 with Fc isotype IgG1 was administered intraperitoneally (HD 1 mg). MIAP410 antibody treatment occurred on D11, D12, and D14. When follow-up was performed 44 days after beginning of treatment, it was observed that iPSCs did not reappear in 1 mouse and teratoma developed in 6 mice.

### Example 6. Killing of Human Hypoimmune Cells in Mice In Vivo with or without Adoptive Transfer of Human NK Cells/Microglia Upon Treatment with MIAP410 Anti-Cd47 Antibody with IgG1 Fc Domain Varies Between Mouse Body and Brain

Protocols were followed as described in Example 4.

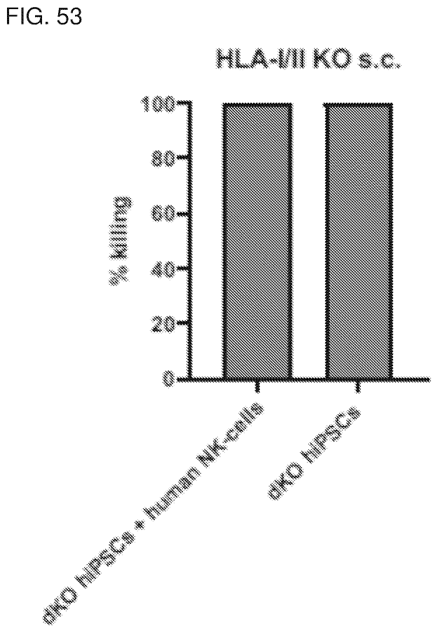

As shown in FIG. 53, when human dKO (B2M−/−/CIITA−/−) cells were injected subcutaneously into NSG mice, human iPSCs were killed by human NK cells when human NK cells were adoptively transferred, while human iPSCs were killed by NSG macrophages when no NK cells were adoptively transferred. The latter is presumably due to “xenogeneic sensing of missing-self” by macrophages.

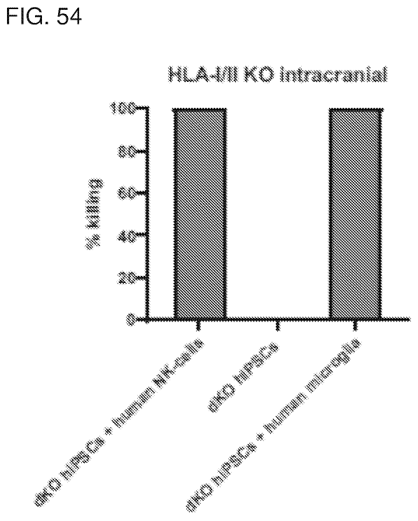

As shown in FIG. 54, when human dKO (B2M−/−/CIITA−/−) cells were injected into the brain of NSG mice, human iPSCs were killed when human NK cells or human microglia were adoptively transferred. However, human iPSCs were not killed by NSG microglia alone when no adoptive transfer of human NK cells or human microglia occurred. The latter is presumably due to a lack of “xenogeneic sensing of missing-self” by microglia.

### A. Sensing of Missing Self in the Brain Depends on Allogeneic Microglia

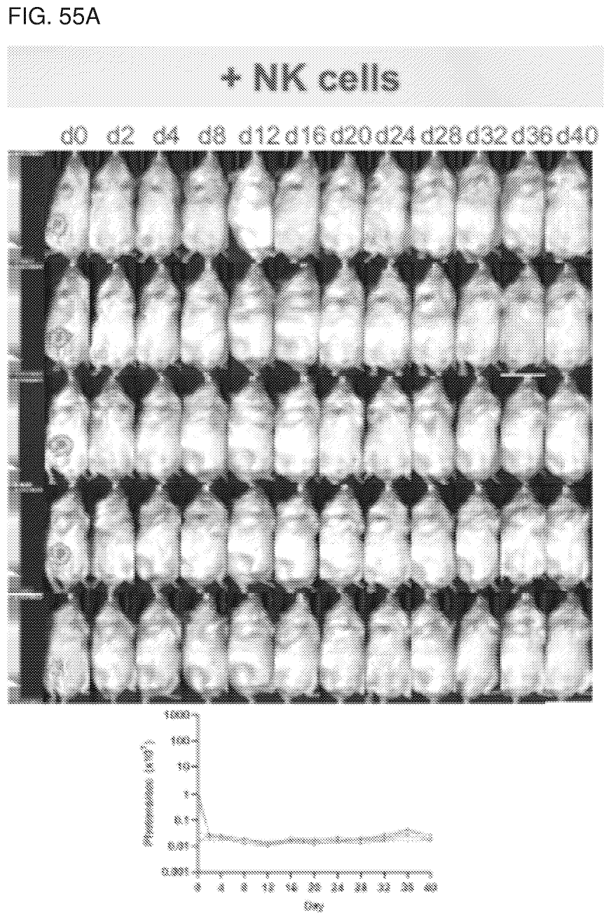

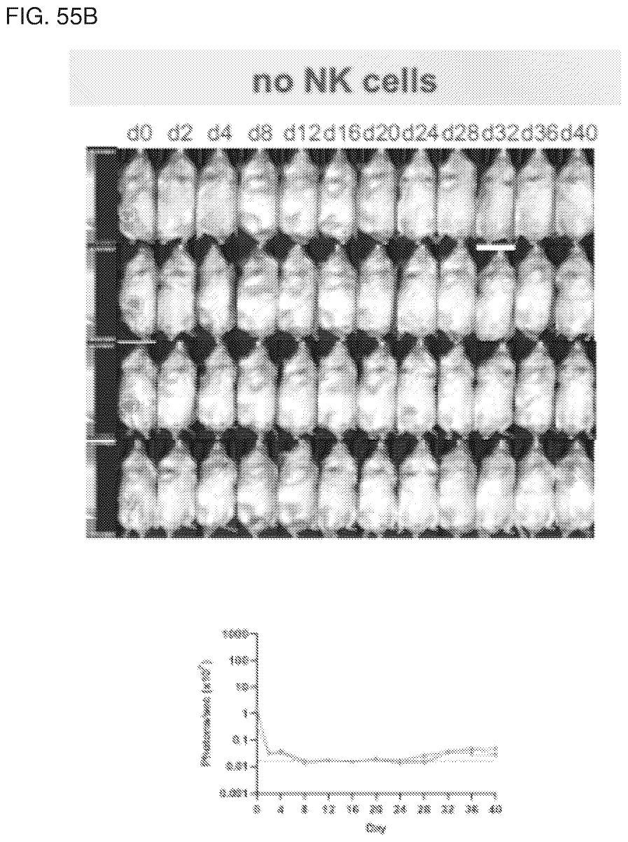

As shown in FIG. 55A-B, 5×10⁴ human dKO (B2M−/−CIITA^{-/-}) cells were injected subcutaneously into NSG mice with or without adoptive transfer of human NK cells. When human NK cells were adoptively transferred, human dKO iPSCs were killed subcutaneously. When no human NK cells were transferred, human dKO iPSCs were killed by NSG macrophages, presumably through “xenogeneic sensing of missing self”.

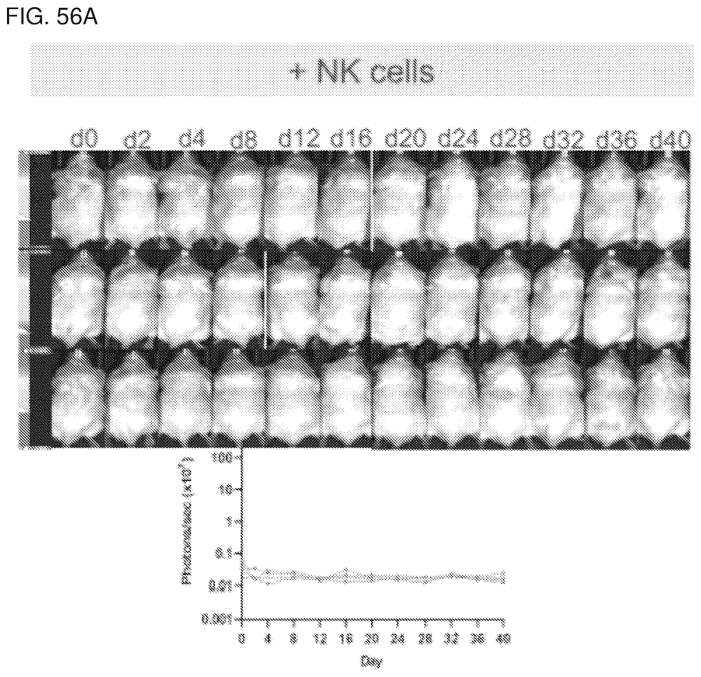

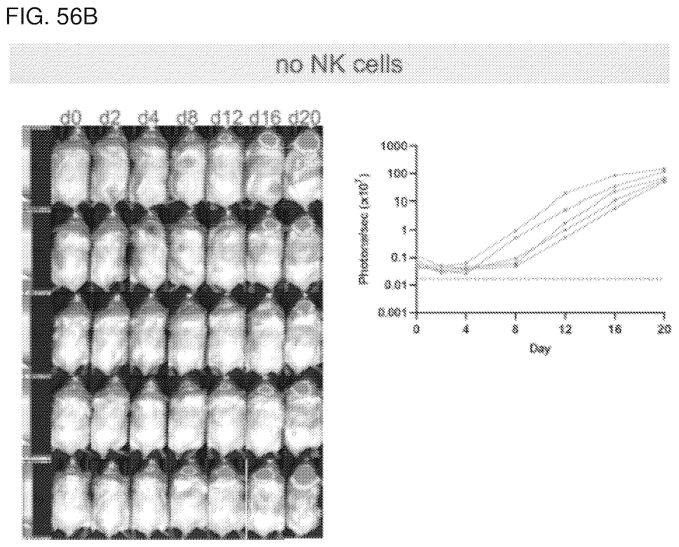

As shown in FIG. 56A-B, 5×10⁴ human dKO (B2M−/−CIITA−/−) cells were injected into the brain of NSG mice with or without adoptive transfer of human NK cells. Human dKO iPSCs were killed in the brain when human NK cells were adoptively transferred, but not by NSG microglia when no human NK cells were transferred.

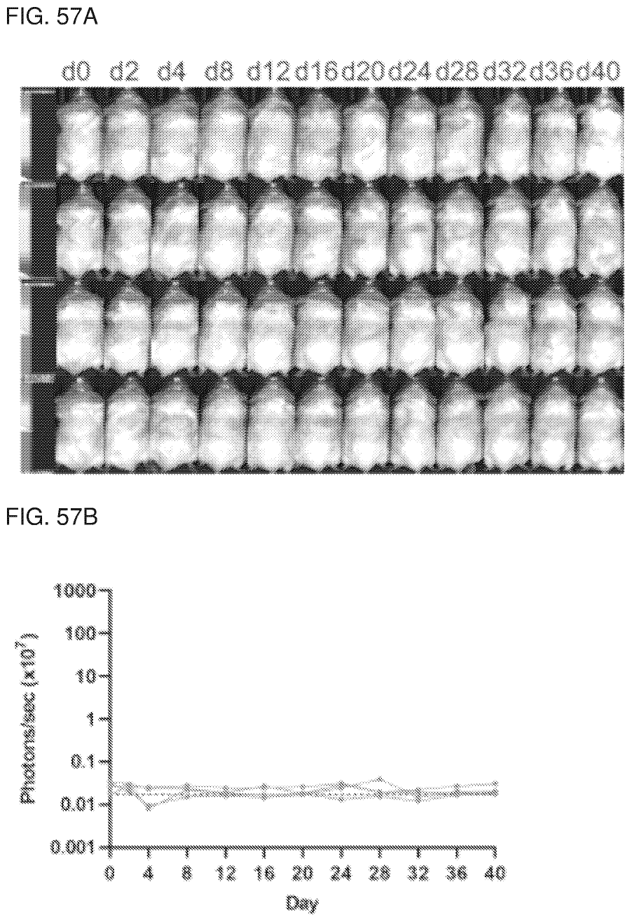

As shown in FIG. 57A-B, when 5×10⁴ human dKO (B2M−/−CIITA−/−) cells were injected into the brain of NSG mice with adoptive transfer of human microglia, human dKO iPSCs were killed in the brain, presumably through allogeneic sensing of “missing self”.

### Example 7. CD47 Protects Human Hypoimmune Cells in Mouse Brain from Microglia-Mediated Killing

Protocols were followed as described in Example 4.

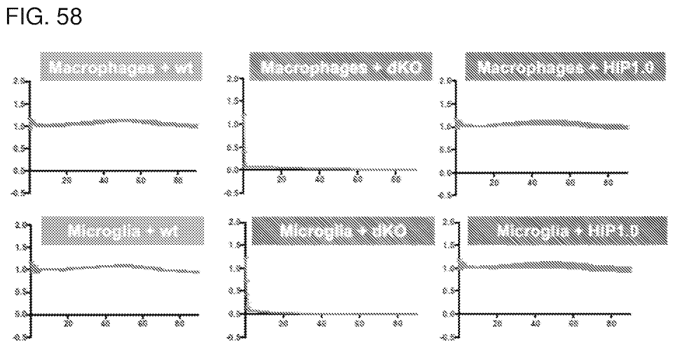

As shown in FIG. 58, when human wt, dKO (B2M−/−CIITA−/−) or HIP 1.0 (B2M−/−CIITA−/−CD47 tg) were co-cultured with allogeneic human macrophages or microglia, CD47 protected dKO cells both from macrophage killing as well as from microglia killing.

### A. Differences in Microglia and Macrophage Killing of dKO Cells

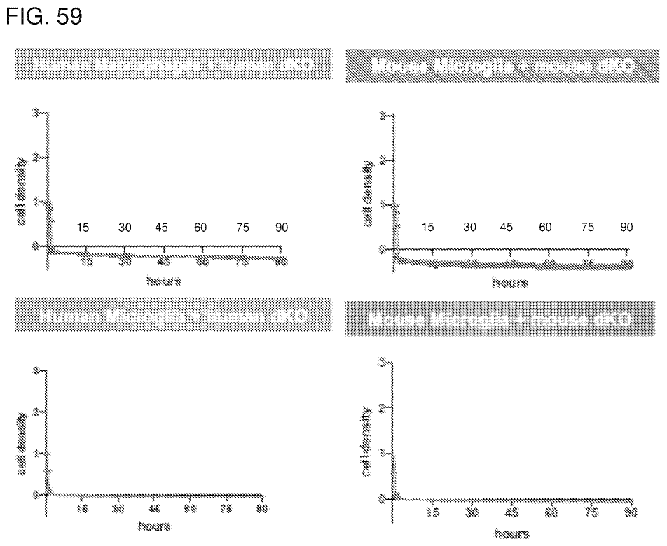

As shown in FIG. 59, when human dKO (B2M−/−CIITA−/−) cells were co-cultured with allogeneic human macrophages or microglia or mouse dKO (B2M−/−CIITA−/−) cells were co-cultured with allogeneic mouse macrophages or microglia, allogeneic macrophages and microglia sensed the missing self and killed the dKO cells.

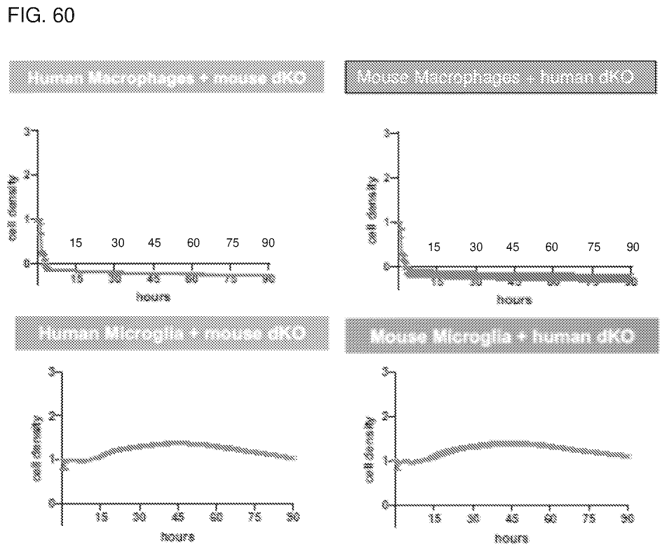

As shown in FIG. 60, when human dKO (B2M−/−CIITA−/−) cells were co-cultured with xenogeneic (cross-species) mouse macrophages or microglia or mouse dKO (B2M−/−CIITA−/−) cells were co-cultured with xenogeneic human macrophages or microglia, xenogeneic macrophages and microglia did not sense the missing self and therefore did not kill the dKO cells.

### B. Dosing Studies

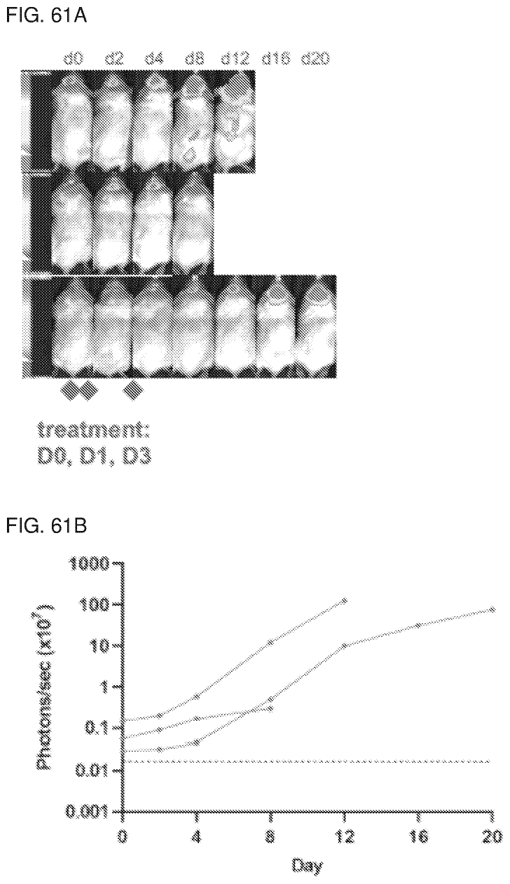

As shown in FIG. 61A-B, when human HIP iPSCs were injected intracranially into NSG mice with adoptive transfer of human NK cells, local HD (mouse IgG1; 1 mg) administration of local IgG1 isotype control on D0, D1, and D3 did not affect HIP survival.

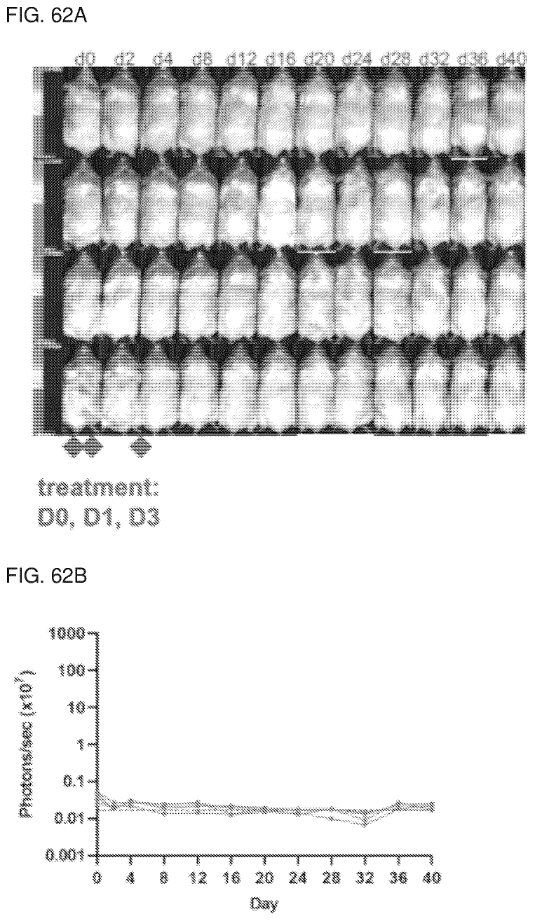

As shown in FIG. 62A-B, when human HIP iPSCs were injected intracranially into NSG mice with adoptive transfer of human NK cells, local HD (1 mg) administration of MIAP410 on D0, D1, and D3 resulted in killing of HIP iPSCs in the brain. Upon 40 day follow-up, it was observed that iPSCs did not reappear.

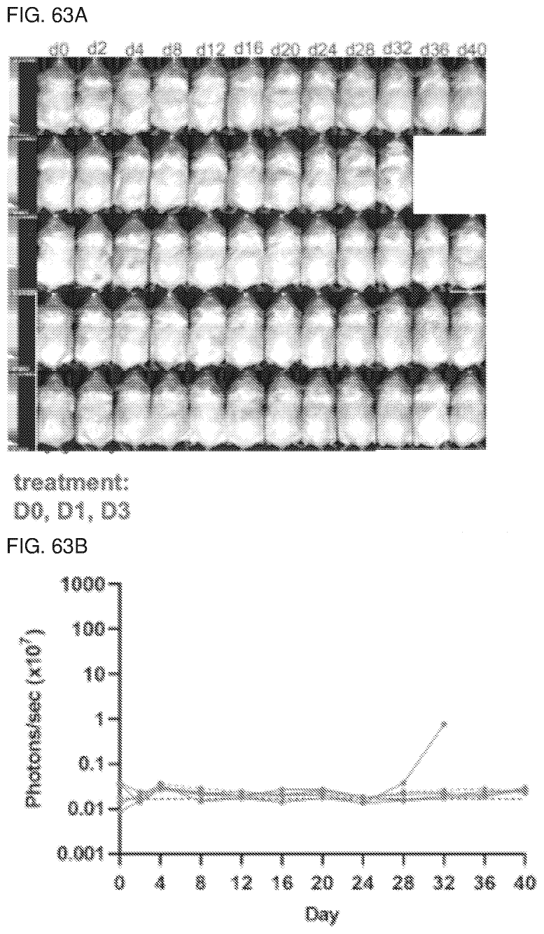

As shown in FIG. 63A-B, when human HIP iPSCs were injected intracranially into NSG mice with adoptive transfer of human NK cells, intraperitoneal HD (1 mg) administration of MIAP410 on D0, D1, and D3, with the blood-brain barrier broken by mannitol injections, resulted in killing of HIP iPSCs in the brain. Upon 40 day follow-up, it was observed that iPSCs did not reappear in 4 out of 5 mice.

### Example 8. Administering B6H12 Anti-CD47 Antibody with Mouse IgG1 Fc Domain Blocks CD47 in Human Hypoimmune Cells In Vitro and/or In Vivo

Protocols were followed as in Example 4.

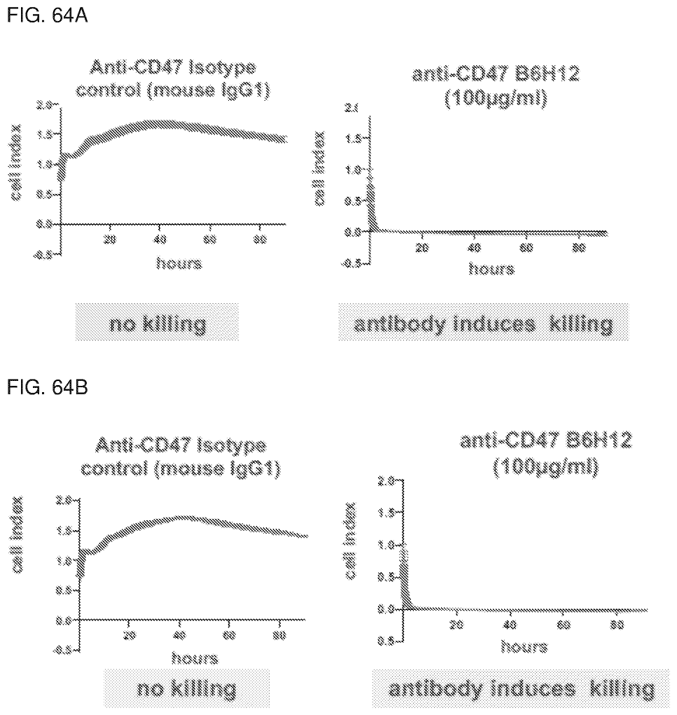

Administering B6H12 anti-CD47 antibody with mouse IgG1 Fc domain blocks CD47 in human hypoimmune cells in the presence of human NK cells and/or macrophages in vitro. FIG. 64A (human HIP iPSCs and human NK cells) and FIG. 64B (human HIP iPSCs and human macrophages) show that administration of 100 μg/ml of anti-CD47 antibody B6H12 in vitro to target human HIP iPSCs resulted in killing by human NK cells and human macrophages.

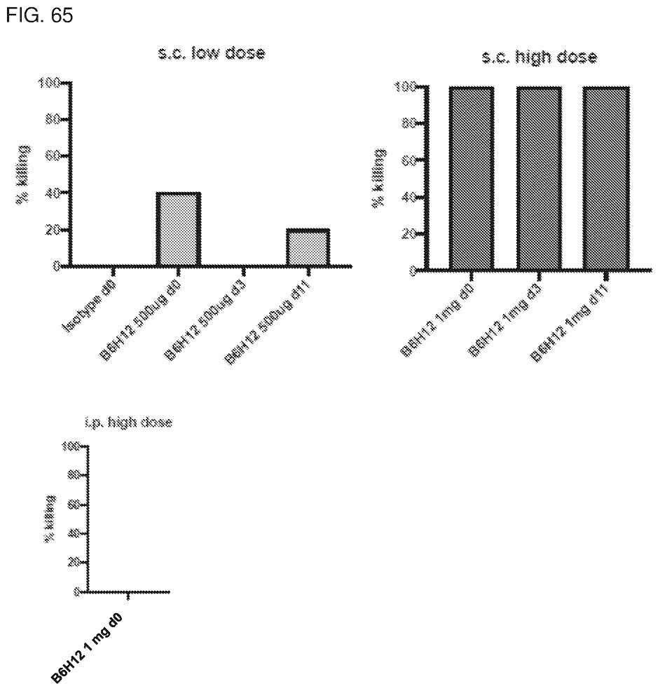

Administering B6H12 anti-CD47 antibody with mouse IgG1 Fc domain blocks CD47 in human hypoimmune cells in vivo with adoptive transfer of human NK cells. As shown in FIG. 65, a subcutaneous higher dose (HD) of 1 mg three times seemed more effective than administering LD (500 μg) of B6H12 anti-CD47 antibody continuously. An intraperitoneal dose seemed ineffective.

A. Variation of Results Depending on when Treatment Begins

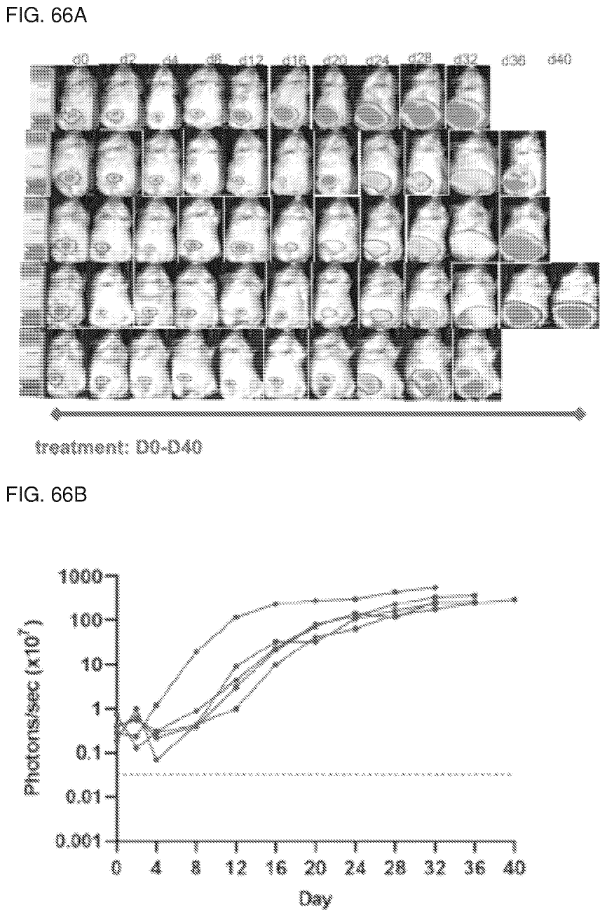

As shown in FIG. 66A-B, human HIP iPSCs formed teratoma in NSG mice with adoptively transferred NK cells. When human HIP iPSCs were injected subcutaneously into NSG mice with adoptive transfer of human NK cells, local LD (500 μg) administration of Fc isotype IgG4 control during the period of D0-D40 did not affect HIP survival.

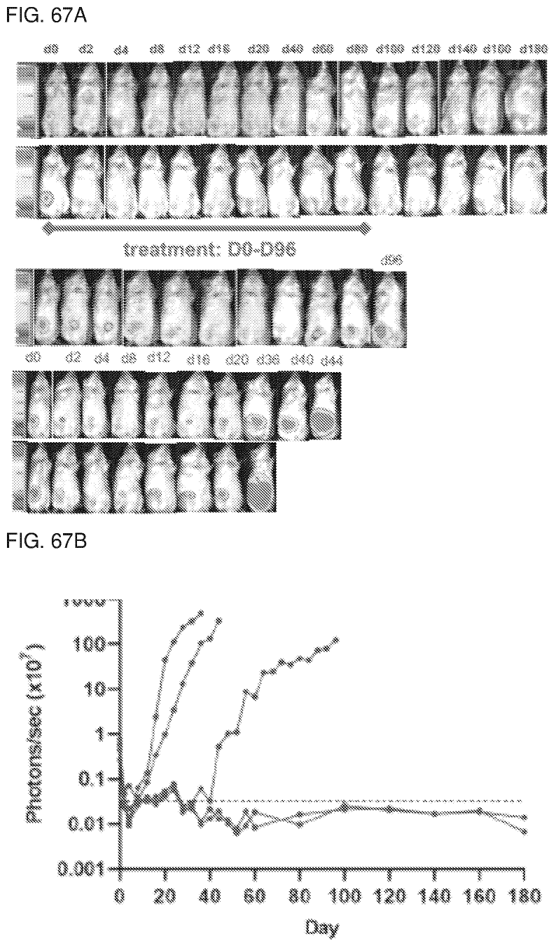

As shown in FIG. 67A-B, human HIP iPSCs were injected subcutaneously into NSG mice with adoptive transfer of human NK cells, and B6H12 anti-CD47 antibody with Fc isotype IgG1 was administered locally (LD 500 μg). B6H12 antibody treatment occurred during the period of D0-D96. It was observed that iPSCs were eliminated in 2 mice and teratoma developed in 3 mice when follow-up was performed 6 months after treatment.

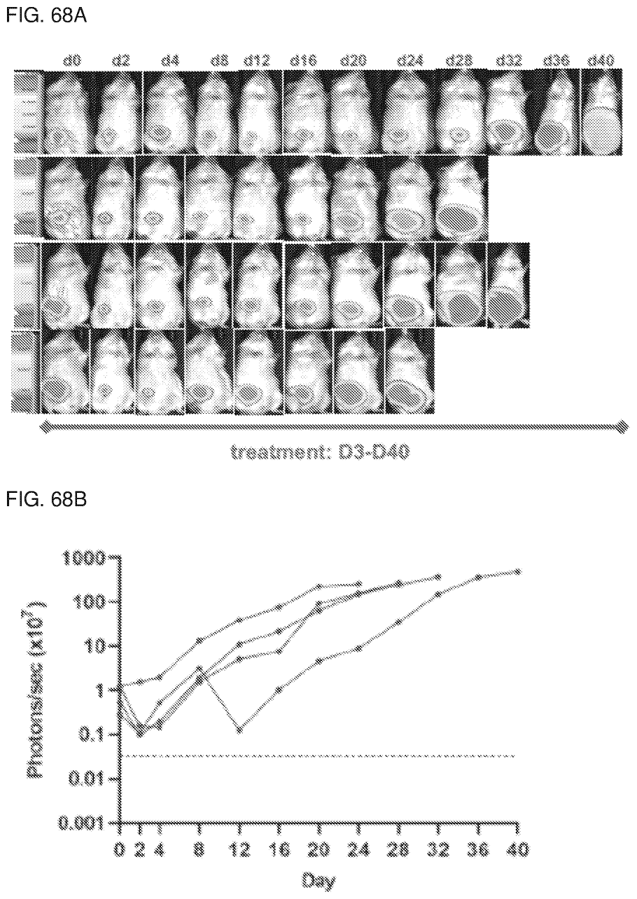

As shown in FIG. 68A-B, human HIP iPSCs were injected subcutaneously into NSG mice with adoptive transfer of human NK cells, and B6H12 with Fc isotype IgG1 was administered locally (LD 500 μg). B6H12 antibody treatment occurred during the period of D3-D40. On follow-up after 40 days from the beginning of treatment, teratoma were observed to have developed in all 4 mice.

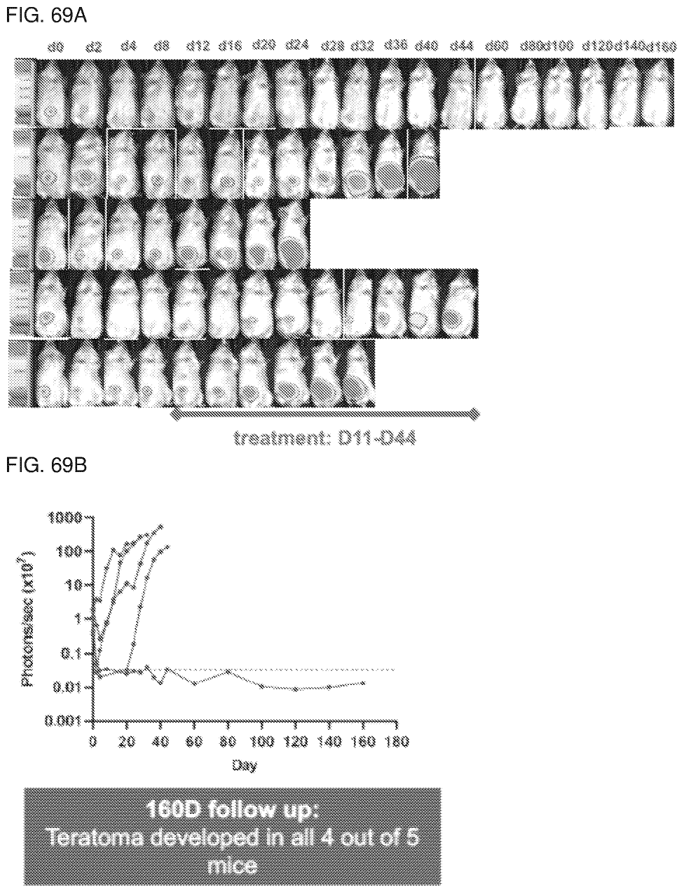

As shown in FIG. 69A-B, human HIP iPSCs were injected subcutaneously into NSG mice with adoptive transfer of human NK cells, and B6H12 with Fc isotype IgG1 was administered locally (LD 500 μg). B6H12 antibody treatment occurred during the period of D11-D44. On follow-up after 160 days from the beginning of treatment, teratoma were observed to have developed in 4 out of 5 mice.

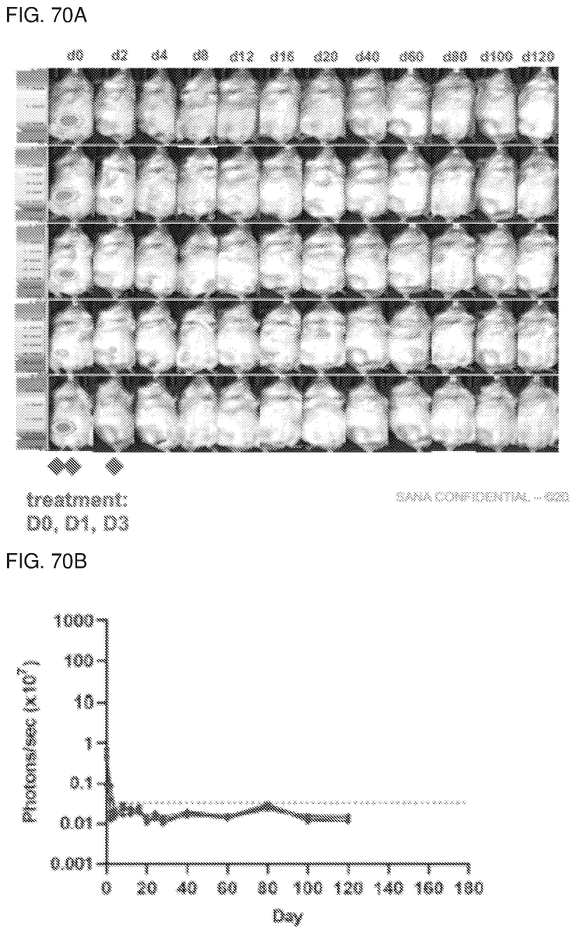

As shown in FIG. 70A-B, when human HIP iPSCs were injected subcutaneously into NSG mice with adoptive transfer of human NK cells, local HD 1 mg administration of B6H12 with Fc isotype IgG1 resulted in killing of HIP iPSCs. B6H12 antibody treatment was performed three times on D0, D1, and D3. On follow-up after 120 days from the beginning of treatment, it was observed that iPSCs did not reappear in any mouse.

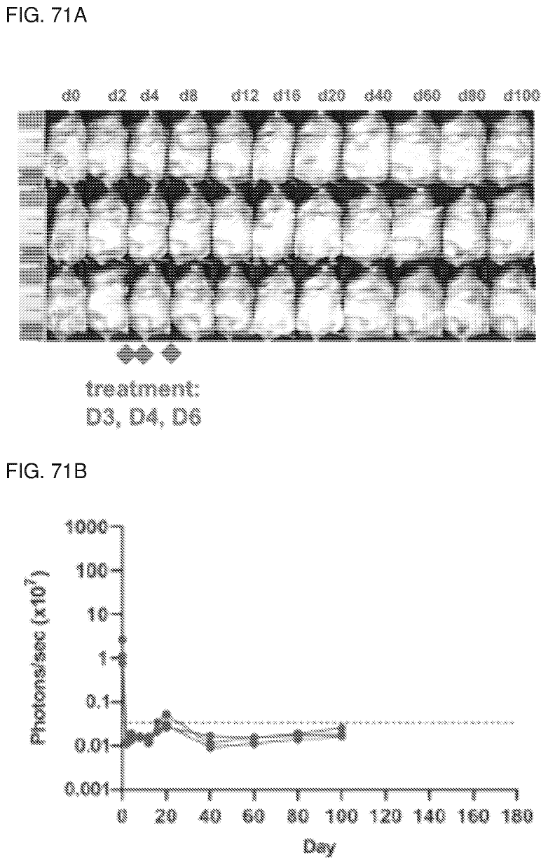

As shown in FIG. 71A-B, when human HIP iPSCs were injected subcutaneously into NSG mice with adoptive transfer of human NK cells, local HD 1 mg administration of B6H12 with Fc isotype IgG1 resulted in killing of HIP iPSCs. B6H12 antibody treatment was performed three times on D3, D4, and D6. On follow-up after 100 days from the beginning of treatment, it was observed that iPSCs did not reappear in any mouse.

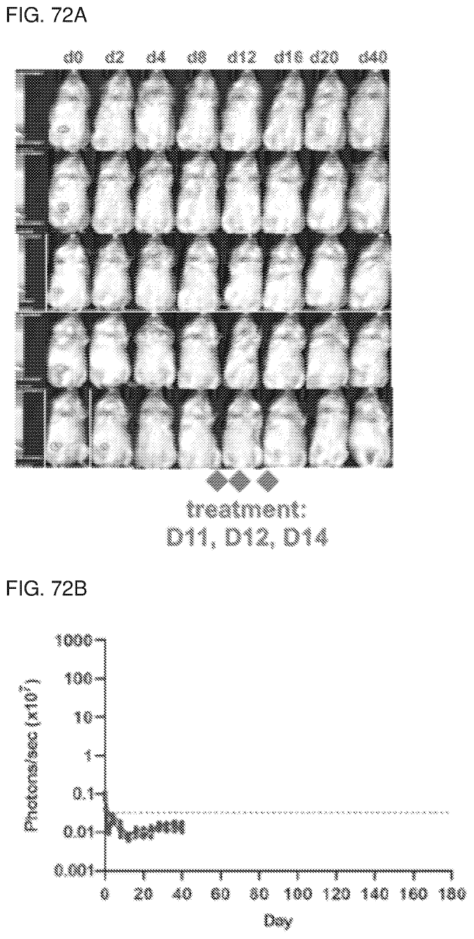

As shown in FIG. 72A-B, when human HIP iPSCs were injected subcutaneously into NSG mice with adoptive transfer of human NK cells, local HD 1 mg administration of B6H12 with Fc isotype IgG1 resulted in killing of HIP iPSCs. B6H12 antibody treatment was performed three times on D11, D12, D14. On follow-up after 40 days from the beginning of treatment, it was observed that iPSCs did not reappear in any mouse.

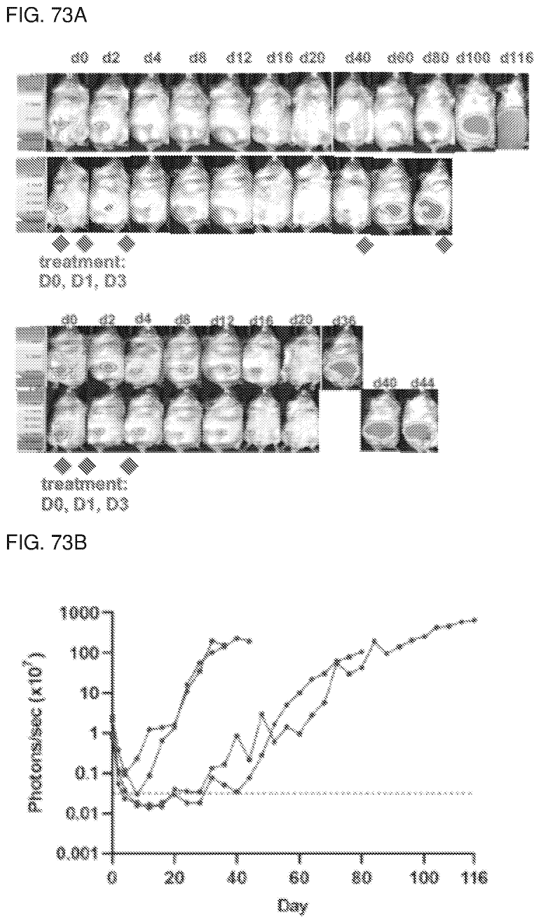

As shown in FIG. 73A-B, human HIP iPSCs were injected subcutaneously into NSG mice with adoptive transfer of human NK cells, and intraperitoneal (HD 1 mg) administration of B6H12 with Fc isotype IgG1 was performed three times on D0, D1, and D3. On follow-up after 120 days from the beginning of treatment, it was observed that teratoma developed in all 4 mice.

### Example 9. Administering Flucytosine and/or Ganciclovir Results in Killing of Hypoimmune Cells In Vitro and/or In Vivo

Protocols were followed as in Example 4.

### A. Small Molecules Flucytosine and Ganciclovir In Vitro

Small molecules Flucytosine and Ganciclovir were studied in vitro for their effects on cytosine deaminase and HsVtk kill switch, respectively. All tested clones were observed to have sufficient CD47 levels to protect from NK cell and macrophage killing (FIG. 74). Pro-drug killing data were also obtained (FIG. 75).

### B. Small Molecules Flucytosine In Vivo

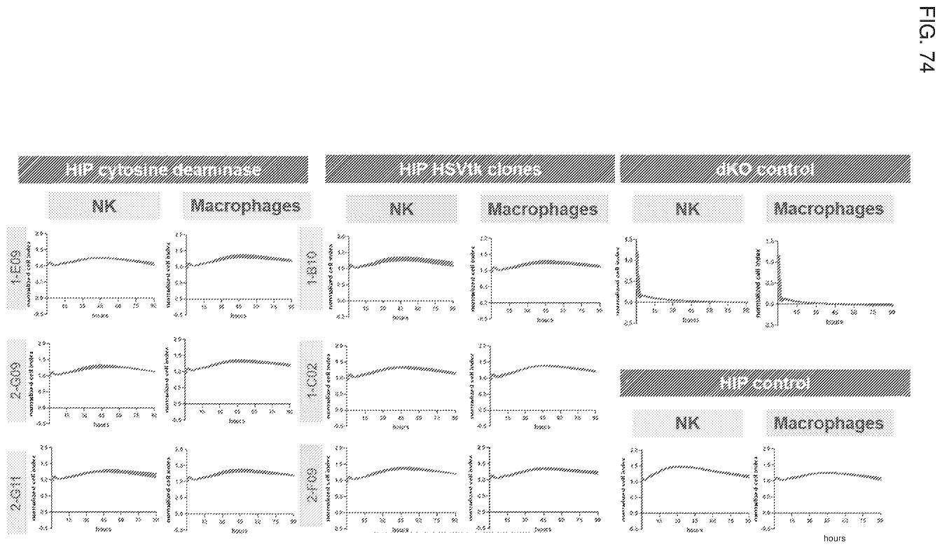

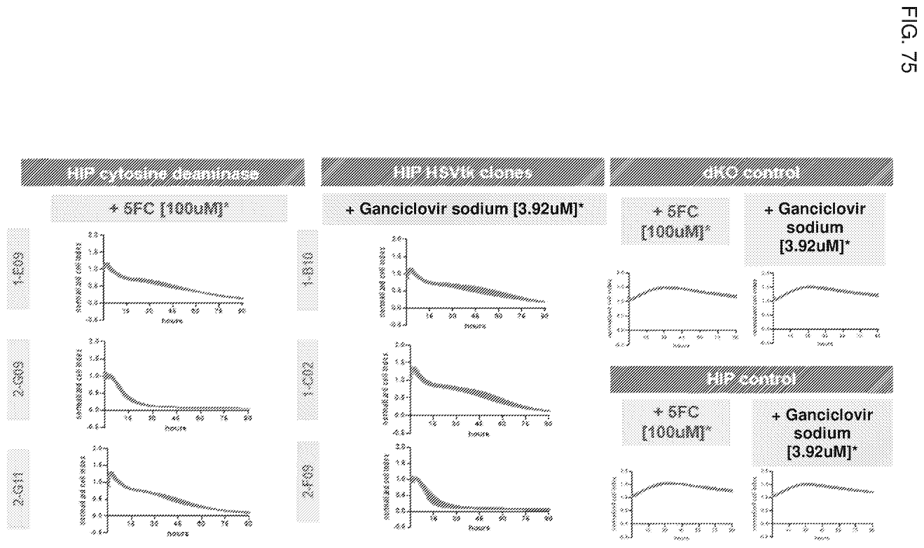

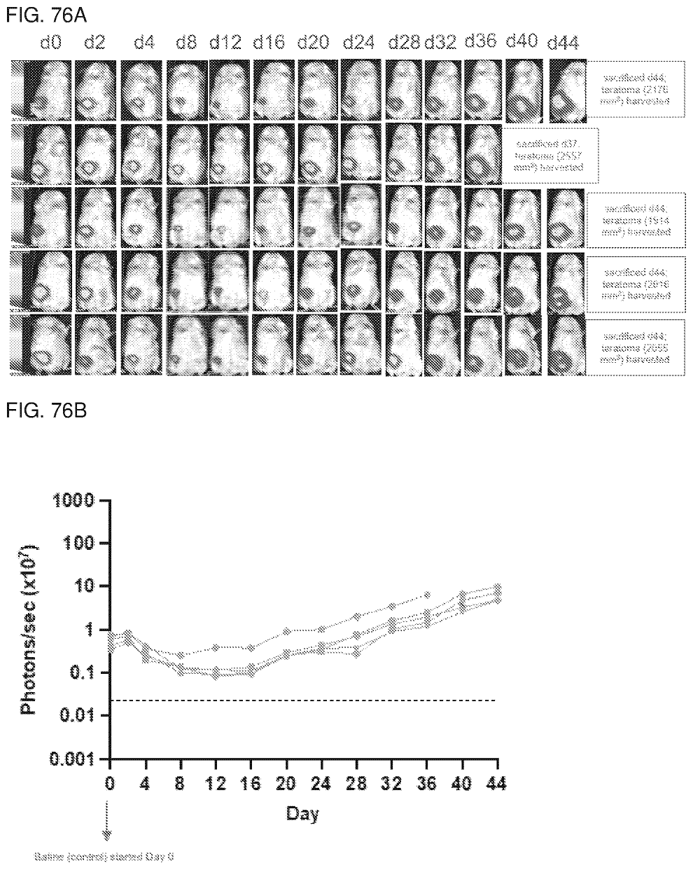

Small molecule Flucytosine was studied in vivo for its effects on the cytosine deaminase kill switch. Human HIP iPSCs (CyD clone 2G11) were injected subcutaneously into Group 5 NSG mice. Saline was used as a control. As shown in FIG. 76A-B, human HIP-CyD iPSCs formed teratoma in NSG mice (saline control). Group 5 mice treatment was as follows: hiPSC HIP CyD clone 2G11 in diluted MG, S.C., with no treatment (saline, IP, DO).

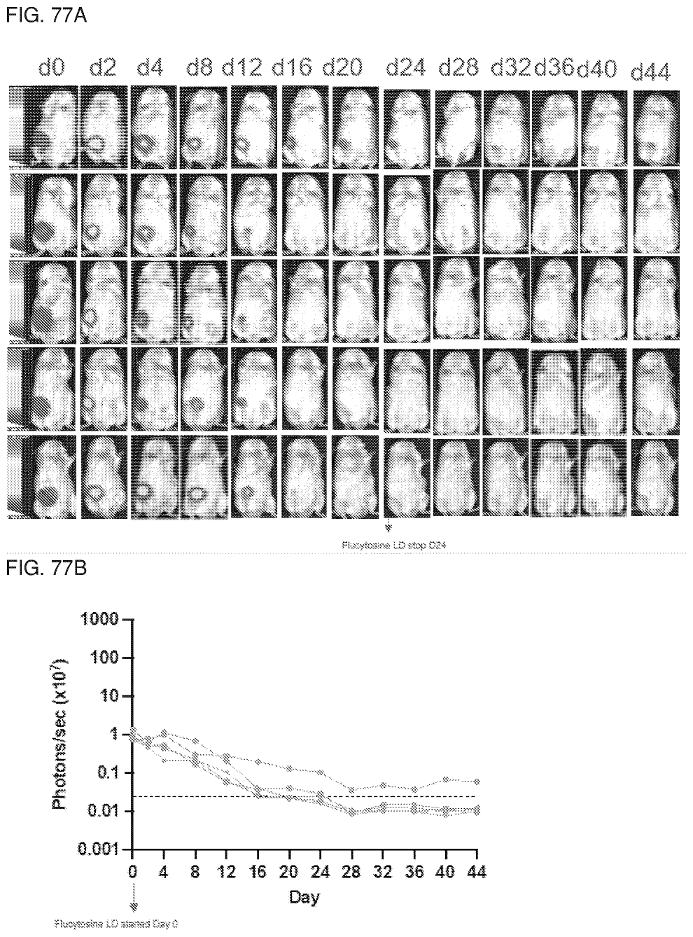

Human HIP iPSCs (CyD clone 2G11) were injected subcutaneously into Group 1 NSG mice. Flucytosine LD (200 mg/kg) treatment was administered daily intraperitoneally. As shown in FIG. 77A-B, dosing with Flucytosine LD resulted in killing of HIP-CyD iPSCs within 16-44 days. Group 1 mice treatment was as follows: 1×10⁶ hiPSC HIP CyD clone 2G11 in diluted MG, S.C.; treatment=200 mg/kg/day Flucytosine, IP (DO).

Human HIP iPSCs (CyD clone 2G11) were injected subcutaneously into Group 2 NSG mice. Flucytosine HD (500 mg/kg) treatment was administered daily intraperitoneally. As shown in FIG. 78A-B, dosing with Flucytosine HD resulted in killing of HIP-CyD iPSCs within 16-32 days, with a 44 day follow up. No major benefit of HD was observed as compared to LD. Group 2 mice treatment was as follows: 1×10⁶ hiPSC HIP CyD clone 2G11 in diluted MG, S.C.; treatment was 500 mg/kg/day Flucytosine, IP (DO).

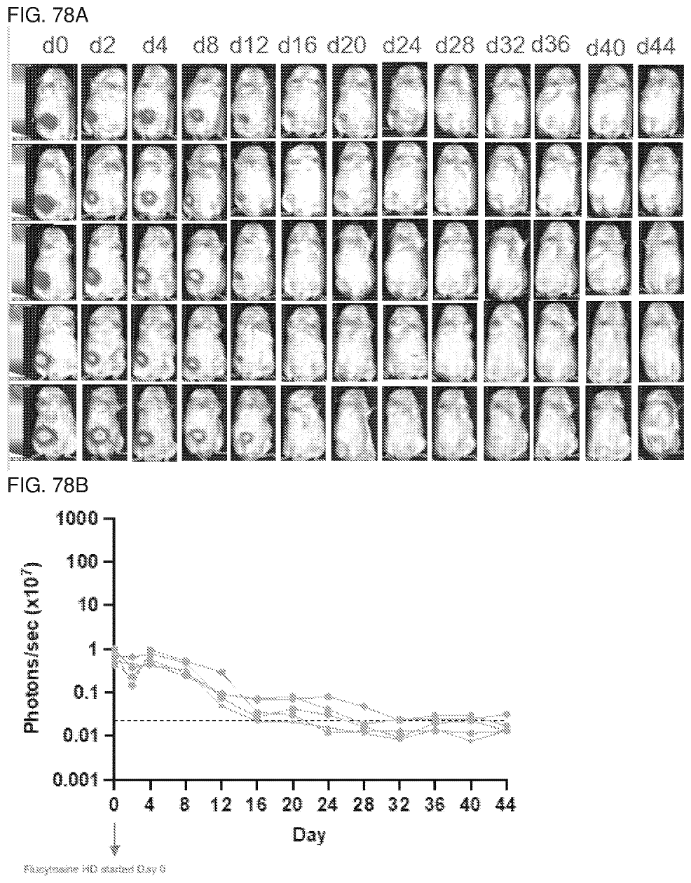

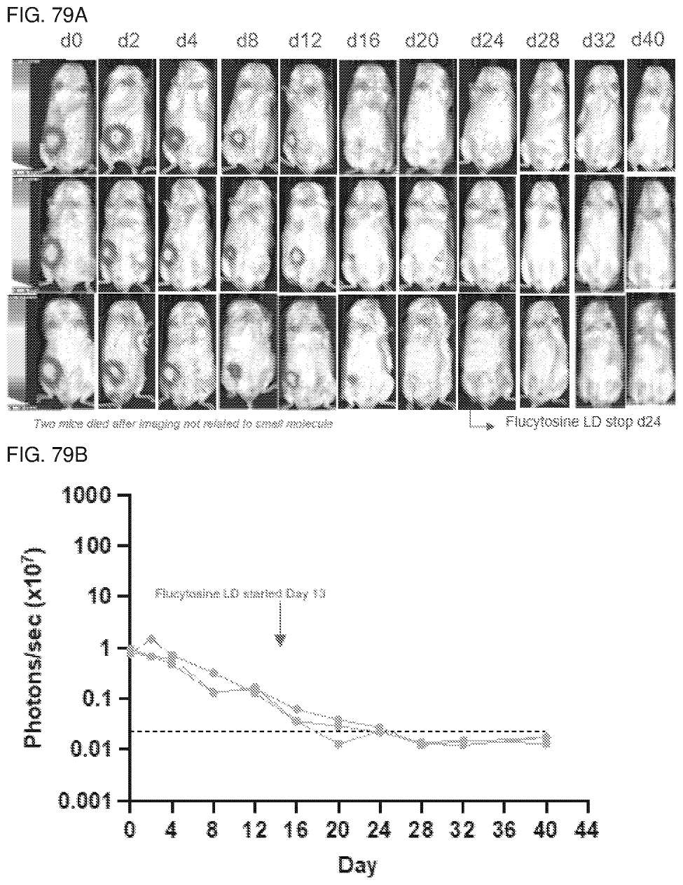

Human HIP iPSCs (CyD clone 2G11) were injected subcutaneously into Group 3 NSG mice. Flucytosine LD (200 mg/kg) treatment was started on Day 13 and then administered daily intraperitoneally. As shown in FIG. 79A-B, dosing with Flucytosine LD resulted in killing of HIP-CyD iPSCs within 3-11 days after starting treatment. Mice were checked during a 40D follow up. Group 3 mice treatment was as follows: 1×10⁶ hiPSC HIP CyD clone 2G11 in diluted MG, S.C.; treatment was 200 mg/kg/day Flucytosine, IP (D13).

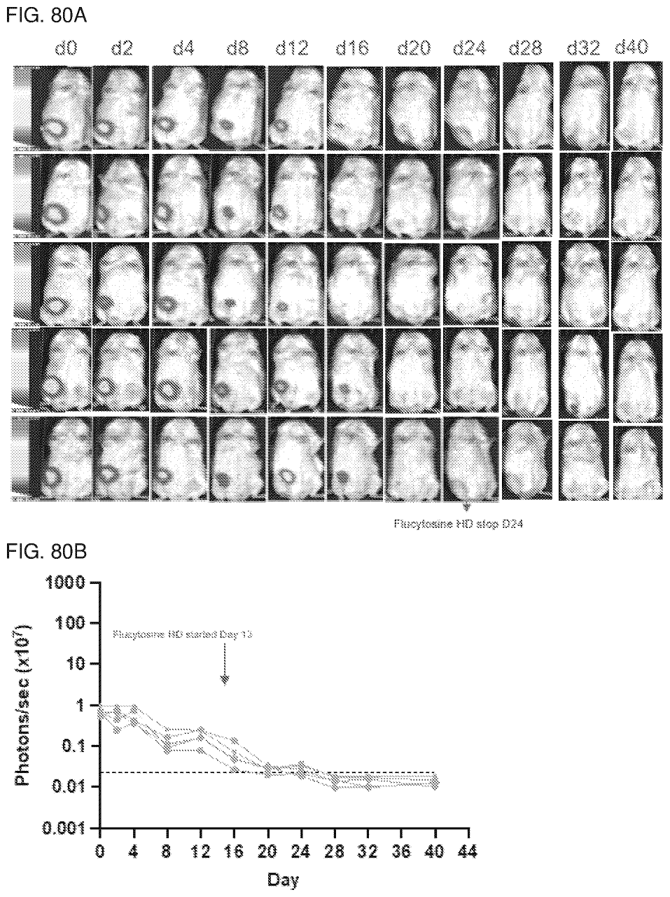

Human HIP iPSCs (CyD clone 2G11) were injected subcutaneously into Group 4 NSG mice. Flucytosine HD (500 mg/kg) treatment was administered daily intraperitoneally, starting on Day 13. As shown in FIG. 80A-B, dosing with Flucytosine HD resulted in killing of HIP-CyD iPSCs within 3-11 days after treatment was started. Mice were checked during a 40D follow up. No benefit of HD was observed compared to LD. Group 4 mice treatment was as follows: 1×10⁶ hiPSC HIP CyD clone 2G11 in diluted MG, S.C.; treatment was 500 mg/kg/day Flucytosine, IP (D13).

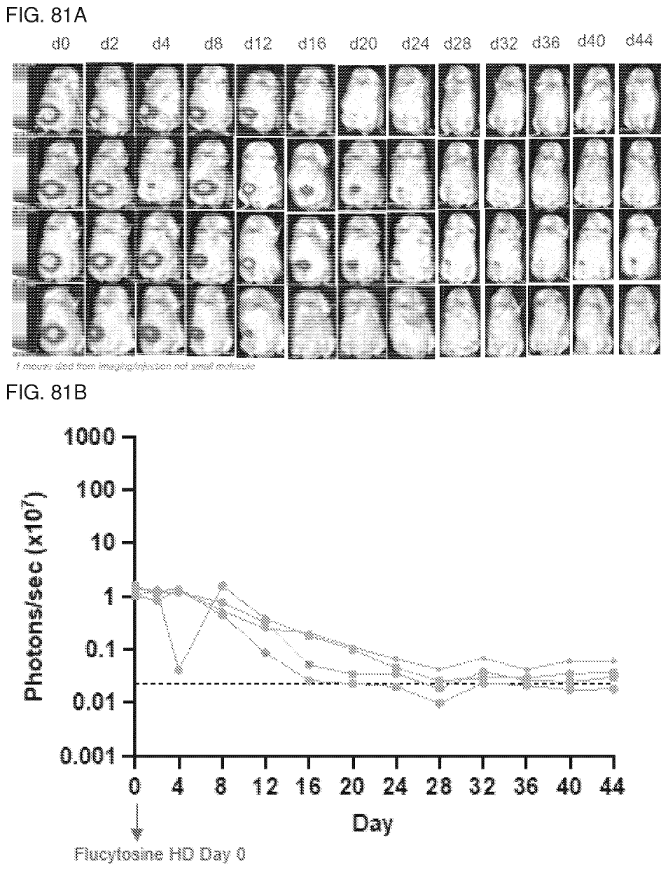

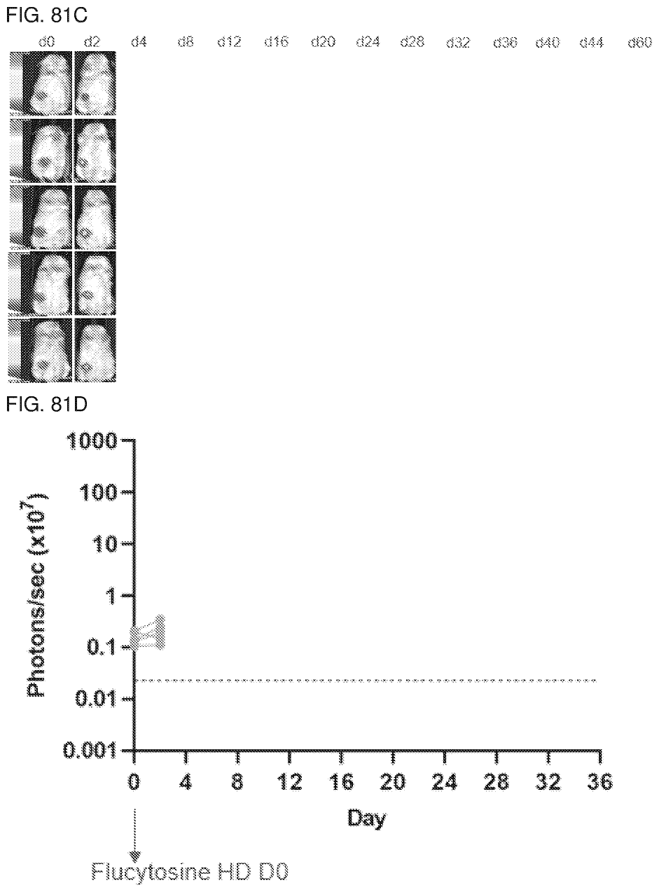

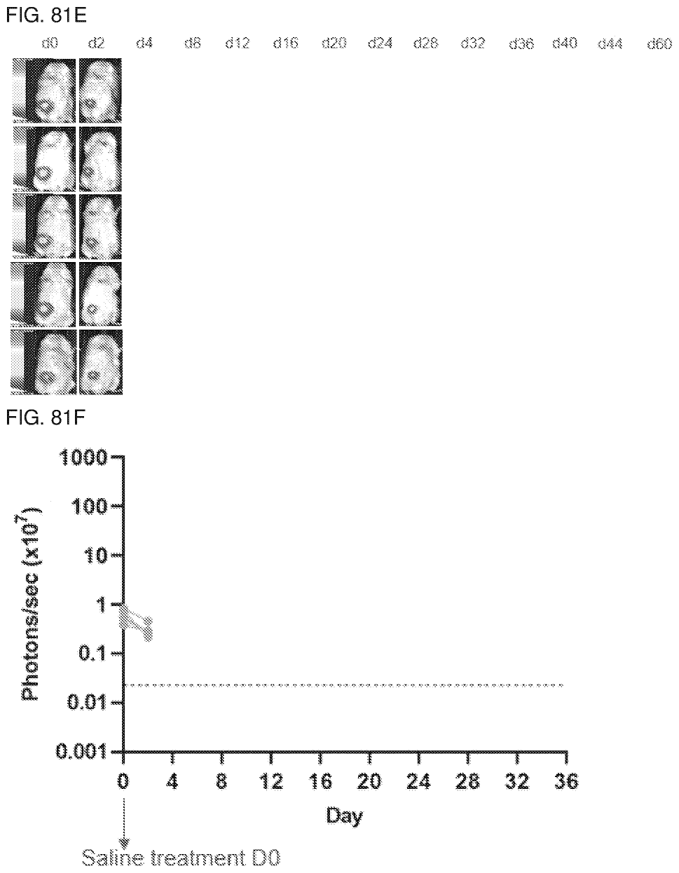

Human HIP iPSCs (clone 15; no kill switch) were injected subcutaneously into Group 6 NSG mice. Flucytosine HD (500 mg/kg) treatment was administered daily intraperitoneally. As shown in FIG. 81A-B, human HIP iPSC survival appeared to be impaired by flucytosine HD although cells had no kill switch. The group was expanded for data validation (FIG. 81C-D and FIG. 81E-F). Group 6 mice treatment was as follows: 1×10⁶ hiPSC HIP clone 15 in diluted MG, S.C.; treatment was 500 mg/kg/day Flucytosine, IP (DO).

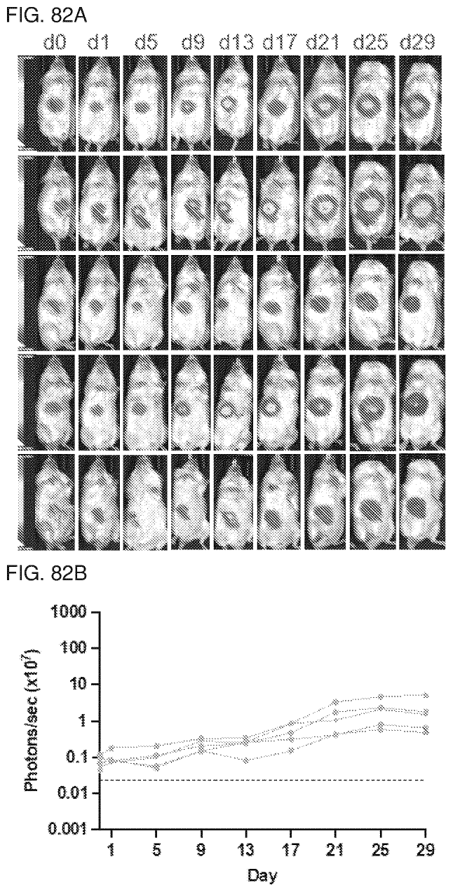

1×10⁶ human HIP iPSCs luc+ (Cytosine deaminase clone 2-G11) were injected subcutaneously into NSG mice. No treatment was performed on this control group. As shown in FIG. 82A-B, it was observed that the cytosine deaminase gene edit did not impact iPSCs, which survived in NSG mice.

### C. Small Molecules Ganciclovir In Vivo

Small Molecule Ganciclovir was studied in vivo for its effects on the HsVtk kill switch. Human HIP iPSCs (HSVTk clone 1-B10) were injected subcutaneously into Group 5 NSG mice. The control group received saline treatment. As shown in FIG. 83A-B, human HIP-HsVtk iPSC survival appeared to be impaired in NSG mice, perhaps due to a kill switch edit. Group 5 mice treatment was as follows: 1×10⁶ hiPSCs HIP HSVtk clone 1-B10 luc+ in diluted MG, S.C.; no treatment (saline, IP, DO).

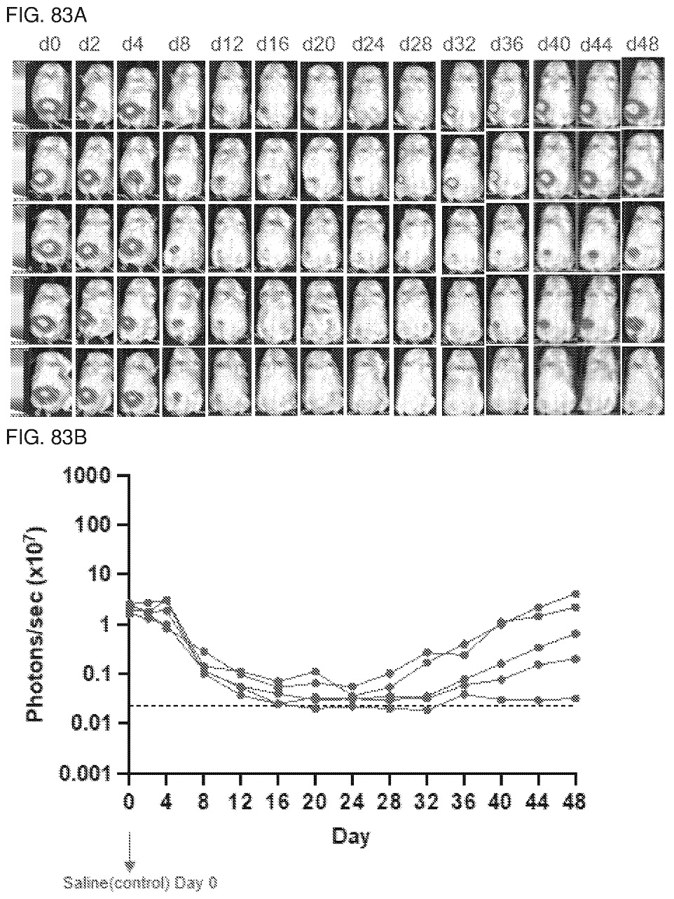

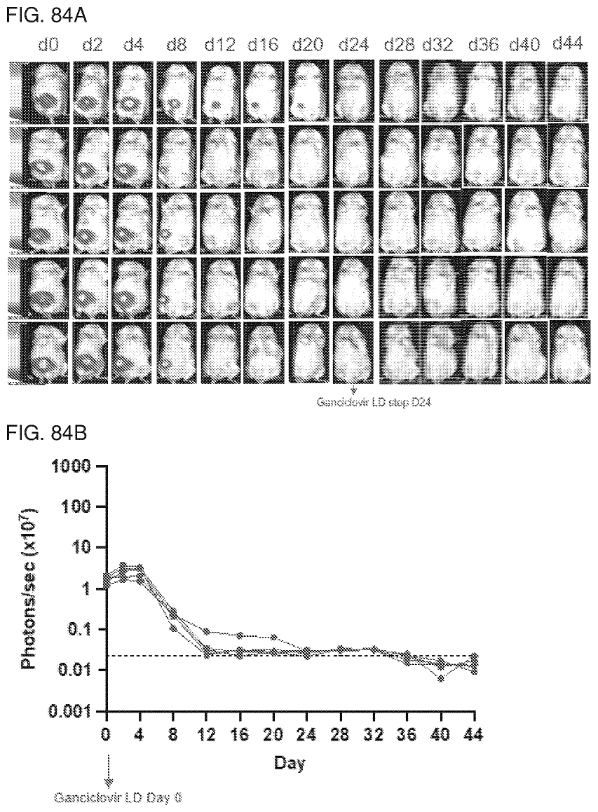

Human HIP iPSCs (HSVTk clone 1-B10) were injected subcutaneously into Group 1 NSG mice. Ganciclovir LD (50 mg/kg) treatment was performed daily intraperitoneally. As shown in FIG. 84A-B, dosing with Ganciclovir LD resulted in killing of HIP-HsVtk iPSCs within 12-24 days. Mice were checked during a 44D follow up. Group 1 mice treatment was as follows: 1×10⁶ hiPSCs HIP HSVtk clone 1-B10 luc+ in diluted MG, S.C.; treatment was 50 mg/kg/day Ganciclovir, IP (DO).

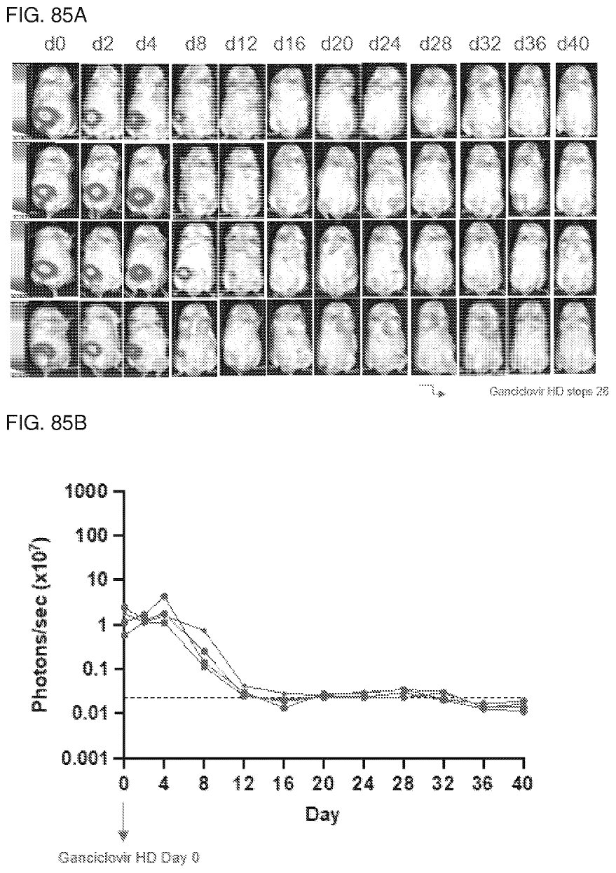

Human HIP iPSCs (HSVTk clone 1-B10) were injected subcutaneously into Group 2 NSG mice. Ganciclovir HD (75 mg/kg) treatment was administered daily intraperitoneally. As shown in FIG. 85A-B, dosing with Ganciclovir HD resulted in killing of HIP-HsVtk iPSCs within 12-16 days. Mice were checked during a 40 d follow up. HD appeared to be slightly beneficial. Group 2 mice treatment was as follows: 1×10⁶ hiPSCs HIP HSVtk clone 1-B10 luc+ in diluted MG, S.C.; treatment was 75 mg/kg/day Ganciclovir, IP (DO).

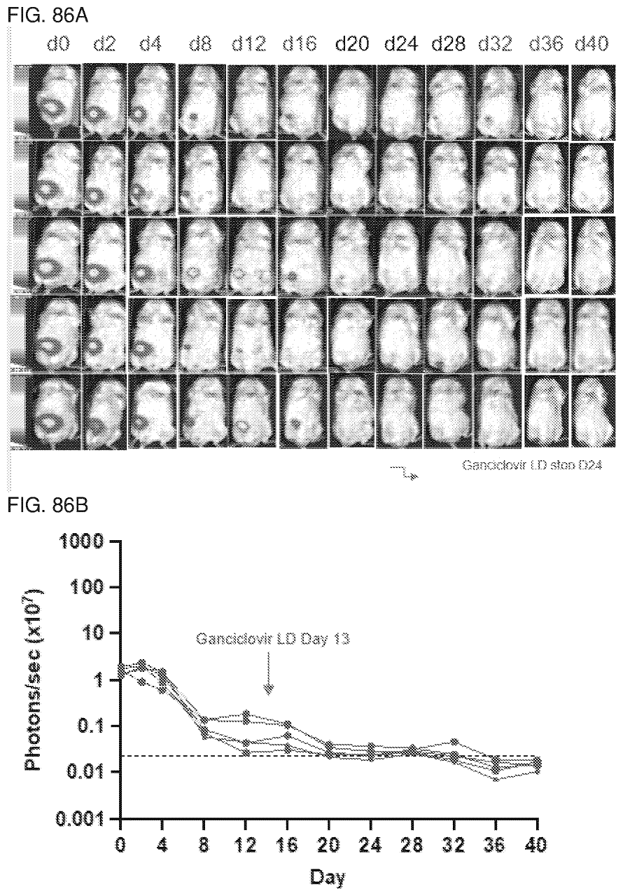

Human HIP iPSCs (HSVTk clone 1-B10) were injected subcutaneously into Group 3 NSG mice. Ganciclovir LD (50 mg/kg) treatment was administered daily intraperitoneally, starting on Day 13. As shown in FIG. 86A-B, dosing with Ganciclovir LD resulted in killing of HIP-HsVtk iPSCs within 7 days after start of treatment. Mice were checked during a 40 d follow up. Group 3 mice treatment was as follows: 1×10⁶ hiPSCs HIP HSVtk clone 1-B10 luc+ in diluted MG, S.C.; treatment was 50 mg/kg/day Ganciclovir, IP (D13).

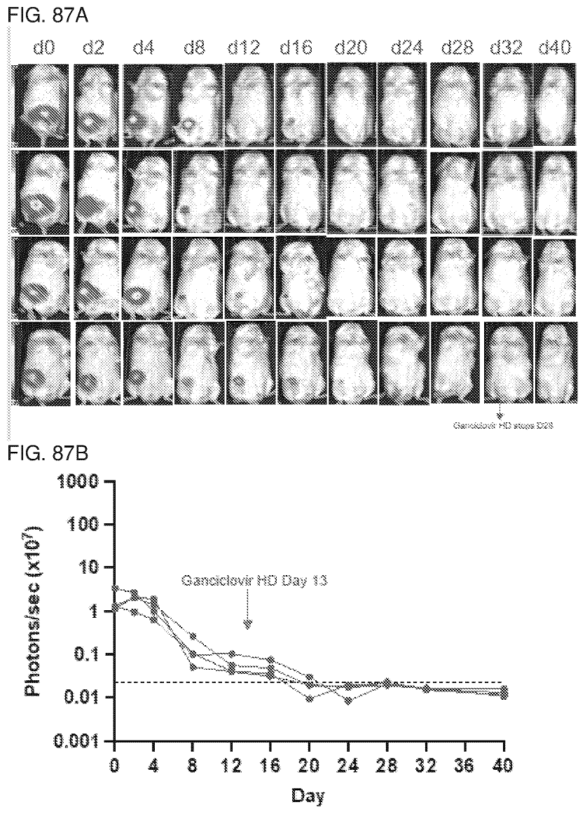

Human HIP iPSCs (HSVTk clone 1-B10) were injected subcutaneously into Group 4 NSG mice. Ganciclovir HD (75 mg/kg) treatment was administered daily intraperitoneally starting on Day 13. As shown in FIG. 87A-B, dosing with Ganciclovir HD resulted in killing of HIP-HsVtk iPSCs within 7 days after start of treatment. Mice were checked during a 40 d follow up. No obvious benefit of HD was observed compared to LD. Group 4 mice treatment was as follows: 1×10⁶ hiPSCs HIP HSVtk clone 1-B10 luc+ in diluted MG, S.C.; treatment was 75 mg/kg/day Ganciclovir, IP (D13).

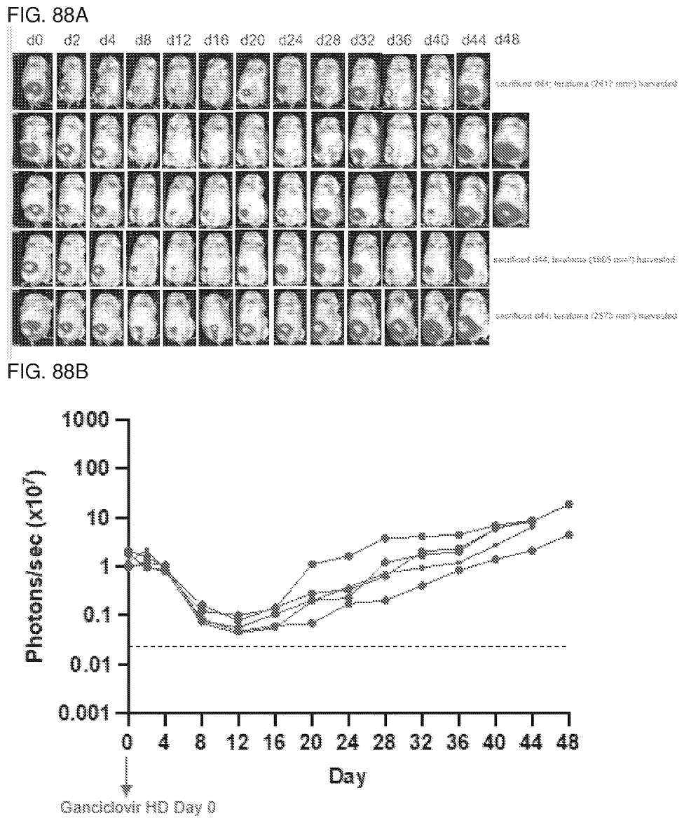

Human HIP iPSCs (clone 15; no kill switch) were injected subcutaneously into Group 6 NSG mice. Ganciclovir HD (75 mg/kg) treatment was administered daily intraperitoneally starting Day 0. As shown in FIG. 88A-B, Ganciclovir HD treatment appeared to have no impact on HIP iPSCs without a kill switch. Group 6 mice treatment was as follows: 1×10⁶ hiPSCs HIP clone 15 luc+ in diluted MG, S.C.; treatment was 75 mg/kg/day Ganciclovir, IP (DO).

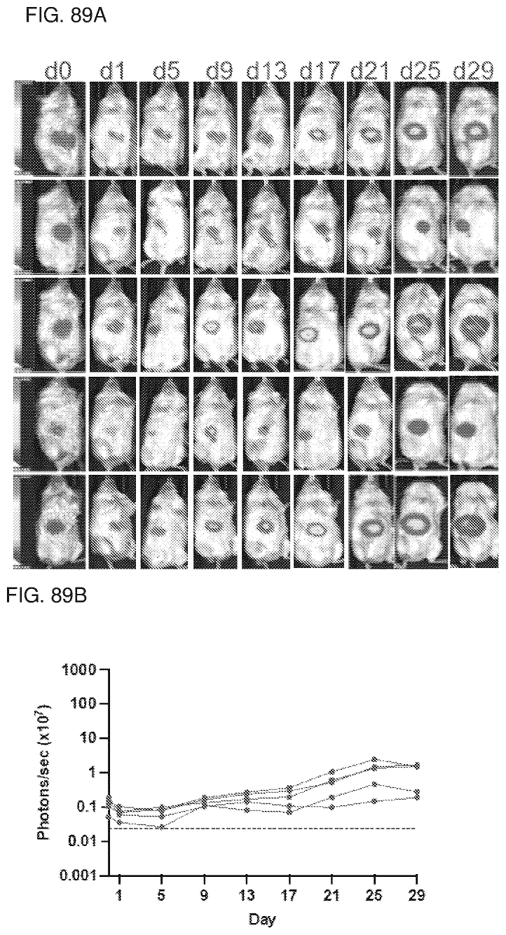

1×10⁶ human HIP iPSCs luc+ (HSVtk clone 1-B10) were injected subcutaneously into NSG mice. No treatment was administered (pilot study). As shown in FIG. 89A-B, for the control group, HSVtk edit did not appear to impact iPSC survival in NSG mice.

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known components and functions have not been shown or described in detail to avoid unnecessarily obscuring the description of the embodiments of the technology. Where the context permits, singular or plural terms may also include the plural or singular term, respectively. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A method comprising administering to a subject in need thereof a CD47-SIRPα blockade agent, wherein the subject was previously administered a population of cells engineered to express an exogenous CD47 polypeptide.

2. A method comprising administering to a subject in need thereof a CD47-SIRPα blockade agent, wherein the subject was previously administered a population of T cells engineered to express an exogenous CD47 polypeptide.

3. A method comprising administering to a subject in need thereof a CD47-SIRPα blockade agent, wherein the subject was previously administered a population of T cells (i) engineered to express an exogenous CD47 polypeptide and at least one chimeric antigen receptor (CAR) and (ii) having reduced expression of MHC class I HLA molecules, MHC class II HLA molecules, T cell receptor (TCR) alpha, and/or TCR beta.

4. A method comprising administering to a subject in need thereof a CD47-SIRPα blockade agent, wherein the subject was previously administered a population of T cells having reduced expression of MHC class I HLA molecules, MHC class II HLA molecules, and TCR alpha and engineered to express an exogenous CD47 polypeptide and a CD19 chimeric antigen receptor (CAR).

5. A method comprising administering to a subject in need thereof a CD47-SIRPα blockade agent, wherein the subject was previously administered a population of pancreatic islet cells engineered to express an exogenous CD47 polypeptide.

6. A method comprising administering to a subject in need thereof a CD47-SIRPα blockade agent, wherein the subject was previously administered a population of pancreatic islet cells (i) engineered to express an exogenous CD47 polypeptide and (ii) having reduced expression of MHC class I HLA and/or MHC class II HLA molecules.

7. A method comprising administering to a subject in need thereof a CD47-SIRPα blockade agent, wherein the subject was previously administered a population of pancreatic islet cells (i) engineered to express exogenous CD47, CD46, and CD59 polypeptides and (ii) having reduced expression of MHC class I HLA and/or MHC class II HLA molecules.

8. A method of reducing a population of cells engineered to express an exogenous CD47 polypeptide in a subject comprising:
(a) administering to the subject a first dose of a CD47-SIRPα blockade agent;
(b) determining a first outcome of the first dose of the CD47-SIRPα blockade agent administered in (a);
(c) optionally administering a second dose of the CD47-SIRPα blockade agent based on the first outcome in (b); and
(d) optionally determining a second outcome of the second dose of the CD47-SIRPα blockade agent administered in (c).

9. A method comprising:
(a) quantifying a population of cells engineered to express an exogenous CD47 polypeptide in a subject;
(b) determining a first dose of a CD47-SIRPα blockade agent that is effective in reducing the population of cells by at least 20%; and
(c) administering the first dose of the CD47-SIRPα blockade agent to the subject.

10. The method of any of claim 2, 3, or 4, wherein the T cells are primary cells.

11. The method of any of claim 2, 3, or 4, wherein the T cells are allogeneic.

12. The method of any of claim 2, 3, or 4, wherein the T cells are differentiated from iPSCs.

13. The method of claim 2, wherein the T cells are further engineered to express a chimeric antigen receptor (CAR).

14. The method of any of claim 3, 4, or 13, wherein the CAR is a CD19 CAR selected from the group consisting of tisagenlecleucel, lisocabtagene maraleucel, axicabtagene ciloleucel, and brexucabtagene autoleucel.

15. The method of any of claim 3, 4, or 13, wherein the CAR is a CD19 CAR comprising the amino acid sequence of SEQ ID NO:117.

16. The method of claim 15, wherein the CD19 CAR is encoded by the nucleic acid sequence of SEQ ID NO:116.

17. The method of any of claim 2, 3, or 4, wherein the T cells are engineered to express at least one additional factor selected from the group consisting of CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8, and a combination thereof.

18. The method of any of claim 5, 6, or 7, wherein the pancreatic islet cells are engineered to express at least one additional factor selected from the group consisting of CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8, and a combination thereof.

19. The method of any of claim 5, 6, or 7, wherein the pancreatic islet cells are engineered to have reduced expression of CD142.

20. The method of any of claim 5, 6, or 7 wherein the pancreatic islet cells are primary cells.

21. The method of any of claim 5, 6, or 7 wherein the pancreatic islet cells are differentiated from iPSCs.

22. The method of any of claim 3, 4, or 13, wherein the CAR and a gene encoding the exogenous CD47 polypeptide were introduced into the T cells in a bicistronic vector.

23. The method of claim 22, wherein the bicistronic vector was introduced into the T cells via a lentivirus.

24. The method of claim 23, wherein the CAR and the gene encoding the exogenous CD47 polypeptide are under the control of a single promoter.

25. The method of claim 8, wherein the first outcome and second outcome are independently selected from the group consisting of: (i) a reduction in the number of cells by between about 10% and 100%, (ii) a reduction in an adverse event by between about 10% and 100%, and (iii) a combination of (i) and (ii).

26. The method of claim 8 or 9, wherein the first dose and/or the second dose is administered:
(i) at 0.05, 0.1, 0.3, 1, 3, or 10 mg/kg;
(ii) once every 12 hours, once every 24 hours, once every 36 hours, or once every 48 hours; and/or
(iii) for between 1 day and 3 weeks.

27. The method of claim 26, wherein the first dose and the second dose are the same.

28. The method of any of claim 1, 8, or 9, wherein the cells are primary cells.

29. The method of claim 28, wherein the primary cells are T cells or pancreatic islet cells.

30. The method of any of claim 1, 8, or 9, wherein the cells are differentiated from iPSCs.

31. The method of any of claim 12, 21, or 30, wherein the differentiated cells are selected from the group consisting of cardiac cells, neural cells, endothelial cells, T cells, pancreatic islet cells, retinal pigmented epithelium cells, hepatocytes, thyroid cells, skin cells, blood cells, primary cells, and epithelial cells.

32. The method of any of claim 1, 8, or 9, wherein the cells are engineered to express at least one additional factor selected from the group consisting of CD16, CD24, CD35, CD39, CD46, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-1, PD-L1, Serpinb9, CCI21, Mfge8, and a combination thereof.

33. The method of any of claim 2, 3, 4, or 29, wherein the T cells are engineered to have reduced expression of TCRα and/or TCRβ.

34. The method of any of claim 2, 3, 4, or 29, wherein the T cells are engineered to have reduced expression of cytotoxic T-lymphocyte-associated protein 4 (CTLA4) and/or programmed cell death (PD1).

35. The method of any of claims 1-9, wherein a gene encoding the exogenous CD47 polypeptide was introduced into the cell via homology directed repair (HDR)-mediated insertion into a genomic locus of the cell.

36. The method of claim 35, wherein the genomic locus is selected from the group consisting of a B2M locus, a CIITA locus, a TRAC locus, a TRBC locus, and a safe harbor locus.

37. The method of claim 36, wherein the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus.

38. The method of any of claim 3, 4, or 13, wherein the CAR binds an antigen selected from the group consisting of CD19, CD20, CD22, CD38, CD123, CD138, BCMA, and a combination thereof.

39. The method of claim 8, wherein the first outcome and/or second outcome is an adverse event.

40. The method of any of claims 1-7, wherein the CD47-SIRPα blockade agent is administered at least one day after the subject was administered the cells.

41. The method of any of claims 1-7, wherein the CD47-SIRPα blockade agent is administered at least one week after the subject was administered the cells.

42. The method of any of claims 1-7, wherein the CD47-SIRPα blockade agent is administered at least one month after the subject was administered the cells.

43. The method of any of claims 1-7, wherein the CD47-SIRPα blockade agent is administered after the subject experiences an adverse event related to the administered cells.

44. The method of claim 39 or 43, wherein the adverse event is selected from the group consisting of hyperproliferation, transformation, tumor formation, cytokine release syndrome, graft-versus-host disease (GVHD), immune effector cell-associated neurotoxicity syndrome (ICANS), inflammation, infection, nausea, vomiting, bleeding, interstitial pneumonitis, respiratory disease, jaundice, weight loss, diarrhea, loss of appetite, cramps, abdominal pain, hepatic veno-occlusive disease (VOD), graft failure, organ damage, infertility, hormonal changes, abnormal growth formation, cataracts, and post-transplant lymphoproliferative disorder (PTLD).

45. The method of any of claims 1-9, wherein the CD47-SIRPα blockade agent comprises a CD47-binding domain.

46. The method of claim 45, wherein the CD47-binding domain comprises signal regulatory protein alpha (SIRPα) or a fragment thereof.

47. The method of any of claims 1-9, wherein the CD47-SIRPα blockade agent comprises an immunoglobulin G (IgG) Fc domain.

48. The method of claim 47, wherein the IgG Fc domain comprises an IgG1 Fc domain.

49. The method of claim 48, wherein the IgG1 Fc domain comprises a fragment of a human antibody.

50. The method of any of claim 1-9, wherein the CD47-SIRPα blockade agent is selected from the group consisting of TTI-621, TTI-622, and ALX148.

51. The method of claim 47, wherein the IgG Fc domain comprises an IgG4 Fc domain.

52. The method of any one of claims 1-9, wherein the CD47-SIRPα blockade agent is an antibody.

53. The method of claim 52, wherein the antibody is selected from the group consisting of MIAP410, B6H12, and Magrolimab.

54. The method of any one of claims 1-7, wherein the CD47-SIRPα blockade agent is administered at a dose effective to reduce the population of cells.

55. The method of claim 54, wherein the population of cells is reduced by between about 10% and 100%.

56. The method of claim 54, wherein the population of cells is eliminated.

57. The method of claim 54, wherein the reduction of the population of cells occurs via an immune response.

58. The method of claim 57, wherein the immune response is NK cell-mediated cell killing, macrophage-mediated cell killing, complement-dependent cytotoxicity (CDC), and/or antibody-dependent cellular cytotoxicity (ADCC) of the cells.

59. The method of any of claims 1-9, wherein the CD47-SIRPα blockade agent is administered to the subject intravenously, subcutaneously, intraperitonially, intramuscularly, or intracranially.

60. The method of claim 59, wherein the CD47-SIRPα blockade agent is administered to the subject at a time interval of between 1-20 days for a period of between 10 days and 6 months.

61. The method of claim 60, wherein the CD47-SIRPα blockade agent is administered to the subject:
(i) at a dose of 0.05, 0.1, 0.3, 1, 3, or 10 mg/kg;
(ii) once every 12 hours, once every 24 hours, once every 36 hours, or once every 48 hours; and/or
(iii) for between 1 day and 3 weeks.

62. The method of any of claims 1-9, further comprising administering IL-2 to the subject.

63. The method of any of claims 1-9, wherein the CD47-SIRPα blockade agent is selected from the group consisting of an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that binds SIRPα, an SIRPα containing fusion protein, and a combination thereof.

64. The method of claim 63, wherein the antibody or fragment thereof that binds CD47 is selected from the group consisting of magrolimab (Hu5F9-G4), CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

65. The method of claim 63, wherein the antibody or fragment thereof that binds CD47 is selected from the group consisting of a single-chain Fv fragment (scFv) against CD47, a Fab against CD47, a VHH nanobody against CD47, a DARPin against CD47, and variants thereof.

66. The method of claim 63, wherein the antibody or fragment thereof that binds SIRPα is selected from the group consisting of ADU-1805, CC-95251, OSE-172 (BI 765063), KWAR23, and P362.

67. The method of claim 63, wherein the antibody or fragment thereof that binds SIRPα is selected from the group consisting of a single-chain Fv fragment (scFv) against SIRPα, a Fab against SIRPα, a VHH nanobody against SIRPα, a DARPin against SIRPα, and variants thereof.

68. The method of claim 63, wherein the SIRPα-containing fusion protein comprises a CD47 binding domain of SIRPα linked to an Fc domain.

69. The method of claim 68, wherein the Fc domain comprises an Fc domain or portion thereof selected from the group consisting of IgG1, IgG2, IgG3, and IgG4.

70. The method of any of claim 1, 2, 5, 8, or 9, wherein the cells have reduced expression of MHC class I HLA and/or MHC class II HLA molecules.

71. The method of any of claim 3, 4, 6, 7, or 70, wherein MHC class I and/or MHC class II expression is knocked out.

72. The method of any of claim 3, 4, 6, 7, or 70, wherein the reduced expression of MHC class I HLA is mediated by reduced expression of B2M and reduced expression of MHC class II is mediated by reduced expression of CIITA.

73. The method of claim 71, wherein B2M and/or CIITA expression is knocked out.

74. The method of any of claims 1-9, wherein the exogenous CD47 polypeptide comprises the amino acid sequence of SEQ ID NO:2 or SEQ ID NO:4.


SEQUENCE LISTING (134 sequences)

SEQ ID NO: 1 - DNA, 972 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 2 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - DNA, 918 bp, Homo sapiens
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 4 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 5 - DNA, 969 bp, Artificial Sequence
  Codon optimized CD47 CDS
atgtggcccctggtcgccgccctgttgctgggctcggcatgctgcggatcagctcagctactgtttaataaaacaaaatctgtagaattcacgttttgtaacgacactgtcgtgatcccatgctttgttactaatatggaggcacaaaacaccactgaagtgtacgtgaagtggaaattcaaaggcagagacatttacacctttgacggcgccctcaacaagtccaccgtgcccactgactttagtagcgcaaaaattgaggtcagccaattactaaaaggagatgcctctttgaagatggacaagagcgatgctgtcagccacacagggaactacacttgtgaagtaacagagttaacccgcgaaggtgaaacgatcatcgagctgaagtatcgagtggtgtcctggttttctccgaacgagaatatccttatcgtaattttcccaattttcgctatcctcctgttctggggccagtttggtatcaagacactcaaatatcggtccggtgggatggatgagaagacaattgccctgcttgttgctggactcgtgatcaccgtcatcgtgattgttggggccatccttttcgtcccaggggagtacagcctgaagaatgctacgggcctgggattaattgtgacctctacagggatactcatcctgcttcactactatgtgttcagtaccgcgattggactgacctccttcgtcattgccatattggtgattcaggtgatagcctacatcctcgccgtggttggcctgagtctctgtatcgcggcgtgcatacccatgcatggccctcttctgatttcagggttgagtatcctcgcactagcacagttgctgggactggtttatatgaaatttgtggcctccaaccagaagactatacagcctcctaggaaggctgtagaggagcccctgaatgcattcaaggaatcaaaaggcatgatgaatgatgaa

SEQ ID NO: 6 - protein, 21 aa, Artificial Sequence
  CD8a signal peptide
MALPVTALLLPLALLLHAARP

SEQ ID NO: 7 - protein, 20 aa, Artificial Sequence
  IgK signal peptide
METDTLLLWVLLLWVPGSTG

SEQ ID NO: 8 - protein, 22 aa, Artificial Sequence
  GMCSFR-Alpha (CSF2RA) signal peptide
MLLLVTSLLLCELPHPAFLLIP

SEQ ID NO: 9 - protein, 45 aa, Artificial Sequence
  CD8a hinge domain
TTTPAPRPPTPAPTIASQPLSLRPEACRPAAGGAVHTRGLDFACD

SEQ ID NO: 10 - protein, 39 aa, Artificial Sequence
  CD28 hinge domain
IEVMYPPPYLDNEKSNGTIIHVKGKHLCPSPLFPGPSKP

SEQ ID NO: 11 - protein, 12 aa, Artificial Sequence
  IgG4 hinge domain
ESKYGPPCPPCP

SEQ ID NO: 12 - protein, 12 aa, Artificial Sequence
  IgG4 hinge domain
ESKYGPPCPSCP

SEQ ID NO: 13 - protein, 229 aa, Artificial Sequence
  IgG4 hinge-CH2-CH3 domain
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 24 aa, Artificial Sequence
  CD8a transmembrane domain
IYIWAPLAGTCGVLLLSLVITLYC

SEQ ID NO: 15 - protein, 27 aa, Artificial Sequence
  CD28 transmembrane domain
FWVLVVVGGVLACYSLLVTVAFIIFWV

SEQ ID NO: 16 - protein, 42 aa, Artificial Sequence
  4-1BB costimulatory domain
KRGRKKLLYIFKQPFMRPVQTTQEEDGCSCRFPEEEEGGCEL

SEQ ID NO: 17 - protein, 41 aa, Artificial Sequence
  CD28 costimulatory domain
RSKRSRLLHSDYMNMTPRRPGPTRKHYQPYAPPRDFAAYRS

SEQ ID NO: 18 - protein, 112 aa, Artificial Sequence
  CD3z signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 245 aa, Artificial Sequence
  Anti-CD19 FMC63 scFv entire sequence, with Whitlow linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 20 - protein, 107 aa, Artificial Sequence
  Anti-CD19 FMC63 scFv light chain variable region
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 21 - protein, 6 aa, Artificial Sequence
  Anti-CD19 FMC63 scFv light chain CDR1
QDISKY

SEQ ID NO: 22 - protein, 3 aa, Artificial Sequence
  Anti-CD19 FMC63 scFv light chain CDR2
HTS

SEQ ID NO: 23 - protein, 9 aa, Artificial Sequence
  Anti-CD19 FMC63 scFv light chain CDR3
QQGNTLPYT

SEQ ID NO: 24 - protein, 18 aa, Artificial Sequence
  Whitlow linker
GSTSGSGKPGSGEGSTKG

SEQ ID NO: 25 - protein, 120 aa, Artificial Sequence
  Anti-CD19 FMC63 scFv heavy chain variable region
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 26 - protein, 8 aa, Artificial Sequence
  Anti-CD19 FMC63 scFv heavy chain CDR1
GVSLPDYG

SEQ ID NO: 27 - protein, 7 aa, Artificial Sequence
  Anti-CD19 FMC63 scFv heavy chain CDR2
IWGSETT

SEQ ID NO: 28 - protein, 14 aa, Artificial Sequence
  Anti-CD19 FMC63 scFv heavy chain CDR3
AKHYYYGGSYAMDY

SEQ ID NO: 29 - protein, 242 aa, Artificial Sequence
  Anti-CD19 FMC63 scFv entire sequence, with 3xG4S linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 30 - protein, 15 aa, Artificial Sequence
  3xG4S linker
GGGGSGGGGSGGGGS

SEQ ID NO: 31 - DNA, 1458 bp, Artificial Sequence
  Tisagenlecleucel CD19 CAR
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - protein, 486 aa, Artificial Sequence
  Tisagenlecleucel CD19 CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1383 bp, Artificial Sequence
  Lisocabtagene maraleucel CD19 CAR
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 34 - protein, 461 aa, Artificial Sequence
  Lisocabtagene maraleucel CD19 CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 1467 bp, Artificial Sequence
  Axicabtagene ciloleucel CD19 CAR
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 36 - protein, 489 aa, Artificial Sequence
  Axicabtagene ciloleucel CD19 CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 246 aa, Artificial Sequence
  Anti-CD20 Leu16 scFv entire sequence, with Whitlow linker
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 38 - protein, 106 aa, Artificial Sequence
  Anti-CD20 Leu16 scFv light chain variable region
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 39 - protein, 10 aa, Artificial Sequence
  Anti-CD20 Leu16 scFv light chain CDR1
RASSSVNYMD

SEQ ID NO: 40 - protein, 7 aa, Artificial Sequence
  Anti-CD20 Leu16 scFv light chain CDR2
ATSNLAS

SEQ ID NO: 41 - protein, 9 aa, Artificial Sequence
  Anti-CD20 Leu16 scFv light chain CDR3
QQWSFNPPT

SEQ ID NO: 42 - protein, 122 aa, Artificial Sequence
  Anti-CD20 Leu16 scFv heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 43 - protein, 5 aa, Artificial Sequence
  Anti-CD20 Leu16 scFv heavy chain CDR1
SYNMH

SEQ ID NO: 44 - protein, 17 aa, Artificial Sequence
  Anti-CD20 Leu16 scFv heavy chain CDR2
AIYPGNGDTSYNQKFKG

SEQ ID NO: 45 - protein, 246 aa, Artificial Sequence
  Anti-CD22 m971 scFv entire sequence, with 3xG4S linker
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 46 - protein, 124 aa, Artificial Sequence
  Anti-CD22 m971 scFv heavy chain variable region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 47 - protein, 10 aa, Artificial Sequence
  Anti-CD22 m971 scFv heavy chain CDR1
GDSVSSNSAA

SEQ ID NO: 48 - protein, 9 aa, Artificial Sequence
  Anti-CD22 m971 scFv heavy chain CDR2
TYYRSKWYN

SEQ ID NO: 49 - protein, 14 aa, Artificial Sequence
  Anti-CD22 m971 scFv heavy chain CDR3
AREVTGDLEDAFDI

SEQ ID NO: 50 - protein, 107 aa, Artificial Sequence
  Anti-CD22 m971 scFv light chain
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 51 - protein, 6 aa, Artificial Sequence
  Anti-CD22 m971 scFv light chain CDR1
QTIWSY

SEQ ID NO: 52 - protein, 3 aa, Artificial Sequence
  Anti-CD22 m971 scFv light chain CDR2
AAS

SEQ ID NO: 53 - protein, 9 aa, Artificial Sequence
  Anti-CD22 m971 scFv light chain CDR3
QQSYSIPQT

SEQ ID NO: 54 - protein, 246 aa, Artificial Sequence
  Anti-CD22 m971-L7 scFv entire sequence, with 3xG4S linker
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 55 - protein, 124 aa, Artificial Sequence
  Anti-CD22 m971-L7 scFv heavy chain variable region
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 56 - protein, 10 aa, Artificial Sequence
  Anti-CD22 m971-L7 scFv heavy chain CDR1
GDSVSSNSVA

SEQ ID NO: 57 - protein, 9 aa, Artificial Sequence
  Anti-CD22 m971-L7 scFv heavy chain CDR2
TYYRSTWYN

SEQ ID NO: 58 - protein, 14 aa, Artificial Sequence
  Anti-CD22 m971-L7 scFv heavy chain CDR3
AREVTGDLEDAFDI

SEQ ID NO: 59 - protein, 107 aa, Artificial Sequence
  Anti-CD22 m971-L7 scFv light chain variable region
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 60 - protein, 6 aa, Artificial Sequence
  Anti-CD22 m971-L7 scFv light chain CDR1
QTIWSY

SEQ ID NO: 61 - protein, 3 aa, Artificial Sequence
  Anti-CD22 m971-L7 scFv light chain CDR2
AAS

SEQ ID NO: 62 - protein, 9 aa, Artificial Sequence
  Anti-CD22 m971-L7 scFv light chain CDR3
QQSYSIPQT

SEQ ID NO: 63 - protein, 246 aa, Artificial Sequence
  Anti-BCMA C11D5.3 scFv entire sequence, with Whitlow linker
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 64 - protein, 111 aa, Artificial Sequence
  Anti-BCMA C11D5.3 scFv light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 65 - protein, 15 aa, Artificial Sequence
  Anti-BCMA C11D5.3 scFv light chain CDR1
RASESVSVIGAHLIH

SEQ ID NO: 66 - protein, 7 aa, Artificial Sequence
  Anti-BCMA C11D5.3 scFv light chain CDR2
LASNLET

SEQ ID NO: 67 - protein, 9 aa, Artificial Sequence
  Anti-BCMA C11D5.3 scFv light chain CDR3
LQSRIFPRT

SEQ ID NO: 68 - protein, 117 aa, Artificial Sequence
  Anti-BCMA C11D5.3 scFv heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 69 - protein, 5 aa, Artificial Sequence
  Anti-BCMA C11D5.3 scFv heavy chain CDR1
DYSIN

SEQ ID NO: 70 - protein, 17 aa, Artificial Sequence
  Anti-BCMA C11D5.3 scFv heavy chain CDR2
WINTETREPAYAYDFRG

SEQ ID NO: 71 - protein, 8 aa, Artificial Sequence
  Anti-BCMA C11D5.3 scFv heavy chain CDR3
DYSYAMDY

SEQ ID NO: 72 - protein, 246 aa, Artificial Sequence
  Anti-BCMA C12A3.2 scFv entire sequence, with Whitlow linker
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 73 - protein, 111 aa, Artificial Sequence
  Anti-BCMA C12A3.2 scFv light chain variable region
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 74 - protein, 15 aa, Artificial Sequence
  Anti-BCMA C12A3.2 scFv light chain CDR1
RASESVTILGSHLIY

SEQ ID NO: 75 - protein, 7 aa, Artificial Sequence
  Anti-BCMA C12A3.2 scFv light chain CDR2
LASNVQT

SEQ ID NO: 76 - protein, 9 aa, Artificial Sequence
  Anti-BCMA C12A3.2 scFv light chain CDR3
LQSRTIPRT

SEQ ID NO: 77 - protein, 117 aa, Artificial Sequence
  Anti-BCMA C12A3.2 scFv heavy chain variable region
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 78 - protein, 5 aa, Artificial Sequence
  Anti-BCMA C12A3.2 scFv heavy chain CDR1
HYSMN

SEQ ID NO: 79 - protein, 17 aa, Artificial Sequence
  Anti-BCMA C12A3.2 scFv heavy chain CDR2
RINTESGVPIYADDFKG

SEQ ID NO: 80 - protein, 8 aa, Artificial Sequence
  Anti-BCMA C12A3.2 scFv heavy chain CDR3
DYLYSLDF

SEQ ID NO: 81 - protein, 121 aa, Artificial Sequence
  Anti-BCMA FHVH33 entire sequence
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 82 - protein, 8 aa, Artificial Sequence
  Anti-BCMA FHVH33 CDR1
GFTFSSYA

SEQ ID NO: 83 - protein, 8 aa, Artificial Sequence
  Anti-BCMA FHVH33 CDR2
ISGSGDYI

SEQ ID NO: 84 - protein, 14 aa, Artificial Sequence
  Anti-BCMA FHVH33 CDR3
AKEGTGANSSLADY

SEQ ID NO: 85 - protein, 21 aa, Artificial Sequence
  T2A peptide
  MISC_FEATURE (1)..(3): GSG linker is optional
GSGEGRGSLLTCGDVEENPGP

SEQ ID NO: 86 - protein, 22 aa, Artificial Sequence
  P2A peptide
  MISC_FEATURE (1)..(3): GSG linker is optional
GSGATNFSLLKQAGDVEENPGP

SEQ ID NO: 87 - protein, 23 aa, Artificial Sequence
  E2A peptide
  MISC_FEATURE (1)..(3): GSG linker is optional
GSGQCTNYALLKLAGDVESNPGP

SEQ ID NO: 88 - protein, 25 aa, Artificial Sequence
  F2A peptide
  MISC_FEATURE (1)..(3): GSG linker is optional
GSGVKQTLNFDLLKLAGDVESNPGP

SEQ ID NO: 89 - protein, 7 aa, Artificial Sequence
  FC1 furin site
  MISC_FEATURE (5)..(7): GSG is optional
RRRRGSG

SEQ ID NO: 90 - protein, 7 aa, Artificial Sequence
  FC2 furin site
  MISC_FEATURE (5)..(7): GSG is optional
RKRRGSG

SEQ ID NO: 91 - protein, 15 aa, Artificial Sequence
  FC3 furin site
  MISC_FEATURE (5)..(7): GSG is optional
  MISC_FEATURE (13)..(15): GSG is optional
RKRRGSGTPDPWGSG

SEQ ID NO: 92 - DNA, 13 bp, Artificial Sequence
  Kozak consensus sequence
  misc_feature (7)..(7): r is a or g
gccgccrccatgg

SEQ ID NO: 93 - DNA, 589 bp, Artificial Sequence
  WPRE sequence
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 94 - RNA, 20 nt, Artificial Sequence
  Exemplary spCas9 1 Complementary region (spacer)
  misc_feature (1)..(20): n at each occurrence is any nucleotide
nnnnnnnnnnnnnnnnnnnn

SEQ ID NO: 95 - RNA, 12 nt, Artificial Sequence
  Exemplary spCas9 1 crRNA repeat region
guuuuagagcua

SEQ ID NO: 96 - RNA, 4 nt, Artificial Sequence
  Exemplary spCas9 1 tetraloop
gaaa

SEQ ID NO: 97 - RNA, 66 nt, Artificial Sequence
  Exemplary spCas9 1 tracrRNA
uagcaaguuaaaauaaggcuaguccguuaucaacuugaaaaaguggcaccgagucggugcuuuuuu

SEQ ID NO: 98 - RNA, 20 nt, Artificial Sequence
  Exemplary spCas9 2 Complementary region (spacer)
  misc_feature (1)..(20): n at each occurrence is any nucleotide
nnnnnnnnnnnnnnnnnnnn

SEQ ID NO: 99 - RNA, 17 nt, Artificial Sequence
  Exemplary spCas9 2 crRNA repeat region
  misc_feature (5)..(5): s is c or g
guuusagagcuaugcug

SEQ ID NO: 100 - RNA, 4 nt, Artificial Sequence
  Exemplary spCas9 2 tetraloop
gaaa

SEQ ID NO: 101 - RNA, 71 nt, Artificial Sequence
  Exemplary spCas9 2 tracrRNA
  misc_feature (15)..(15): s is c or g
cagcauagcaaguusaaauaaggcuaguccguuaucaacuugaaaaaguggcaccgagucggugcuuuuuu

SEQ ID NO: 102 - RNA, 20 nt, Artificial Sequence
  Exemplary saCas9 Complementary region (spacer)
  misc_feature (1)..(20): n at each occurrence is any nucleotide
nnnnnnnnnnnnnnnnnnnn

SEQ ID NO: 103 - RNA, 14 nt, Artificial Sequence
  Exemplary saCas9 crRNA repeat region
guuuuaguacucug

SEQ ID NO: 104 - RNA, 4 nt, Artificial Sequence
  Exemplary saCas9 tetraloop
gaaa

SEQ ID NO: 105 - RNA, 64 nt, Artificial Sequence
  Exemplary saCas9 tracrRNA
cagaaucuacuaaaacaaggcaaaaugccguguuuaucucgucaacuuguuggcgagauuuuuu

SEQ ID NO: 106 - RNA, 88 nt, Artificial Sequence
  Exemplary AkCas12b tracrRNA
gucgucuauaggacggcgaggacaacgggaagugccaaugugcucuuuccaagagcaaacaccccguuggcuucaagaugaccgcucg

SEQ ID NO: 107 - RNA, 4 nt, Artificial Sequence
  Exemplary AkCas12b tetraloop
aaaa

SEQ ID NO: 108 - RNA, 23 nt, Artificial Sequence
  Exemplary AkCas12b crRNA repeat region
cgagcggucugagaaguggcacu

SEQ ID NO: 109 - RNA, 20 nt, Artificial Sequence
  Exemplary AkCas12b Complementary region (spacer)
  misc_feature (1)..(20): n at each occurrence is any nucleotide
nnnnnnnnnnnnnnnnnnnn

SEQ ID NO: 110 - DNA, 20 bp, Artificial Sequence
  GET000046 guide
accccacagtggggccacta

SEQ ID NO: 111 - DNA, 20 bp, Artificial Sequence
  GET000047 guide
tgttggaaggatgaggaaat

SEQ ID NO: 112 - DNA, 20 bp, Artificial Sequence
  GET000048 guide
tcactatgctgccgcccagt

SEQ ID NO: 113 - protein, 42 aa, Artificial Sequence
  CD28 hinge domain
AAAIEVMYPPPYLDNEKSNGTIIHVKGKHLCPSPLFPGPSKP

SEQ ID NO: 114 - protein, 28 aa, Artificial Sequence
  CD28 transmembrane domain
MFWVLVVVGGVLACYSLLVTVAFIIFWV

SEQ ID NO: 115 - protein, 112 aa, Artificial Sequence
  CD3z signaling domain with Q to K mutation at position 14
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - DNA, 1467 bp, Artificial Sequence
  Exemplary CD19 CAR
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 117 - protein, 489 aa, Artificial Sequence
  Exemplary CD19 CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 243 aa, Artificial Sequence
  Anti-BCMA CT103A scFv entire sequence, with Whitlow linker
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 119 - protein, 106 aa, Artificial Sequence
  Anti-BCMA CT103A scFv light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 120 - protein, 6 aa, Artificial Sequence
  Anti-BCMA CT103A scFv light chain CDR1
QSISSY

SEQ ID NO: 121 - protein, 3 aa, Artificial Sequence
  Anti-BCMA CT103A scFv light chain CDR2
AAS

SEQ ID NO: 122 - protein, 8 aa, Artificial Sequence
  Anti-BCMA CT103A scFv light chain CDR3
QQKYDLLT

SEQ ID NO: 123 - protein, 119 aa, Artificial Sequence
  Anti-BCMA CT103A scFv heavy chain variable region
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 124 - protein, 10 aa, Artificial Sequence
  Anti-BCMA CT103A scFv heavy chain CDR1
GGSISSSSYY

SEQ ID NO: 125 - protein, 7 aa, Artificial Sequence
  Anti-BCMA CT103A scFv heavy chain CDR2
ISYSGST

SEQ ID NO: 126 - protein, 11 aa, Artificial Sequence
  Anti-BCMA CT103A scFv heavy chain CDR3
ARDRGDTILDV

SEQ ID NO: 127 - DNA, 1503 bp, Artificial Sequence
  Exemplary BCMA CAR
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 128 - protein, 501 aa, Artificial Sequence
  Exemplary BCMA CAR
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 507 aa, Homo sapiens
  MISC_FEATURE (32)..(137): N-terminal V domain
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 130 - protein, 330 aa, Homo sapiens [sequence omitted]

SEQ ID NO: 131 - protein, 327 aa, Homo sapiens
  MISC_FEATURE (99)..(327): Fc domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 132 - protein, 327 aa, Homo sapiens
  MISC_FEATURE (99)..(327): Fc domain
ASFKGPSVFPLVPCSRSTSESTAALGCLVKDYFPEPVTVSWNSCALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVRVLTVLHQDWLNGKEYKCKVSNKGLPSSIEKTISKAKGQPREPQVYTLPPSQEEMTKNQVSLTCLVKGFYPSDIAVEWESNGQPEDNYKTTPPVLDSDGSFFLYSRLTVDKSRWQEGNVFSCSVMHEALHNHYTQKSLSLSPGK

SEQ ID NO: 133 - protein, 345 aa, Artificial Sequence
  TTI-621
  MISC_FEATURE (1)..(118): SIRPa component
  MISC_FEATURE (119)..(345): IgG1 Fc component
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 347 aa, Artificial Sequence
  TTI-622
  MISC_FEATURE (1)..(118): SIRPa component
  MISC_FEATURE (119)..(347): IgG4 Fc component
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK